(19) United States
(12) Patent Application Publication
(10) Pub. No.: US 2025/0236880 A1
(43) Pub. Date: Jul. 24, 2025

(54) METHODS AND MEANS OF INCREASING THE WATER USE EFFICIENCY OF PLANTS

(71) Applicant: Performance Plants Inc., Kingston (CA)

(72) Inventors: Jiangxin WAN, Bath (CA); Yafan HUANG, Bath (CA); Shujun YANG, Kingston (CA); Monika KUZMA, Battersea (CA)

(21) Appl. No.: 18/885,237

(22) Filed: Sep. 13, 2024

Related U.S. Application Data
(62) Division of application No. 16/678,306, filed on Nov. 8, 2019, now Pat. No. 12,123,008.
Application No. 16/678,306 is a continuation of application No. 16/019,077, filed on Jun. 26, 2018, now Pat. No. 10,508,283.
Application No. 16/019,077 is a continuation of application No. 15/266,276, filed on Sep. 15, 2016, now Pat. No. 10,036,035.
Application No. 15/266,276 is a continuation of application No. 12/483,660, filed on Jun. 12, 2009, now Pat. No. 9,453,238.
(60) Provisional application No. 61/132,067, filed on Jun. 13, 2008.

(51) Int. Cl. C12N 15/82; C12N 9/12

(52) U.S. Cl. CPC C12N 15/8273; C12N 9/1205; C12N 15/8218; C12N 15/8227; C12N 15/8261; C12N 15/8271; Y02A 40/146

(57) ABSTRACT

The invention relates to methods of producing a desired phenotype in a plant by manipulation of gene expression within the plant. The method relates to means which inhibit the level of PK220 gene expression or activity, wherein a desired phenotype such as increased water use efficiency relative to a wild type control plant. The invention also relates to nucleic acid sequences and constructs useful such methods and methods of generating and isolating plants having decreased PK220 expression or activity.

### REFERENCE TO RELATED APPLICATIONS

This application is a divisional application of U.S. patent application Ser. No. 16/678,306, filed on Nov. 8, 2019, which is a continuation of U.S. patent application Ser. No. 16/019,077, filed on Jun. 26, 2018, now U.S. Pat. No. 10,508,283, which is a continuation of U.S. patent application Ser. No. 15/266,276, filed on Sep. 15, 2016, now U.S. Pat. No. 10,036,035, which is a continuation of U.S. patent application Ser. No. 12/483,660, filed on Jun. 12, 2009, now U.S. Pat. No. 9,453,238, which claims the benefit of U.S. Ser. No. 61/132,067, filed Jun. 13, 2008, the contents of each of which are incorporated herein by reference in their entirety.

### REFERENCE TO AN ELECTRONIC SEQUENCE LISTING

The contents of the electronic sequence listing (PREP_017_D03US_SeqList_ST26.xml; Size: 285,992 bytes; and Date of Creation: Sep. 13, 2024) are herein incorporated by reference in its entirety.

### FIELD OF THE INVENTION

The invention is in the field of plant molecular biology and relates to transgenic plants having novel phenotypes, methods of producing such plants and polynucleotides and polypeptides useful in such methods. More specifically, the invention relates to inhibition of a protein kinase and transgenic plants having inhibited protein kinase activity.

### BACKGROUND OF THE INVENTION

Water is essential for plant survival, growth and reproduction. Assimilation of carbon dioxide by photosynthesis is directly linked to water loss through the stomata. Crop productivity which is closely linked to biomass production is dependent on plant water use efficiency (WUE) especially in water limited conditions (Passioura 1994 and Sinclair 1994, in Physiology and Determination of Crop Yield). Water use efficiency over a period of plant's growth can be calculated as the ratio of biomass produced per unit of water transpired (Sinclair 1994). Instantaneous measurements of water use efficiency can also be obtained as the ratio of carbon dioxide assimilation to transpiration using gas exchange measurements (Farquhar and Sharkey 1994, in Physiology and Determination of Crop Yield). Since there is a close correlation between crop productivity and water use efficiency, many attempts have been made to study and understand this relationship and the genetic components involved. To maximize the productivity and yield of a crop, efforts have been made to try to improve the water use efficiency of plants (Condon et al., 2002, Araus et al., 2002, Davies et al., 2002). Higher water use efficiency can be achieved either by increasing the biomass production and carbon dioxide assimilation or by reducing the transpiration water loss. Reduced transpiration, especially under non-limiting water conditions can be associated with reduced growth rate and therefore reduced crop productivity. This poses a dilemma on how to improve crop productivity and yield under water limited conditions but also maintain it under irrigated or non-limited water conditions (Condon et al., 2002).

Improvements to water use efficiency, to date, have used plant breeding methods whereby high water use efficiency varieties were crossed with the more productive but lower water use efficiency varieties in hope of improvements in crop yield under water limited conditions (Condon et al., 2002, Araus et al., 2002). Quantitative trait loci (QTL) approaches to identifying the components of water use efficiency have been the most common methods historically used (Mian et al., 1996, Martin et al., 1989, Thumma et al., 2001, Price et al., 2002), and more recently attempts have been made to engineer improved plants by molecular genetic means.

The first gene associated with water use efficiency was ERECTA. The ERECTA gene was first identified as a gene functioning in inflorescence development and organ morphogenesis (Torii et al., 1996). It was later found by QTL mapping to be a major contributor to transpiration efficiency, defined as water transpired per carbon dioxide assimilated, an opposite indicator to water use efficiency in Arabidopsis (Masle et al., 2005). ERECTA encodes a putative leucin-rich repeat receptor-like kinase (LRR-RLK). The regulatory mechanism of LRR-RLK is yet to be understood although it was suggested due to, at least in part, the effects on stomatal density, epidermal cell expansion, mesophyll cell proliferation and cell-cell contact. The normal transpiration efficiency was restored upon complementation using wild type ERECTA in mutant eracta. However, it is not known whether overexpression of ERECTA in transgenic Arabidopsis will result in reduced transpiration efficiency or enhanced water use efficiency. It is the only report showing a plant receptor-like kinase to be involved in transpiration efficiency or water use efficiency.

Another Arabidopsis gene implicated in water use efficiency is the HARDY gene, found through the phenotypic screening of an activation tagged mutant collection (Karaba et al., 2007). Overexpression of HARDY in rice resulted in improved water use efficiency by enhancing photosynthetic assimilation and reducing transpiration. The transgenic rice with increased expression of HARDY exhibited increased shoot biomass under optimal water conditions and increased root biomass under water limited conditions. Overexpression of HARDY in Arabidopsis resulted in thicker leaves with more mesophyll cells and in rice increased leaf biomass and bundle sheet cells. These modifications contributed to enhanced photosynthetic activity and efficiency (Karaba et al., 2007).

Protein kinases are a large family of enzymes that modify proteins by addition of phosphate groups (phosphorylation). Protein kinases constitute about 2% of all eukaryotic genes, many of which mediate the response of eukaryotic cells to external stimuli. All single subunit protein kinases contain a common catalytic domain near the carboxyl terminus while the amino terminus plays a regulatory role.

Plant receptor-like kinases are serine/threonine protein kinases with a predicted signal peptide at the amino terminus, a single transmembrane region and a cytoplasmic kinase domain. There are more than 610 RLKs potentially encoded in Arabidopsis (Shiu and Bleecker 2001). Receptor-like kinases are often part of a signaling cascade. They interpret extracellular signals, through ligand binding, and phosphorylate targets in a signaling cascade which in turn affect downstream cell processes, such as gene expression (Hardie 1999).

Identification of genes that can be manipulated to provide beneficial characteristics is highly desirable. So too are means and methods of utilizing the identified genes to effect the desirable characteristics. The receptor-like kinase identified as At2g25220 in the TAIR database is one serine/threonine kinase, and a member of the large gene family of receptor-like kinases with over 600 members in Arabidopsis (Shiu et al., 2001). However, except for annotation of the sequence as a kinase no function or role for the At2g25220 gene has been disclosed. In the present invention a high water use efficiency gene (HWE) has been identified that when its expression or activity is inhibited results in beneficial phenotypes, such as, enhancement of plant biomass accumulation relative to the water used. This occurs under both water limited and non-limited conditions and ensures better growth and therefore greater productivity of the plants.

### SUMMARY OF THE INVENTION

This invention is bases upon the discovery of a mutation in the PK220 gene that results in a plant with an altered phenotype such for example, increased water use efficiency, increased drought tolerance, reduced sensitivity to cold temperature and reduced inhibition of seedling growth in low nitrogen conditions compared to plants without the mutation.

More specifically, the invention relates to the identification of a mutant plant that comprises a mutation in the PK220 gene also referred to herein as the HWE gene. The PK220 gene is a receptor-like protein kinase. Inhibition of the expression or activity of the PK220 gene in plants provides beneficial phenotypes such as improved water use efficiency in a plant. The improved water use efficiency phenotype results in plants having improved drought tolerance.

In one aspect the invention provides a method of producing a transgenic plant, by transforming a plant, a plant tissue culture, or a plant cell with a vector containing a nucleic acid construct that inhibits the expression or activity of a PK220 gene to obtain a plant, tissue culture or a plant cell with decreased PK220 expression or activity and growing the plant or regenerating a plant from the plant tissue culture or plant cell. wherein a plant having increased water use efficiency is produced.

Accordingly, the present invention provides a method of producing a plant having an improved property, wherein the method includes inhibiting the expression or activity of an endogenous PK220 gene, wherein a plant is produced having an advantageous phenotype or improved property. In a particular embodiment, the present invention provides a method for producing plants having increased water use efficiency, wherein the method includes include generation of transgenic plants and modification of plants genome using the methods described herein.

Water use efficiency refers to the ratio between the amounts of biomass produced per unit water transpired when measured gravimetrically and the ratio of photosynthetic rate to the rate of transpiration when measured using gas exchange quantification of a leaf or shoot. As used herein, the term “increased water use efficiency” refers to a plant water use efficiency that is 2, 4, 5, 6, 8, 10, 20 or more fold greater as compared to the water use efficiency of a corresponding wild-type plant. For example, a plant having increased water use efficiency as compared to a wild-type plant may have 5%, 10%, 15%, 20%, 25%, 30%, 40%, 50%, 60% 70%, 75% or greater water use efficiency than the corresponding wild-type plant.

The methods of the invention involve inhibiting or reduced the expression or activity of an endogenous gene, such as PK220, wherein a plant is produced having an advantageous phenotype or improved property, such as increased water use efficiency. In one aspect, the invention provides a method of producing a plant having increased water use efficiency relative to a wild-type plant, by introducing into a plant cell a nucleic acid construct that inhibits or reduces the expression or activity of PK220. For example, a plant having increased water use efficiency relative to a wild type plant is produced by a) providing a nucleic acid construct containing a promoter operably linked to a nucleic acid construct that inhibits PK220 activity; b) inserting the nucleic construct into a vector; c) transforming a plant, tissue culture, or a plant cell with the vector to obtain a plant, tissue culture or a plant cell with decreased PK220 activity; d) growing the plant or regenerating a plant from the tissue culture or plant cell, wherein a plant having increased water use efficiency relative to a wild type plant is produced. The construct includes a promoter such as a constitutive promoter, a tissue specific promoter or an inducible promoter. Preferably, the tissue specific promoter is a root promoter. A preferable inducible promoter is a drought inducible promoter.

The term “nucleic acid construct” refers to a full length gene sequence or portion thereof, wherein a portion is preferably at least 19, 20, 21, 22, 23, 24, 25, 30, 40, 50, 60, 70, 75, 80, 90, 100, or 150 nucleotides in length, or the compliment thereof. Alternatively it may be an oligonucleotide, single or double stranded and made up of DNA or RNA or a DNA-RNA duplex. In a particular embodiment, the nucleic acid construct contains the full length PK220 gene sequence, or a portion thereof, wherein the portion of the PK220 sequence is at least 19, 20, 21, 22, 23, 24, 25, 30, 40, 50, 60, 70, 75, 80, 90, 100, or 150 nucleotides in length, or its compliment.

Also provided by the invention is a transgenic plant having an advantageous phenotype or improved property such as increased water use efficiency, produced by the methods described herein.

In another aspect the invention provides a plant having a non-naturally occurring mutation in an PK220 gene, wherein the plant has decreased PK220 expression or activity and the plant has increased water use efficiency relative to a wild-type control. Decreased PK220 expression or activity refers to a 2, 3, 4, 5, 6, 7, 8, 9, 10, 15, 20, 25, 30, 40, 50, 60, or 75-fold reduction or greater, at the DNA, RNA or protein level of an PK220 gene as compared to wild-type PK220, or a 2, 3, 4, 5, 6, 7, 8, 9, 10, 15, 20, 25, 30, 40, 50, 60 or 75 fold reduction of PK220 activity as compared to wild-type PK220 activity. PK220 activity includes but is not limited kinase activity at serine and or threonine amino acid residues of substrate polypeptides, where it participates in phosphorylation reactions.

The invention further provides a transgenic seed produced by the transgenic plant(s) of the invention, wherein the seed produces plant having an advantageous phenotype or improved property such as for example, increased water use efficiency relative to a wild-type plant.

In another embodiment, the invention provides nucleic acids for expression of nucleic acids in a plant cell to produce a transgenic plant having an advantageous phenotype or improved property such as increased water use efficiency.

Exemplary sequences encoding a wild type PK220 gene or portion thereof that find use in aspects of the present invention are described in SEQ ID NO's: 1, 7, 9, 11, 12, 13, 24, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 84, 86, 88, 90, 92, 94, 96, 98, 100, 153, 161 and 193. Exemplary sequences encoding a mutated PK220 gene are described in SEQ ID NO's:3 and 5. Exemplary sequences that are useful for constructs to downregulate PK220 expression or activity are described in SEQ ID NO's: 12, 13, 147, 149, 153, 161, 168 and 174. The invention further provides compositions which contain the nucleic acids of the invention for expression in a plant cell to produce the transgenic plants described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from and are encompassed by the following detailed description and claims.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

For convenience, before further description of the present invention, certain terms employed in the specification, examples and claims are defined herein. These definitions should be read in light of the remainder of the disclosure and as understood by a person of ordinary skill in the art.

A “promoter sequence”, or “promoter”, means a nucleic acid sequence capable of inducing transcription of an operably linked gene sequence in a plant cell. Promoters include for example (but not limited to) constitutive promoters, tissue specific promoters such as a root promoter, an inducible promoters such as a drought inducible promoter or an endogenous promoters such as a promoter normally associated with a gene of interest, i.e. a PK220 gene

The term “expression cassette” means a vector construct wherein a gene or nucleic acid sequence is transcribed. Additionally, the expressed mRNA may be translated into a polypeptide.

The terms “expression” or “overexpression” are used interchangeably and mean the expression of a gene such that the transgene is expressed. The total level of expression in a cell may be elevated relative to a wild-type cell.

The term “non-naturally occurring mutation” refers to any method that introduces mutations into a plant or plant population. For example, chemical mutagenesis such as ethane methyl sulfonate or methanesulfonic acid ethyl ester, fast neutron mutagenesis, DNA insertional means such as a T-DNA insertion or site directed mutagenesis methods.

The term “drought stress” refers to a condition where plant growth or productivity is inhibited relative to a plant where water is not limiting. The term “water-stress” is used synonymously and interchangeably with the drought water stress.

The term “drought tolerance” refers to the ability of a plant to outperform a wildtype plant under drought stress conditions or water limited conditions or to use less water during grow and development relative to a wildtype plant.

The “term water use efficiency” is an expression of the ratio between the amounts of biomass produced per unit water transpired when measured gravimetrically and the ratio of photosynthetic rate to the rate of transpiration when measured using gas exchange quantification of a leaf or shoot.

The term “dry weight” means plant tissue that has been dried to remove the majority of the cellular water and is used synonymously and interchangeably with the term biomass.

The term “null” is defined as a segregated sibling of a transgenic line that has lost the inserted transgene and is therefore used a control line.

A number of various standard abbreviations have been used throughout the disclosure, such as g, gram; WT, wild-type; DW, dry weight; WUE, water use efficiency; d, day.

The term “hwe116” means a plant having a mutation in a PK220 gene.

The HWE gene is referred to as a PK220 gene sequence and a protein encoded by a PK220 gene is referred to as a PK220 polypeptide or protein. The terms HWE and PK220 are synonymous.

The term “PK220 nucleic acid” refers to at least a portion of a PK220 nucleic acid. Similarly the term “PK220 protein” or “PK220 polypeptide” refers to at least a portion thereof. A portion is of at least 21 nucleotides in length with respect to a nucleic acid and a portion of a protein or polypeptide is at least 7 amino acids. The term “AtPK220” refers to an Arabidopsis thaliana PK220 gene, the term “BnPK220” refers to a Brassica napus PK220 gene.

The invention is based in part on the discovery of plants having an improved agronomic property, for example, increased water use efficiency, increased drought tolerance, reduced sensitivity to cold temperature and reduced inhibition of seedling growth in low nitrogen conditions relative to a wild type control. The gene responsible for the beneficial phenotype has been determined and shown to be an inhibited PK220 gene.

Methods of producing a plant, including a mutant plant, a transgenic plant or genetically modified plant, having increased water use efficiency are disclosed herein.

Specifically the invention identifies a PK220 gene that when expression or activity of the PK220 gene is inhibited, a plant having a beneficial phenotype is obtained.

### Determining Homology Between Two or More Sequences

To determine the percent homology between two amino acid sequences or between two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch (1970). Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the coding sequence portion of the DNA sequence shown in SEQ ID NO:1.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

### Inhibition of Endogenous PK220 Expression and Activity

An aspect of the invention pertains to means and methods of inhibiting or reducing PK220 gene expression and activity, optionally, resulting in an inhibition or reduction of PK220 protein expression and activity. The term “PK220 expression or activity” embraces both these levels of inhibition or reduction. Decreased PK220 expression or activity refers to a 2, 3, 4, 5, 6, 7, 8, 9, 10, 15, 20, 25, 30, 40, 50, 60, or 75-fold reduction or greater, at the DNA, RNA or protein level of an PK220 gene as compared to wild-type PK220, or a 2, 3, 4, 5, 6, 7, 8, 9, 10, 15, 20, 25, 30, 40, 50, 60 or 75 fold reduction of PK220 protein activity as compared to wild-type PK220 activity. PK220 protein activity includes but is not limited kinase activity at serine and or threonine amino acid residues of substrate polypeptides, where it participates in phosphorylation reactions. Methods of measuring serine/threonine kinase activity are known to those in the art.

There are numerous methods known to those skilled in the art of achieving such inhibition that effect a variety of steps in a gene expression pathway, for example transcriptional regulation, post transcriptional and translational regulation. Such methods include, but are not limited to, antisense methods, RNAi constructs, including all hairpin constructs and RNAi constructs useful for inhibition by dsRNA-directed DNA methylation or inhibition by mRNA degradation or inhibition of translation, microRNA (miRNA), including artificial miRNA (amiRNA) (Schwab et al., 2006) technologies, mutagenesis and TILLING methods, in vivo site specific mutagenesis techniques and dominant/negative inhibition approaches.

A preferred method of gene inhibition involves RNA inhibition (RNAi) also known as hairpin constructs. A portion of the gene to inhibit is used and cloned in a sense and antisense direction having a spacer separating the sense and antisense portions. The size of the gene portions should be at least 20 nucleotides in length and the spacer may be a little as 13 nucleotides (Kennerdell and Carthew, 2000) in length and may be an intron sequence, a coding or non-coding sequence.

Antisense is a common approach wherein the target gene, or a portion thereof, is expressed in an antisense orientation resulting in inhibition of the endogenous gene expression and activity. The antisense portions need not be a full length gene nor be 100% identical. Provided that the antisense is at least about 70% or more identical to the endogenous target gene and of least 19, 20, 21, 22, 23, 24, 25, 30, 40, 50, 60, 70, 75, 80, 90, 100, or 150 nucleotides in length. Preferably, 50 nucleotides or greater in length the desired inhibition will be obtained.

Sequences encoding a wild type PK220 gene or portion thereof that are useful in preparing constructs for PK220 inhibition include for example, SEQ ID NO's: 1, 7, 9, 11, 12, 13, 24, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 84, 86, 88, 90, 92, 94, 96, 98, 100, 153, 161 and 193. Exemplary sequences that are useful for constructs to downregulate PK220 expression or activity are described in SEQ ID NO's: 12, 13, 147, 149, 153, 161, 168 and 174.

When using an antisense strategy of down-regulation, inhibition of endogenous gene activity can be selectively targeted to the gene or genes of choice by proper selection of a fragment or portion for antisense expression. Selection of a sequence that is present in the target gene sequence and not present in related genes (non-target gene) or is less than 70% conserved in the non-target sequences results in specificity of gene inhibition.

Alternatively, amiRNA inhibition can be used to inhibit gene expression and activity in a more specific manner than other RNAi methods. In contrast to siRNA that requires a perfect match between the small RNA and the target mRNA, amiRNA allows up to 5 mismatches with no more than 2 consecutive mismatches. The construction of amiRNA needs to meet certain criteria described in Schawab et al. (2006). This provides a method to down-regulate a target gene expression or activity using a gene portion comprising of at least a 21 nucleotide sequence of PK220.

Dominant/negative inhibition is analogous to competitive inhibition of biochemical reactions. Expression of a modified or mutant polypeptide that lacks full functionality competes with the wild type or endogenous polypeptide thereby reducing the total gene/protein activity. For example an expressed protein may bind to a protein complex or enzyme subunit to produce a non-functional complex. Alternatively the expressed protein may bind substrate but not have activity to perform the native function. Expression of sufficient levels of non active protein will reduce or inhibit the overall function.

Expression of PK220 genes that produce a PK220 protein that is deficient in activity can be used for dominant/negative down-regulation of gene activity. This is analogous to competitive inhibition. A PK220 polypeptide is produced that, for example, may associate with or bind to a target molecule but lacks endogenous activity. An example of such an inactive PK220 is the AtPK220 sequence isolated from the hwe116 mutant and disclosed as SEQ ID NO:3. A target molecule may be an interacting protein of a nucleic acid sequence. In this manner the endogenous PK220 protein is effectively diluted and downstream responses will be attenuated.

In vivo site specific mutagenesis is available whereby one can introduce a mutation into a cells genome to create a specific mutation. The method as essentially described in Dong et al. (2006) or US patent application publication number 20060162024 which refer to the methods of oligonucleotide-directed gene repair. Alternatively one may use chimeric RNA/DNA oligonucleotides essentially as described Beetham (1999). Accordingly, a premature stop codon may be generated in the cells' endogenous gene thereby producing a specific null mutant. Alternatively, the mutation may interfere with splicing of the initial transcript thereby creating a non-translatable mRNA or a mRNA that produces an altered polypeptide which does not possess endogenous activity. Preferable mutations that result loss or reduction of PK220 expression or activity include a C to T conversion at nucleotide position 874 when numbered in accordance with SEQ ID NOs: 1 or 3 or a nucleotide mutation that results in an amino acid change from a Leucine (L) codon (CTT) to a Phenylalanine (F) codon (TTT) at amino acid position 292 when numbered in accordance with SEQ ID NOs: 2 or 4.

TILLING is a method of isolating mutations in a known gene from an EMS-mutagenized population. The population is screened by methods essentially as described in (Greene et al., 2003).

Other strategies of gene inhibition will be apparent to the skilled worker including those not discussed here and those developed in the future.

### Identification of AtPK220 Homologues

Homologues of Arabidopsis thaliana PK220 (AtPK220) were identified using database sequence search tools, such as the Basic Local Alignment Search Tool (BLAST) (Altschul et al., 1990 and Altschul et al., 1997). The tblastn or blastn sequence analysis programs were employed using the BLOSUM-62 scoring matrix (Henikoff and Henikoff, 1992). The output of a BLAST report provides a score that takes into account the alignment of similar or identical residues and any gaps needed in order to align the sequences. The scoring matrix assigns a score for aligning any possible pair of sequences. The P values reflect how many times one expects to see a score occur by chance. Higher scores are preferred and a low threshold P value threshold is preferred. These are the sequence identity criteria. The tblastn sequence analysis program was used to query a polypeptide sequence against six-way translations of sequences in a nucleotide database. Hits with a P value less than −25, preferably less than −70, and more preferably less than −100, were identified as homologous sequences (exemplary selected sequence criteria). The blastn sequence analysis program was used to query a nucleotide sequence against a nucleotide sequence database. In this case too, higher scores were preferred and a preferred threshold P value was less than −13, preferably less than −50, and more preferably less than −100.

A PK220 gene can be isolated via standard PCR amplification techniques. Use of primers to conserved regions of a PK220 gene and PCR amplification produces a fragment or full length copy of the desired gene. Template may be DNA, genomic or a cDNA library, or RNA or mRNA for use with reverse transcriptase PCR (RtPCR) techniques. Conserved regions can be identified using sequence comparison tools such as BLAST or CLUSTALW for example. Suitable primers have been used and described elsewhere in this application.

Alternatively, a fragment of a sequence from a PK220 gene is ³²P-radiolabeled by random priming (Sambrook et al., 1989) and used to screen a plant genomic library (the exemplary test polynucleotides). As an example, total plant DNA from Arabidopsis thaliana, Nicotiana tabacum, Lycopersicon pimpinellifolium, Prunus avium, Prunus cerasus, Cucumis sativus, or Oryza sativa are isolated according to Stockinger et al. (Stockinger et al., 1996). Approximately 2 to 10 μg of each DNA sample are restriction digested, transferred to nylon membrane (Micron Separations, Westboro, Mass) and hybridized. Hybridization conditions are: 42° C. in 50% formamide, 5×SSC, 20 mM phosphate buffer 1×Denhardt's, 10% dextran sulfate, and 100 μg/ml herring sperm DNA. Four low stringency washes at RT in 2×SSC, 0.05% sodium sarcosyl and 0.02% sodium pyrophosphate are performed prior to high stringency washes at 55° C. in 0.2.times.SSC, 0.05% sodium sarcosyl and 0.01% sodium pyrophosphate. High stringency washes are performed until no counts are detected in the washout according to Walling et al. (Walling et al., 1988). Positive isolates are identified, purified and sequenced. Other methods are available for hybridization, for example the ExpressHyb™ hybridization solution available from Clonetech.

### PK220 Recombinant Expression Vectors and Host Cells

Another aspect of the invention pertains to vectors, preferably expression vectors, containing a nucleic acid encoding a PK220 protein, a PK220 gene or genomic sequence or portions thereof and analogs or homologs thereof. As used herein the term expression vector includes vectors which are designed to provide transcription of the nucleic acid sequence. Transcribed sequences may be designed to inhibit the endogenous expression or activity of an endogenous gene activity correlating to the transcribed sequence. Optionally, the transcribed nucleic acid need not be translated but rather inhibits the endogenous gene expression as in antisense or hairpin down-regulation methodology. Alternatively, the transcribed nucleic acid may be translated into a polypeptide or protein product. The polypeptide may be a non-full length, mutant or modified variant of the endogenous protein. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel (1990). Regulatory sequences include those that direct constitutive expression of a nucleotide sequence in many types of host cell and those that direct expression of the nucleotide sequence only in certain host cells (e.g., tissue-specific regulatory sequences) or inducible promoters (e.g., induced in response to abiotic factors such as environmental conditions, heat, drought, nutrient status or physiological status of the cell or biotic such as pathogen responsive). Examples of suitable promoters include for example constitutive promoters, ABA inducible promoters, tissue specific promoters and abiotic or biotic inducible promoters. It will be appreciated by those skilled in the art that the design of the expression vector can depend on such factors as the choice of the host cell to be transformed, the level of expression of protein desired as well as timing and location of expression, etc. The expression vectors of the invention can be introduced into host cells to thereby produce proteins or peptides, including fusion proteins or peptides, encoded by nucleic acids as described herein (e.g., PK220 proteins, mutant forms of PK220 proteins, fusion proteins, etc.).

The recombinant expression vectors of the invention can be designed for expression of PK220 genes, PK220 proteins, or portions thereof, in prokaryotic or eukaryotic cells. For example, PK220 genes or PK220 proteins can be expressed in bacterial cells such as Escherichia coli, insect cells (using baculovirus expression vectors), yeast cells, plant cells or mammalian cells. Suitable host cells are discussed further in Goeddel (1990). Alternatively, the recombinant expression vector can be transcribed and translated in vitro, for example using T7 promoter regulatory sequences and T7 polymerase.

In one embodiment, a nucleic acid of the invention is expressed in plants cells using a plant expression vector. Examples of plant expression vectors systems include tumor inducing (Ti) plasmid or portion thereof found in Agrobacterium, cauliflower mosaic virus (CaMV) DNA and vectors such as pBI121.

For expression in plants, the recombinant expression cassette will contain in addition to the PK220 nucleic acids, a promoter region that functions in a plant cell, a transcription initiation site (if the coding sequence to transcribed lacks one), and optionally a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Examples of suitable promoters include promoters from plant viruses such as the 35S promoter from cauliflower mosaic virus (CaMV) (Odell et al., 1985), promoters from genes such as rice actin (McElroy et al., 1990), ubiquitin (Christensen et al., 1992; pEMU (Last et al., 1991), MAS (Velten et al., 1984), maize H3 histone (Lepetit et al., 1992); and Atanassvoa et al., 1992), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as Rsyn7, SCP and UCP promoters, ribulose-1,3-diphosphate carboxylase, fruit-specific promoters, heat shock promoters, seed-specific promoters and other transcription initiation regions from various plant genes, for example, including the various opine initiation regions, such as for example, octopine, mannopine, and nopaline. In some cases a promoter associated with the gene of interest (e.g. PK220) may be used to express a construct targeting the gene of interest, for example the native AtPK220 promoter (P_{PK}). Additional regulatory elements that may be connected to a PK220 encoding nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements of PK220 gene are known and include, but are not limited to, 3′ termination and/or polyadenylation regions such as those of the Agrobacterium tumefaciens nopaline synthase (nos) gene (Bevan et al., 1983); the potato proteinase inhibitor II (PINII) gene (Keil et al., 1986) and hereby incorporated by reference); and An et al. (1989); and the CaMV 19S gene (Mogen et al., 1990).

Plant signal sequences, including, but not limited to, signal-peptide encoding DNA/RNA sequences which target proteins to the extracellular matrix of the plant cell (Dratewka-Kos et al., 1989) and the Nicotiana plumbaginfolia extension gene (De Loose et al., 1991), or signal peptides which target proteins to the vacuole like the sweet potato sporamin gene (Matsuoka et al., 1991) and the barley lectin gene (Wilkins et al., 1990), or signals which cause proteins to be secreted such as that of PRIb (Lund et al., 1992), or those which target proteins to the plastids such as that of rapeseed enoyl-ACP reductase (Verwoert et al., 1994) are useful in the invention.

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art. For example, the promoter associated with a coding sequence identified in the TAIR data base as At2g44790 (P₄₇₉₀) is a root specific promoter. Especially useful in connection with the nucleic acids of the present invention are expression systems which are operable in plants. These include systems which are under control of a tissue-specific promoter, as well as those which involve promoters that are operable in all plant tissues.

Organ-specific promoters are also well known. For example, the chalcone synthase-A gene (van der Meer et al., 1990) or the dihydroflavonol-4-reductase (dfr) promoter (Elomaa et al., 1998) direct expression in specific floral tissues. Also available are the patatin class I promoter is transcriptionally activated only in the potato tuber and can be used to target gene expression in the tuber (Bevan, 1986). Another potato-specific promoter is the granule-bound starch synthase (GBSS) promoter (Visser et al., 1991).

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, 1986).

The resulting expression system or cassette is ligated into or otherwise constructed to be included in a recombinant vector which is appropriate for plant transformation. The vector may also contain a selectable marker gene by which transformed plant cells can be identified in culture. The marker gene may encode antibiotic resistance. These markers include resistance to G418, hygromycin, bleomycin, kanamycin, and gentamicin. Alternatively the marker gene may encode a herbicide tolerance gene that provides tolerance to glufosinate or glyphosate type herbicides. After transforming the plant cells, those cells having the vector will be identified by their ability to grow on a medium containing the particular antibiotic or herbicide. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

A host cell of the invention, such as a prokaryotic or eukaryotic host cell in culture, can be used to produce (i.e., express) a polypeptide of the invention encoded in an open reading frame of a polynucleotide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

A number of cell types may act as suitable host cell for expression of a polypeptide encoded by an open reading frame in a polynucleotide of the invention. Plant host cells include, for example, plant cells that could function as suitable hosts for the expression of a polynucleotide of the invention include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutum and Glycine max.

Expression of PK220 nucleic acids encoding a PK220 protein that is not fully functional can be useful in a dominant/negative inhibition method. A PK220 variant polypeptide, or portion thereof, is expressed in a plant such that it has partial functionality. The variant polypeptide may for example have the ability to bind other molecules but does not permit proper activity of the complex, resulting in overall inhibition of PK220 activity.

### Transformed Plants Cells and Transgenic Plants

The invention includes a protoplast, plants cell, plant tissue and plant (e.g., monocot or dicot) transformed with a PK220 nucleic acid, a vector containing a PK220 nucleic acid or an expression vector containing a PK220 nucleic acid. As used herein, “plant” is meant to include not only a whole plant but also a portion thereof (i.e., cells, and tissues, including for example, leaves, stems, shoots, roots, flowers, fruits and seeds).

The plant can be any plant type including, for example, species from the genera Arabidopsis, Brassica, Oryza, Zea, Sorghum, Brachypodium, Miscanthus, Gossypium, Triticum, Glycine, Pisum, Phaseolus, Lycopersicon, Trifolium, Cannabis, Cucurbita, Rosa, Vitis, Juglans, Fragaria, Lotus, Medicago, Onobrychis, Trigonella, Vigna, Citrus, Linum, Geranium, Manihot, Daucus, Raphanus, Sinapis, Atropa, Capsicum, Datura, Hyoscyamus, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Lolium, Avena, Hordeum, Secale, Picea, Caco, and Populus.

The invention also includes cells, tissues, including for example, leaves, stems, shoots, roots, flowers, fruits and seeds and the progeny derived from the transformed plant.

Numerous methods for introducing foreign genes into plants are known and can be used to insert a gene into a plant host, including biological and physical plant transformation protocols (See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88; and Andrew Bent in, Clough S J and Bent A F, (1998) “Floral dipping: a simplified method for Agrobacterium-mediated transformation of Arabidopsis thaliana”). The methods chosen vary with the host plant, and include chemical transfection methods such as calcium phosphate, polyethylene glycol (PEG) transformation, microorganism-mediated gene transfer such as Agrobacterium (Horsch et al., 1985), electroporation, protoplast transformation, micro-injection, flower dipping and biolistic bombardment.

### Agrobacterium-Mediated Transformation

The most widely utilized method for introducing an expression vector into plants is based on the natural transformation system of Agrobacterium tumefaciens and A. rhizogenes which are plant pathogenic bacteria which genetically transform plant cells. The Ti and Ri plasmids of A. tumefaciens and A. rhizogenes, respectfully, carry genes responsible for genetic transformation of plants (See, for example, Kado, 1991). Descriptions of the Agrobacterium vector systems and methods for Agrobacterium-mediated gene transfer are provided in Gruber et al. (1993). and Moloney et al., (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the plant has both developing flowers and open flowers. The plants are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

### Direct Gene Transfer

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford et al., 1993; Klein et al., 1992).

Plant transformation can also be achieved by the Aerosol Beam Injector (ABI) method described in U.S. Pat. Nos. 5,240,842, 6,809,232. Aerosol beam technology is used to accelerate wet or dry particles to speeds enabling the particles to penetrate living cells. Aerosol beam technology employs the jet expansion of an inert gas as it passes from a region of higher gas pressure to a region of lower gas pressure through a small orifice. The expanding gas accelerates aerosol droplets, containing nucleic acid molecules to be introduced into a cell or tissue. The accelerated particles are positioned to impact a preferred target, for example a plant cell. The particles are constructed as droplets of a sufficiently small size so that the cell survives the penetration. The transformed cell or tissue is grown to produce a plant by standard techniques known to those in the applicable art.

### Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregate and can transmit the PK220 gene construct to its progeny. A more preferred transgenic plant is homozygous for the gene construct, and transmits that gene construct to all offspring on sexual mating. Seed from a transgenic plant may be grown in the field or greenhouse, and resulting sexually mature transgenic plants are self-pollinated to generate true breeding plants. The progeny from these plants become true breeding lines that are evaluated for decreased expression of the PK220 gene.

### Method of Producing Transgenic Plants

Also included in the invention are methods of producing a transgenic plant having increased water use efficiency, reduced sensitivity to cold temperature and reduced inhibition of seedling growth in low nitrogen conditions, relative to a wild type plant. The method includes introducing into one or more plant cells a compound that inhibits or reduces PK220 expression or activity in the plant to generate a transgenic plant cell and regenerating a transgenic plant from the transgenic cell. The compound can be, e.g., (i) a PK220 polypeptide; (ii) a PK220 nucleic acid, analog, homologue, orthologue, portion, variant or complement thereof; (iii) a nucleic acid that decreases expression of a PK220 nucleic acid. A nucleic acid that decreases expression of a PK220 nucleic acid may include promoters or enhancer elements. The PK220 nucleic acid can be either endogenous or exogenous, for example an Arabidoposis PK220 nucleic acid may be introduced into a Brassica or corn species. Preferably, the compound is a PK220 nucleic acid sequence endogenous to the species being transformed. Alternatively, the compound is a PK220 nucleic acid sequence exogenous to the species being transformed and having at least 70%, 75%, 80%, 85%, 90% or greater homology to the endogenous target sequence.

In various aspects the transgenic plant has an altered phenotype as compared to a wild type plant (i.e., untransformed). By altered phenotype is meant that the plant has a one or more characteristic that is different from the wild type plant. For example, when the transgenic plant has been contacted with a compound that decreases the expression or activity of a PK220 nucleic acid, the plant has a phenotype such as increased water use efficiency, reduced sensitivity to cold temperature and reduced inhibition of seedling growth in low nitrogen conditions, relative to a wild type plant.

The plant can be any plant type including, for example, species from the genera Arabidopsis, Brassica, Oryza, Zea, Sorghum, Brachypodium, Miscanthus, Gossypium, Triticum, Glycine, Pisum, Phaseolus, Lycopersicon, Trifolium, Cannabis, Cucurbita, Rosa, Vitis, Juglans, Fragaria, Lotus, Medicago, Onobrychis, Trigonella, Vigna, Citrus, Linum, Geranium, Manihot, Daucus, Raphanus, Sinapis, Atropa, Capsicum, Datura, Hyoscyamus, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Lolium, Avena, Hordeum, Secale, Picea, Caco, and Populus.

### Examples

### Identification of High Water Use Efficiency Mutant Hwe116

An Arabidopsis EMS mutant (Columbia background) was identified initially as having drought tolerant properties. The mutant was tested for water use efficiency under optimal and drought conditions. The result showed that the drought tolerant nature of this mutant is due to its higher water use efficiency under both water stressed and optimal water conditions. Thus, this mutant is named hwe116.

### Map Based Cloning of Hwe116

A F2 population was generated by crossing the hwe116 mutant to the Landsberg erecta (Ler) ecotype of Arabidopsis thaliana and the resulting population was used for map-based cloning by assaying for drought tolerance and subsequently confirming the presence of the higher water use efficiency trait in the mutant. The water-loss per unit dry weight of the F2 plants was measured over a 5-day drought treatment and the data was normalized for QTL analysis relative to the hwe116 mutant and the two wild type ecotypes, Landsberg erecta and Columbia. Leaf tissues were collected from all F2 and control plants used in the phenotyping experiments for genotyping. QTL analysis was conducted using MAPMAKER 3.0 and WinQTLCart 2.5. To further specify the mutations within the QTL peak, celery endonuclease I (CEL I) was used.

### Mutation Detection Using CEL I Nuclease

Celery endonuclease I (CEL I), cleaves DNA with high specificity at sites of base-pair substitution that creates a mismatch between wild type and mutant alleles and has been reportedly used for detecting mutations in EMS mutants (Yang et al., 2000; Oleykowski et al., 1998).

DNA fragments of about 5 kb were amplified by optimized PCR using hwe116 or parent Columbia genomic DNA as template. Equal amounts of the amplified products were mixed together and then subjected to a cycle of denaturing and annealing to form heteroduplex DNA. Incubation with CEL I at 42° C. for 20 minutes cleaves the heteroduplex DNA at points of mutation, and DNA fragments were visualized by 1% agarose gel electrophoresis and ethidium bromide staining.

Using this method a 5 kb PCR product was amplified using primers SEQ ID NO:102 and SEQ ID NO:104, and templates: hwe116, and the control Columbia type. The heteroduplexes formed PCR products resulted in smaller fragments (1.4 and 3.6 kb) after CEL I digestion. Overlapping sub-fragments (about 3 kb) were amplified using primers SEQ ID NO:104 and SEQ ID NO:105 to more narrowly define the mutation location. The sub-fragment was sequenced and a C nucleotide was found to have been mutated to T nucleotide in hwe116.

The mutation of interest was identified as a C to T conversion at nucleotide position 874 of SEQ ID NO's:1 and 3 that resulted in an amino acid change from a Leucine (L) codon (CTT) to a Phenylalanine (F) codon (TTT) at amino acid position 292. The gene harboring the mutation was identified as a Serine/Threonine protein kinase (Ser/Thr PK). The wild type gene was identified as being identical to Genbank Accession Number At2g25220. This Ser/Thr protein kinase is referred to as AtPK220 herein, and the mutated form identified in hwe116 is referred to as AtPK220L292F.

### Transcriptional Evaluation

Northern analysis and RT-PCR indicate that the expression level and transcript size of the AtPK220 gene in hwe116 is unchanged relative to the wild type control.

### Initial Cloning of Partial AtPK220L292F and AtPK220 Sequences

Based on the TAIR annotation, partial sequences of AtPK220L292F (AtPK220L292F(p)) and partial AtPK220 (AtPK220(p)) were amplified by RT-PCRs using the primers SEQ ID NO:106 and SEQ ID NO:107 which included BamHI and PstI restriction sites for cloning and template RNA isolated from hwe116 and the control plant (Columbia), respectively). The resulting partial AtPK220L292F nucleotide sequence is shown as SEQ ID NO:5 and the corresponding amino acid sequence as SEQ ID NO:6. The resulting partial AtPK220 nucleotide sequence is shown as SEQ ID NO:7 and the corresponding amino acid sequence as SEQ ID NO:8.

Kinase Activity Assay of a Partial AtPK220L292F Protein Expressed in E. coli

The PCR products were digested with BamHI and PstI, and inserted into the expression vector: pMAL-c2 (New England Biolabs, Beverly, MA) to form an in-frame fusion protein with the malE gene for expression of the maltose-binding protein: MBP-AtPK220L292F(p) and MBP-AtPK220(p). The fusion proteins were expressed in E. coli and purified using amylose-affinity chromatography as described by the manufacturer (New England Biolabs). Fractions containing the fusion proteins were pooled and concentrated (Centriprep-30 concentrator, Amicon). SDS-PAGE was used to analyze the expression level, size and purity of the fusion proteins.

Activity assays were carried out according to (Huang et al., 2000). The kinase autophosphorylation assay mixtures (30 μl) contained kinase reaction buffer (50 mM Tris, pH 7.5, 10 mM MgCl₂, 10 mM MnCl₂), 1 μCi [γ-³²P] ATP and 10 ng of purified AtPK220L292F(p) or MBP-AtPK220(p). For the trans-phosphorylation assays, myelin basic protein (3 μg) was added to each assay. The reactions were started by the addition of the enzymes. After incubation at room temperature for 30 min, the reactions were terminated by the addition of 30 μl of Laemmli sample buffer (Laemmli, 1970). The samples were heated at 95° C. for 5 min and then loaded on a 15% SDS-polyacrylamide gel. The gels were stained with Coomassie blue R-250, then de-stained and dried. The ³²P-labeled bands were detected using Kodak X-Omat AR film.

The wild type MBP-AtPK220(p) fusion protein was able to phosphorylate the artificial substrate in the in vitro activity assay, indicating that the assay system was effective and the MBP-AtPK220(p) fusion protein was capable of activity. In contrast, the hwe116 mutant form, MBP-AtPK220L292F(p), was unable to catalyse phosphorylation of the model substrate. The single point mutation is sufficient to abolish activity of the AtPK220(p) gene from hwe116.

Isolation of Full-Length cDNA Sequence of AtPK220

The annotation of AtPK220 (At2g25220) in the TAIR database identifies a 5′ start codon, termination signal and 3′ UTR sequence. Analysis of the 5′ portion of the annotated sequence suggested an alternative 5′ sequence and start codon location. To determine the AtPK220 genes' 5′ region and the likely start codon SMART RACE (Rapid Amplification of cDNA Ends, CloneTech) was performed.

A specific primer, SEQ ID NO:108, was designed for the 5′ RACE and yielded a 450 bp PCR product. Sequence data obtained of the 450 bp 5′ RACE product indicated that the TAIR annotation of AtPK220 was missing the 5′ 186 bp that included 39 bp of 5′ UTR sequence and 147 bp of coding sequence. An intron of 324 bp, located 8 bp upstream of the TAIR identified ATG start codon of AtPK220 was also missing from the genomic annotation in TAIR.

Compiling the 5′ RACE results and TAIR database annotation yields the full-length cDNA of AtPK220 (SEQ ID NO:9). The sequence was determined to be 1542 bp in length, which included 39 bp of 5′ UTR, 204 bp of 3′UTR, and 1299 bp of coding region. The AtPK220 coding region is identified as SEQ ID NO:1 and encodes a protein of 432 amino acids and is identified as SEQ ID NO:2. Comparison of AtPK220 to its closest homolog, At4g32000, shows an additional sequence of 51 bp is present in AtPK220, that includes the sequence of nucleotides 368-418 of SEQ ID NO:9. This sequence provides a target sequence for down-regulation constructs designed to specifically down-regulate the AtPK220 gene but not non-target genes such as At4g32000.

Sequence analysis of AtPK220 indicates that this Ser/Thr PK belongs to a receptor-like protein kinase family, possessing a signal peptide (1-29), an extracellular domain (30-67), a single transmembrane domain (68-88), an ATP-binding domain (152-175 as determined by Prosite) a Ser/Thr protein kinase active-site domain (267-279 as determined by the InterPro method) and an activation loop (289-298, 303-316).

### Rescue of the Hwe116 Mutant by AtPK220

Constructs for the expression of wild-type AtPK220 were generated and transformed into the hwe116 mutant. The construct was constitutively expressed from a CaMV 35S promoter and referred to as 35S-AtPK220.

### 35S-AtPK220

The primer pair SEQ ID NO:109 and SEQ ID NO:110 was used to amplify a fragment comprising the full length open reading frame (ORF) of AtPK220. The primer pair SEQ ID NO:111 and SEQ ID NO:110 was used to amplify a fragment comprising a portion of AtPK220 ORF. The amplified fragments were digested with restriction enzymes SmaI and BamHI and cloned into a pEGAD vector digested with the same restriction enzymes. The fragment comprising the full length open reading frame of AtPK220 resulting from the PCR and subsequent restriction digestion is disclosed as SEQ ID NO:10. The fragment comprising a portion of the AtPK220 ORF resulting from the PCR and subsequent restriction digestion is disclosed as SEQ ID NO:11.

The 35S-AtPK220 construct was transformed into Arabidopsis hwe116. The transgenic lines were recovered and advanced to T3 homozygous lines. These lines are tested for their drought tolerance and water use efficiency characteristics. The 35S-AtPK220 construct restores the wild type phenotypes.

### T-DNA Knockout Lines and Physiology Assessment

SALK T-DNA knockout lines of AtPK220 and two close homologous genes in which are identified as TAIR Accession numbers AT4G32000 (SEQ ID NO:16) and AT5G11020 (SEQ ID NO:18) were obtained from ABRC and advanced to homozygosity. They are listed as follows;
AtPK220: SALK 147838;
AtPK32000 (AT4G32000): SALK_060167, SALK_029937 and SALK_121979;
AtPK11020 (AT5G11020): SAIL_1260_H05.

Analysis of gene expression levels by either RT-PCR or Northern analysis demonstrated that the target genes in the knockout lines was either significantly reduced or completely abolished. These knockout lines were used for physiological assessment. Only the knockout line of AtPK220 (SALK_147838) showed significant drought tolerance and higher water use efficiency, indicating that AtPK220 is the target gene and responsible for the water use efficiency phenotype of hwe116. The closely related genes AT4G32000 and AT5G11020 are not functionally redundant and inhibition of these genes is insufficient to generate the hwe116 phenotype.

### Inhibition of the Protein Activity for PK220 in Arabidopsis

Inhibition of gene activity can be achieved by a variety of technical means, for example, antisense expression, RNAi or hairpin constructs, in vivo mutagenesis, dominant negative approaches or generation of a mutant population and selection of appropriate lines by screening means. Provided are examples of said means to produce plants having inhibited PK220 gene expression and or activity.

### Down-Regulation of PK220 by RNAi

Constructs were designed for RNAi inhibition of PK220 using hairpin (HP) constructs. The constructs comprised a 288 bp or a 154 bp of AtPK220 cDNA sequence to produce constructs referred to as (270)PK220 and (150)PK220. The 288 bp (270)PK220 fragment comprises 10 bp of intron sequence that was included in the PCR primer during construction of these PCR products. Vector constructs using these fragments can be made to drive expression under the control of a promoter of choice that will be apparent to one of skill in the art. In these examples a constitutive promoter (35S CaMV), or the native AtPK220 promoter (P_{PK}) was used. Two fragments, or portions, of the AtPK220 gene were selected, first a 288 bp fragment At(270)PK220 (SEQ ID NO:13) and second a 154 bp fragment At(150)PK220 (SEQ ID NO:12) were selected from a divergent region of AtPK220 as compared to its closest homologue At4g32000.

### 35S-HP-At(270)PK220 and 35S-HP-At(150)PK220

The hairpin constructs (HP) 35S-HP-At(270)PK220 and 35S-HP-At(150)PK220 constructs were generated as follows. The sense fragments of (270)PK220 and (150)PK220 were amplified by RT-PCR using primer pairs of SEQ ID NO:134/SEQ ID NO:115 and SEQ ID NO:114/SEQ ID NO:115, respectively. The PCR products were digested with SacI, and inserted into a binary vector pBI121tGUS at the SacI site, respectively. The resulting vectors were then used to subclone the antisense fragments of (270)PK220 and (150)PK220 that were derived from RT-PCR products amplified using primer pairs of SEQ ID NO:112/SEQ ID NO:117, and SEQ ID NO:116/SEQ ID NO:117, respectively. Both the vector and PCR products were digested with BamHI and XbaI for subcloning.

P_{PK}-HP-At(270)PK220 and P_{PK}-HP-At(150)PK220

The P_{PK}-HP-At(270)PK220 and P_{PK}-HP-At(150)PK220 constructs were made from 35S-HP-At(270)PK220 or 35S-HP-At(150)PK220 respectively by replacing the 35S promoter sequence with AtPK220 promoter sequence (SEQ ID NO:14). The 35S promoter sequence was removed from 35S-HP-At(270)PK220 and 35S-HP-At(150)PK220 by Hind III and Xba I double digestion. The linearized plasmid was then treated with Klenow fragment of DNA polymerase I to generate blunt ends and self-ligated to form a new plasmid, in which XbaI site was restored while Hind III was gone. By using this restored XbaI site, a Nhe I DNA fragment of AtPK220 promoter was cloned upstream of HP-At(270) and HP-At(150) sequence to produce the final plasmids of P_{PK}-HP-At(270)PK220 and P_{PK}-HP-At(150)PK220. AtPK220 promoter sequence (SEQ ID NO:14) was amplified by PCR from Arabidopsis (Columbia) genome using primer pairs of SEQ ID NO:135/SEQ ID NO:136.

### P₄₇₉₀-HP-At(270)PK220

To specifically down-regulate endogenous AtPK220, a strong root promoter P₄₇₉₀ was identified and found to be highly expressed in the roots of Arabidopsis, particularly in the endodermis, pericycle, and stele. The P₄₇₉₀ promoter is associated with a coding sequence identified as At2g44790 and the expression characteristics of P₄₇₉₀ are similar to that of wild type AtPK220 expression. The P₄₇₉₀ was used to replace the constitutive 35S promoter in 35S-HP-At(270)PK220. The promoter of At2g44790 was amplified using Arabidopsis (Col) genomic DNA as template and primers SEQ ID NO:151 and SEQ ID NO:152. The amplified promoter fragment has the length of 1475 base-pairs right upstream the ATG start codon of At2g44790 according to TAIR annotation. The 1475 bp-P₄₇₉₀ fragment is identified a SEQ ID NO:150. Hind III and Xba I restriction sites were introduced to the 5′ and 3′ end of the promoter fragment by primer design. The promoter sequence was then used to replace the 35S promoter in 35S-HP-At(270)PK plasmids by HindIII/XbaI double digestion, which resulted in the final constructs of pBI-P4790-HP-At(270)PK.

### Down-Regulation of BnPK220 in Brassica Using RNAi

### 35S-HP-Bn(340)PK

To down-regulate the AtPK220 homolog in Brassica species, a hairpin construct was made using a 338 bp fragment of BnPK220 (SEQ ID NO; 153) as the sense and anti-sense portions, and pBI300tGUS as the vector. Two pairs of primers SEQ ID NO:154 and SEQ ID NO:155; and SEQ ID NO:156 and SEQ ID NO:157 with unique restriction sites were designed according to BnPK220 sequence. A PCR fragment of 338 bp in length was amplified using Brassica napus cDNA as the template and the two pairs of primers, respectively. The SacI fragment was then inserted into pBI300tGUS at the SacI site downstream of the tGUS spacer in an antisense orientation. The resulting plasmid was subsequently used for cloning of a XbaI-BamI fragment in a sense orientation at the XbaI and BamHI sites. The vector pBI121tGUS was modified within the NPT II selectable marker gene and named pBI300. The NPT II gene in the vector pBI121 contains a point mutation (G to T at position 3383, amino acid change E182D). To restore the gene with its WT version, the NheI-BstBI fragment (positions 2715-3648) was replaced with the corresponding NheI-BstBI fragment from plasmid pRD400 (PNAS, 87:3435-3439, 1990; Gene, 122:383-384, 1992).

### P₄₇₉₀-HP-Bn(340)PK

The P₄₇₉₀ promoter of At2g44790 was used to control expression of a hairpin construct to down-regulate endogenous BnPK220 in Brassica. The plasmid of 35S-HP-Bn(340)PK was digested with HindIII and XbaI to replace the 35S promoter with the P₄₇₉₀ promoter.

### Down-Regulation of PK220 by Antisense

The construct 35S-antisenseAtPK220 was made to down-regulate expression of AtPK220 via antisense. The antisense fragment was generated using PCR and the primer pair SEQ ID NO:106/SEQ ID NO:113. The synthesised product was digested with BamHI and XbaI to yield a 1177 bp sequence comprising 1160 bp of AtPK220 (SEQ ID NO:11). Included at the 5′ end were 10 bp of intron sequence and at the 3′end, 7 bp of 3′ UTR sequence, which were retained from the PCR primers. The 1177 bp fragment was cloned in an antisense orientation to the 35S promoter in pBI121w/oGUS at the BamHI and XbaI.

### Down-Regulation of PK220 by AmiRNA

An artificial microRNA (amiRNA) construct was also made to down-regulate the expression of AtPK220 in Arabidopsis. An Arabidopsis genomic DNA fragment containing microRNA319a gene (SEQ ID NO:148), was amplified by PCR using Arabidopsis (Col) genomic DNA as template and primers listed as SEQ ID NO:141 and SEQ ID NO:142. The backbone of miR319a was then used to construct amiRPK220 (SEQ ID NO:149), in which a 21 bp fragment of miRNA319a gene in both antisense and sense orientations was replaced by a 21 bp DNA fragment of AtPK220 using recombinant PCR. Three pairs of primers: SEQ ID NO:141/SEQ ID NO:144; SEQ ID NO:143/SEQ ID NO:146 and SEQ ID NO:145/SEQ ID NO:142 were designed for the construction. The final PCR product was digested with BamHI and XbaI, and subsequently cloned into pBI121w/o GUS for transformation into Arabidopsis or other plant species of choice.

### Inhibition of PK220 Via Dominant-Negative Strategy

### 35S-AtPK220L292F

For expression of a non-functional AtPK220 sequence the AtPK220L292F from hwe116 was PCR amplified by RT-PCR using forward and reverse primers SEQ ID NO:118 and SEQ ID NO:110. The PCR product was digested with the restriction enzymes BamHI and XbaI (SEQ ID NO:121) and ligated into the binary vector pBI121w/oGUS. The sequence of SEQ ID NO:121 comprises the AtPK220L292F open reading frame (SEQ ID NO:3) and an additional 3 bp at the 5′ end and 7 bp at the 3′ end that are derived from UTR sequences (SEQ ID NO:121). The final construct, 35S-AtPK220L292F, was used to generate Arabidopsis and Brassica transgenic plants that were advanced to homozygosity for physiology assessment. Additionally, the vector is used to transform a plant species of choice and can be a dicot or a monocot.

### P₄₇₉₀-AtPK220L292F

The HindIII-XbaI fragment of the root promoter P₄₇₉₀ was used to replace 35S promoter in pBI300, and then AtPK220L292F sequence was put downstream P₄₇₉₀ by XbaI and BamHI digestion to generate the P₄₇₉₀-driven dominant-negative construct. The resulting plasmid was then used for Brassica transformation. Additionally, the vector is used to transform a plant species of choice and can be a dicot or a monocot.

### Down-Regulation of AtPK220 Homologs in a Monocot Species Using RNAi

### P_{BduBQ}-HP-Bd(272)PK

An expression cassette was constructed and inserted into two different vector backbones, the first being into the PacI-AscI sites of pUCAP and the second being into the PacI-AscI sites of pBF012. pBF012 is identical to pBINPLUS/ARS except that the potato-Ubi3 driven NPTII cassette has been excised via FseI digestion followed by self-ligation.

Brachypodium distachyon PK220 (BdPK220) was amplified using primer combinations SEQ ID NO:158 (bWET XbaI F) plus SEQ ID NO:159 (bWET BamHI R) having XbaI or BamHI sites respectively in the primers and SEQ ID NO:158 (bWET XbaI F) plus SEQ ID NO:160 (bWET ClaI R) having XbaI or ClaI sites respectively in the primers. PCR products were digested with the indicated restriction enzymes giving a 272 bp fragment (SEQ ID NO:161).

The hairpin spacer sequence, BdWx intron 1 (SEQ ID NO:164), was amplified with SEQ ID NO:162 (bWx BamHI F) plus SEQ ID NO:163 (bWx ClaI R) primers having BamHI or ClaI sites respectively in the primers and digested with the indicated restriction enzymes. The B. distachyon Wx gene is a homologue of the rice GBSS waxy gene, although the introns show little conservation.

The three fragments were ligated together into the XbaI site of the pUCAP MCS resulting in BdWx intron 1 sequence being flanked by Bd(272)PK220 target sequences in opposite orientations. The B. distachyon ubiquitin (BdUBQ) promoter contains an internal BamHI site, so the RNAi cassette was amplified with primers SEQ ID NO:200 (bWET BamHI end1) and SEQ ID NO:165 (bWET BamHI end2) which create BamHI cohesive ends without the need for BamHI digestion. The BamHI RNAi fragment was then ligated into the BamHI site of pUCAP already containing BdUBQ promoter and BdUBQT terminator resulting in the intermediate clone pBF067. The pBF067 complete insert was amplified with SEQ ID NO:166 (BdUBQ PvuI F) and SEQ ID NO:167 (BdUBQT PacI R), digested with PvuI and PacI and subsequently ligated into the PacI site of pUCAP or pBF012 vectors already containing a BdGOS2 driven mutant NPTII selectable marker in the AscI-PacI sites, resulting in pBF108 and pBF109, respectively. This mutant NPTII gene is commonly found in cloning vectors. There is only a single base pair difference from the wild type.

This cassette is in the PacI-AscI sites of pUCAP for the shuttle/bombardment vector pBF108 and in the Pac-AscI sites of pBF012 for the binary vector pBF109.

### P_{BdUBQ}-HP-Pv(251)PK

An expression cassette was constructed and inserted into two different vector backbones, the first being into the PacI-AscI sites of pUCAP and the second being into the PacI-AscI sites of pBF012. A fragment of Panicum virgatum PK220 being 251 bp in length (Pv(251)PK220) and identified as SEQ ID NO:168 was amplified using primer combinations SEQ ID NO:169 (PvWET XbaI F) plus SEQ ID NO:170 (PvWET BamHI R) and SEQ ID NO:169 (PvWET XbaI F) plus SEQ ID NO: 171 (PvWET ClaI R). PCR products were digested with the indicated restriction enzymes. No sequence information exists regarding the PvWx intron 1 so the BdWx intron 1 was used as the spacer sequence in this construct. This sequence was amplified with SEQ ID NO:162 (bWx BamHI F) plus SEQ ID NO:163 (bWx ClaI R) primers and digested with the indicated restriction enzymes.

The three fragments were then ligated together into the XbaI site of the pUCAP MCS resulting in BdWx intron 1 sequence being flanked by Pv(251)PK220 target sequences in opposite orientations. No PvUBQ promoter sequence was available so the BdUBQ promoter and terminator are used in this construct. The BdUBQ promoter contains an internal BamHI site, so the RNAi cassette was amplified with primers SEQ ID NO:172 (PvWET BamHI end1) and SEQ ID NO:173 (PvWET BamHI end2) which create BamHI cohesive ends without the need for BamHI digestion. The BamHI RNAi fragment was then ligated into the BamHI site of pUCAP already containing BdUBQ promoter and BdUBQT terminator resulting in the intermediate clone pBF152. The pBF152 complete insert was amplified with SEQ ID NO:166 (BdUBQ PvuI F) and SEQ ID NO:167 (BdUBQT PacI R), digested with PvuI and PacI and subsequently ligated into the PacI site of pUCAP or pBF012 vectors already containing BdGOS2 driven wildtype NPTII in the AscI-PacI sites, resulting in pBF169 and pBF170, respectively.

### P_{SbUBQ}-HP-Sb(261)PK

An expression cassette was constructed and inserted into two different vector backbones, the first being into the PacI-AscI sites of pUCAP and the second being into the PacI-AscI sites of pBF012. A fragment of Sorghum bicolor PK220 (SbPK220) being 261 bp in length (Sb(261)PK220) and identified as SEQ ID NO:174 was amplified using primer combinations SEQ ID NO:175 (SbWET XbaI F) plus SEQ ID NO:176 (SbWET BamHI R) and SEQ ID NO:175 (SbWET XbaI F) plus SEQ ID NO:177 (SbWET ClaI R). PCR products were digested with the indicated restriction enzymes to give a Sb(261)PK220 fragment. The hairpin spacer sequence, SbWx intron 1 (SEQ ID NO:178), was amplified with primers SEQ ID NO:179 (SbWx BamHI) plus SEQ ID NO:180 (SbWx ClaI R) and digested with the indicated restriction enzymes. The three fragments were then ligated together into the XbaI site of the pUCAP MCS resulting in SbWx intron 1 sequence being flanked by SbWET target sequences in opposite orientations. BamHI cohesive ends were added to the RNAi cassette via amplification with primers SEQ ID NO:181 (SbWET BamHI end1) and SEQ ID NO:182 (SbWET BamHI end2). The BamHI RNAi fragment was then ligated into the BamHI site of pUCAP already containing SbUBQ promoter and SbUBQT terminator resulting in the intermediate clone pBF151. The pBF151 complete insert was amplified with SEQ ID NO:192 (SbUBQ PvuI F) and SEQ ID NO:167 (BdUBQT PacI R), digested with PvuI and PacI and subsequently ligated into the PacI site of pUCAP or pBF012 vectors already containing BdGOS2 driven wildtype NPTII in the AscI-PacI sites, resulting in pBF158 and pBF171, respectively.

A SbGOS2 promoter was identified from the Sorghum genome sequence was amplified and using the primer pair SEQ ID NO:184 (SbGOS2 HindIII F) and SEQ ID NO:185 (SbGOS2 HindIII R) a 1000 bp fragment of the GOS2 promoter, identified as SEQ ID NO:183, was PCR amplified and cloned using the HindIII restriction sites.

A SbUBQ promoter was identified from the Sorghum genome sequence was amplified and using the primer pair SEQ ID NO:187 (SbUBQ PstI F) and SEQ ID NO:188 (SbUBQ PstI R) a 1000 bp fragment of the UBQ promoter, identified as SEQ ID NO:186, was PCR amplified and cloned using the PstI restriction sites.

A SbUBQ terminator was identified from the Sorghum genome sequence was amplified and using the primer pair SEQ ID NO:190 (SbUBQT KpnI F) and SEQ ID NO:191 (SbUBQT KpnI R) a 239 bp fragment of the UBQ terminator, identified as SEQ ID NO:189, was PCR amplified and cloned using the KpnI restriction sites.

Miscanthus giganteus (MgPK220) RNAi

Expression constructs designed to down regulate via a hairpin strategy can be devised following the same strategy as described above. Resulting in a construct that may comprise the following elements, a BdGOS2-wtNPTII-BdUBQT selectable marker cassette and a BdUBQ-(MgPK220 hairpin-RNAi cassette)-BdUBQT in a vector of choice such as pUCAP and pBF012

### AtPK220 Promoter Isolation and Cloning

The AtPK220 promoter was isolated using a PCR approach using Arabidopsis (Columbia ecotype) genomic DNA as template. The 5′ primer, SEQ ID NO:119, was designed near the adjacent gene and the 3′ primer, SEQ ID NO:120, located 25 bp upstream of the ATG start codon of the AtPK220 gene. The amplified product was digested with BamHI and SmaI and cloned into pBI101. The digested fragment, SEQ ID NO:14, was 1510 bp in length. The resulting construct was named P_{AtPK220}-GUS.

### AtPK220 Promoter Activity Analysis Using GUS Assay

P_{AtPK220}-GUS was transformed into Arabidopsis plants using flower dipping, and the transgenic plants were advanced to T3 homozygorsity. Various tissues including young seedlings and leaves, stems, flowers, siliques, and roots from T3 flowering plants were collected, stained in X-Gluc solution at 37 C overnight, de-stained with ethanol solution, and examined under a microscope. The results showed that the promoter of AtPK220 was expressed mainly in endodermis and pericycle cells of root tissue and was also found in leaf trichomes and seed coat of developing seeds. Of significance was the observation that expression of P_{AtPK220}-GUS was suppressed by water stress.

### Sub-Cellular Localisation of AtPK220 Proteins in Arabidopsis

Expression of a full length wild type AtPK220-GFP fusion protein in transgenic Arabidopsis was used to locate the sub-cellular localization of the native protein. The primer pair SEQ ID NO:109 and SEQ ID NO:110 produced a fragment that was digested with SmaI and BamHI to yield a fragment comprising the full length open reading frame of AtPK220 and is disclosed as SEQ ID NO:10 and cloned downstream, in frame with the green fluorescence protein (GFP) in a pEGAD plasmid at the SmaI and BamHI sites. Additionally, the AtPK220 coding sequence was amplified using primer pair SEQ ID NO:198 and SEQ ID NO:199 and inserted upstream and in frame with GFP by AgeI digestion of pEGAD plasmid and the amplified AtPK220 fragment.

The 35S-GFP-AtPK220 and 35S-AtPK220-GFP constructs were transformed into Arabidopsis plants and homozygous transgenic plants (root tissues) were used for visual screening of GFP signal under confocal microscope. Green fluorescence was detected along plasma membrane, suggesting that AtPK220 protein was associated with plasma membrane in roots and that AtPK220 possibly functions as receptor kinase to sense or transduce environmental signals.

Isolation of BnPK220 from Brassica napus by 5′ and 3′ RACE

To isolate the homologous gene of AtPK220 from canola, a blast search (BLASTn) of NCBI Nucleotide Collection (nr/nt, est) and TIGR (DFCI) Brassica napus EST Database was done using AtPK220 sequence. Based on the sequences with highest similarity, a pair of primers, SEQ ID NO:122 and SEQ ID NO:123 were designed and used to PCR amplify a partial fragment of BnPK220. Both mRNA and genomic DNA isolated from Brassica leaves were used as template for these amplifications. A DNA fragment of about 500 bp was obtained by PCR from canola genomic DNA template. Sequence analysis of this PCR product showed that it shares a high identity with AtPK220 in nucleotide sequence as well in the intron organisation.

Based on the partial sequence of BnPK220, 5′ and 3′ RACE was performed to isolate the full length BnPK220 cDNA. For 3′ RACE a forward primer, SEQ ID NO:124 and a nested primer, SEQ ID NO:125, were used. For 5′ RACE a reverse primer, SEQ ID NO:126, and its nest primer, SEQ ID NO:127, were designed. RACE-ready cDNA for either 5′ RACE or 3′ RACE was made from RNA isolated from young Brassica leaves.

The 5′ RACE yielded an amplified DNA of about 650 bp in length; and 3′ RACE yielded a DNA of about 1 kb in size. Sequencing of these two RACE fragments showed high sequence similarity with AtPK220. A full-length mRNA of BnPK220 sequence was assembled by combining 5′RACE, partial BnPK220 fragment and 3′ RACE results.

A full length BnPK220 cDNA was amplified by RT-PCR using the PCR primers SEQ ID NO:128 and SEQ ID NO:129. This cDNA comprises an ORF of 1302 nucleotides (SEQ ID NO:25) and encodes a protein of 433 amino acids (SEQ ID NO:26). Another full length BnPK220 cDNA was also amplified by the RT-PCR using cDNA made from B. napus. This cDNA (SEQ ID NO: 193) is 98.6% identical to SEQ ID NO:25, and encodes a protein (SEQ ID NO:194) of 99.3% identical to SEQ ID NO:26.

Isolation of Full-Length GmPK220 from Soybean by 5′ RACE

A Blastn search of NCBI EST database, a homolog of AtPK220 was found as a soybean (Glycine max) EST, CX709060.1. From this homolog, a unigene cluster of 13 ESTs was retrieved from a soybean EST database. A contig was then assembled from these 13 ESTs, which covers a majority of the gene sequence.

The full-length sequence of GmPK220 (SEQ ID NO:41) was determined by combining the assembled contig, 5′ RACE and 3′ RACE results. The 5′ RACE was performed using the primers of SEQ ID NO:130 for primary RACE PCR and SEQ ID NO:131 for nested RACE PCR. The 3′ RACE was performed using the primers of SEQ ID NO:137 for primary RACE PCR and SEQ ID NO:138 for nested RACE PCR. GmPK220 encodes a protein as shown in SEQ ID NO:42.

### Isolation of OsPK220 (Rice) Sequence by Database Mining

The rice genome (Oryza sativa, japonica cultivar) has been completely sequenced and is publically available. The homolog of AtPK220 in rice was determined by BLAST search of a rice EST database and by BLASTP search of a genomic sequence database. The target having the highest score was identified as Accession number Os05g0319700.

Os05g0319700 is abbreviated as OsPK220, and disclosed as SEQ ID NO:59, which encodes a protein disclosed as SEQ ID NO:60.

### Isolation of ZmPK220 (Corn) Sequence

Two candidate homologs were found by BLAST search of the TIGR EST database, one a unigene Accession number TC333547 and the second Accession number C0439063.

Accession number TC333547 is 2125 nucleotides in length and contains an open reading frame of 1377 nucleotides (SEQ ID NO:77) encoding a protein of 458 amino acids (SEQ ID NO:78). This translated protein is full-length and is larger than AtPK220 protein. The C-terminal kinase domain is highly conserved between the Arabidopsis and corn protein sequence, however, the N-terminal sequence is more variable.

CO439063 is a short EST sequence and is missing 5′ terminal sequence. The missing sequence was obtained by RACE methods. Two 5′ RACE primers were designed based on the alignment between AtPK220 and C0439063. The primary 5′ RACE primer is SEQ ID NO:132 and the nested 5′ RACE primer is SEQ ID NO:133. The 3′ RACE was also performed using the primers of SEQ ID NO:139 for primary RACE PCR and SEQ ID NO:140 for nested RACE PCR. The ZmPK220 (SEQ ID NO:79) sequence was assembled based on 5′ RACE, 3′ RACE results and C0439063 EST sequences. The corresponding protein sequence was listed as SEQ ID NO:80.

Sequence analysis shows that C0439063 has higher sequence similarity with rice OsPK220 than TC333547.

Isolation of BdPK220 sequence from Brachipodium distachyon (Bd)

Brachipodium is one of the model monocot plants for functional genomic research. A contig was assembled from public ESTs or GSSs, and it covers a 3′portion of BdPK220 according to homologue alignment. RACE using Bd81RAR1 primer (SEQ ID NO: 195) and Bd81RAR2 primer (SEQ ID NO: 196) designed from the contig and using Brachipodium leaf cDNA produced a unique fragment of about 650 bp. The assembling of the RACE sequence and the contig gave the full length BdPK220 sequence (SEQ ID NO:24), which encodes a protein of 461 amino acids (SEQ ID NO: 197).

### Determination of GsPK220 (Cotton) Sequence by Database Mining

A BLAST search of a cotton (Gossypium) TIGR-EST database identified a sequence cluster identified as Accession number TC79117, that has high similarity with AtPK220. This cluster has two overlapping ESTs, TC79117 which is referred herein as GsPK220) and consists of an open reading frame of 1086 nucleotides (SEQ ID NO:81). The largest open reading frame encodes a protein of 361 amino acids (SEQ ID NO: 82).

### Drought Tolerant Phenotype of Hwe116 Mutant Found Under Water Limited Conditions and High Water Use Efficiency Under Both Drought and Optimal Conditions

Two groups of plants were grown (5 plants per 3″ pot filled with the same amount of soil-less mix) under optimal conditions in a growth chamber (22 C, 18 hr light, 150 uE, 70% relative humidity) until first day of flower (n=6 per entry per treatment). At first flower all plants were supplied with the same amount of water (optimal levels) but one group of plants was used for the optimal treatment and the other for drought treatments. In the optimal treatment the pots were weighed daily to determine daily water loss and then watered back up to optimal levels. In the drought treatment, pots were weighed daily to determine water loss and allowed to dry out. Plants were harvested on days 0, 2 and 4 of drought and optimal treatments for shoot biomass determinations. Lower water loss relative to shoot dry weight (DW) as compared to control, under drought conditions indicates a drought tolerant phenotype. The ratio of shoot dry weight accumulated to water lost during the treatment period provides a measure of water use efficiency (WUE). The hwe116 plants were delayed in flowering by 1 to 2 days. Water loss relative to shoot biomass was significantly lower (by 22%) in hwe116 than parent control under drought conditions. This result indicates that the mutant is drought tolerant. It has also been found that under optimal conditions the water loss relative to shoot DW was also significantly lower in the mutant (by 41%) as compared to the parent control. This result is consistent with higher water use efficiency phenotype. Calculations of water use efficiency showed that under both drought (Table 1) and optimal (Table 2) conditions hwe116 mutant uses water more efficiently because it accumulated more shoot biomass with less water (drought) or the same amount of biomass with less water (optimal).

TABLE 1
Water Use Efficiency (WUE) under drought conditions

| | shoot DW | | WUE |
|---|---|---|---|
| | accumulated- | water lost- | (g shootDW acc/ |
| Entry | day 0 to 4 (g) | day 0 to 4 (g) | kg water lost) |
| hwe116 | 0.146 | 56.5 | 2.58 (+13%) |
| Parent | 0.134 | 58.6 | 2.28 |

TABLE 2
Water Use Efficiency (WUE) under optimal conditions

| | shoot DW | | |
|---|---|---|---|
| | accumulated- | water lost- | WUE (g shootDW acc/ |
| entry | day 0 to 4 (g) | day 0 to 4 (g) | kg water lost) |
| hwe116 | 0.276 | 92.3 | 2.99 (+22%) |
| Parent | 0.271 | 110.6 | 2.45 |

The final result of enhanced water use efficiency in the mutant is greater shoot DW biomass as shown in Table 3 (harvested on day 4 from 1^{st} flower).

TABLE 3
Final shoot DW biomass

| | Drought- | | Optimal- | |
|---|---|---|---|---|
| | shoot DW (g) | | shoot DW (g) | |
| entry | Mean | S.E. | Mean | S.E. |
| hwe116 | 0.354 | 0.014 | 0.449 | 0.017 |
| parent | 0.300 | 0.011 | 0.414 | 0.011 |
| hwe116 as % of parent | 118% | | 108% | |

### The Hwe116 Mutant Maintains Higher Soil Water Content During Drought Treatment, Reaches Water-Stress Conditions Later and Shows Yield Protection Following Drought Stress During Flowering Relative to Control Plants.

An experiment was set up with 5 plants per 4″ pot filled with the same amount of soilless mix. Two groups of plants (optimal and drought) were grown under optimal conditions in a growth chamber (22 C, 18 hr light, 150 uE, 70% relative humidity) until first day of flower (n=9 per entry and per group). At first flower all plants were supplied with the same amount of water and further water was withdrawn for the drought treated group of plants. The optimal group was watered daily as before. Pots in the drought treated group were weighed daily for 6 days of treatment to determine soil water content. After 6 days of drought treatment plants were re-watered and allowed to complete their lifecycle as the optimal group under optimal conditions. At maturity the seeds were harvested from each pot and the seed yield was determined for both optimal and drought treated plants. The results of changes in soil water content during the drought treatments were determined. Soil water content was measured as percentage of initial amount of water in the pot. The results indicate that the mutant was able to retain water in pots longer and therefore it reached the stress level (around 25% soil water content) 1 day later and wilted 1 day later than control. This treatment caused a yield reduction of 17% from optimal levels in the mutant, whereas in control the yield reduction was 41%. Therefore the mutant demonstrated a yield protection of 24% relative to control, following a drought treatment.

### The Hwe116 Mutant Seedlings Showed Less Sensitivity to Cold Stress.

Two groups of plants with 8 replicates per entry were grown with 3 plants per 3″ pot under optimal conditions of 22° C. and short days to prolong vegetative growth and delay flowering (10 hr light 150 uE, and 14 hr dark), 70% relative humidity in a growth chamber. At 10 days of age (3 days post-transplanting of seedlings into soil from agar plates) the cold treatment group was placed in a chamber at 8° C. for 11 more days of growth while the optimal group was maintained at 22° C. Plants were harvested for shoot dry weight (DW) determinations at 21 days of age. The results are shown in Table 4. The hwe116 mutant had smaller seedlings under optimal conditions than those of controls but after cold exposure the shoot DW was equivalent to that of the parent and as percentage of the optimal DW it was higher than that of both controls by 9 and 15% indicating that the growth of the mutant was not as inhibited by cold as that of controls.

TABLE 4
shoot dry weight under optimal and cold conditions.

| | optimal (22° C.) | | Cold (8° C.) | | |
|---|---|---|---|---|---|
| | shoot DW (mg) | | shoot DW (mg) | | shoot DW |
| Entry | Mean | S.E. | Mean | S.E. | % of optimal |
| hwe116 | 6.65 | 0.30 | 2.85 | 0.13 | 43% |
| parent | 9.16 | 0.21 | 2.58 | 0.11 | 28% |
| WT | 9.30 | 0.20 | 3.18 | 0.21 | 34% |

The Hwe116 Mutant has Thicker Leaves and Higher Chlorophyll Content Per Leaf Area. The Mutant Showed Delayed Leaf Senescence and Resistance to Oxidative Stress.

Plants were grown 1 per 3″ pot under optimal growth conditions in a growth chamber (16 hr light, 300 uE, 22° C., 70% relative humidity). Early into flowering three leaf disks (86.6 um2 each) were taken from three youngest fully developed leaves and placed in petri dishes containing filter paper with 5 uM N,N′-Dimethyl-4,4′-bipyridinium dichloride (paraquat) solution as an oxidizing agent. Plates with leaf disks were placed under continuous light of 150 uE for 25 hours. This resulted in chlorophyll bleaching. The differences between the mutant and controls in the extent of bleaching were quantified by measuring chlorophyll content of the leaf disks. A leaf disk was also removed from leaves that have not been exposed to paraquat treatment and optimal chlorophyll content was determined. These disks were also weighed. The results showed that the mutant had higher total chlorophyll content per leaf surface area (Table 5), however the leaves of this mutant are thicker (leaf disks were 15 to 24% heavier in the mutant compared to those of controls). Chlorophyll content per gram of fresh leaf tissue was, therefore, not different. There were no differences between chlorophyll a to b ratios between the mutant and controls. The hwe116 mutant showed resistance to the oxidative stress as indicated by 5 to 7% higher chlorophyll content following paraquat treatment (Table 5). Leaf senescence was also delayed in the hwe116 mutant (data not shown).

TABLE 5
Effect of oxidative stress on chlorophyll content of leaves.

| | Optimal | | 5 uM paraquat in 24 hr light | | |
|---|---|---|---|---|---|
| | Chl (a + b) − (mg/m2) | | Chl (a + b) − (mg/m2) | | % of |
| Entry | Mean | Std Err | Mean | Std Err | opt |
| hwe116 | 303.7 | 6.7 | 67.9 | 4.4 | 20% |
| Parent | 259.6 | 4.3 | 39.5 | 5.9 | 15% |
| WT | 250.2 | 5.7 | 32.1 | 2.9 | 13% |

### The Growth of Mutant Hwe116 Seedlings Showed Less Inhibition on Low Nitrogen Containing Media.

Twelve seedlings were grown on an agar plate (6 plates per entry) containing 12 MS growth media with optimal (20 mM) or low (0.3 mM) nitrogen content. Plates were placed in a growth room with an 18 hr light period (100 uE) for 6 days in a vertical position, then plates were placed horizontally and seedlings were grown for another 4 days before the shoots were harvested. The average seedling shoot DW after 10 days of growth was calculated per plate. The results are shown in Table 6. The shoot DW of hwe116 mutant grown under optimal conditions was significantly reduced but when grown on low nitrogen there were no differences. The shoot DW on low nitrogen in the mutant was 3 to 7% greater than in controls when compared to the optimal nitrogen levels. This indicates that the mutant may have better nitrogen use efficiency.

TABLE 6
Effect of nitrogen on seedling shoot DW

| | Average seedling shoot DW (mg) | | | | |
|---|---|---|---|---|---|
| | Optimal nitrogen | | Low nitrogen | | |
| Entry | Mean | S.E. | Mean | S.E. | % Opt |
| hwe116 | 1.03 | 0.03 | 0.23 | 0.01 | 22 |
| Parent | 1.34 | 0.04 | 0.20 | 0.01 | 15 |
| WT | 1.22 | 0.03 | 0.23 | 0.02 | 19 |

### Knockout Mutant of PK220 Showed Drought Tolerant Trends and Higher Water Use Efficiency Under Drought Treatment.

Plant lines obtained from the SALK institute that were T-DNA knockouts in the AtPK220 gene (SALK_147838) were grown (5 per 3″ pot) under optimal conditions in a growth chamber (18 hr light, 150 uE, 22° C., 60% relative humidity) until first open flower (n=8 per entry and per harvest). The drought treatment was started by watering all plants with the same amount of water and cessation of further watering. Pots were weighed daily and plants were harvested for shoot DW determinations on days 0, 2 and 4 of the drought treatment. The result showed that water lost from pots in 2 days relative to shoot DW on day 2 was significantly lower (by 13%) for the knockout mutant and its shoot DW was also significantly greater (by 24%) on day 2 as compared to control wild-type. This result is consistent with drought tolerant phenotype.

The results showed that the water use efficiency of the knockout mutant was greater than that of the control-WT as the knockout mutant was able to accumulate more shoot biomass in the 2 days of treatment while using the same amount of water as control (Table 7).

TABLE 7
Water use efficiency under drought treatment

| | | | WUE |
|---|---|---|---|
| entry | g water lost | g shoot DW gain | (g shoot/kg water) |
| PK220- | 43.1 | 0.059 | 1.37 |
| knockout | | | |
| WT | 42.9 | 0.035 | 0.82 |

### Transgenic Lines of 35S-HP-At(270)PK220 Construct in Arabidopsis Showed Drought Tolerance.

Plants were grown (5 per 3″ pot and 8 pots per entry per harvest) under optimal conditions in a growth chamber (18 hr light, 150 uE, 22° C., 60% relative humidity) until first day of flower. The drought treatment was started by watering all pots with the same amount of water and cessation of further watering. Pots were weighed daily for water loss determinations and plants were harvested for shoot biomass on day 4 of drought treatment. The results (Table 8) showed that 11 out of 13 transgenic lines demonstrated a drought tolerant phenotype (having a lower water loss over 2 days relative to shoot biomass on day 4). Four of the lines showed a slight delay in flowering (1 day), as did the hwe116 mutant. The final shoot biomass on day 4 was greater for most of the transgenic lines as compared to control WT. These results are indicative of a drought tolerant phenotype in the transgenic lines down-regulated in PK220 expression. As examples, the reduction in expression level of AtPK220 for the top 3 performing lines: 65-4, 38-5, and 59-3, are 75%, 47% and 58%.

TABLE 8
Drought tolerance and shoot DW (day 4) for 35S-HP-At(270)PK220
transgenic lines relative to wild type (WT) and the
hwe116 mutant relative to parent control.

| | drought tolerance | shoot DW |
|---|---|---|
| entry | % of control | % of control |
| 65-4 | 119% | 132% |
| 38-5 | 116% | 124% |
| 59-3 | 112% | 119% |
| 33-7 | 111% | 114% |
| 54-11 | 108% | 115% |
| 56-3 | 107% | 115% |
| 43-11 | 107% | 113% |
| 23-8 | 106% | 111% |
| 12-2 | 106% | 110% |
| 63-4 | 104% | 110% |
| 32-1 | 104% | 109% |
| 30-3 | 101% | 104% |
| 74-2 | 101% | 107% |
| WT | 100% | 100% |
| hwe116 | 186% | 106% |
| parent | 100% | 100% |

Drought Tolerance of 35S-HP-At(270)PK220 Transgenic Lines in Arabidopsis and Enhanced Water Use Efficiency were Confirmed.

The transgenic lines of 35S-HP-At(270)PK220 were grown with 5 per 3″ pot under optimal conditions in a growth chamber (18 hr light, 150 uE, 22° C., 60% relative humidity) until first flower (n=8). Drought treatment was started at first flower by watering all the pots with the same amount of water and cessation of further watering. The pots were weighed daily for the 4 days of drought treatment and plants were harvested on days 0, 2 and 4 of treatment. The results confirmed that water lost in 2 days relative to shoot biomass on day 2 was lower in five transgenic lines relative to controls, confirming their drought tolerant phenotype (Table 9). The shoot DW on day 2 was greater in 5 of the transgenic lines.

TABLE 9
Drought tolerance and shoot DW for 35S-
HP-At(270)PK220 transgenic lines

| | drought | |
|---|---|---|
| | tolerance | shoot DW |
| entry | % of WT | % of WT |
| 59-3 | 110% | 105% |
| 65-4 | 110% | 98% |
| 38-5 | 107% | 109% |
| 33-7 | 103% | 106% |
| 56-3 | 102% | 95% |
| 54-11 | 101% | 103% |
| null (65-1) | 99% | 99% |
| WT | 100% | 100% |

The water use efficiency was greater than that of controls during the 4 days of drought treatment for three transgenic lines and this enhanced water use efficiency was due to greater shoot DW accumulation (Table 10).

TABLE 10
Water use efficiency between day 0 and 4 of the drought
treatment in transgenic lines of 35S-HP-At(270)PK220.

| | shoot DW | | WUE (g shoot/ |
|---|---|---|---|
| | accumulated (g) | water lost (g) | kg water) |
| entry | d0-d4 | d0 to d4 | d0 to d4 |
| 65-4 | 0.090 | 62.5 | 1.44 (+22 to 33%) |
| 12-2 | 0.079 | 62.2 | 1.27 (+7 to 17%) |
| 56-3 | 0.079 | 62.7 | 1.25 (+6 to 16%) |
| null (65-1) | 0.068 | 62.7 | 1.08 |
| WT | 0.073 | 61.9 | 1.18 |

### Transgenic Lines of 35S-HP-At(270)PK220 in Arabidopsis had Lower Water Loss Relative to Shoot Biomass and Enhanced WUE Under Optimal Conditions.

Plants of 35S-HP-At(270)PK220 transgenic lines 65-7 and 59-5, WT Columbia, hwe116 mutant and its parent were grown (5 per 3″ pot) under optimal conditions in a growth chamber (22° C., 18 hr light—200 uE, 60% relative humidity) until first flower (n=8 per entry, per harvest). At first flower all pots in the water limited group were watered with the same amount of water (to a pot weight of 120 g in first 4 days and to 130 g for last 3 days (as plants grew larger they required more water). Pots were weighed daily to determine daily water loss and plants were harvested on day 0 and day 7 of this treatment. Water use efficiency (WUE) was calculated from the ratio of shoot biomass accumulated to water lost. The results are shown in Table 11.

TABLE 11
Water Use Efficiency under optimal conditions

| | shoot DW | | WUE (g shoot/ |
|---|---|---|---|
| | accumulated (g) | water lost (g) | kg water) |
| entry | d0-d4 | d0 to d4 | d0 to d4 |
| 59-5 | 0.514 | 223 | 3.31 (+4%) |
| 65-4 | 0.671 | 276 | 2.43 (+9%) |
| WT | 0.517 | 232 | 2.23 |
| hwe116 | 0.420 | 191 | 2.19 (5%) |
| parent | 0.421 | 202 | 2.08 |

The results show that under optimal water conditions the two transgenic lines and the mutant had enhanced water use efficiency.

Growth Rates of the 35S-HP-At(270)PK220 Transgenic Arabidopsis were Greater than Those of Controls During Both Optimal and Water Limited Conditions.

Plants of 35S-HP-At(270)PK220 transgenic line 65-4 and WT Columbia were grown (5 per 3″ pot) under optimal conditions in a growth chamber (22° C., 18 hr light—150 uE, 60% relative humidity) until first flower (n=8 per entry, per treatment and per harvest). At first flower all pots in the water limited group were watered with the same amount of water (to a pot weight of 95 g), and further watering was stopped for 2 days. It took 2 days for the water limited group of plants to reach about 30% of initial soil water content (about 55 g total pot weight), referred to as pre-treatment. At that time the water limited treatment was deemed to have started (day 0 of treatment) and plants were watered daily up to a total pot weight of 55 g for 3 days, and up to 65 g in the following 4 days (until day 7 of treatment). The optimal group was maintained under optimal conditions by watering the pots daily up to 100 g total pot weight in the 2 pre-treatment days, the first 3 days of treatment and then up to 130 g in the last 4 days of treatment (as plants grew larger they required more water). The daily water loss from the pots was measured for all the plants and plants in both groups were harvested on days 0, 1, 2, 3, 5, and 7 of treatment for shoot dry weight determinations. The water loss relative to the shoot biomass (drought tolerant phenotype) was calculated over the initial two days before the start of treatment, during the first 3 days of treatment and during the last 4 days of treatment. The results under both optimal (Table 12) and water limited (Table 13) conditions are shown. The transgenic line 65-4 lost less water relative to shoot biomass than WT in both optimal and water limited conditions. Under limited water conditions this is consistent with enhanced drought tolerance phenotype.

TABLE 12
Water loss in g/shoot DW in g under optimal conditions.

| Entry | pre-treatment | d0-d3 | d3-d7 |
|---|---|---|---|
| 65-4 | 231 ± 9 | 162 ± 3 | 237 ± 5 |
| WT | 275 ± 8 | 178 ± 7 | 243 ± 6 |

TABLE 13
Water loss in g/shoot DW in g and Drought tolerance
(as percentage of WT) under water limited conditions.

| | pre-treatment | d0-d3 | d3-d7 |
|---|---|---|---|
| | (drought toler. | (drought toler. | (drought toler. |
| Entry | in % of WT) | in % of WT) | in % of WT) |
| 65-4 | 174 ± 2 (108%) | 83 ± 2 (115%) | 153 ± 6 (113%) |
| WT | 189 ± 4 (100%) | 97 ± 4 (100%) | 175 ± 4 (100%) |

Growth rates of the plants were calculated over the seven days of both treatments. The results showed that transgenic line 65-4 had larger plants (up to 24%) than the wild type throughout the treatment under both conditions. The growth rate (shoot dry weight accumulated per day over the 7 days of treatment) was slightly greater for the transgenic line under both optimal and water limited conditions (63.3 and 21.3 mg shoot/day, respectively) than that of WT control (58.3 and 20.4 mg shoot/day, respectively).

The Transgenic Line of 35S-HP-At(270)PK220 Arabidopsis and the Hwe116 Mutant Grow Better Under Limited Nitrogen Conditions than Controls.

The 35S-HP-At(270)PK220 transgenic line 65-5, its segregated null control (null 65-1) and wild-type (WT) plus the hwe116 mutant and its parent control were analyzed for growth characteristics of young seedling under optimal and limited nitrogen conditions. Nitrogen content refers to the available nitrogen for plant growth, including nitrate and ammonium sources. Seedlings were grown on agar plates (10 per plate and 5 plates per entry and per treatment) containing either optimal nutrients (including 20 mM nitrogen) or low (limiting to growth) nitrogen (optimal all nutrients except for nitrogen being 0.5 mM). Plates were placed in a growth chamber at 18 hr lights of 200 uE and 22° C. Seedlings were grown for 14 days before being harvested for shoot biomass (8 seedlings) and chlorophyll determinations (2 seedlings). On optimal plates there were no differences in average seedling shoot biomass except for the hwe116 mutant, as shown before had slightly smaller seedling shoot DW (not significant). On low nitrogen the hwe116 mutant had significantly bigger seedling shoot DW and showed 30% less inhibition in growth as compared to its parent. The transgenic line 65-5 showed slightly greater shoot DW than controls and was 5% to 7% less inhibited in growth than the controls (Table 14).

TABLE 14
Effect of nitrogen on seedling shoot DW

| | Average seedling shoot DW (mg) | | | | |
|---|---|---|---|---|---|
| | Optimal N (20 mM) | | Low N (0.5 mM) | | |
| entry | Mean | Std Err | Mean | Std Err | % of opt |
| 65-5 | 5.3 | 0.1 | 2.9 | 0.1 | 56% |
| WT | 5.5 | 0.3 | 2.8 | 0.1 | 51% |
| hwe116 | 4.8 | 0.2 | 3.8 | 0.3 | 80% |
| parent | 5.1 | 0.2 | 2.6 | 0.1 | 50% |

The total chlorophyll content of seedling shoots grown under low N levels reflected the shoot DW results. Chlorophyll content is very closely linked to available N and one of the major symptoms of N-deficiency in plants is leaf chlorosis or bleaching. Table 15 shows that chlorophyll content of the transgenic line 65-5 and the mutant hwe116 was reduced less than that of the controls.

TABLE 15
Effects of nitrogen on seedling shoot total chlorophyll content

| | seedling shoot chlorophyll content (ug/g) | | | | |
|---|---|---|---|---|---|
| | Optimal N (20 mM) | | Low N (0.5 mM) | | |
| entry | Mean | Std Err | Mean | Std Err | % of opt |
| 65-5 | 902 | 35 | 244 | 22 | 27% |
| WT | 854 | 102 | 156 | 17 | 18% |
| hwe116 | 1006 | 51 | 376 | 37 | 37% |
| parent | 836 | 59 | 208 | 47 | 25% |

These results confirmed that the hwe116 mutant grew better on limited nitrogen and the transgenic line showed the same trends. Therefore, down-regulation of the PK220 gene in plants appears to result in increased nitrogen use efficiency (accumulation of more biomass per unit of available nitrogen).

The Transgenic Line of 35S-HP-At(270)PK220 Arabidopsis and the Hwe116 Mutant Germinate Faster and have Higher Rates of Germination in the Cold.

Germination under cold (10° C.) conditions was assessed in the transgenic line 65-5 carrying the 35S-HP-At(270)PK220 construct relative to WT-control and that of the hwe116 mutant relative to its parental control on agar plates containing optimal growth media. Four plates per entry with 30 seeds each were prepared and placed in the chamber at 10° C., 18 hr light (200 uE). Germination (emergence of the radicle) scored as a percentage of viable seeds, was noted twice daily for 5 days starting with day 5 from placing of seeds on plates (no germination before day 5). Once no further changes were observed in germination all plates were placed in a chamber at 22° C. to check for viability of the seeds that had not germinated. All entries showed 98 to 100% seed viability, the hwe116 mutant had 94%. viability. The results of the germination assessment at 10° C. (Table 16) indicate that the transgenic line 65-5 germinated sooner than it's WT-control. The hwe116 mutant had higher rates of germination in the cold than its parent control. These data, together with the evidence that the mutant grows better under cold conditions are indicative of a greater seed and seedling vigor under cold stress

TABLE 16
percentage germination of viable seeds at 10° C.

| | | Hours @ 10° C. | | | | | | | | | | % Viable |
|---|---|---|---|---|---|---|---|---|---|---|---|---|
| entry | #reps | 114.5 | 121 | 139 | 145 | 163 | 169 | 188.5 | 212.5 | 235 | 241 | Seed |
| 65-5 | 4 | 15.1 | 32.8 | 75.7 | 80.7 | 90.0 | 90.8 | 90.8 | 92.5 | 93.3 | 94.2 | 99.2 |
| WT | 4 | 5.9 | 16.0 | 55.4 | 62.9 | 78.1 | 79.0 | 80.7 | 80.7 | 81.5 | 81.5 | 98.4 |
| hwe116 | 4 | 15.9 | 28.4 | 67.5 | 81.4 | 94.2 | 98.0 | 99.0 | 99.0 | 100.0 | 100.0 | 94.0 |
| Parent | 4 | 6.7 | 26.7 | 72.5 | 77.5 | 85.0 | 85.0 | 85.9 | 85.9 | 85.9 | 85.9 | 100.0 |

### Gas Exchange Measurements Support Higher WUE in Transgenic 35S-HP-At(270)PK220 Arabidopsis Under Optimal Conditions

Plants of two transgenic lines and WT were grown in four inch diameter pots (one per pot) under optimal conditions in a growth chamber at 18 hr light (200 uE), 22° C., 60% RH. Eight days from first open flower gas exchange measurements were made on the youngest, fully developed leaf of 10 to 11 replicates per entry. Photosynthesis and transpiration rates were measured inside the growth chamber at the ambient growth light and temperature conditions and 400 ppm carbon dioxide using Li-6400 and Arabidopsis leaf cuvette. From the ratio of photosynthesis to transpiration instantaneous water use efficiency (WUE) was calculated. The results are shown in Table 17. The WUE in the transgenic lines was 11 and 18% greater than that of the WT. This data is consistent with the WUE measurements over a period of few days using the ratio of biomass accumulated to water lost in transpiration.

TABLE 17
Photosynthesis (umol carbon dioxide/m2/s), transpiration (mmol
H2O/m2/s) and WUE measured under optimal growth conditions.

| | Phots. | | Trans. | | WUE | |
|---|---|---|---|---|---|---|
| | (umol/ | Photos. | (mmol/ | Trans. | (Photos/ | WUE |
| entry | m2/s) | (% WT) | m2/s) | (% WT) | Trans) | (% WT) |
| 59-6 | 3.9 ± 0.2 | 105% | 4.2 ± 0.5 | 95% | 1.03 ± 0.11 | 118% |
| 65-5 | 3.6 ± 0.2 | 97% | 3.8 ± 0.4 | 86% | 0.97 ± 0.13 | 111% |
| WT | 3.7 ± 0.2 | | 4.4 ± 0.2 | | 0.87 ± 0.05 | |

### Drought Tolerance of 35S-HP-At(270)PK220 Transgenic Arabidopsis Results in Seed Yield and Biomass Protection Following Drought Stress.

Plants of two transgenic lines and the WT were grown (5 per 3 inch pot containing equal amount of soil) under optimal conditions in a growth chamber (22 C, 18 hr light of 200 uE, 60% RH) until first open flower. At first flower the drought treatment was applied to half of the plants while the other half was maintained under optimal conditions until maturity. The drought treatment consisted of watering all the plants to the same saturated water level. Plants were then weighed daily to monitor water loss from the pots and their water content was equalized daily by watering all pots to the level of the heaviest pot. As a result the soil water content was declining and reached stress levels with plants wilting on day 4. Plants were maintained at that stress level for another 2 days and on day 6 all plants were re-watered and maintained under optimal conditions for the rest of their life cycle. At maturity both optimal and drought plants were harvested for seed and shoot biomass. The impact of drought stress on both seed yield and shoot biomass was determined by comparing the optimal and drought treated plants. The results are shown in Table 18. Under optimal conditions the seed yield and the final shoot biomass of the transgenic lines was 7 to 10% higher than that of the WT. Following the drought stress during flowering the reduction in seed yield and the shoot biomass were not as great in transgenic plants as in the WT, resulting in seed yield protection of 5-7% and shoot biomass protection of 4%. The protection was calculated as the difference between the transgenics and WT in seed yield or shoot biomass a percentage of optimal.

TABLE 18
Seed yield and final shoot biomass from
optimal and drought stressed plants, n = 10

| | | | Seed | | | |
|---|---|---|---|---|---|---|
| | Seed | Shoot | yield- | | Shoot | |
| | yield- | DW- | drought | % of | DW- | % of |
| entry | opt (g) | opt (g) | (g) | opt | drought (g) | opt |
| 59-6 | 1.29 ± 0.05 | 2.96 ± 0.13 | 1.06 ± 0.03 | 82% | 2.37 ± 0.07 | 80% |
| 65-5 | 1.27 ± 0.03 | 2.89 ± 0.08 | 1.01 ± 0.02 | 80% | 2.32 ± 0.06 | 80% |
| WT | 1.18 ± 0.04 | 2.69 ± 0.10 | 0.89 ± 0.02 | 75% | 2.04 ± 0.05 | 76% |

### Over-Expression of Wild Type AtPK220 in Hwe116.2 Background can Restore the WT Phenotype

Transgenic plants of 35S-AtPK220 (in hwe116.2) were grown (5 per 3 inch pot) under optimal conditions in a growth chamber as described above until the first open flower. Drought treatment was applied by watering all plants to the same saturated level. Further watering was withheld. Plants were weighed daily to determine the daily water loss and all plants were harvested on day 4 of treatment by which time all plants showed wilting. The water loss relative to final shoot biomass was used to calculate drought tolerance where that of WT was assumed at 100%. The data are shown in Table 19. Three transgenic lines showed a reduction in drought tolerance from the mutant levels as indicated by increased water loss relative to shoot biomass. The three transgenic lines also flowered earlier than the mutant line and similar to the time that the WT lines flowered. These results support the conclusion that the AtPK220 gene mutation in hwe116.2 is responsible for the altered phenotypes observed and expression of a WT gene restore the WT characteristics of a mutant plant.

TABLE 19
Water loss relative to shoot biomass and drought tolerance, n = 8

| | | Water lost in | Drought tolerance |
|---|---|---|---|
| entry | Days to flower | 3 d/shoot DW d4 | (% of WT) |
| 28-4 | 20.9 ± 0.1 | 155.1 ± 3.1 | 111% |
| 2-4 | 21.8 ± 0.1 | 164.7 ± 2.4 | 105% |
| 7-11 | 21.6 ± 0.1 | 177.9 ± 4.4 | 97% |
| hwe116.2 | 23.1 ± 0.2 | 134.9 ± 3.6 | 117% |
| WT | 20.8 ± 0.2 | 173.4 ± 5.1 | 100% |

Down Regulation of AtPK220 with the AtPK220-Promoter (P_{PK}) in Arabidopsis Results in Enhanced Drought Tolerance of Plants

Arabidopsis plants of P_{PK}-HP-At(270)PK220 were grown (5 per 3 inch pot) under optimal conditions in a growth chamber as mentioned above until the first open flower. Drought treatment was applied then by watering all plants to the same saturated level. Further water was withheld. Plants were weighed daily to determine the daily water loss and all plants were harvested on day 4 of treatment (all plants were wilted). The water loss relative to final shoot biomass was used to calculate drought tolerance where that of WT was assumed at 100%. The results of this study are shown in Table 20.

TABLE 20
Water loss relative to shoot biomass and drought tolerance, n = 8

| | | Water lost in | Drought tolerance |
|---|---|---|---|
| entry | Days to flower | 3 d/shootDW d4 | (% WT) |
| 14-04 | 22 | 158 ± 5 | 116% |
| 15-06 | 20 | 183 ± 8 | 104% |
| 45-3 | 20 | 185 ± 9 | 103% |
| WT | 20 | 190 ± 9 | 100% |

One of the transgenic lines, 14-04, showed significantly greater drought tolerance than the wild type control as indicated by lower water loss relative to shoot biomass. This result is supported by data from line 14-04 that showed nearly complete inhibition of PK220 gene expression. The expression of AtPK220 was reduced by nearly 96% in the roots compared to WT. These results indicate that down regulation of PK220 in the roots is sufficient to achieve significant drought tolerance phenotype and presumably enhanced water use efficiency.

Overexpression of Brassica napus PK220 in the Arabidopsis Hwe116 Mutant can Restore the WT Phenotype

Transgenic plants of 35S-BnPK220 (in hwe116) plus two null controls (segregated siblings of the transgenic lines without the transgene, therefore hwe116 mutant) were grown (5 per 3 inch pot) under optimal conditions in a growth chamber as mentioned above until the first open flower. Drought treatment was applied then by watering all plants to the same saturated level. Further water was withheld. Plants were weighed daily to determine the daily water loss and all plants were harvested on day 4 of treatment (all plants were wilted). The water loss relative to final shoot biomass was used to calculate drought tolerance where that of WT was assumed at 100%. The results of this study are shown in Table 21. The results indicate that 6 lines had a reduction of 8% or more in drought tolerance as compared to the nulls (the hwe116 mutant background) and therefore restoration towards the WT phenotype. This indicates that BnPK220 is functional and can work in the Arabidopsis.

TABLE 21
Water loss relative to shoot DW and drought tolerance, n = 8

| | Water lost in | Drought tolerance |
|---|---|---|
| entry | 3 d/shoot DW d4 | (% of null) |
| 106-11 | 148 ± 6 | 98% |
| 67-6 | 150 ± 4 | 97% |
| 51-6 | 152 ± 4 | 96% |
| 5-1 | 152 ± 2 | 95% |
| 74-12 | 157 ± 5 | 92% |
| 38-7 | 160 ± 5 | 90% |
| 70-2 | 161 ± 2 | 89% |
| 97-3 | 164 ± 5 | 87% |
| 31-6 | 165 ± 4 | 87% |
| 93-8 | 172 ± 4 | 82% |
| Null 38-10 | 146 ± 3 | 100% |
| Null 90-7 | 135 ± 5 | 107% |

### Transgenic Brassica Lines Having a 35S-AtPK220L292F Construct Showed Drought Tolerance and Higher Water Use Efficiency

Down regulation of endogenous PK220 activity was demonstrated using a dominant negative strategy by expression of the mutant allele of the AtPK220 gene in Brassica napus. Three Brassica napus transgenic lines having the Arabidopsis mutant AtPK220L292F gene and one null control line (a segregated sibling of the transgenic line lacking the transgene) per line were grown in 4.5 inch diameter pots containing equal amounts of soilless mix (Sunshine Professional Organic Mix #7) under optimal conditions of 16 hr light (400 uE) and 22 C day/18 C night temperature. At the four leaf stage, two treatments were applied. In the optimal treatment plants were watered to saturation and pots were covered with plastic bags to prevent any water loss from the pots due to evaporation. These plants were weighed daily for 7 days to determine the water loss from the pots due to transpiration and the same amount of water was added back daily to each pot to maintain the plants under optimal water conditions. In the drought treatment all plants were watered to saturation levels. Pots were covered with plastic and were weighed daily. However, these pots were watered daily to the level of the heaviest pots. This treatment went for 7 days with the soil water content gradually reaching stress levels. Plants started to wilt by day 5. At the end of the 7 days both groups of plants were harvested for shoot biomass determinations.

Gas exchange measurements were done on drought treated plants of two transgenic lines plus their nulls on days 3 and 4 of the treatment. Photosynthesis and transpiration were measured on leaf 3 under steady state growth conditions of 400 uE light, 400 ppm carbon dioxide and 22 C using Li-6400. From the ratio of photosynthesis to transpiration, water use efficiency (WUE) was calculated. The drought treated plants were used to calculate the drought tolerance (as percentage of their nulls). This was done using the ratio of cumulative daily transpirational water loss between days 3 and 7, relative to the final shoot dry weight and normalizing it to the nulls (set at 100%).

The results in Table 22 indicate that transgenic lines had strong trends toward greater drought tolerance. This was a result of lower water loss relative to shoot dry weight, a phenotype present also under optimal conditions.

The gas exchange data (Table 23) showed that on both days 3 and 4 of the drought treatment the transgenic plants had slightly higher WUE than controls (4 to 16%).

Water use efficiency calculated from the ratio of photosynthesis to transpiration provides only a single point, instantaneous measurement rather than cumulative measurement over the period of treatment and as a result may be of lesser magnitude.

In conclusion, the data with transgenic 35S-AtPK220L292F Brassica plants indicate that water use efficiency technology is transferable to Brassica when using a AtPK220L292F gene from a heterologous species.

TABLE 22
Water loss between days 3 and 7 relative to final shoot
dry weight under optimal and drought treatment. Drought
tolerance (% of the appropriate null). n = 8

| | optimal-g | drought-g | |
|---|---|---|---|
| | water lost | water lost | |
| | d3-7/g | d3-7/g | Drought tolerance |
| entry | shootDW d7 | shootDW d7 | (% of null) |
| Tr-05 | 172 ± 6 | 121 ± 5 | 109% |
| Null-05 | 190 ± 4 | 133 ± 7 | 100% |
| Tr-27 | 194 ± 6 | 134 ± 7 | 113% |
| Null-27 | 205 ± 8 | 155 ± 11 | 100% |
| Tr-09 | 171 ± 10 | 129 ± 3 | 113% |
| Null-09 | 178 ± 17 | 149 ± 13 | 100% |

TABLE 23
Photosynthesis (umol
carbon dioxide/m2/s), Transpiration (mmol H2O/m2/s) and
WUE (Photos/Trans on days 3 and 4 of drought treatment. n = 8

| | Photos | Trans. | WUE | Photos. | Trans. | WUE |
|---|---|---|---|---|---|---|
| entry | D3 | D3 | D3 | D4 | D4 | D4 |
| Tr-05 | 13.4 ± 1.2 | 2.1 ± 0.2 | 6.6 ± 0.2 | 11.6 ± 1.3 | 1.9 ± 0.2 | 6.1 ± 0.4 |
| | | | (116% | | | (104% |
| | | | of null) | | | of null) |
| Null-05 | 14.4 ± 1.1 | 2.5 ± 0.2 | 5.7 ± 0.2 | 12.6 ± 1.2 | 2.2 ± 0.2 | 5.9 ± 0.3 |
| Tr-27 | 14.1 ± 0.7 | 2.4 ± 0.2 | 5.9 ± 0.3 | 11.4 ± 1.6 | 1.9 ± 0.3 | 6.2 ± 0.5 |
| | | | (105% | | | (108% |
| | | | of null) | | | of null) |
| Null-27 | 14.1 ± 1.3 | 2.5 ± 0.1 | 5.6 ± 0.5 | 13.7 ± 1.0 | 2.4 ± 0.1 | 5.7 ± 0.4 |

SEQUENCE ID REFERENCE CHART

| SPECIES | SEQ ID NO: | REFERENCE | | |
|---|---|---|---|---|
| ARABIDOPSIS THALIANA | SEQIDNO: 1 | AtPK220 | NT | 1299 |
| ARABIDOPSIS THALIANA | SEQIDNO: 2 | AtPK220 | AA | 432 |
| ARABIDOPSIS THALIANA | SEQIDNO: 3 | AtPK220L292F | NT | 1299 |
| ARABIDOPSIS THALIANA | SEQIDNO: 4 | AtPK220L292F | AA | 432 |
| ARABIDOPSIS THALIANA | SEQIDNO: 5 | AtPK220L292F_partial | NT | 1160 |
| ARABIDOPSIS THALIANA | SEQIDNO: 6 | AtPK220L292F_partial_orf | AA | 383 |
| ARABIDOPSIS THALIANA | SEQIDNO: 7 | AtPK220_partial | NT | 1160 |
| ARABIDOPSIS THALIANA | SEQIDNO: 8 | AtPK220_partial_orf | AA | 383 |
| ARABIDOPSIS THALIANA | SEQIDNO: 9 | AtPK220_with_UTR | NT | 1542 |
| ARABIDOPSIS THALIANA | SEQIDNO: 10 | AtPK220_for_35s-AtPK220 | NT | 1309 |
| ARABIDOPSIS THALIANA | SEQIDNO: 11 | AtPK220_partial | NT | 1177 |
| ARABIDOPSIS THALIANA | SEQIDNO: 12 | At(150)PK | NT | 154 |
| ARABIDOPSIS THALIANA | SEQIDNO: 13 | At(270)PK | NT | 288 |
| ARABIDOPSIS THALIANA | SEQIDNO: 14 | AtPK220_promoter | NT | 1510 |
| ARABIDOPSIS THALIANA | SEQIDNO: 15 | At4g32000_UTR | NT | 157 |
| ARABIDOPSIS THALIANA | SEQIDNO: 16 | At4g32000 | NT | 1257 |
| ARABIDOPSIS THALIANA | SEQIDNO: 17 | At4g32000 | AA | 418 |
| ARABIDOPSIS THALIANA | SEQIDNO: 18 | At5g11020 | NT | 1302 |
| ARABIDOPSIS THALIANA | SEQIDNO: 19 | At5g11020 | AA | 433 |
| ARABIDOPSIS THALIANA | SEQIDNO: 20 | At2g25440 | NT | 2016 |
| ARABIDOPSIS THALIANA | SEQIDNO: 21 | At2g25440 | AA | 671 |
| ARABIDOPSIS THALIANA | SEQIDNO: 22 | At2g23890 | NT | 1662 |
| ARABIDOPSIS THALIANA | SEQIDNO: 23 | At2g23890 | AA | 553 |
| BRACHYPODIUM | SEQIDNO: 24 | BdPK220 | NT | 1386 |
| DISTACHYON | | | | |
| BRASSICA NAPUS | SEQIDNO: 25 | BnPK220 | NT | 1302 |
| BRASSICA NAPUS | SEQIDNO: 26 | BnPK220 | AA | 433 |
| CICHORIUM ENDIVIA | SEQIDNO: 27 | EL362007.1 | NT | 657 |
| CICHORIUM ENDIVIA | SEQIDNO: 28 | EL362007.1_ORF | AA | 218 |
| CITRUS CLEMENTINA | SEQIDNO: 29 | CX290402.1 | NT | 474 |
| CITRUS CLEMENTINA | SEQIDNO: 30 | CX290402.1_ORF | AA | 157 |
| CITRUS SINENSIS | SEQIDNO: 31 | CK934154.1 | NT | 770 |
| CITRUS SINENSIS | SEQIDNO: 32 | CK934154.1_ORF | AA | 257 |
| COFFEA CANEPHORA | SEQIDNO: 33 | DV708241.1 | NT | 621 |
| COFFEA CANEPHORA | SEQIDNO: 34 | DV708241.1_ORF | AA | 206 |
| EUCALYPTUS GUNNII | SEQIDNO: 35 | CT986101.1 | NT | 411 |
| EUCALYPTUS GUNNII | SEQIDNO: 36 | CT986101.1_ORF | AA | 136 |
| FESTUCA ARUNDINACEA | SEQIDNO: 37 | DT714073 | NT | 522 |
| FESTUCA ARUNDINACEA | SEQIDNO: 38 | DT714073_ORF | AA | 173 |
| GINKGO BILOBA | SEQIDNO: 39 | EX942240.1 | NT | 740 |
| GINKGO BILOBA | SEQIDNO: 40 | EX942240.1_ORF | AA | 247 |
| GLYCINE MAX | SEQIDNO: 41 | GmPK220 | NT | 1254 |
| GLYCINE MAX | SEQIDNO: 42 | GmPK220 | AA | 418 |
| HELIANTHUS | SEQIDNO: 43 | EE622910.1 | NT | 702 |
| ARGOPHYLLUS | | | | |
| HELIANTHUS | SEQIDNO: 44 | EE622910.1_ORF | AA | 233 |
| ARGOPHYLLUS | | | | |
| HELIANTHUS CILIARIS | SEQIDNO: 45 | EL429543.1 | NT | 752 |
| HELIANTHUS CILIARIS | SEQIDNO: 46 | EL429543.1_ORF | AA | 251 |
| HELIANTHUS EXILIS | SEQIDNO: 47 | EE654885.1 | NT | 630 |
| HELIANTHUS EXILIS | SEQIDNO: 48 | EE654885.1_ORF | AA | 209 |
| HORDEUM VULGARE | SEQIDNO: 49 | TC151622 | NT | 780 |
| HORDEUM VULGARE | SEQIDNO: 50 | TC151622_ORF | AA | 259 |
| IPOMOEA BATATAS | SEQIDNO: 51 | EE883089.1 | NT | 816 |
| IPOMOEA BATATAS | SEQIDNO: 52 | EE883089.1_ORF | AA | 272 |
| LACTUCA SATIVA | SEQIDNO: 53 | DW125133.1 | NT | 867 |
| LACTUCA SATIVA | SEQIDNO: 54 | DW125133.1_ORF | AA | 288 |
| MEDICAGO TRUNCATULA | SEQIDNO: 55 | Contig | NT | 804 |
| MEDICAGO TRUNCATULA | SEQIDNO: 56 | Contig | AA | 267 |
| NICOTIANA TABACUM | SEQIDNO: 57 | BP131484.1 | NT | 636 |
| NICOTIANA TABACUM | SEQIDNO: 58 | BP131484.1 | AA | 211 |
| ORYZA SATIVA | SEQIDNO: 59 | NM_001061720.1 | NT | 1437 |
| ORYZA SATIVA | SEQIDNO: 60 | NP_001055185.1 | AA | 478 |
| PHYSCOMITRELLA | SEQIDNO: 61 | EDQ75046.1_cds | NT | 891 |
| PHYSCOMITRELLA | SEQIDNO: 62 | EDQ75046.1 | AA | 297 |
| PICEA | SEQIDNO: 63 | TC12392 | NT | 1065 |
| PICEA | SEQIDNO: 64 | TC12392_orf | AA | 354 |
| PINUS | SEQIDNO: 65 | CT578985.1 | NT | 596 |
| PINUS | SEQIDNO: 66 | CT578985.1_ORF | AA | 199 |
| POPULUS | SEQIDNO: 67 | TC76879 | NT | 1377 |
| POPULUS | SEQIDNO: 68 | TC76879_ORF | AA | 459 |
| SACCHARUM | SEQIDNO: 69 | TC46535 | NT | 693 |
| OFFICINARUM | | | | |
| SACCHARUM | SEQIDNO: 70 | TC46535_ORF | AA | 230 |
| OFFICINARUM | | | | |
| TRIPHYSARIA | SEQIDNO: 71 | DR169688.1 | NT | 414 |
| VERSICOLOR | | | | |
| TRIPHYSARIA | SEQIDNO: 72 | DR169688.1_ORF | AA | 137 |
| VERSICOLOR | | | | |
| TRITICUM AESTIVUM | SEQIDNO: 73 | TC254793 | NT | 1140 |
| TRITICUM AESTIVUM | SEQIDNO: 74 | TC254793_ORF | AA | 380 |
| VITIS VINIFERA | SEQIDNO: 75 | CAO44295.1_cds | NT | 978 |
| VITIS VINIFERA | SEQIDNO: 76 | CAO44295.1 | AA | 325 |
| ZEA MAYS | SEQIDNO: 77 | TC333547 | NT | 1377 |
| ZEA MAYS | SEQIDNO: 78 | TC333547_ORF | AA | 458 |
| ZEA MAYS | SEQIDNO: 79 | ZmPK220 | NT | 1188 |
| ZEA MAYS | SEQIDNO: 80 | ZmPK220 | AA | 396 |
| GOSSYPIUM | SEQIDNO: 81 | TC79117 | NT | 1086 |
| GOSSYPIUM | SEQIDNO: 82 | TC79117_ORF | AA | 361 |
| SOLANUM | SEQIDNO: 83 | Contig3 | NT | 1089 |
| LYCOPERSICUM | | | | |
| AQUILEGIA | SEQIDNO: 84 | DR918821 | NT | 875 |
| AQUILEGIA | SEQIDNO: 85 | DR918821_ORF | AA | 292 |
| CENTAUREA MACULOSA | SEQIDNO: 86 | EL933228.1 | NT | 696 |
| CENTAUREA MACULOSA | SEQIDNO: 87 | EL933228.1_ORF | AA | 231 |
| CICHORIUM INTYBUS | SEQIDNO: 88 | EH693146.1 | NT | 842 |
| CICHORIUM INTYBUS | SEQIDNO: 89 | EH693146.1_ORF | AA | 281 |
| CUCUMIS MELO | SEQIDNO: 90 | AM742189.1 | NT | 495 |
| CUCUMIS MELO | SEQIDNO: 91 | AM742189.1_ORF | AA | 164 |
| ERAGROSTIS CURVULA | SEQIDNO: 92 | EH186232.1 | NT | 375 |
| ERAGROSTIS CURVULA | SEQIDNO: 93 | EH186232.1_ORF | AA | 124 |
| GERBERA HYBRID | SEQIDNO: 94 | AJ753651.1 | NT | 414 |
| GERBERA HYBRID | SEQIDNO: 95 | AJ753651.1_ORF | AA | 137 |
| HELIANTHUS PARADOXUS | SEQIDNO: 96 | EL488199.1 | NT | 498 |
| HELIANTHUS PARADOXUS | SEQIDNO: 97 | EL488199.1_ORF | AA | 165 |
| IPOMOEA NIL | SEQIDNO: 98 | BJ566706.1 | NT | 612 |
| IPOMOEA NIL | SEQIDNO: 99 | BJ566706.1_ORF | AA | 203 |
| NUPHAR ADVENA | SEQIDNO: 100 | DT603238.1 | NT | 708 |
| NUPHAR ADVENA | SEQIDNO: 101 | DT603238.1_ORF | AA | 235 |
| SYNTHETIC PRIMER | SEQIDNO: 102 | 747F | NT | 30 |
| SYNTHETIC PRIMER | SEQIDNO: 103 | 747R | NT | 34 |
| SYNTHETIC PRIMER | SEQIDNO: 104 | C747F2 | NT | 32 |
| SYNTHETIC PRIMER | SEQIDNO: 105 | C747R2 | NT | 31 |
| SYNTHETIC PRIMER | SEQIDNO: 106 | A220BamF1 | NT | 42 |
| SYNTHETIC PRIMER | SEQIDNO: 107 | A220PstR | NT | 40 |
| SYNTHETIC PRIMER | SEQIDNO: 108 | K188R | NT | 30 |
| SYNTHETIC PRIMER | SEQIDNO: 109 | A220A1SmaF2 | NT | 53 |
| SYNTHETIC PRIMER | SEQIDNO: 110 | A220BamR | NT | 38 |
| SYNTHETIC PRIMER | SEQIDNO: 111 | A220SmaF | NT | 41 |
| SYNTHETIC PRIMER | SEQIDNO: 112 | A220BamF2 | NT | 41 |
| SYNTHETIC PRIMER | SEQIDNO: 113 | A220XbaR | NT | 39 |
| SYNTHETIC PRIMER | SEQIDNO: 114 | K116SacF | NT | 35 |
| SYNTHETIC PRIMER | SEQIDNO: 115 | K270SacR | NT | 37 |
| SYNTHETIC PRIMER | SEQIDNO: 116 | K116BamF | NT | 37 |
| SYNTHETIC PRIMER | SEQIDNO: 117 | K270XbaR | NT | 40 |
| SYNTHETIC PRIMER | SEQIDNO: 118 | PK81A1XbaF | NT | 52 |
| SYNTHETIC PRIMER | SEQIDNO: 119 | K81PmBamF | NT | 47 |
| SYNTHETIC PRIMER | SEQIDNO: 120 | Pm81SmaR2 | NT | 41 |
| ARABIDOPSIS THALIANA | SEQIDNO: 121 | AtPK220L292F_with_UTR | NT | 1309 |
| SYNTHETIC PRIMER | SEQIDNO: 122 | Bn81F | NT | 25 |
| SYNTHETIC PRIMER | SEQIDNO: 123 | Bn81R | NT | 32 |
| SYNTHETIC PRIMER | SEQIDNO: 124 | Bn81RAF1 | NT | 32 |
| SYNTHETIC PRIMER | SEQIDNO: 125 | Bn81RAF2 | NT | 32 |
| SYNTHETIC PRIMER | SEQIDNO: 126 | Bn81RAR1 | NT | 31 |
| SYNTHETIC PRIMER | SEQIDNO: 127 | Bn81RAR2 | NT | 37 |
| SYNTHETIC PRIMER | SEQIDNO: 128 | Bn81F1 | NT | 28 |
| SYNTHETIC PRIMER | SEQIDNO: 129 | Bn81R1 | NT | 27 |
| SYNTHETIC PRIMER | SEQIDNO: 130 | Gm81RAR1 | NT | 27 |
| SYNTHETIC PRIMER | SEQIDNO: 131 | Gm81RAR2 | NT | 29 |
| SYNTHETIC PRIMER | SEQIDNO: 132 | Cn81RAR1 | NT | 29 |
| SYNTHETIC PRIMER | SEQIDNO: 133 | Cn81RAR2 | NT | 28 |
| SYNTHETIC PRIMER | SEQIDNO: 134 | A220SacF | NT | 41 |
| SYNTHETIC PRIMER | SEQIDNO: 135 | Pm81NheF | NT | 47 |
| SYNTHETIC PRIMER | SEQIDNO: 136 | Pm81NheR | NT | 43 |
| SYNTHETIC PRIMER | SEQIDNO: 137 | Gm81RAF1 | NT | 29 |
| SYNTHETIC PRIMER | SEQIDNO: 138 | Gm81RAF2 | NT | 31 |
| SYNTHETIC PRIMER | SEQIDNO: 139 | Zm81RAF1 | NT | 31 |
| SYNTHETIC PRIMER | SEQIDNO: 140 | Zm81RAF2 | NT | 27 |
| SYNTHETIC PRIMER | SEQIDNO: 141 | MiR319XbaF | NT | 31 |
| SYNTHETIC PRIMER | SEQIDNO: 142 | MiR319BamR | NT | 33 |
| SYNTHETIC PRIMER | SEQIDNO: 143 | MiPK220F1 | NT | 40 |
| SYNTHETIC PRIMER | SEQIDNO: 144 | MiPK220R1 | NT | 35 |
| SYNTHETIC PRIMER | SEQIDNO: 145 | MiPK220F2 | NT | 35 |
| SYNTHETIC PRIMER | SEQIDNO: 146 | MiPK220R2 | NT | 42 |
| ARTIFICIAL SEQUENCE | SEQIDNO: 147 | Synthesized_gene_fragment | NT | 21 |
| ARABIDOPSIS THALIANA | SEQIDNO: 148 | At4g23713_w_genomic | NT | 399 |
| ARTIFICIAL SEQUENCE | SEQIDNO: 149 | Artificial_micro_RNA_construct | NT | 399 |
| ARABIDOPSIS THALIANA | SEQIDNO: 150 | Promoter At2g44790 | NT | 1475 |
| SYNTHETIC PRIMER | SEQIDNO: 151 | P790-H3-F | NT | 31 |
| SYNTHETIC PRIMER | SEQIDNO: 152 | P790-Xb-R | NT | 31 |
| BRASSICA NAPUS | SEQIDNO: 153 | BnPK220 | NT | 338 |
| SYNTHETIC PRIMER | SEQIDNO: 154 | Bn340BamF | NT | 38 |
| SYNTHETIC PRIMER | SEQIDNO: 155 | Bn340XbaR | NT | 37 |
| SYNTHETIC PRIMER | SEQIDNO: 156 | Bn340SacF | NT | 38 |
| SYNTHETIC PRIMER | SEQIDNO: 157 | Bn340SacR | NT | 37 |
| SYNTHETIC PRIMER | SEQIDNO: 158 | bWET Xbal F | NT | 24 |
| SYNTHETIC PRIMER | SEQIDNO: 159 | bWET BamHI R | NT | 28 |
| SYNTHETIC PRIMER | SEQIDNO: 160 | bWET Clal R | NT | 28 |
| BRACHYPODIUM | SEQIDNO: 161 | BdPK220 | NT | 272 |
| DISTACHYON | | | | |
| SYNTHETIC PRIMER | SEQIDNO: 162 | bWx BamHI F | NT | 31 |
| SYNTHETIC PRIMER | SEQIDNO: 163 | bWx Clal R | NT | 30 |
| BRACHYPODIUM | SEQIDNO: 164 | BdWx intron 1 | NT | 1174 |
| DISTACHYON | | | | |
| SYNTHETIC PRIMER | SEQIDNO: 165 | bWET BamHI end2 | NT | 22 |
| SYNTHETIC PRIMER | SEQIDNO: 166 | BdUBQ Pvul F | NT | 30 |
| SYNTHETIC PRIMER | SEQIDNO: 167 | BdUBQT Pacl R | NT | 28 |
| PANICUM VIRGATUM | SEQIDNO: 168 | Pv(251)PK220 | NT | 251 |
| SYNTHETIC PRIMER | SEQIDNO: 169 | PvWET Xbal F | NT | 27 |
| SYNTHETIC PRIMER | SEQIDNO: 170 | PvWET BamHI R | NT | 27 |
| SYNTHETIC PRIMER | SEQIDNO: 171 | PvWET Clal R | NT | 27 |
| SYNTHETIC PRIMER | SEQIDNO: 172 | PvWET BamHI end1 | NT | 25 |
| SYNTHETIC PRIMER | SEQIDNO: 173 | PvWET BamHI end2 | NT | 21 |
| SORGHUN BICOLOR | SEQIDNO: 174 | Sb(261)PK220 | NT | 261 |
| SYNTHETIC PRIMER | SEQIDNO: 175 | SbWET Xbal F | NT | 26 |
| SYNTHETIC PRIMER | SEQIDNO: 176 | SbWET BamHI R | NT | 28 |
| SYNTHETIC PRIMER | SEQIDNO: 177 | SbWET Clal R | NT | 28 |
| SORGHUN BICOLOR | SEQIDNO: 178 | SbWx intron 1 | NT | 273 |
| SYNTHETIC PRIMER | SEQIDNO: 179 | SbWx BamHI | NT | 30 |
| SYNTHETIC PRIMER | SEQIDNO: 180 | SbWx Clal R | NT | 34 |
| SYNTHETIC PRIMER | SEQIDNO: 181 | SbWET BamHI end1 | NT | 24 |
| SYNTHETIC PRIMER | SEQIDNO: 182 | SbWET BamHI end2 | NT | 21 |
| SORGHUN BICOLOR | SEQIDNO: 183 | SbGOS2 promoter | NT | 1000 |
| SYNTHETIC PRIMER | SEQIDNO: 184 | SbGOS2 HindIII F | NT | 28 |
| SYNTHETIC PRIMER | SEQIDNO: 185 | SbGOS2 HindIII R | NT | 30 |
| SORGHUN BICOLOR | SEQIDNO: 186 | SbUBQ promoter | NT | 1000 |
| SYNTHETIC PRIMER | SEQIDNO: 187 | SbUBQ Pstl F | NT | 26 |
| SYNTHETIC PRIMER | SEQIDNO: 188 | SbUBQ Pstl R | NT | 28 |
| SORGHUN BICOLOR | SEQIDNO: 189 | SbUBQ terminator | NT | 239 |
| SYNTHETIC PRIMER | SEQIDNO: 190 | SbUBQT Kpnl F | NT | 27 |
| SYNTHETIC PRIMER | SEQIDNO: 191 | SbUBQT Kpnl R | NT | 27 |
| SYNTHETIC PRIMER | SEQIDNO: 192 | SbUBQ Pvul F | NT | 34 |
| BRASSICA NAPUS | SEQIDNO: 193 | BnPK220 | NT | 1302 |
| BRASSICA NAPUS | SEQIDNO: 194 | BnPK220 | AA | 433 |
| SYNTHETIC PRIMER | SEQIDNO: 195 | Bd81RAR1 | NT | 31 |
| SYNTHETIC PRIMER | SEQIDNO: 196 | Bd81RAR2 | NT | 32 |
| BRACHYPODIUM | SEQIDNO: 197 | BdPK220 | AA | 461 |
| DISTACHYON | | | | |
| SYNTHETIC PRIMER | SEQIDNO: 198 | A200A1AgeF | NT | 53 |
| SYNTHETIC PRIMER | SEQIDNO: 199 | A220AgeR | NT | 39 |
| SYNTHETIC PRIMER | SEQIDNO: 200 | bWET BamHI end1 | NT | 18 |

Sequences
>SEQ ID NO: 1
ATGAGAGAGCTTCTTCTTCTTCTTCTTCTTCATTTTCAGTCTCTAATTCTTTTGATGATCTTCATCACT
GTCTCTGCTTCTTCTGCTTCAAATCCTTCTTTAGCTCCTGTTTACTCTTCCATGGCTACATTCTCTCCT
CGAATCCAAATGGGAAGTGGTGAAGAAGATAGATTTGATGCTCATAAGAAACTTCTGATTGGTCTC
ATAATCAGTTTCTCTTCTCTTGGCCTTATAATCTTGTTCTGTTTTGGCTTTTGGGTTTATCGCAAGAA
CCAATCTCCAAAATCCATCAACAACTCAGATTCTGAGAGTGGGAATTCATTTTCCTTGTTAATGAGA
CGACTTGGCTCGATTAAAACTCAGAGAAGAACTTCTATCCAAAAGGGTTACGTGCAATTTTTCGAT
ATCAAGACCCTCGAGAAAGCGACAGGCGGTTTTAAAGAAAGTAGTGTAATCGGACAAGGCGGTTT
CGGATGCGTTTACAAGGGTTGTTTGGACAATAACGTTAAAGCAGCGGTCAAGAAGATCGAGAACG
TTAGCCAAGAAGCAAAACGAGAATTTCAGAATGAAGTTGACTTGTTGAGCAAGATCCATCACTCGA
ACGTTATATCATTGTTGGGCTCTGCAAGCGAAATCAACTCGAGTTTCATCGTTTATGAGCTTATGGA
GAAAGGATCATTAGATGAACAGTTACATGGGCCTTCTCGTGGATCAGCTCTAACATGGCACATGCG
TATGAAGATTGCTCTTGATACAGCTAGAGGACTAGAGTATCTCCATGAGCATTGTCGTCCACCAGT
TATCCACAGAGATTTGAAATCTTCGAATATTCTTCTTGATTCTTCCTTCAACGCCAAGATTTCAGAT
TTCGGTCTTGCTGTATCGCTGGATGAACATGGCAAGAACAACATTAAACTCTCTGGGACACTTGGT
TATGTTGCCCCGGAATACCTCCTTGACGGAAAACTGACGGATAAGAGTGATGTTTATGCATTTGGG
GTAGTTCTGCTTGAACTCTTGTTGGGTAGACGACCAGTTGAAAAATTAACTCCAGCTCAATGCCAA
TCTCTTGTAACTTGGGCAATGCCACAACTTACCGATAGATCCAAGCTTCCAAACATTGTGGATGCC
GTTATAAAAGATACAATGGATCTCAAACACTTATACCAGGTAGCAGCCATGGCTGTGTTGTGCGTG
CAGCCAGAACCAAGTTACCGGCCGTTGATAACCGATGTTCTTCACTCACTTGTTCCACTGGTTCCGG
TAGAGCTAGGAGGGACTCTCCGGTTAACAAGATGA
>SEQ ID NO: 2
MRELLLLLLLHFQSLILLMIFITVSASSASNPSLAPVYSSMATFSPRIQMGSGEEDRFDAHKKLLIGLIISFS
SLGLIILFCFGFWVYRKNQSPKSINNSDSESGNSFSLLMRRLGSIKTQRRTSIQKGYVQFFDIKTLEKATG
GFKESSVIGQGGFGCVYKGCLDNNVKAAVKKIENVSQEAKREFQNEVDLLSKIHHSNVISLLGSASEINS
SFIVYELMEKGSLDEQLHGPSRGSALTWHMRMKIALDTARGLEYLHEHCRPPVIHRDLKSSNILLDSSFN
AKISDFGLAVSLDEHGKNNIKLSGTLGYVAPEYLLDGKLTDKSDVYAFGVVLLELLLGRRPVEKLTPAQ
CQSLVTWAMPQLTDRSKLPNIVDAVIKDTMDLKHLYQVAAMAVLCVQPEPSYRPLITDVLHSLVPLVP
VELGGTLRLTR
>SEQ ID NO: 3
ATGAGAGAGCTTCTTCTTCTTCTTCTTCTTCATTTTCAGTCTCTAATTCTTTTGATGATCTTCATCACT
GTCTCTGCTTCTTCTGCTTCAAATCCTTCTTTAGCTCCTGTTTACTCTTCCATGGCTACATTCTCTCCT
CGAATCCAAATGGGAAGTGGTGAAGAAGATAGATTTGATGCTCATAAGAAACTTCTGATTGGTCTC
ATAATCAGTTTCTCTTCTCTTGGCCTTATAATCTTGTTCTGTTTTGGCTTTTGGGTTTATCGCAAGAA
CCAATCTCCAAAATCCATCAACAACTCAGATTCTGAGAGTGGGAATTCATTTTCCTTGTTAATGAGA
CGACTTGGCTCGATTAAAACTCAGAGAAGAACTTCTATCCAAAAGGGTTACGTGCAATTTTTCGAT
ATCAAGACCCTCGAGAAAGCGACAGGCGGTTTTAAAGAAAGTAGTGTAATCGGACAAGGCGGTTT
CGGATGCGTTTACAAGGGTTGTTTGGACAATAACGTTAAAGCAGCGGTCAAGAAGATCGAGAACG
TTAGCCAAGAAGCAAAACGAGAATTTCAGAATGAAGTTGACTTGTTGAGCAAGATCCATCACTCGA
ACGTTATATCATTGTTGGGCTCTGCAAGCGAAATCAACTCGAGTTTCATCGTTTATGAGCTTATGGA
GAAAGGATCATTAGATGAACAGTTACATGGGCCTTCTCGTGGATCAGCTCTAACATGGCACATGCG
TATGAAGATTGCTCTTGATACAGCTAGAGGACTAGAGTATCTCCATGAGCATTGTCGTCCACCAGT
TATCCACAGAGATTTGAAATCTTCGAATATTCTTCTTGATTCTTCCTTCAACGCCAAGATTTCAGAT
TTCGGTTTTGCTGTATCGCTGGATGAACATGGCAAGAACAACATTAAACTCTCTGGGACACTTGGTT
ATGTTGCCCCGGAATACCTCCTTGACGGAAAACTGACGGATAAGAGTGATGTTTATGCATTTGGGG
TAGTTCTGCTTGAACTCTTGTTGGGTAGACGACCAGTTGAAAAATTAACTCCAGCTCAATGCCAATC
TCTTGTAACTTGGGCAATGCCACAACTTACCGATAGATCCAAGCTTCCAAACATTGTGGATGCCGTT
ATAAAAGATACAATGGATCTCAAACACTTATACCAGGTAGCAGCCATGGCTGTGTTGTGCGTGCAG
CCAGAACCAAGTTACCGGCCGTTGATAACCGATGTTCTTCACTCACTTGTTCCACTGGTTCCGGTAG
AGCTAGGAGGGACTCTCCGGTTAACAAGATGA
>SEQ ID NO: 4
MRELLLLLLLHFQSLILLMIFITVSASSASNPSLAPVYSSMATFSPRIQMGSGEEDRFDAHKKLLIGLIISFS
SLGLIILFCFGFWVYRKNQSPKSINNSDSESGNSFSLLMRRLGSIKTQRRTSIQKGYVQFFDIKTLEKATG
GFKESSVIGQGGFGCVYKGCLDNNVKAAVKKIENVSQEAKREFQNEVDLLSKIHHSNVISLLGSASEINS
SFIVYELMEKGSLDEQLHGPSRGSALTWHMRMKIALDTARGLEYLHEHCRPPVIHRDLKSSNILLDSSFN
AKISDFGFAVSLDEHGKNNIKLSGTLGYVAPEYLLDGKLTDKSDVYAFGVVLLELLLGRRPVEKLTPAQ
CQSLVTWAMPQLTDRSKLPNIVDAVIKDTMDLKHLYQVAAMAVLCVQPEPSYRPLITDVLHSLVPLVP
VELGGTLRLTR
>SEQ ID NO: 5
ATGGGAAGTGGTGAAGAAGATAGATTTGATGCTCATAAGAAACTTCTGATTGGTCTCATAATCAGT
TTCTCTTCTCTTGGCCTTATAATCTTGTTCTGTTTTGGCTTTTGGGTTTATCGCAAGAACCAATCTCC
AAAATCCATCAACAACTCAGATTCTGAGAGTGGGAATTCATTTTCCTTGTTAATGAGACGACTTGG
CTCGATTAAAACTCAGAGAAGAACTTCTATCCAAAAGGGTTACGTGCAATTTTTCGATATCAAGAC
CCTCGAGAAAGCGACAGGCGGTTTTAAAGAAAGTAGTGTAATCGGACAAGGCGGTTTCGGATGCG
TTTACAAGGGTTGTTTGGACAATAACGTTAAAGCAGCGGTCAAGAAGATCGAGAACGTTAGCCAA
GAAGCAAAACGAGAATTTCAGAATGAAGTTGACTTGTTGAGCAAGATCCATCACTCGAACGTTATA
TCATTGTTGGGCTCTGCAAGCGAAATCAACTCGAGTTTCATCGTTTATGAGCTTATGGAGAAAGGA
TCATTAGATGAACAGTTACATGGGCCTTCTCGTGGATCAGCTCTAACATGGCACATGCGTATGAAG
ATTGCTCTTGATACAGCTAGAGGACTAGAGTATCTCCATGAGCATTGTCGTCCACCAGTTATCCACA
GAGATTTGAAATCTTCGAATATTCTTCTTGATTCTTCCTTCAACGCCAAGATTTCAGATTTCGGTTTT
GCTGTATCGCTGGATGAACATGGCAAGAACAACATTAAACTCTCTGGGACACTTGGTTATGTTGCC
CCGGAATACCTCCTTGACGGAAAACTGACGGATAAGAGTGATGTTTATGCATTTGGGGTAGTTCTG
CTTGAACTCTTGTTGGGTAGACGACCAGTTGAAAAATTAACTCCAGCTCAATGCCAATCTCTTGTAA
CTTGGGCAATGCCACAACTTACCGATAGATCCAAGCTTCCAAACATTGTGGATGCCGTTATAAAAG
ATACAATGGATCTCAAACACTTATACCAGGTAGCAGCCATGGCTGTGTTGTGCGTGCAGCCAGAAC
CAAGTTACCGGCCGTTGATAACCGATGTTCTTCACTCACTTGTTCCACTGGTTCCGGTAGAGCTAGG
AGGGACTCTCCGGTTAACAAGATGATTCACAGA
>SEQ ID NO: 6
MGSGEEDRFDAHKKLLIGLIISFSSLGLIILFCFGFWVYRKNQSPKSINNSDSESGNSFSLLMRRLGSIKTQ
RRTSIQKGYVQFFDIKTLEKATGGFKESSVIGQGGFGCVYKGCLDNNVKAAVKKIENVSQEAKREFQNE
VDLLSKIHHSNVISLLGSASEINSSFIVYELMEKGSLDEQLHGPSRGSALTWHMRMKIALDTARGLEYLH
EHCRPPVIHRDLKSSNILLDSSFNAKISDFGFAVSLDEHGKNNIKLSGTLGYVAPEYLLDGKLTDKSDVY
AFGVVLLELLLGRRPVEKLTPAQCQSLVTWAMPQLTDRSKLPNIVDAVIKDTMDLKHLYQVAAMAVL
CVQPEPSYRPLITDVLHSLVPLVPVELGGTLRLTR
>SEQ ID NO: 7
ATGGGAAGTGGTGAAGAAGATAGATTTGATGCTCATAAGAAACTTCTGATTGGTCTCATAATCAGT
TTCTCTTCTCTTGGCCTTATAATCTTGTTCTGTTTTGGCTTTTGGGTTTATCGCAAGAACCAATCTCC
AAAATCCATCAACAACTCAGATTCTGAGAGTGGGAATTCATTTTCCTTGTTAATGAGACGACTTGG
CTCGATTAAAACTCAGAGAAGAACTTCTATCCAAAAGGGTTACGTGCAATTTTTCGATATCAAGAC
CCTCGAGAAAGCGACAGGCGGTTTTAAAGAAAGTAGTGTAATCGGACAAGGCGGTTTCGGATGCG
TTTACAAGGGTTGTTTGGACAATAACGTTAAAGCAGCGGTCAAGAAGATCGAGAACGTTAGCCAA
GAAGCAAAACGAGAATTTCAGAATGAAGTTGACTTGTTGAGCAAGATCCATCACTCGAACGTTATA
TCATTGTTGGGCTCTGCAAGCGAAATCAACTCGAGTTTCATCGTTTATGAGCTTATGGAGAAAGGA
TCATTAGATGAACAGTTACATGGGCCTTCTCGTGGATCAGCTCTAACATGGCACATGCGTATGAAG
ATTGCTCTTGATACAGCTAGAGGACTAGAGTATCTCCATGAGCATTGTCGTCCACCAGTTATCCACA
GAGATTTGAAATCTTCGAATATTCTTCTTGATTCTTCCTTCAACGCCAAGATTTCAGATTTCGGTCTT
GCTGTATCGCTGGATGAACATGGCAAGAACAACATTAAACTCTCTGGGACACTTGGTTATGTTGCC
CCGGAATACCTCCTTGACGGAAAACTGACGGATAAGAGTGATGTTTATGCATTTGGGGTAGTTCTG
CTTGAACTCTTGTTGGGTAGACGACCAGTTGAAAAATTAACTCCAGCTCAATGCCAATCTCTTGTAA
CTTGGGCAATGCCACAACTTACCGATAGATCCAAGCTTCCAAACATTGTGGATGCCGTTATAAAAG
ATACAATGGATCTCAAACACTTATACCAGGTAGCAGCCATGGCTGTGTTGTGCGTGCAGCCAGAAC
CAAGTTACCGGCCGTTGATAACCGATGTTCTTCACTCACTTGTTCCACTGGTTCCGGTAGAGCTAGG
AGGGACTCTCCGGTTAACAAGATGATTCACAGA
>SEQ ID NO: 8
MGSGEEDRFDAHKKLLIGLIISFSSLGLIILFCFGFWVYRKNQSPKSINNSDSESGNSFSLLMRRLGSIKTQ
RRTSIQKGYVQFFDIKTLEKATGGFKESSVIGQGGFGCVYKGCLDNNVKAAVKKIENVSQEAKREFQNE
VDLLSKIHHSNVISLLGSASEINSSFIVYELMEKGSLDEQLHGPSRGSALTWHMRMKIALDTARGLEYLH
EHCRPPVIHRDLKSSNILLDSSFNAKISDFGLAVSLDEHGKNNIKLSGTLGYVAPEYLLDGKLTDKSDVY
AFGVVLLELLLGRRPVEKLTPAQCQSLVTWAMPQLTDRSKLPNIVDAVIKDTMDLKHLYQVAAMAVL
CVQPEPSYRPLITDVLHSLVPLVPVELGGTLRLTR
>SEQ ID NO: 9
ATCAAAAACTTTTCTTTTCTTAGCAAAAAAAACAAAAAAATGAGAGAGCTTCTTCTTCTTCTTCTTC
TTCATTTTCAGTCTCTAATTCTTTTGATGATCTTCATCACTGTCTCTGCTTCTTCTGCTTCAAATCCTT
CTTTAGCTCCTGTTTACTCTTCCATGGCTACATTCTCTCCTCGAATCCAAATGGGAAGTGGTGAAGA
AGATAGATTTGATGCTCATAAGAAACTTCTGATTGGTCTCATAATCAGTTTCTCTTCTCTTGGCCTT
ATAATCTTGTTCTGTTTTGGCTTTTGGGTTTATCGCAAGAACCAATCTCCAAAATCCATCAACAACT
CAGATTCTGAGAGTGGGAATTCATTTTCCTTGTTAATGAGACGACTTGGCTCGATTAAAACTCAGA
GAAGAACTTCTATCCAAAAGGGTTACGTGCAATTTTTCGATATCAAGACCCTCGAGAAAGCGACAG
GCGGTTTTAAAGAAAGTAGTGTAATCGGACAAGGCGGTTTCGGATGCGTTTACAAGGGTTGTTTGG
ACAATAACGTTAAAGCAGCGGTCAAGAAGATCGAGAACGTTAGCCAAGAAGCAAAACGAGAATTT
CAGAATGAAGTTGACTTGTTGAGCAAGATCCATCACTCGAACGTTATATCATTGTTGGGCTCTGCA
AGCGAAATCAACTCGAGTTTCATCGTTTATGAGCTTATGGAGAAAGGATCATTAGATGAACAGTTA
CATGGGCCTTCTCGTGGATCAGCTCTAACATGGCACATGCGTATGAAGATTGCTCTTGATACAGCT
AGAGGACTAGAGTATCTCCATGAGCATTGTCGTCCACCAGTTATCCACAGAGATTTGAAATCTTCG
AATATTCTTCTTGATTCTTCCTTCAACGCCAAGATTTCAGATTTCGGTCTTGCTGTATCGCTGGATGA
ACATGGCAAGAACAACATTAAACTCTCTGGGACACTTGGTTATGTTGCCCCGGAATACCTCCTTGA
CGGAAAACTGACGGATAAGAGTGATGTTTATGCATTTGGGGTAGTTCTGCTTGAACTCTTGTTGGG
TAGACGACCAGTTGAAAAATTAACTCCAGCTCAATGCCAATCTCTTGTAACTTGGGCAATGCCACA
ACTTACCGATAGATCCAAGCTTCCAAACATTGTGGATGCCGTTATAAAAGATACAATGGATCTCAA
ACACTTATACCAGGTAGCAGCCATGGCTGTGTTGTGCGTGCAGCCAGAACCAAGTTACCGGCCGTT
GATAACCGATGTTCTTCACTCACTTGTTCCACTGGTTCCGGTAGAGCTAGGAGGGACTCTCCGGTTA
ACAAGATGATTCACAGAAACACGCCAAAAGAAATCCAAAGCCATTTAGATGATTTTCTTTTATCCT
TTGCCTTTATATTTTTTTGTATAGGGTTATGATCCACTCATCTGAAAGTTTGGGGGTAAGAATGTGA
GAATATAAGTTTTCAGGGTTGTTGAGTTCTATATAATTATATTTGTTTCTTTTTATTGTCAAATATAA
TTATATTTTTGT
>SEQ ID NO: 10
AAAATGAGAGAGCTTCTTCTTCTTCTTCTTCTTCATTTTCAGTCTCTAATTCTTTTGATGATCTTCATC
ACTGTCTCTGCTTCTTCTGCTTCAAATCCTTCTTTAGCTCCTGTTTACTCTTCCATGGCTACATTCTCT
CCTCGAATCCAAATGGGAAGTGGTGAAGAAGATAGATTTGATGCTCATAAGAAACTTCTGATTGGT
CTCATAATCAGTTTCTCTTCTCTTGGCCTTATAATCTTGTTCTGTTTTGGCTTTTGGGTTTATCGCAA
GAACCAATCTCCAAAATCCATCAACAACTCAGATTCTGAGAGTGGGAATTCATTTTCCTTGTTAATG
AGACGACTTGGCTCGATTAAAACTCAGAGAAGAACTTCTATCCAAAAGGGTTACGTGCAATTTTTC
GATATCAAGACCCTCGAGAAAGCGACAGGCGGTTTTAAAGAAAGTAGTGTAATCGGACAAGGCGG
TTTCGGATGCGTTTACAAGGGTTGTTTGGACAATAACGTTAAAGCAGCGGTCAAGAAGATCGAGAA
CGTTAGCCAAGAAGCAAAACGAGAATTTCAGAATGAAGTTGACTTGTTGAGCAAGATCCATCACTC
GAACGTTATATCATTGTTGGGCTCTGCAAGCGAAATCAACTCGAGTTTCATCGTTTATGAGCTTATG
GAGAAAGGATCATTAGATGAACAGTTACATGGGCCTTCTCGTGGATCAGCTCTAACATGGCACATG
CGTATGAAGATTGCTCTTGATACAGCTAGAGGACTAGAGTATCTCCATGAGCATTGTCGTCCACCA
GTTATCCACAGAGATTTGAAATCTTCGAATATTCTTCTTGATTCTTCCTTCAACGCCAAGATTTCAG
ATTTCGGTCTTGCTGTATCGCTGGATGAACATGGCAAGAACAACATTAAACTCTCTGGGACACTTG
GTTATGTTGCCCCGGAATACCTCCTTGACGGAAAACTGACGGATAAGAGTGATGTTTATGCATTTG
GGGTAGTTCTGCTTGAACTCTTGTTGGGTAGACGACCAGTTGAAAAATTAACTCCAGCTCAATGCC
AATCTCTTGTAACTTGGGCAATGCCACAACTTACCGATAGATCCAAGCTTCCAAACATTGTGGATG
CCGTTATAAAAGATACAATGGATCTCAAACACTTATACCAGGTAGCAGCCATGGCTGTGTTGTGCG
TGCAGCCAGAACCAAGTTACCGGCCGTTGATAACCGATGTTCTTCACTCACTTGTTCCACTGGTTCC
GGTAGAGCTAGGAGGGACTCTCCGGTTAACAAGATGATTCACAG
>SEQ ID NO: 11
TCTGTGTCAGGAATCCAAATGGGAAGTGGTGAAGAAGATAGATTTGATGCTCATAAGAAACTTCTG
ATTGGTCTCATAATCAGTTTCTCTTCTCTTGGCCTTATAATCTTGTTCTGTTTTGGCTTTTGGGTTTAT
CGCAAGAACCAATCTCCAAAATCCATCAACAACTCAGATTCTGAGAGTGGGAATTCATTTTCCTTG
TTAATGAGACGACTTGGCTCGATTAAAACTCAGAGAAGAACTTCTATCCAAAAGGGTTACGTGCAA
TTTTTCGATATCAAGACCCTCGAGAAAGCGACAGGCGGTTTTAAAGAAAGTAGTGTAATCGGACAA
GGCGGTTTCGGATGCGTTTACAAGGGTTGTTTGGACAATAACGTTAAAGCAGCGGTCAAGAAGATC
GAGAACGTTAGCCAAGAAGCAAAACGAGAATTTCAGAATGAAGTTGACTTGTTGAGCAAGATCCA
TCACTCGAACGTTATATCATTGTTGGGCTCTGCAAGCGAAATCAACTCGAGTTTCATCGTTTATGAG
CTTATGGAGAAAGGATCATTAGATGAACAGTTACATGGGCCTTCTCGTGGATCAGCTCTAACATGG
CACATGCGTATGAAGATTGCTCTTGATACAGCTAGAGGACTAGAGTATCTCCATGAGCATTGTCGT
CCACCAGTTATCCACAGAGATTTGAAATCTTCGAATATTCTTCTTGATTCTTCCTTCAACGCCAAGA
TTTCAGATTTCGGTCTTGCTGTATCGCTGGATGAACATGGCAAGAACAACATTAAACTCTCTGGGA
CACTTGGTTATGTTGCCCCGGAATACCTCCTTGACGGAAAACTGACGGATAAGAGTGATGTTTATG
CATTTGGGGTAGTTCTGCTTGAACTCTTGTTGGGTAGACGACCAGTTGAAAAATTAACTCCAGCTCA
ATGCCAATCTCTTGTAACTTGGGCAATGCCACAACTTACCGATAGATCCAAGCTTCCAAACATTGT
GGATGCCGTTATAAAAGATACAATGGATCTCAAACACTTATACCAGGTAGCAGCCATGGCTGTGTT
GTGCGTGCAGCCAGAACCAAGTTACCGGCCGTTGATAACCGATGTTCTTCACTCACTTGTTCCACTG
GTTCCGGTAGAGCTAGGAGGGACTCTCCGGTTAACAAGATGATTCACAG
>SEQ ID NO: 12
TCGCAAGAACCAATCTCCAAAATCCATCAACAACTCAGATTCTGAGAGTGGGAATTCATTTTCCTT
GTTAATGAGACGACTTGGCTCGATTAAAACTCAGAGAAGAACTTCTATCCAAAAGGGTTACGTGCA
ATTTTTCGATATCAAGACCCTC
>SEQ ID NO: 13
TCTGTGTCAGGAATCCAAATGGGAAGTGGTGAAGAAGATAGATTTGATGCTCATAAGAAACTTCTG
ATTGGTCTCATAATCAGTTTCTCTTCTCTTGGCCTTATAATCTTGTTCTGTTTTGGCTTTTGGGTTTAT
CGCAAGAACCAATCTCCAAAATCCATCAACAACTCAGATTCTGAGAGTGGGAATTCATTTTCCTTG
TTAATGAGACGACTTGGCTCGATTAAAACTCAGAGAAGAACTTCTATCCAAAAGGGTTACGTGCAA
TTTTTCGATATCAAGACCCTC
>SEQ ID NO: 14
TGTTAAAAGCGATTTATAATTTACACCGTTTTGGTGTATATTTCTATCTATCCTTTTACAAGACCTAT
ATATGTTATGTTATGGTGGTGTACTATTTTAAGTGAGCGACATAGTATTTTCTTCATATAGCTAATT
AATCAACAACAATTTCCCAACTTACAACTATTTGCGTACTTTAAACTTATATTGAAAGAGAACTAC
AAAATTATTTTTTTGTACAAGAGAATTATGGTCTTCGGATCAATAATTTCTCTAGATATAATATGTA
AAGCCAACCCTATAATTTGTAAAATCCATGATTTGATATAATTTTCTTTTAAAATTGTGAATTGGCA
GACAAAAACAACATTACATTTTGATTTAAATTCATAACTTTGACTTGCTAAGGAAACACCATGATT
CATTTTTTGTCATTTGTTACATCATCACTAGAAATATTTGATCTAACTTTATTATGATAATAGACTAC
ATACTACATATGCAGTTACGATTTTAAATACTACATATTTAAGCGTGTTTAAACTGTAACCATATCA
TATAAAATGACATATCTAAAAGTGATTTTCAATATTTTGATATGATATGTGTTGTAGCACGGATAAT
GATCTAATTTTTAAGTAATAAGCTTGTTCATTACAAAAGAGAAGAAAGTAGTATTGGGCCATGATT
ATGTAAGGACAAAATAGGAAGATGTGGAAGAAGCCATTCGAGGGTTTTATTACAAAAACAGAGTA
TATAATTGGTCATAATGTTTTATTCACTTAATTTAACATTATTGCATTATATTTTCATGAACACATAT
TTCTTTAACTAAAAATATACACATATTTCTTATTGTAGATGAAGTGAAAAGAACAATATTTGGGTTC
ACATCTATGGGTGAATCCTTTTAATCACCCCCTAAAATAAAAAAGGTGCCATATTTCTATTTTTAGA
GAAAGATATAGAGCACCATTGGAGTGGTTTTGCTCCAAATATAGAGTTTAGAGAAATATATAATAC
ACCATTGGAGATGCTCTAAAATGAATTTATTTATTTATTTAGATGGAAGATTCTAATTGGTTAGAAA
AAGAGGAAGTGAATAATAGGATTCACCTATAAGAGTGAACCCAAGTATTTTTAAGAGATAATGTGT
AAAGTAAATAGATGGTCATTGTGTGAATTATGAATAGAACCATGGTTTTCCATTTTTAATTGCTTAA
CATAGGGTAATCAACAATGGGGTTTAATATGTCAATAGACAATAGTAAAGAAAGTATTTGATCTAT
CCCAAATCTTTCTTCGTTCGTTAGTTCATCACTTTCTTTCTTTTTGGTTATATTAATGGTAGAGAACT
AAAAATTCAACTTTTTATTCAAAAGCTCCCTTTCTCTTTCCCTCCTTTATTTGCCATAAAAGTGATTT
CAAGAAGACAGCGAGAGAGAAAGTGATAGTTCGTTCACTCTTCGCTTTCTCAAGAATTTCAAAACA
CCAAAAAAGTCTTTAGATTGAATTTCATCAAAAACTTTTC
>SEQ ID NO: 15
AGACAAGAAAAAAGGAAACAAAATTTTATGAAAGAGATCTCCATTAGAGAAAGAGAGAGCGAGA
GAGAGATTAATCTTGGAAGAGCAATCTCACATTCTCACACTGCTCTTAGAAAATCTCTCTTTCACCA
TTAAAAATCCCAAAGAGTCTGGAGAA
>SEQ ID NO: 16
ATGGGAAAGATTCTTCATCTTCTTCTTCTTCTTCTTAAGGTCTCTGTTCTTGAATTCATCATTAGTGT
TTCTGCTTTTACTTCACCTGCTTCACAGCCTTCTCTTTCTCCTGTTTACACTTCCATGGCTTCCTTTTC
TCCAGGGATCCACATGGGCAAAGGCCAAGAACACAAGTTAGATGCACACAAGAAACTTCTAATCG
CTCTCATAATCACCTCATCTTCTCTAGGACTAATACTTGTATCTTGTTTATGCTTTTGGGTTTATTGG
TCTAAGAAATCTCCCAAAAACACCAAGAACTCAGGTGAGAGTAGGATTTCATTATCCAAGAAGGG
CTTTGTGCAGTCCTTCGATTACAAGACACTAGAGAAAGCAACAGGCGGTTTCAAAGACGGTAATCT
TATAGGACGAGGCGGGTTCGGAGATGTTTACAAGGCCTGTTTAGGCAACAACACTCTAGCAGCAGT
CAAAAAGATCGAAAACGTTAGTCAAGAAGCAAAACGAGAATTTCAGAATGAAGTTGATTTGTTGA
GCAAGATTCACCACCCGAACATCATCTCATTGTTTGGATATGGAAATGAACTCAGTTCGAGTTTTAT
CGTCTACGAGCTGATGGAAAGCGGATCATTGGATACACAGTTACACGGACCTTCTCGGGGATCGGC
TTTAACATGGCACATGCGGATGAAGATTGCTCTTGATACAGCAAGAGCTGTTGAGTATCTCCACGA
GCGTTGTCGTCCTCCGGTTATCCACAGAGATCTTAAATCGTCAAATATTCTCCTTGATTCTTCCTTCA
ACGCCAAGATTTCGGATTTTGGTCTTGCGGTAATGGTGGGGGCTCACGGCAAAAACAACATTAAAC
TATCAGGAACACTTGGTTATGTTGCTCCAGAATATCTCCTAGATGGAAAATTGACGGATAAGAGTG
ATGTTTATGCGTTTGGTGTGGTTTTACTTGAACTCTTGTTAGGAAGACGGCCGGTTGAGAAATTGAG
TTCGGTTCAGTGTCAATCTCTTGTCACTTGGGCAATGCCCCAACTTACGGATAGATCAAAGCTTCCG
AAAATCGTGGATCCGGTTATCAAAGATACAATGGATCATAAGCACTTATACCAGGTGGCAGCCGTG
GCAGTGCTTTGTGTACAACCAGAACCGAGTTATCGACCGTTGATAACCGATGTTCTTCACTCACTAG
TTCCATTGGTTCCGGTAGAGCTAGGAGGGACTCTCCGGTTAATACCATCATCGTCTTGA
>SEQ ID NO: 17
MGKILHLLLLLLKVSVLEFIISVSAFTSPASQPSLSPVYTSMASFSPGIHMGKGQEHKLDAHKKLLIALIIT
SSSLGLILVSCLCFWVYWSKKSPKNTKNSGESRISLSKKGFVQSFDYKTLEKATGGFKDGNLIGRGGFG
DVYKACLGNNTLAAVKKIENVSQEAKREFQNEVDLLSKIHHPNIISLFGYGNELSSSFIVYELMESGSLD
TQLHGPSRGSALTWHMRMKIALDTARAVEYLHERCRPPVIHRDLKSSNILLDSSFNAKISDFGLAVMVG
AHGKNNIKLSGTLGYVAPEYLLDGKLTDKSDVYAFGVVLLELLLGRRPVEKLSSVQCQSLVTWAMPQL
TDRSKLPKIVDPVIKDTMDHKHLYQVAAVAVLCVQPEPSYRPLITDVLHSLVPLVPVELGGTLRLIPSSS
>SEQ ID NO: 18
ATGAAGCAAATTGTTATAACAGCTCTTGTTTTACTACAAGCTTATGTTCTTCATCAATCCACATGTG
TTATGTCCCTTACTACACAAGAATCTCCTTCTCCTCAACCTTCTGCTTTCACTCCCGCCTTATCTCCT
GATTATCAACAGAGAGAGAAGGAATTGCATAAACAAGAGAGTAACAACATGAGACTGGTTATTTC
ACTAGCAGCTACATTTTCCTTAGTTGGTATAATCTTACTTTGCTCTCTGCTTTATTGGTTTTGCCATA
GGAGAAGAAACCTCAAGAGCTCAGGTTGTGGGTGTAGTGGAATCACATTCTTGAATCGGTTTAGTC
GCTCAAAAACATTAGACAAGAGAACTACAAAGCAGGGAACAGTGTCATTGATCGATTACAATATA
CTAGAAGAAGGAACTAGTGGTTTCAAGGAGAGTAACATTTTGGGTCAAGGTGGATTTGGATGTGTA
TATTCTGCCACATTAGAGAACAACATTTCAGCTGCGGTTAAGAAGCTAGACTGTGCCAATGAAGAT
GCAGCAAAGGAATTTAAGAGTGAGGTTGAGATATTGAGTAAGCTCCAGCACCCGAATATAATATC
CCTTTTGGGTTATAGCACGAATGATACTGCGAGATTCATTGTCTATGAGCTGATGCCAAACGTTTCT
CTGGAATCTCATTTACACGGATCTTCTCAGGGTTCGGCGATCACATGGCCTATGAGGATGAAGATT
GCTCTTGATGTAACAAGGGGATTAGAATATTTGCATGAACATTGTCATCCAGCAATCATTCACAGG
GACTTGAAATCATCCAACATCTTATTAGATAGCAATTTCAATGCTAAGATTTCAGATTTTGGTCTAG
CTGTTGTTGATGGGCCAAAGAACAAGAACCATAAACTTTCCGGGACAGTTGGCTACGTTGCACCAG
AGTATCTTCTCAACGGCCAATTGACAGAAAAGAGCGACGTGTATGCTTTTGGAGTAGTGTTATTAG
AGCTTTTACTCGGGAAAAAACCTGTGGAGAAACTAGCTCCCGGTGAATGCCAATCCATCATCACTT
GGGCAATGCCTTATCTCACTGATAGAACCAAGTTACCAAGCGTCATAGATCCTGCGATTAAAGATA
CGATGGACTTGAAACACCTTTACCAGGTAGCGGCAGTGGCGATTTTGTGCGTGCAGCCAGAACCGA
GTTATAGACCGTTGATTACAGACGTCTTGCATTCTCTTATACCTTTGGTTCCAATGGAACTTGGTGG
AACCTTAAAAACCATCAAATGTGCTTCAATGGATCACTGTTAA
>SEQ ID NO: 19
MKQIVITALVLLQAYVLHQSTCVMSLTTQESPSPQPSAFTPALSPDYQQREKELHKQESNNMRLVISLA
ATFSLVGIILLCSLLYWFCHRRRNLKSSGCGCSGITFLNRFSRSKTLDKRTTKQGTVSLIDYNILEEGTSGF
KESNILGQGGFGCVYSATLENNISAAVKKLDCANEDAAKEFKSEVEILSKLQHPNIISLLGYSTNDTARFI
VYELMPNVSLESHLHGSSQGSAITWPMRMKIALDVTRGLEYLHEHCHPAIIHRDLKSSNILLDSNFNAKI
SDFGLAVVDGPKNKNHKLSGTVGYVAPEYLLNGQLIEKSDVYAFGVVLLELLLGKKPVEKLAPGECQ
SIITWAMPYLTDRTKLPSVIDPAIKDTMDLKHLYQVAAVAILCVQPEPSYRPLITDVLHSLIPLVPMELGG
TLKTIKCASMDHC
>SEQ ID NO: 20
ATGAAGACTATGTCCAAATCGTCTTTGCGTTTGCATTTTCTCTCGCTACTCTTACTTTGTTGTGTCTC
CCCTTCAAGCTTTGTCATTATAAGATTCATTACACATAATCATTTTGATGGTCTAGTACGTTGTCATC
CCCACAAGTTTCAAGCCCTTACGCAGTTCAAGAACGAGTTTGATACCCGCCGTTGCAACCACAGTA
ACTACTTTAATGGAATCTGGTGTGATAACTCCAAGGTGCGGTCACAAAGCTACGACTACGGGACTG
TCTCAGTGGAACTCTCAAATCAAACAGTAGCCTCTTCCAGTTTCATCATCTTCGCTACCTTGATCTC
TCTCACAACAACTTCACCTCCTCTTCCCTCCCTTCCGAGTTTGTTTCCCACTTTGCGGAATCTAACCA
AGCTCACAGTTTTAGACCTTTCTCATAATCACTTCTCCGGAACTTTGAAGCCCAACAATAGCCTCTT
TGAGTTACACCACCTTCGTTACCTTAATCTCGAGGTCAACAACTTCAGTTCCTCACTCCCTTCCGAG
TTTGGCTATCTCAACAATTTACAGCACTGTGGCCTCAAAGAGTTCCCAAACATATTCAAGACCCTTA
AAAAAATGGAGGCTATAGACGTATCCAACAATAGAATCAACGGGAAAATCCCTGAGTGGTTATGG
AGCCTTCCTCTTCTTCATTTAGTGAATATTTTAAATAATTCTTTTGACGGTTTCGAAGGATCAACGG
AAGTTTTAGTAAATTCATCGGTTCGGATATTACTTTTGGAGTCAAACAACTTTGAAGGAGCACTTCC
TAGTCTACCACACTCTATCAACGCCTTCTCCGCGGGTCATAACAATTTCACTGGAGAGATACCTCTT
TCAATCTGCACCAGAACCTCACTTGGTGTCCTTGATCTAAACTACAACAACCTCATTGGTCCGGTTT
CTCAATGTTTGAGTAATGTCACGTTTGTAAATCTCCGGAAAAACAATTTGGAAGGAACTATTCCTG
AGACTTTCATTGTCGGTTCCTCGATAAGGACACTTGATGTTGGATACAATCGACTAACGGGAAAGC
TTCCAAGGTCTCTTTTGAACTGCTCATCTCTAGAGTTTCTAAGCGTTGACAACAACAGAATCAAAGA
CACATTTCCTTTCTGGCTCAAGGCTTTACCAAAGTTACAAGTCCTTACCCTAAGTTCAAACAAGTTT
TATGGTCCTATATCTCCTCCTCATCAAGGTCCTCTCGGGTTTCCAGAGCTGAGAATACTTGAGATAT
CTGATAATAAGTTTACTGGAAGCTTGTCGTCAAGATACTTTGAGAATTGGAAAGCATCGTCCGCCA
TGATGAATGAATATGTGGGTTTATATATGGTTTACGAGAAGAATCCTTATGGTGTAGTTGTCTATAC
CTTTTTGGATCGTATAGATTTGAAATACAAAGGTCTAAACATGGAGCAAGCGAGGGTTCTCACTTC
CTACAGCGCCATTGATTTTTCTAGAAATCTACTTGAAGGAAATATTCCTGAATCCATTGGACTTTTA
AAGGCATTGATTGCACTAAACTTATCGAACAACGCTTTTACAGGCCATATTCCTCAGTCTTTGGCAA
ATCTTAAGGAGCTCCAGTCACTAGACATGTCTAGGAACCAACTCTCAGGGACTATTCCTAATGGAC
TCAAGCAACTCTCGTTTTTGGCTTACATAAGTGTGTCTCATAACCAACTCAAGGGTGAAATACCAC
AAGGAACACAAATTACTGGGCAATTGAAATCTTCCTTTGAAGGGAATGTAGGACTTTGTGGTCTTC
CTCTCGAGGAAAGGTGCTTCGACAATAGTGCATCTCCAACGCAGCACCACAAGCAAGACGAAGAA
GAAGAAGAAGAACAAGTGTTACACTGGAAAGCGGTGGCAATGGGGTATGGACCTGGATTGTTGGT
TGGATTTGCAATTGCATATGTCATTGCTTCATACAAGCCGGAGTGGCTAACCAAGATAATTGGTCC
GAATAAGCGCAGAAACTAG
>SEQ ID NO: 21
MKTMSKSSLRLHFLSLLLLCCVSPSSFVIIRFITHNHFDGLVRCHPHKFQALTQFKNEFDTRRCNHSNYF
NGIWCDNSKVRSQSYDYGTVSVELSNQTVASSSFIIFATLISLTTTSPPLPSLPSLFPTLRNLTKLTVLDLS
HNHFSGTLKPNNSLFELHHLRYLNLEVNNFSSSLPSEFGYLNNLQHCGLKEFPNIFKTLKKMEAIDVSNN
RINGKIPEWLWSLPLLHLVNILNNSFDGFEGSTEVLVNSSVRILLLESNNFEGALPSLPHSINAFSAGHNN
FTGEIPLSICTRTSLGVLDLNYNNLIGPVSQCLSNVTFVNLRKNNLEGTIPETFIVGSSIRTLDVGYNRLTG
KLPRSLLNCSSLEFLSVDNNRIKDTFPFWLKALPKLQVLTLSSNKFYGPISPPHQGPLGFPELRILEISDNK
FTGSLSSRYFENWKASSAMMNEYVGLYMVYEKNPYGVVVYTFLDRIDLKYKGLNMEQARVLTSYSAI
DFSRNLLEGNIPESIGLLKALIALNLSNNAFTGHIPQSLANLKELQSLDMSRNQLSGTIPNGLKQLSFLAYI
SVSHNQLKGEIPQGTQITGQLKSSFEGNVGLCGLPLEERCFDNSASPTQHHKQDEEEEEEQVLHWKAVA
MGYGPGLLVGFAIAYVIASYKPEWLTKIIGPNKRRN
>SEQ ID NO: 22
ATGACTTCCTCTCGCCGTCTTCTTCTTCCTCTCGGAGCATCGCTCACTAGAGGAAGATTTTCTTCCGA
TCAAATCCGAAATGGATTTCTAAGAAACTTCCGTGGATTCGCCACCGTAACTTCGTCGGAACCGGC
CTTAGCCAATCTGGAAGCGAAATATGCCGTAGCGTTGCCAGAATGTTCAACAGTAGAGGACGAGA
TCACGAAGATCCGTCATGAATTCGAGTTAGCGAAACAGAGGTTTCTTAATATCCCTGAAGCTATTA
ATAGTATGCCGAAGATGAATCCTCAAGGGATATATGTGAATAAGAATCTGAGATTGGATAATATAC
AAGTTTATGGATTTGATTATGATTACACTTTGGCACATTACTCTTCTCACTTACAGAGTTTGATCTAT
GATCTTGCCAAGAAACATATGGTTAATGAGTTTAGATATCCTGATGTTTGCACTCAGTTTGAGTATG
ATCCTACTTTTCCAATCCGTGGGTTGTACTATGATAAACTAAAAGGATGCCTCATGAAATTGGATTT
CTTCGGTTCAATCGAGCCAGATGGGTGTTATTTTGGTCGTCGTAAGCTTAGTAGGAAGGAAATAGA
AAGCATGTATGGAACGCGGCACATAGGTCGTGATCAAGCGAGAGGTTTGGTGGGATTGATGGATTT
CTTCTGTTTTAGCGAGGCGTGTCTTATAGCAGACATGGTGCAATATTTTGTTGACGCCAAACTTGAG
TTTGATGCCTCTAACATCTACAATGATGTCAATCGTGCTATTCAACATGTCCATAGAAGTGGATTGG
TTCATAGAGGAATTCTTGCTGATCCCAACAGATATTTGCTAAAAAATGGTCAGCTTCTACGTTTCCT
GAGAATGCTAAAAGATAAAGGAAAGAAGCTTTTTTTGCTGACCAACTCTCCGTATAATTTTGTTGA
TGGCGGAATGCGCTTTCTAATGGAGGAATCTTTTGGCTTCGGAGATTCCTGGCGAGAACTCTTTGAT
GTTGTGATTGCTAAAGCAAATAAACCAGAATTTTACACATCTGAGCACCCTTTCCGTTGTTATGATT
CGGAGAGGGATAATTTGGCATTTACAAAAGTGGATGCATTTGACCCAAAGAAAGTTTATTATCATG
GTTGTCTTAAATCCTTCCTTGAAATCACAAAGTGGCATGGCCCTGAGGTGATTTATTTCGGAGATCA
CTTATTTAGTGATCTAAGAGGGCCTTCAAAAGCTGGTTGGCGAACTGCTGCCATAATTCATGAGCT
CGAGCGAGAGATACAGATACAAAATGATGATAGCTACCGGTTTGAGCAGGCCAAGTTCCATATTAT
CCAAGAGTTACTCGGTAGATTTCACGCGACTGTATCAAACAATCAGAGAAGTGAAGCATGCCAATC
ACTTTTGGATGAGCTGAACAATGCGAGGCAGAGAGCAAGAGACACGATGAAACAAATGTTCAACA
GATCGTTTGGAGCTACATTTGTCACAGACACTGGTCAAGAATCAGCATTCTCTTATCACATCCACCA
ATACGCAGACGTTTATACCAGTAAACCTGAGAACTTTCTGTTATACCGACCTGAAGCCTGGCTTCA
CGTTCCTTACGATATCAAGATCATGCCACATCATGTCAAGGTTGCTTCAACCCTTTTCAAAACCTGA
>SEQ ID NO: 23
MTSSRRLLLPLGASLTRGRFSSDQIRNGFLRNFRGFATVTSSEPALANLEAKYAVALPECSTVEDEITKIR
HEFELAKQRFLNIPEAINSMPKMNPQGIYVNKNLRLDNIQVYGFDYDYTLAHYSSHLQSLIYDLAKKHM
VNEFRYPDVCTQFEYDPTFPIRGLYYDKLKGCLMKLDFFGSIEPDGCYFGRRKLSRKEIESMYGTRHIGR
DQARGLVGLMDFFCFSEACLIADMVQYFVDAKLEFDASNIYNDVNRAIQHVHRSGLVHRGILADPNRY
LLKNGQLLRFLRMLKDKGKKLFLLTNSPYNFVDGGMRFLMEESFGFGDSWRELFDVVIAKANKPEFYT
SEHPFRCYDSERDNLAFTKVDAFDPKKVYYHGCLKSFLEITKWHGPEVIYFGDHLFSDLRGPSKAGWRT
AAIIHELEREIQIQNDDSYRFEQAKFHIIQELLGRFHATVSNNQRSEACQSLLDELNNARQRARDTMKQM
FNRSFGATFVTDTGQESAFSYHIHQYADVYTSKPENFLLYRPEAWLHVPYDIKIMPHHVKVASTLFKT
>SEQ ID NO: 24
ATGGAGATTCCGGCGGCGCCGCCGCCTCCATTGCCGGTGCTGTGCTCGTACGTCGTCTTC
TTGCTGCTGCTGTCTTCGTGCTCACTGGCCAGAGGGAGGATCGCGGTTTCTTCCCCGGGC
CCGTCGCCTGTGGCCGCCGCCGTTACAGCCAATGAGACCGCTTCATCCTCTTCTTCTCCG
GTGTTTCCGGCCGCTCCTCCCGTCGTGATCACAGTGGTGAGGCACCACCATTACCACCGG
GAGCTGGTCATCTCCGCTGTCCTCGCCTGCGTCGCCACCGCCATGATCCTCCTCTCCACA
CTCTACGCCTGGACGATGTGGCGGCGGTCTCGCCGGACCCCCCACGGCGGCAAGGGCCGC
GGCCGGAGATCAGGGATCACACTGGTGCCAATCCTGAGCAAGTTCAATTCAGTGAAGATG
AGCAGGAAGGGGGGCCTTGTGACGATGATCGAGTACCCGTCGCTGGAGGCGGCGACAGGC
AAGTTCGGCGAGAGCAATGTGCTCGGTGTCGGCGGCTTCGGTTGCGTTTATAAGGCGGCG
TTTGATGGCGGTGCCACCGCCGCCGTGAAGAGGCTTGAAGGCGGCGGGCCGGATTGCGAG
AAGGAATTCGAGAATGAGCTGGATTTGCTTGGCAGGATCAGGCACCCAAACATAGTGTCT
CTCCTGGGCTTCTGTGTCCATGGTGGCAATCACTACATTGTTTATGAGCTCATGGAGAAG
GGATCATTGGAGACACAGCTGCATGGGTCTTCACATGGATCTGCTCTGAGCTGGCACGTT
CGGATGAAGATCGCGCTCGATACGGCGAGGGGATTAGAGTATCTTCATGAGCACTGCAAT
CCACCTGTGATCCATAGGGATCTGAAACCTTCTAATATACTTTTAGATTCAGACTTCAAT
GCTAAGATTGCAGATTTTGGCCTTGCGGTCACCGGTGGGAATCTCAACAAAGGGAACCTG
AAGCTTTCCGGGACCTTGGGTTATGTAGCCCCTGAGTACTTATTAGATGGGAAGTTGACT
GAGAAGAGCGATGTATACGCATTTGGAGTAGTGCTTCTAGAGCTCCTGATGGGAAGGAAG
CCTGTTGAGAAAATGTCACCATCTCAGTGCCAATCAATTGTGTCATGGGCTATGCCTCAG
CTGACCGACAGATCGAAGCTCCCCAACATAATTGACCTGGTGATCAAGGACACCATGGAC
CCAAAACACTTGTACCAAGTTGCAGCAGTGGCTGTTCTATGTGTGCAGCCCGAACCGAGC
TACAGACCACTGATAACAGATGTTCTCCACTCTCTTGTTCCTCTAGTGCCTGCGGAGCTC
GGAGGAACACTCAGGGTTGCAGAGCCACCTTCACCTTCTCCAGACCAAAGACATTATCCT
TGTTGA
>SEQ ID NO: 25
ATGAAGAAACTGGTTCATCTTCAGTTTTTGTTTCTTGTCAAGATCTTTGCTACTCAATTCCTCACTCC
TTCTTCATCATCTTTTGCTGCTTCAAATCCTTCTATAGCTCCTGTTTACACCTCCATGACTACTTTCTC
TCCAGGAATTCAAATGGGAAGTGGTGAAGAACACAGATTAGATGCACATAAGAAACTCCTGATTG
GTCTTATAATCAGTTCCTCTTCTCTTGGTATCATAATCTTGATTTGCTTTGGCTTCTGGATGTACTGT
CGCAAGAAAGCTCCCAAACCCATCAAGATTCCGGATGCCGAGAGTGGGACTTCATCATTTTCAATG
TTTGTGAGGCGGCTAAGCTCAATTAAAACTCACAGAACATCTAGCAATCAGGGTTATGTGCAGCGT
TTCGATTCCAAGACGCTAGAGAAAGCGACAGGCGGTTTCAAAGACAGTAATGTAATCGGACAGGG
CGGTTTCGGATGCGTTTACAAGGCTTCTTTGGACAGCAACACTAAAGCAGCGGTTAAAAAGATCGA
AAACGTTACCCAAGAAGCAAAACGAGAATTTCAGAATGAAGTTGAGCTGTTGAGCAAGATCCAGC
ACTCCAATATTATATCATTGTTGGGCTCTGCAAGTGAAATCAACTCGAGTTTCGTCGTTTATGAGTT
GATGGAGAAAGGATCCTTAGATGATCAGTTACATGGACCTTCGTGTGGATCCGCTCTAACATGGCA
TATGCGTATGAAGATTGCTCTAGATACAGCTAGAGGACTAGAGTATCTCCATGAACATTGTCGTCC
ACCAGTTATCCACAGGGACCTGAAATCGTCTAATATTCTTCTTGATTCTTCCTTCAATGCCAAGATT
TCAGATTTTGGTCTGGCTGTATCGGTTGGAGTGCATGGGAGTAACAACATTAAACTCTCTGGGACA
CTTGGTTATGTTGCCCCGGAATATCTCCTAGACGGAAAGTTGACGGATAAGAGTGATGTCTATGCA
TTTGGGGTGGTTCTTCTTGAACTTTTGTTGGGTAGGCGGCCGGTTGAGAAATTGAGTCCATCTCAGT
GTCAATCTCTTGTGACTTGGGCAATGCCACAACTTACCGATAGATCGAAACTCCCAAACATCGTGG
ATCCGGTTATAAAAGATACAATGGATCTTAAGCACTTATACCAAGTAGCAGCCATGGCTGTGCTGT
GCGTACAGCCAGAACCGAGTTACCGGCCGCTGATAACCGATGTTCTTCATTCACTTGTTCCATTGGT
TCCGGTAGAGCTAGGAGGGACTCTCCGGTTAACCCGATGA
>SEQ ID NO: 26
MKKLVHLQFLFLVKIFATQFLTPSSSSFAASNPSIAPVYTSMTTFSPGIQMGSGEEHRLDAHKKLLIGLIIS
SSSLGIIILICFGFWMYCRKKAPKPIKIPDAESGTSSFSMFVRRLSSIKTHRTSSNQGYVQRFDSKTLEKAT
GGFKDSNVIGQGGFGCVYKASLDSNTKAAVKKIENVTQEAKREFQNEVELLSKIQHSNIISLLGSASEIN
SSFVVYELMEKGSLDDQLHGPSCGSALTWHMRMKIALDTARGLEYLHEHCRPPVIHRDLKSSNILLDSS
FNAKISDFGLAVSVGVHGSNNIKLSGTLGYVAPEYLLDGKLTDKSDVYAFGVVLLELLLGRRPVEKLSP
SQCQSLVTWAMPQLTDRSKLPNIVDPVIKDTMDLKHLYQVAAMAVLCVQPEPSYRPLITDVLHSLVPL
VPVELGGTLRLTR
>SEQ ID NO: 27
ATTTTTGGTGTTGAAATGATGCACAACGGATCTTTGGAATCCCAATTGCATGGTCCGTCTCATGGAA
CTGGCTTAAGCTGGCAGCATCGAATGAAAATTGCACTTGATATTGCACGAGGACTAGAGTATCTTC
ACGAGCGCTGTACCCCGCCTGTGATTCATAGAGATCTGAAATCGTCCAACATTCTTCTAGGTTCGA
ACTACAATGCTAAACTTTCTGATTTCGGGCTCGCGATTACTGGTGGGATTCAGGGCAAGAACAACG
TAAAGCTTTCGGGAACATTAGGTTATGTAGCTCCAGAATACCTCTTAGATGGTAAACTTACTGATA
AAAGTGATGTTTATGCGTTTGGAGTTGTACTTCTTGAACTTTTGATAGGTAGAAAACCAGTGGAGA
AAATGTCACCATCTCAATGCCAATCTATCGTTACATGGGCAATGCCTCAACTAACCGACCGATCAA
AGCTTCCTAACATCGTTGATCCCGTGATTAGAGATACAATGGACTTGAAGCACTTGTATCAAGTTG
CTGCGGTTGCTGTGCTATGTGTACAACCGGAACCGAGTTACAGGCCATTGATAACAGATGTTTTGC
ATTCGTTCATCCCACTTGTACCTGTTGAGCTTGGAGGGTCGCTAAGAGTTACCGAATCTTGA
>SEQ ID NO: 28
IFGVEMMHNGSLESQLHGPSHGTGLSWQHRMKIALDIARGLEYLHERCTPPVIHRDLKSSNILLGSNYN
AKLSDFGLAITGGIQGKNNVKLSGTLGYVAPEYLLDGKLTDKSDVYAFGVVLLELLIGRKPVEKMSPSQ
CQSIVTWAMPQLTDRSKLPNIVDPVIRDTMDLKHLYQVAAVAVLCVQPEPSYRPLITDVLHSFIPLVPVE
LGGSLRVTES
>SEQ ID NO: 29
AATTCGGCACGAGGGCTGGATTCCAGTTTTAATGCAAAGCTTTCAGATTTTGGCCTTTCTGTGACTG
CTGGAACCCAGAGTAGGAATGTTAAGATCTCTGGAACTCTGGGTTATGTTGCCCCGGAGTACCTAT
TAGAAGGAAAACTAACTGATAAAAGTGATGTATATGCTTTCGGAGTTGTATTGCTGGAACTTTTGA
TGGGGAGAAGGCCTGTGGAAAAGATGTCACCAACTCAATGTCAATCAATGGTCACATGGGCCATG
CCTCAGCTCACCGATAGATCAAAGCTTCCAAACATTGTGGATCCAGTAATTAGAGACACAATGGAT
TTAAAGCACTTATACCAGGTAGCCGCTGTGGCAGTGCTATGTATACAACCTGAACCAAGTTATAGG
CCATTGATAACCGACGTTCTGCATTCCCTCATTCCTCTTGTACCTACCGACCTTGGAGGGTCACTCC
GAGTGACCTAA
>SEQ ID NO: 30
NSARGLDSSFNAKLSDFGLSVTAGTQSRNVKISGTLGYVAPEYLLEGKLTDKSDVYAFGVVLLELLMG
RRPVEKMSPTQCQSMVTWAMPQLTDRSKLPNIVDPVIRDTMDLKHLYQVAAVAVLCIQPEPSYRPLITD
VLHSLIPLVPTDLGGSLRVT
>SEQ ID NO: 31
GGATTGTGTTTGTGGCTTTATCATTTGAAGTACTCCTTCAAATCCAGTAACAAGAATGCAAAGAGC
AAAGATTCTGAGAATGGAGTTGTGTTATCATCATTTTTGGGCAAATTCACTTCTGTGAGGATGGTTA
GTAAGAAGGGATCTGCTATTTCATTTATTGAGTATAAGCTGTTAGAGAAAGCCACCGACAGTTTTC
ATGAGAGTAATATATTGGGTGAGGGTGGATTTGGATGTGTTTACAAGGCTAAATTGGATGATAACT
TGCACGTCGCTGTCAAAAAATTAGATTGTGCAACACAAGATGCCGGCAGAGAATTTGAGAATGAG
GTGGATTTGCTGAGTAATATTCACCACCCAAATGTTGTTTGTCTGTTGGGTTATAGTGCTCATGATG
ACACAAGGTTTATTGTTTATGAATTGATGGAAAATCGGTCCCTTGATATTCAATTGCATGGTCCTTC
TCATGGATCAGCATTGACTTGGCATATGCGAATGAAAATTGCTCTTGATACCGCTAGAGGATTAGA
ATATTTACATGAGCACTGCAACCCTGCAGTCATTCATAGAGATCTGAAATCCTCCAATATACTTCTA
GATTCCAAGTTTAATGCTAAGCTCTCAGATTTTGGTCTTGCCATAACCGATGGATCCCAAAACAAG
AACAATCTTAAGCTTTCGGGCACTTTGGGATATGTGGCTCCCGAGTATCTTTTAGATGGTAAATTGA
CAGACAAGAGTGATGTCTATGCTTTTGGAGTTGTGCTTCT
>SEQ ID NO: 32
GLCLWLYHLKYSFKSSNKNAKSKDSENGVVLSSFLGKFTSVRMVSKKGSAISFIEYKLLEKATDSFHES
NILGEGGFGCVYKAKLDDNLHVAVKKLDCATQDAGREFENEVDLLSNIHHPNVVCLLGYSAHDDTRFI
VYELMENRSLDIQLHGPSHGSALTWHMRMKIALDTARGLEYLHEHCNPAVIHRDLKSSNILLDSKFNA
KLSDFGLAITDGSQNKNNLKLSGTLGYVAPEYLLDGKLTDKSDVYAFGVVLL
>SEQ ID NO: 33
GCATTGACATGGCATCTTAGGATGAAAATTGCCCTTGATGTAGCTAGAGGATTAGAATTTTTGCAT
GAGCACTGCCACCCAGCAGTGATCCATAGAGATCTGAAATCATCTAATATCCTTCTGGATTCAAAT
CTCAATGCTAAGCTATCTGATTTTGGTCTTGCCATTCTTGATGGGGCTCAAAATAAGAACAACATCA
AGCTTTCTGGAACCTTGGGCTATGTAGCTCCAGAGTACCTCTTAGATGGTAAATTGACTGACAAGA
GTGATGTTTATGCTTTTGGAGTGGTGCTTTTGGAGCTTCTCCTGAGAAGAAAGCCTGTGGAGAAGCT
GGCACCAGCTCAATGCCAATCTATAGTCACATGGGCTATGCCTCAGCTGACAGATAGATCAAAGCT
TCCAAACATCGTGGATCCTGTGATTAGAAATGCTATGGATATAAAGCACTTATTCCAGGTTGCTGC
AGTCGCTGTGCTATGCGTGCAGCCTGAACCAAGCTATCGACCACTGATAACAGATGTGTTGCATTC
CCTTGTTCCCCTTGTTCCTATGGAGCTTGGCGGGACGCTCAGAGTTGAACGACCTGCTTCTGTGACC
TCTCTGTTGATTGATTCTACCTGA
>SEQ ID NO: 34
ALTWHLRMKIALDVARGLEFLHEHCHPAVIHRDLKSSNILLDSNLNAKLSDFGLAILDGAQNKNNIKLS
GTLGYVAPEYLLDGKLTDKSDVYAFGVVLLELLLRRKPVEKLAPAQCQSIVTWAMPQLTDRSKLPNIV
DPVIRNAMDIKHLFQVAAVAVLCVQPEPSYRPLITDVLHSLVPLVPMELGGTLRVERPASVTSLLIDST
>SEQ ID NO: 35
ACTGAGGTGACCCGGAAGAAAAACAGGGTAAAGCTATCGGGCACTTTGGGTTATGTAGCCCCAGA
ATATGTCTTGGATGGTAAATTGACTGATAAGAGTGATGTCTATGCCTTTGGAGTTGTGCTTTTGGAG
CTCCTTTTGAGAAGAAGGCCTCTTGAGATAGTAGCACCCACTCAGTGCCAGTCTATTGTTACATGG
GCCATGCCTCAGCTGACCGACCGAACTAAGCTTCCAGATATTGTGGATCCTGTAATTAGAGATGCG
ATGGATGTCAAGCACTTATACCAGGCAGCTGCTGTTGCTGTTTTGTGTCTGCAACCAGAACCGATCT
ACCGGCCACTGATAACGGATGTACTCCACTCTCTCATTCCACTTGTACCCGTTGAACTTGGGGGAAC
GCTGAAGACCTAG
>SEQ ID NO: 36
TEVTRKKNRVKLSGTLGYVAPEYVLDGKLTDKSDVYAFGVVLLELLLRRRPLEIVAPTQCQSIVTWAM
PQLTDRTKLPDIVDPVIRDAMDVKHLYQAAAVAVLCLQPEPIYRPLITDVLHSLIPLVPVELGGTLKT
>SEQ ID NO: 37
ACGAGGCCTCGTGCCATACTTTTGGATTCAGATTTCAATGCCAAGATTTCGGATTTCGGTCTTGCAG
TGTCAAGTGGAAATCGCACCAAAGGTAATCTGAAGCTTTCCGGAACTTTGGGCTATGTTGCTCCTG
AGTACTTATTAGACGGGAAGTTGACAGAGAAGAGTGATGTATATGCGTTCGGAGTAGTACTTCTTG
AGCTTTTGTTAGGAAGGAGGCCAATTGAGAAGATGGCCCCATCTCAATGCCAATCAATTGTTACAT
GGGCCATGCCTCAGCTAATTGACAGATCAAAGCTCCCAACCATAATTGACCCCGTGATCAGGAACA
CGATGGACCTGAAGCACTTGTACCAAGTTGCTGCAGTGGCTGTGCTCTGTGTGCAGCCAGAACCAA
GTTATAGGCCACTAATCACAGATGTGCTCCACTCTCTGATTCCCCTGGTGCCCATGGAGCTCGGAG
GGTCACTGAGGGCTACCTTGGAATCGCCTCGCGTATCACAACATCGTTCTCCCTGCTGA
>SEQ ID NO: 38
TRPRAILLDSDFNAKISDFGLAVSSGNRTKGNLKLSGTLGYVAPEYLLDGKLTEKSDVYAFGVVLLELL
LGRRPIEKMAPSQCQSIVTWAMPQLIDRSKLPTIIDPVIRNTMDLKHLYQVAAVAVLCVQPEPSYRPLIT
DVLHSLIPLVPMELGGSLRATLESPRVSQHRSPC
>SEQ ID NO: 39
CCTTTATTGAATAGATTGAACTCCTTCCGTGGTTCTAGGAGAAAGGGATGTGCATATATAATTGAAT
ATTCTCTGCTGCAAGCAGCCACAAATAATTTTAGTACAAGTGACATCCTTGGAGAGGGTGGTTTTG
GGTGTGTATACAGAGCTAGGTTAGATGATGATTTCTTTGCTGCTGTGAAGAAGTTAGATGAGGGCA
GCAAGCAGGCTGAGTATGAATTTCAGAATGAAGTTGAACTAATGAGCAAAATCAGACATCCAAAT
CTTGTTTCTTTGCTGGGGTTCTGCATTCATGGGAAGACTCGGTTGCTAGTCTACGAGCTCATGCAAA
ATGGTTCTTTGGAAGACCAATTACATGGGCCATCTCATGGATCCGCACTTACATGGTACCTGCGCAT
GAAAATAGCCCTTGATTCAGCAAGGGGTCTAGAACACTTGCACGAGCACTGCAATCCTGCTGTGAT
TCATCGTGATTTCAAATCATCAAATATCCTTCTGGATGCAAGCTTCAATGCCAAGCTTTCAGATTTT
GGTCTTGCAGTAACAGCTGCAGGAGGTATTGGTAATGCTAATGTCGAGCTACTGGGCACTTTGGGA
TATGTAGCTCCAGAATACCTGCTTGATGGCAAGTTGACGGAGAAAAGTGATGTCTATGGATTTGGA
GTTGTTCTTTTGGAGCTAATTATGGGAAGAAAGCCAGTTGATAAATCTGTGGCAACTGAAAGTCAA
TCGCTAGTTTC
>SEQ ID NO: 40
PLLNRLNSFRGSRRKGCAYIIEYSLLQAATNNFSTSDILGEGGFGCVYRARLDDDFFAAVKKLDEGSKQ
AEYEFQNEVELMSKIRHPNLVSLLGFCIHGKTRLLVYELMQNGSLEDQLHGPSHGSALTWYLRMKIAL
DSARGLEHLHEHCNPAVIHRDFKSSNILLDASFNAKLSDFGLAVTAAGGIGNANVELLGTLGYVAPEYL
LDGKLTEKSDVYGFGVVLLELIMGRKPVDKSVATESQSLVS
>SEQ ID NO: 41
ATGAAAATGAAGCTTCTCCTCATGCTTCTTCTTCTTGTTCTTCTTCTTCACCAACCCATTTGGGCTGC
AGACCCTCCTGCTTCTTCTCCTGCTTTATCTCCAGGGGAGGAGCAGCATCACCGGAATAATAAAGT
GGTAATAGCTATCGTCGTAGCCACCACTGCACTTGCTGCACTCATTTTCAGTTTCTTATGCTTCTGG
GTTTATCATCATACCAAGTATCCAACAAAATCCAAATTCAAATCCAAAAATTTTCGAAGTCCAGAT
GCAGAGAAGGGGATCACCTTAGCACCGTTTGTGAGTAAATTCAGTTCCATCAAGATTGTTGGCATG
GACGGGTATGTTCCAATAATTGACTATAAGCAAATAGAAAAAACGACCAATAATTTTCAAGAAAG
TAACATCTTGGGTGAGGGCGGTTTTGGACGTGTTTACAAGGCTTGTTTGGATCATAACTTGGATGTT
GCAGTCAAAAAACTACATTGTGAGACTCAACATGCTGAGAGAGAATTTGAGAACGAGGTGAATAT
GTTAAGCAAAATTCAGCATCCGAATATAATATCTTTACTGGGTTGTAGCATGGATGGTTACACGAG
GCTCGTTGTCTATGAGCTGATGCATAATGGATCATTGGAAGCTCAGTTACATGGACCTTCTCATGGC
TCGGCATTGACTTGGCACATGAGGATGAAGATTGCTCTTGACACAGCAAGAGGATTAGAATATCTG
CACGAGCACTGTCACCCTGCAGTGATCCATAGGGATATGAAATCTTCTAATATTCTCTTAGATGCA
AACTTCAATGCCAAGCTGTCTGATTTTGGTCTTGCCTTAACTGATGGGTCCCAAAGCAAGAAGAAC
ATTAAACTATCGGGTACCTTGGGATACGTAGCACCGGAGTATCTTCTAGATGGTAAATTAAGTGAT
AAAAGTGATGTCTATGCTTTTGGGGTTGTGCTATTGGAGCTCCTACTAGGAAGGAAGCCAGTAGAA
AAACTGGTACCAGCTCAATGCCAATCTATTGTCACATGGGCCATGCCACACCTCACGGACAGATCC
AAGCTTCCAAGCATTGTGGATCCAGTGATTAAGAATACAATGGATCCCAAGCACTTGTACCAGGTT
GCTGCTGTAGCTGTGCTGTGCGTGCAACCAGAACCTAGTTACCGTCCACTGATCATTGATGTTCTTC
ACTCACTCATCCCTCTTGTTCCCATTGAGCTTGGAGGAACACTAAGAGTTTCACAAGTAATT
>SEQ ID NO: 42
MKMKLLLMLLLLVLLLHQPIWAADPPASSPALSPGEEQHHRNNKVVIAIVVATTALAALIFSFLCFWVY
HHTKYPTKSKFKSKNFRSPDAEKGITLAPFVSKFSSIKIVGMDGYVPIIDYKQIEKTTNNFQESNILGEGGF
GRVYKACLDHNLDVAVKKLHCETQHAEREFENEVNMLSKIQHPNIISLLGCSMDGYTRLVVYELMHN
GSLEAQLHGPSHGSALTWHMRMKIALDTARGLEYLHEHCHPAVIHRDMKSSNILLDANFNAKLSDFGL
ALTDGSQSKKNIKLSGTLGYVAPEYLLDGKLSDKSDVYAFGVVLLELLLGRKPVEKLVPAQCQSIVTW
AMPHLTDRSKLPSIVDPVIKNTMDPKHLYQVAAVAVLCVQPEPSYRPLIIDVLHSLIPLVPIELGGTLRVS
QVI
>SEQ ID NO: 43
ACTCAAGCATCAAAATATTGTAAATCTTTTGGGTATTGTGTTCATGATGACACAAGGTTTTTGGTCT
ATGAAATGATGCATCAAGGCTCTTTGGACTCACAATTGCATGGACCAACTCATGGAACCGCATTAA
CCTGGCATCGAAGAATGAAAGTCGCACTTGATATTGCTCGAGGATTAGAGTATCTTCATGAACGAT
GCAACCCGCCTGTGATTCATAGAGATCTTAAGTCATCGAACATTTTGCTAGATTCCAATTTCAATGC
TAAAATTTCGAATTTTGCACTTGCTACCACTGAGCTCCATGCGAAGAACAAAGTTAAGCTTTCGGCT
ACTTCTGGTTATTTGGCTCCGGAATACCTATCAGAAGGTAAACTTACCGATAAAAGCGACGTATAT
GCATTCGGAGTAGTACTTCTTGGGCTTTTAATCGGTAGAAAACCAGTGGAGAAAATGTCACCATCT
TTATTTCAATCTATTGTCACATGGGCAATGCCTCAGTTAACAGACCGGTCAAAGCTTCCAAACATCG
TTGACCCTGTGATTAGAGATACAATGGACCTGAAGCACTTATATCAAGTTGCTGCTGTAGCCGTAC
TTTGCGTGCAACCCGAACCAAGTTACAGACCGTTGATTACAGACGTACTACACTCATTCATTCCACT
CGTACCCGTTGATCTTGGAGGGTCATTAAGAGCTTAA
>SEQ ID NO: 44
TQASKYCKSFGYCVHDDTRFLVYEMMHQGSLDSQLHGPTHGTALTWHRRMKVALDIARGLEYLHER
CNPPVIHRDLKSSNILLDSNFNAKISNFALATTELHAKNKVKLSATSGYLAPEYLSEGKLTDKSDVYAFG
VVLLGLLIGRKPVEKMSPSLFQSIVTWAMPQLTDRSKLPNIVDPVIRDTMDLKHLYQVAAVAVLCVQPE
PSYRPLITDVLHSFIPLVPVDLGGSLRA
>SEQ ID NO: 45
CGATCATTTCGTTGCGGCTGTAAAAAACTCCATGGTCCAGAACCAGATGCCCAAAAAGGGTTTGAG
AATGAAGTAGATTGGTTAGGTAAACTCAAGCATCAAAATATTGTAAATTTTTTGGGTTATTGTGTTC
ATGATGACACAAGGTTTTTGGTCTATGAAATGATGCATCAAGGCTCTTTGGACTCACAATTGCATG
GACCAACTCATGGAACCGCATTAACCTGGCATCGAAGAATGAAAGTCGCACTTGATATTGCTCGAG
GATTAGAGTATCTTCATGAACGATGCAACCCGCCTGTGATTCATAGAGATCTCAAGTCATCGAACA
TTTTGCTAGATTCCAATTTCAATGCTAAAATTTCGAATTTTGCACTTGCTACCACTGAGCTCCATGC
GAAGAACAAAGTTAAGCTTTCGGGTACTTCTGGTTATTTGGCTCCGGAATACCTATCCGAAGGTAA
ACTTACCGATAAAAGTGATGTATATGCATTCGGAGTAGTACTTCTTGAGCTTTTAATCGGTAGAAA
ACCAGTGGAGAAAATGTCACCATCTTTATTTCAATCTATTGTCACATGGGCAATGCCTCAGCTAAC
AGACCGGTCAAAGCTTCCAAACATTGTTGACCCTGTGATTAGAGATACAATGGACCTGAAGCACTT
GTATCAAGTTGCTGCTGTAGCCGTACTTTGCGTGCAACCCGAACCAAGTTACAGACCGTTGATTAC
AGACGTACTACACTCATTCATTCC
>SEQ ID NO: 46
RSFRCGCKKLHGPEPDAQKGFENEVDWLGKLKHQNIVNFLGYCVHDDTRFLVYEMMHQGSLDSQLH
GPTHGTALTWHRRMKVALDIARGLEYLHERCNPPVIHRDLKSSNILLDSNFNAKISNFALATIELHAKN
KVKLSGTSGYLAPEYLSEGKLTDKSDVYAFGVVLLELLIGRKPVEKMSPSLFQSIVTWAMPQLTDRSKL
PNIVDPVIRDTMDLKHLYQVAAVAVLCVQPEPSYRPLITDVLHSFIP
>SEQ ID NO: 47
ATGATGCATCAAGACTCTTTGGACTCACAATTGCATGGACCAACTCATGGAACCGCATTAACCTGG
CATCGAAGAATGAAAGTCGCACTTGATATTGCTCGAGGATTAGAGTATCTTCATGAACGATGCAAC
CCGCCTGTGATTCATAGAGATCTCAAGTCATCGAACATTTTGCTAGATTCCAATTTCAATGCTAAAA
TTTCGAATTTTGCACTTGCTACCACTGAGCTCCATGCGAAGAACAAAGTTAAGCTTTCGGGTACTTC
TGGTTATTTGGCTCCGGAATACCTATCCGAAGGTAAACTTACCGATAAAAGTGATGTATATGCATT
CGGAGTAGTACTTCTTGAGCTTTTAATCGGTAGAAAACCAGTGGAGAAAATGTCACCATCTTTATTT
CAATCTATTGTCACATGGGCAATGCCTCAGCTAACAGACCGGTCAAAGCTTCCAAACATTGTTGAC
CCTGTGATTAGAGATACAATGGACCTGAAGCACTTGTATCAAGTTGCTGCTGTAGCCGTACTTTGC
GTGCAACCCGAACCAAGTTACAGACCGTTGATTACAGACGTACTACACTCATTCATTCCACTCGTA
CCCGTTGATCTTGGAGGGTCATTAAGAGCTTAA
>SEQ ID NO: 48
MMHQDSLDSQLHGPTHGTALTWHRRMKVALDIARGLEYLHERCNPPVIHRDLKSSNILLDSNFNAKIS
NFALATTELHAKNKVKLSGTSGYLAPEYLSEGKLTDKSDVYAFGVVLLELLIGRKPVEKMSPSLFQSIV
TWAMPQLTDRSKLPNIVDPVIRDTMDLKHLYQVAAVAVLCVQPEPSYRPLITDVLHSFIPLVPVDLGGS
LRA
>SEQ ID NO: 49
AATTTGAGAGGTGAGCTGGATTTGCTTCAGAGGATTCAGCATTCGAATATAGTGTCCCTTGTGGGC
TTCTGCATTCATGAGGAGAACCGCTTCATTGTTTATGAGCTGATGGTGAATGGATCACTTGAAACA
CAGCTTCATGGGCCATCACATGGATCAGCTCTGAGTTGGCACATTCGGATGAAGATTGCTCTTGAT
ACAGCAAGGGGATTGGAGTATCTTCACGAGCACTGCAATCCACCAATCATCCATAGGGATCTGAAG
TCGTCTAACATACTTTTGAATTCAGACTTTAATGCAAAGATTTCAGATTTTGGCCTTGCAGTGACAA
GTGGAAATCGCAGCAAAGGGAATCTGAAGCTTTCCGGTACTTTGGGTTATGTTGCCCCTGAGTACT
TACTAGATGGGAAGTTGACTGAGAAGAGCGATGTATATGCATTTGGAGTAGTACTTCTTGAGCTTC
TTTTGGGAAGGAGGCCAGTTGAGAAGATGGCACCATCTCAGTGTCAATCAATTGTTACATGGGCCA
TGCCCCAGCTAATTGACAGATCCAAGCTCCCTACCATAATCGACCCCGTGATCAGGGACACGATGG
ATCGGAAGCACTTGTACCAAGTTGCTGCAGTGGCTGTGCTCTGCGTGCAGCCAGAACCAAGCTACA
GGCCACTGATCACAGATGTCCTCCACTCTCTGATTCCCCTGGTGCCCATGGACCTTGGAGGGACGCT
GAGGATCAACCCGGAATCGCCTTGCACGACACGAAATCAATCTCCCTGCTGA
>SEQ ID NO: 50
NLRGELDLLQRIQHSNIVSLVGFCIHEENRFIVYELMVNGSLETQLHGPSHGSALSWHIRMKIALDTARG
LEYLHEHCNPPIIHRDLKSSNILLNSDFNAKISDFGLAVTSGNRSKGNLKLSGTLGYVAPEYLLDGKLTE
KSDVYAFGVVLLELLLGRRPVEKMAPSQCQSIVTWAMPQLIDRSKLPTIIDPVIRDTMDRKHLYQVAAV
AVLCVQPEPSYRPLITDVLHSLIPLVPMDLGGTLRINPESPCTTRNQSPC
>SEQ ID NO: 51
CGGGGGCTCTTATCACTCATTGCTGCTGCTACTGCACTGGGTACAAGCTTATTGCTCATGGGTTGCT
TCTGGATTTATCATAGAAAGAAAATCCACAAATCTCATGACATTATTCATAGCCCAGATGTAGTTA
AAGGTCTTGCATTATCCTCATATATTAGCAAATACAACTCCTTCAAGTCGAATTGTGTGAAACGAC
ATGTCTCGTTGTGGGAGTACAATACACTCGAGTCGGCCACAAATAGTTTTCAAGAAAGCGAGATCT
TGGGTGGAGGGGGGTTCGGGCTTGTGTACAAGGGAAAACTAGAAGACAACTTGTATGTAGCTGTG
AAGAGGCTGGAAGTTGGAAGACAAAACGCAATTAAAGAATTCGAGGCTGAAATAGAGGTATTGGG
CACGATTCAGCACCCGAATATAATTTCGTTGTTGGGATATAGCATTCATGCTGACACGAGGCTGCT
AGTTTATGAACTGATGCAGAATGGATCTCTGGAGTATCAACTACATGGACCTTCCCATGGATCAGC
ATTAGCGTGGCATAATAGATTGAAAATCGCACTTGATACAGCAAGGGGATTAGAATATTTACATGA
ACATTGCAAACCACCAGTTATCCATAGAGATCTGAAATCCTCCAATATTCTTCTAGATGCCAACTTC
AATGCCAAGATCTCAGATTTTGGTCTTGCTGTGCGCGATGGGGCTCAAAACAAAAATAACATTAAG
CTCTCGGGAACCGTTGGCTATGTAGCTCCAGAATACCTATTAGATGGAATACTAACAGATAAAAGT
GATGTTTATGGCTTCCGAGTTGTA
>SEQ ID NO: 52
RGLLSLIAAATALGTSLLLMGCFWIYHRKKIHKSHDIIHSPDVVKGLALSSYISKYNSFKSNCVKRHVSL
WEYNTLESATNSFQESEILGGGGFGLVYKGKLEDNLYVAVKRLEVGRQNAIKEFEAEIEVLGTIQHPNII
SLLGYSIHADTRLLVYELMQNGSLEYQLHGPSHGSALAWHNRLKIALDTARGLEYLHEHCKPPVIHRDL
KSSNILLDANFNAKISDFGLAVRDGAQNKNNIKLSGTVGYVAPEYLLDGILTDKSDVYGFRVV
>SEQ ID NO: 53
GGGGATATACGTGTAGAATCAGCAACAAATAACTTCGGTGAAAGCGAGATATTAGGCGTAGGTGG
ATTTGGATGCGTGTATAAAGCTCGACTCGATGATAATTTGCATGTAGCTGTTAAAAGATTAGATGG
TATTAGTCAAGACGCCATTAAAGAATTCCAGACGGAGGTGGATCTATTGAGTAAAATTCATCATCC
GAATATCATCACCTTATTGGGATATTGTGTTAATGATGAAACCAAGCTTCTTGTTTATGAACTGATG
CATAATGGATCTTTAGAAACTCAATTACATGGGCCTTCCAGTGGATCCAATTTAACATGGCATTGC
AGGATGAAGATTGCTCTAGATACAGCAAGAGGATTAGAATATTTGCATGAGAACTGCAAACCATC
GGTGATTCATAGAGATCTGAAATCATCTAATATCCTTCTGGATTCCAGCTTCAATGCTAAGCTTTCA
GATTTTGGTCTTGCTATAATGGATGGGGCCCAGAACAAAAACAACATTAAGCTTTCAGGGACATTG
GGTTATGTAGCTCCCGAGTATCTTTTAGATGGAAAATTGACGGATAAAAGTGACGTGTATGCGTTT
GGAGTTGTGCTTTTAGAGCTTTTACTTGGAAGGCGACCTGTAGAAAAATTAGCAGAGTCGCAATGC
CAATCTATTGTCACTTGGGCTATGCCACAATTAACAGACAGATCAAAGCTTCCGAATATTGTAGAT
CCCGTGATCAGATACACAATGGATCTCAAGCACCTGTACCAAGTTGCTGCGGTGGCTGTGTTATGT
GTACAACCCGGACCAAGCTACCGGCCATTTATAAACCGACGTCTTGCATTCTCTGATCCCTCTTGTT
CCCCGTGA
>SEQ ID NO: 54
GDIRVESATNNFGESEILGVGGFGCVYKARLDDNLHVAVKRLDGISQDAIKEFQIEVDLLSKIHHPNIITL
LGYCVNDETKLLVYELMHNGSLETQLHGPSSGSNLTWHCRMKIALDTARGLEYLHENCKPSVIHRDLK
SSNILLDSSFNAKLSDFGLAIMDGAQNKNNIKLSGTLGYVAPEYLLDGKLTDKSDVYAFGVVLLELLLG
RRPVEKLAESQCQSIVTWAMPQLTDRSKLPNIVDPVIRYTMDLKHLYQVAAVAVLCVQPGPSYRPFINR
RLAFSDPSCSP
>SEQ ID NO: 55
AAGTTGAACTGTGAATGTCAATATGCTGAGAGAGAATTTGAGAATGAGGTGGATTTGTTAAGTAAA
ATTCAACATCCAAATGTAATTTCTCTACTGGGCTGTAGCAGTAATGAGGATTCAAGGTTTATTGTCT
ATGAGTTGATGCAAAATGGATCATTGGAAACTCAATTACATGGACCATCTCATGGCTCAGCATTGA
CTTGGCATATGAGGATGAAGATTGCTCTTGACACAGCTAGAGGTTTAAAATATCTGCATGAGCACT
GCTACCCTGCAGTGATCCATAGAGATCTGAAATCTTCTAATATTCTTTTAGATGCAAACTTCAATGC
CAAGCTTTCTGATTTTGGTCTTGCAATAACTGATGGGTCCCAAAACAAGAATAACATCAAGCTTTC
AGGCACATTGGGGTATGTTGCCCCGGAGTATCTTTTAGATGGTAAATTGACAGATAAAAGTGATGT
GTATGCTTTTGGAGTTGTGCTTCTTGAGCTTCTATTAGGAAGAAAGCCTGTGGAAAAACTTACACCA
TCTCAATGCCAGTCTATTGTCACATGGGCCATGCCACAGCTCACAGACAGATCCAAGCTTCCAAAC
ATTGTGGATAATGTGATTAAGAATACAATGGATCCTAAGCACTTATACCAGGTTGCTGCTGTGGCT
GTATTATGTGTGCAACCAGAGCCGTGCTACCGCCCTTTGATTGCAGATGTTCTACACTCCCTCATCC
CTCTTGTACCTGTTGAGCTTGGAGGAACACTCAGAGTTGCACAAGTGACGCAGCAACCTAAGAATT
CTAGTTAA
>SEQ ID NO: 56
KLNCECQYAEREFENEVDLLSKIQHPNVISLLGCSSNEDSRFIVYELMQNGSLETQLHGPSHGSALTWH
MRMKIALDTARGLKYLHEHCYPAVIHRDLKSSNILLDANFNAKLSDFGLAITDGSQNKNNIKLSGTLGY
VAPEYLLDGKLTDKSDVYAFGVVLLELLLGRKPVEKLTPSQCQSIVTWAMPQLTDRSKLPNIVDNVIKN
TMDPKHLYQVAAVAVLCVQPEPCYRPLIADVLHSLIPLVPVELGGTLRVAQVTQQPKNSS
>SEQ ID NO: 57
CAGTTGCATGGACCTCCTCGTGGATCAGCTTTGAATTGGCATCTTCGCATGGAAATTGCATTGGATG
TGGCTAGGGGACTAGAATACCTCCATGAGCGCTGTAACCCCCCTGTAATCCATAGAGATCTCAAAT
CGTCTAATGTTCTATTGGATTCCTACTTCAATGCAAAGCTTTCTGACTTTTGGCCTAGCTATAGCTG
GATGGAACTTAAACAAGAGCACCGTAAAGTCTTTCGGGAACTCTGGGATATGTGGCTCCAGAGTTA
CCTCTTAGATGGGAAATTAACTGATAAGAGTGATGTCTATGCTTTCGGCATTATACTTCTGGAGCTT
CTAATGGGGAGAAGACCATTGGAGAAACTAGCAGGAGCTCAGTGCCAATCTATCGTCACATGGGC
AATGCCACAGCTTACTGACAGGTCAAAGCTCCCAAATATTGTTGATCCTGTCATCAGAAACGGAAT
GGGCCTCAAGCACTTGTATCAAGTTGCTGCTGTAGCCGTGCTATGTGTACAACCAGAACCAAGTTA
CCGACCACTGATAACAGATGTCCTGCACTCCTTCATTCCCCTTGTACCAATTGAGCTTGGTGGGTCC
TTGAGAGTTGTGGATTCTGCATTATCTGTTAACGCATAA
>SEQ ID NO: 58
QLHGPPRGSALNWHLRMEIALDVARGLEYLHERCNPPVIHRDLKSSNVLLDSYFNAKLSDFWPSYSWM
ELKQEHRKVFRELWDMWLQSYLLDGKLTDKSDVYAFGIILLELLMGRRPLEKLAGAQCQSIVTWAMP
QLTDRSKLPNIVDPVIRNGMGLKHLYQVAAVAVLCVQPEPSYRPLITDVLHSFIPLVPIELGGSLRVVDS
ALSVNA
>SEQ ID NO: 59
ATGGAGATGGCGCTAACTCCATTGCCGCTCCTGTGTTCGTCCGTCTTGTTCTTGGTGCTATCTTCGTG
CTCGTTGGCCAATGGGAGGGATACGCCTTCTTCTTCTTCTTCTTCTTCTTCTTCTTCTTCTTCTTCTTC
TTCTTCTTCTTCTTCTTCTTCTTCTCCGGCGACGTCTACTGTGGCCACCGGCATTTCCGCCGCCGCCG
CCGCCGCCGCCAATGGGACGGCCGCCTTGTCTTCGGCAGTTCCGGCGCCTCCGCCTGTTGTGATCGT
AGTGCACCACCATTTCCACCGCGAGCTGGTCATCGCCGCCGTCCTCGCCTGCATCGCCACCGTCAC
GATCTTCCTTTCCACGCTCTACGCTTGGACACTATGGCGGCGATCTCGCCGGAGCACCGGCGGCAA
GGTCACCAGGAGCTCAGACGCAGCGAAGGGGATCAAGCTGGTGCCGATCTTGAGCAGGTTCAACT
CGGTGAAGATGAGCAGGAAGAGGCTGGTTGGGATGTTCGAGTACCCGTCGCTGGAGGCAGCGACA
GAGAAGTTCAGCGAGAGCAACATGCTCGGTGTCGGCGGGTTTGGCCGCGTCTACAAGGCGGCGTTC
GACGCCGGAGTTACCGCGGCGGTGAAGCGGCTCGACGGCGGCGGGCCCGACTGCGAGAAGGAATT
CGAGAATGAGCTGGATTTGCTTGGCAGGATCAGGCACCCCAACATTGTGTCCCTCTTGGGCTTCTGT
ATCCATGAGGGGAATCACTACATTGTTTATGAGCTGATGGAGAAGGGATCACTGGAAACACAGCTT
CATGGGTCTTCACATGGATCAACTCTGAGCTGGCACATCCGGATGAAGATCGCCCTTGACACGGCC
AGGGGATTAGAGTACCTTCATGAGCACTGCAGTCCACCAGTGATCCATAGGGATCTGAAATCGTCT
AACATACTTTTGGATTCAGACTTCAATGCTAAGATTGCAGATTTTGGTCTTGCTGTGTCTAGTGGGA
GTGTCAACAAAGGGAGTGTGAAGCTCTCCGGGACCTTGGGTTATGTAGCTCCTGAGTACTTGTTGG
ATGGGAAGTTGACTGAAAAGAGCGATGTATACGCGTTCGGAGTAGTGCTTCTAGAGCTCCTTATGG
GGAGGAAGCCTGTTGAGAAGATGTCACCATCTCAGTGCCAATCAATTGTGACATGGGCAATGCCAC
AGTTGACCGACAGATCGAAGCTCCCCAGCATAGTTGACCCAGTGATCAAGGACACCATGGATCCA
AAACACCTGTACCAAGTTGCAGCAGTGGCTGTTCTATGCGTGCAGGCTGAACCAAGCTACAGGCCA
CTGATCACAGATGTGCTCCACTCTCTTGTTCCTCTAGTGCCGACGGAGCTCGGAGGAACACTAAGA
GCTGGAGAGCCACCTTCCCCGAACCTGAGGAATTCTCCATGCTGA
>SEQ ID NO: 60
MEMALTPLPLLCSSVLFLVLSSCSLANGRDTPSSSSSSSSSSSSSSSSSSSSSSPATSTVATGISAAAAAAAN
GTAALSSAVPAPPPVVIVVHHHFHRELVIAAVLACIATVTIFLSTLYAWTLWRRSRRSTGGKVTRSSDAA
KGIKLVPILSRFNSVKMSRKRLVGMFEYPSLEAATEKFSESNMLGVGGFGRVYKAAFDAGVTAAVKRL
DGGGPDCEKEFENELDLLGRIRHPNIVSLLGFCIHEGNHYIVYELMEKGSLETQLHGSSHGSTLSWHIRM
KIALDTARGLEYLHEHCSPPVIHRDLKSSNILLDSDFNAKIADFGLAVSSGSVNKGSVKLSGTLGYVAPE
YLLDGKLTEKSDVYAFGVVLLELLMGRKPVEKMSPSQCQSIVTWAMPQLTDRSKLPSIVDPVIKDTMD
PKHLYQVAAVAVLCVQAEPSYRPLITDVLHSLVPLVPTELGGTLRAGEPPSPNLRNSPC
>SEQ ID NO: 61
TACTCTCTTTTACAAACTGCTACGAACAACTTCAGCTCCTCCAATTTGCTGGGCGAGGGAAGTTTCG
GGCATGTGTATAAAGCGAGACTCGATTATGATGTCTATGCCGCTGTAAAGAGACTTACCAGCGTAG
GAAAACAGCCCCAAAAAGAACTCCAGGGAGAGGTGGATCTGATGTGCAAGATAAGACATCCCAAC
TTGGTGGCTCTCCTGGGCTATTCAAATGACGGCCCAGAGCCCTTGGTTGTGTACGAGCTCATGCAG
AATGGTTCACTTCATGATCAGCTTCATGGCCCCTCATGCGGGAGTGCACTCACCTGGTACCTACGAC
TAAAGATTGCTCTTGAAGCTGCCAGCAGAGGACTGGAGCACCTGCATGAAAGCTGCAAGCCTGCA
ATAATCCACAGAGACTTCAAGGCATCCAACATCCTCTTGGACGCCAGCTTCAATGCGAAGGTGTCC
GACTTTGGTATAGCGGTAGCTCTGGAGGAAGGTGGCGTGGTGAAAGACGACGTACAAGTGCAAGG
CACCTTCGGGTACATTGCTCCTGAGTACCTGATGGACGGGACATTGACAGAGAAGAGTGATGTTTA
CGGATTTGGAGTAGTATTGCTTGAGCTGCTGACAGGCAGACTGCCCATTGATACGTCCTTACCACTC
GGATCGCAATCTCTAGTGACATGGGTAACACCCATACTAACTAACCGAGCAAAGCTGATGGAAGTT
ATCGACCCCACCCTTCAAGATACGCTGAACGTGAAGCAACTTCACCAGGTGGCCGCAGTGGCAGTC
CTTTGCGTCCAAGCGGAACCCAGCTACCGCCCTCTCATCGCCGACGTGGTTCAGTCACTGGCTCCGC
TGGTGCCTCAAGAGCTCGGCGGCGCATTGCGA
>SEQ ID NO: 62
YSLLQTATNNFSSSNLLGEGSFGHVYKARLDYDVYAAVKRLTSVGKQPQKELQGEVDLMCKIRHPNLV
ALLGYSNDGPEPLVVYELMQNGSLHDQLHGPSCGSALTWYLRLKIALEAASRGLEHLHESCKPAIIHRD
FKASNILLDASFNAKVSDFGIAVALEEGGVVKDDVQVQGTFGYIAPEYLMDGTLIEKSDVYGFGVVLL
ELLTGRLPIDTSLPLGSQSLVTWVTPILTNRAKLMEVIDPTLQDTLNVKQLHQVAAVAVLCVQAEPSYR
PLIADVVQSLAPLVPQELGGALR
>SEQ ID NO: 63
ACCTCAGATGCCTATAGGGGTATTCCACTCATGCCTCTCCTGAATCGTTTGAACTCCCGTATTTCCA
AGAAGAAGGGATGTGCAACTGCAATTGAATATTCTAAGCTGCAAGCAGCTACAAATAACTTCAGC
AGCAATAACATTCTTGGAGAGGGTGGATTTGCGTGTGTATACAAGGCCATGTTTGATGATGATTCC
TTTGCTGCTGTGAAGAAGCTAGATGAGGGTAGCAGACAGGCTGAGCATGAATTTCAGAATGAAGT
GGAGCTGATGAGCAAAATCCGACATCCAAACCTTGTTTCTTTGCTTGGGTTCTGCTCTCATGAAAAT
ACACGGTTCTTAGTATATGATCTGATGCAGAATGGCTCTTTGGAAGACCAATTACATGGGCCATCT
CACGGATCTGCACTTACATGGTTTTTGCGCATAAAGATAGCACTTGATTCAGCAAGGGGTCTAGAA
CACTTGCATGAGCACTGCAACCCTGCAGTGATTCATCGAGATTTCAAATCATCAAATATTCTTCTTG
ATGCAAGCTTCAACGCCAAGCTTTCAGATTTTGGTCTTGCAGTAACAAGTGCAGGATGTGCTGGCA
ATACAAATATTGATCTAGTAGGGACATTGGGATATGTAGCTCCAGAATACCTACTTGATGGTAAAT
TGACAGAGAAAAGTGATGTCTATGCATATGGAGTTGTTTTGTTGGAGCTACTTTTTGGAAGAAAGC
CAATTGATAAATCTCTACCAAGTGAATGCCAATCTCTCATTTCTTGGGCAATGCCACAGCTAACAG
ATAGAGAAAAGCTCCCAACTATAGTAGACCCCATGATCAAAGGCACAATGAACTTGAAACACCTA
TATCAAGTAGCAGCTGTTGCAATGCTATGTGTGCAGCCAGAACCCAGTTACAGGCCATTAATAGCT
GACGTTGTGCACTCTCTCATTCCTCTCGTACCAATAGAACTCGGGGGAACTTTAAAGCTCTCTAATG
CACGACCCACTGAGATGAAGTTATTTACTTCTTCCCAATGCAGTGTTGAGATTGCTTCCAACCCAAA
ATTGTGA
>SEQ ID NO: 64
TSDAYRGIPLMPLLNRLNSRISKKKGCATAIEYSKLQAATNNFSSNNILGEGGFACVYKAMFDDDSFAA
VKKLDEGSRQAEHEFQNEVELMSKIRHPNLVSLLGFCSHENTRFLVYDLMQNGSLEDQLHGPSHGSAL
TWFLRIKIALDSARGLEHLHEHCNPAVIHRDFKSSNILLDASFNAKLSDFGLAVTSAGCAGNTNIDLVGT
LGYVAPEYLLDGKLTEKSDVYAYGVVLLELLFGRKPIDKSLPSECQSLISWAMPQLTDREKLPTIVDPMI
KGTMNLKHLYQVAAVAMLCVQPEPSYRPLIADVVHSLIPLVPIELGGTLKLSNARPTEMKLFTSSQCSV
EIASNPKL
>SEQ ID NO: 65
AATTCGGCACGAGGAGAACACTTGCACGAGCACTGCAACCCTGCAGTGATTCACCGAGATTTCAAA
TCATCAAATATTCTTCTTGATGCAAGCTTCAACGCCAAGCTTTCAGATTTTGGTCTTGCAGTAAAAA
GTGCAGGATGTGCTGGTAACACAAATATTGATCTAGTAGGGACATTGGGATATGTAGCTCCAGAAT
ACATGCTTGATGGTAAATTGACAGAGAAAAGTGATGTCTATGCATATGGAGTTGTTTTGTTAGAGC
TACTTTTTGGAAGAAAGCCAATTGATAAATCTCTACCAAGTGAATGCCAATCTCTCATTTCTTGGGC
AATGCCACAGCTAACAGATAGAGAAAAGCTCCCGACTATAATAGATCCCATGATCAAAGGCGCAA
TGAACTTGAAACACCTATATCAAGTGGCAGCTGTTGCAGTGCTATGTGTGCAGCCAGAACCCAGTT
ACAGGCCATTAATAGCTGACGTTGTGCACTCTCTCATTCCTCTCGTACCAGTAGAACTTGGGGGAA
CATTAAAGTCATCACCCACTGAGATGAAGTCATTTGCTTCTTCCCAATGCAGTGCCCACGTTGCTTC
>SEQ ID NO: 66
NSARGEHLHEHCNPAVIHRDFKSSNILLDASFNAKLSDFGLAVKSAGCAGNTNIDLVGTLGYVAPEYML
DGKLIEKSDVYAYGVVLLELLFGRKPIDKSLPSECQSLISWAMPQLTDREKLPTIIDPMIKGAMNLKHLY
QVAAVAVLCVQPEPSYRPLIADVVHSLIPLVPVELGGTLKSSPIEMKSFASSQCSAHVAS
>SEQ ID NO: 67
ATGTTCTTGTTTCCTAAAACAGTTCCTATTTGGTTTTTTCATCTGTGTCTAGTAGCAGTTCATGCCAT
ACAAGAAGACCCACCTGTCCCTTCACCATCTCCCTCTCTCATTTCTCCTATTTCAACTTCAATGGCTG
CCTTCTCTCCAGGGGTTGAATCGGAAATGGGAATCAAAGACCACCCCCAGCATGATGACCTCCACA
GGAAAATAATCTTGTTGCTCACTGTTGCTTGTTGCATACTTGTTATCATCCTTCTTTCTTTGTGTTCTT
GTTTCATTTACTATAAGAAGTCCTCACAAAAGAAAAAAGCTACTCGGTGTTCAGATGTGGAGAAAG
GGCTTTCATTGGCACCATTTTTGGGCAAATTCAGTTCCTTGAAAATGGTTAGTAATAGGGGATCTGT
TTCATTAATTGAGTATAAGATACTAGAGAAAGGAACAAACAATTTTGGCGATGATAAATTGTTGGG
AAAGGGAGGATTTGGACGTGTATATAAGGCTGTAATGGAAGATGACTCAAGTGCTGCAGTCAAGA
AACTAGACTGCGCAACTGATGATGCGCAGAGAGAATTTGAGAATGAGGTGGATTTGTTAAGCAAA
TTTCACCATCCAAATATAATTTCTATTGTGGGTTTTAGTGTTCATGAGGAGATGGGGTTCATTATTT
ATGAGTTAATGCCAAATGGGTGCCTTGAAGATCTACTGCATGGACCTTCTCGTGGATCTTCACTAA
ATTGGCATTTAAGGTTGAAAATTGCTCTTGATACAGCAAGAGGATTAGAATATCTGCATGAATTCT
GCAAGCCAGCAGTGATCCATAGAGATCTGAAATCATCGAATATTCTTTTGGACGCCAACTTCAATG
CCAAGCTGTCAGATTTTGGTCTTGCTGTAGCTGATAGCTCTCATAACAAGAAAAAGCTCAAGCTTTC
AGGCACTGTGGGTTATGTAGCCCCAGAGTATATGTTAGATGGTGAATTGACGGATAAGAGTGATGT
CTATGCTTTTGGAGTTGTGCTTCTAGAGCTTCTATTAGGAAGAAGGCCTGTAGAAAAACTGACACC
AGCTCATTGCCAATCTATAGTAACATGGGCCATGCCTCAGCTCACTAACAGAGCTGTGCTTCCAAC
CCTTGTGGATCCTGTGATCAGAGATTCAGTAGATGAGAAGTACTTGTTCCAGGTTGCAGCAGTAGC
CGTGTTGTGTATTCAACCAGAGCCAAGTTACCGCCCTCTCATAACAGATGTTGTGCACTCTCTCGTC
CCATTAGTTCCTCTTGAGCTTGGAGGGACACTAAGAGTTCCACAGCCTACAACTCCCAGAGGTCAA
CGACAAGGCCCATCAAAGAAACTGTTTTTGGATGGTGCTGCCTCTGCT
>SEQ ID NO: 68
MFLFPKTVPIWFFHLCLVAVHAIQEDPPVPSPSPSLISPISTSMAAFSPGVESEMGIKDHPQHDDLHRKIIL
LLTVACCILVIILLSLCSCFIYYKKSSQKKKATRCSDVEKGLSLAPFLGKFSSLKMVSNRGSVSLIEYKILE
KGTNNFGDDKLLGKGGFGRVYKAVMEDDSSAAVKKLDCATDDAQREFENEVDLLSKFHHPNIISIVGF
SVHEEMGFIIYELMPNGCLEDLLHGPSRGSSLNWHLRLKIALDTARGLEYLHEFCKPAVIHRDLKSSNIL
LDANFNAKLSDFGLAVADSSHNKKKLKLSGTVGYVAPEYMLDGELTDKSDVYAFGVVLLELLLGRRP
VEKLTPAHCQSIVTWAMPQLTNRAVLPTLVDPVIRDSVDEKYLFQVAAVAVLCIQPEPSYRPLITDVVH
SLVPLVPLELGGTLRVPQPTTPRGQRQGPSKKLFLDGAASA
>SEQ ID NO: 69
GCTGCTGCGGTGAAGAGATTGGATGGTGGGGCTGGGGCACATGATTGCGAGAAGGAATTCGAGAA
TGAGTTAGATTTGCTTGGAAAGATTCGGCATCCGAACATTGTGTCCCTTGTGGGCTTCTGTATTCAT
GAGGAGAACCGTTTCATTGTTTATGAGCTGATAGAGAATGGGTCGTTGGATTCACAACTTCATGGG
CCATCACATGGTTCAGCTCTGAGCTGGCATATTCGGATGAAGATTGCTCTTGACACGGCAAGGGGA
TTAGAGTACCTGCATGAGCACTGCAACCCACCAGTTATCCATAGGGATCTGAAGTCATCTAACATA
CTTTTAGATTCAGACTTCAGTGCTAAGATTTCAGATTTTGGCCTTGCGGTGATTAGTGGGAATCACA
GCAAAGGGAATTTAAAGCTTTCTGGGACTATGGGCTATGTGGCCCCTGAGTACTTATTGGATGGGA
AGTTGACTGAGAAGAGCGATGTATATGCGTTTGGGGTGGTACTTCTAGAACTTCTACTGGGAAGGA
AACCTGTTGAGAAGATGGCACAATCTCAATGCCAATCAATTGTTACATGGGCCATGCCTCAGCTAA
CTGATAGATCCAAACTCCCTAACATAATTGATCCCATGATCAAGAACACAATGGATCTGAAACACT
TGTACCAAGTTGCTGCAATGGCTGTGCTCTGA
>SEQ ID NO: 70
AAAVKRLDGGAGAHDCEKEFENELDLLGKIRHPNIVSLVGFCIHEENRFIVYELIENGSLDSQLHGPSHG
SALSWHIRMKIALDTARGLEYLHEHCNPPVIHRDLKSSNILLDSDFSAKISDFGLAVISGNHSKGNLKLSG
TMGYVAPEYLLDGKLTEKSDVYAFGVVLLELLLGRKPVEKMAQSQCQSIVTWAMPQLTDRSKLPNIID
PMIKNTMDLKHLYQVAAMAVL
>SEQ ID NO: 71
ACCCTCGGTTATGTAGCTCCTGAGTATCTGTTAGATGGTAAGTTAACAGAGAAAAGCGATGTGTAT
GGGTTTGGAGTAGTGTTACTCGAGCTTCTGCTTGGGAAGAAGCCTATGGAGAAAGTGGCAACAACA
GCAACTCAGTGCCAGATGATAGTCACATGGACCATGCCTCAGCTCACTGACAGAACGAAACTTCCG
AATATCGTGGATCCGGTGATCAGAAACTCCATGGATTTAAAGCACTTGTACCAGGTTGCTGCTGTG
GCAGTATTGTGTGTGCAGCCAGAACCGAGTTATCGGCCATTGATAACTGATATTTTGCATTCTCTTG
TGCCCCTTGTCCCTGTTGAGCTTGGTGGGACGCTCAGGAACTCGATAACAATGGCTACAACAACAA
TATCTCCTGAAAGCTAA
>SEQ ID NO: 72
TLGYVAPEYLLDGKLTEKSDVYGFGVVLLELLLGKKPMEKVATTATQCQMIVTWTMPQLTDRTKLPNI
VDPVIRNSMDLKHLYQVAAVAVLCVQPEPSYRPLITDILHSLVPLVPVELGGTLRNSITMATTTISPES
>SEQ ID NO: 73
CGGCACGAGGGGCTGGTGGCCATGATCGAGTACCCGTCGCTGGAGGCGGCGACGGGCAAGTTCAG
CGAGAGCAACGTGCTCGGCGTCGGCGGGTTCGGCTGCGTCTACAAGGCGGCGTTCGACGGCGGCG
CCACCGCCGCCGTGAAGAGGCTCGAAGGCGGCGAGCCGGACTGCGAGAAGGAGTTCGAGAATGAG
CTGGACTTGCTTGGCAGGATCAGGCACCCAAACATAGTGTCCCTCCTGGGCTTCTGCGTCCATGGT
GGCAATCACTACATTGTTTATGAGCTCATGGAGAAGGGATCATTGGAGACACAACTGCATGGGCCT
TCACATGGATCGGCTATGAGCTGGCACGTCCGGATGAAGATCGCGCTCGACACGGCGAGGGGATT
AGAGTATCTTCATGAGCACTGCAATCCACCAGTCATCCATAGGGATCTGAAATCGTCTAATATACT
CTTGGATTCAGACTTCAATGCTAAGATTGCAGATTTTGGCCTTGCAGTGACAAGTGGGAATCTTGA
CAAAGGGAACCTGAAGATCTCTGGGACCTTGGGATATGTAGCTCCCGAGTACTTATTAGATGGGAA
GTTGACCGAGAAGAGCGACGTCTACGCGTTTGGAGTAGTGCTTCTAGAGCTCCTGATGGGGAGGAA
GCCTGTTGAGAAGATGTCACCATCTCAGTGCCAATCAATTGTGTCATGGGCCATGCCTCAGCTAAC
CGACAGATCGAAGCTACCCAACATCATCGACCCGGTGATCAAGGACACAATGGACCCAAAGCATT
TATACCAAGTTGCGGCGGTGGCCGTTCTATGCGTGCAGCCCGAACCGAGTTACAGACCGCTGATAA
CAGACGTTCTCCACTCCCTTGTTCCTCTGGTACCCGCGGATCTCGGGGGGAACGCTCAGAGTTACA
GAGCCGCATTCTCCACACCAAATGTACCATCCCTCTTGAGAAGTGATCCTACAAGTTTCGTCGAAG
CGGGGAAAGCGAATNTATACGGTCCAGCGGTAGATGGCTGTTATTTTGGTACTTATATCTCACCCT
GTCCTGCTGCTTATCTTAGGATGAGTGANGAGCTCCNACCTGCTGCTTTTGCTGGTTGGGCAGAGA
GAATACAGTTCTGGTTAGGATTG
>SEQ ID NO: 74
RHEGLVAMIEYPSLEAATGKFSESNVLGVGGFGCVYKAAFDGGATAAVKRLEGGEPDCEKEFENELDL
LGRIRHPNIVSLLGFCVHGGNHYIVYELMEKGSLETQLHGPSHGSAMSWHVRMKIALDTARGLEYLHE
HCNPPVIHRDLKSSNILLDSDFNAKIADFGLAVTSGNLDKGNLKISGTLGYVAPEYLLDGKLTEKSDVY
AFGVVLLELLMGRKPVEKMSPSQCQSIVSWAMPQLTDRSKLPNIIDPVIKDTMDPKHLYQVAAVAVLC
VQPEPSYRPLITDVLHSLVPLVPADLGGNAQSYRAAFSTPNVPSLLRSDPTSFVEAGKANXYGPAVDGC
YFGTYISPCPAAYLRMSXELXPAAFAGWAERIQFWLGL
>SEQ ID NO: 75
ATGAAAGTGATTGGGAGAAAGGGTTATGTCTCTTTTATTGATTATAAGGTACTAGAAACTGCAACA
AACAATTTTCAGGAAAGTAATATCCTGGGTGAGGGCGGGTTTGGTTGCGTCTACAAGGCGCGGTTG
GATGATAACTCCCATGTGGCTGTGAAGAAGATAGATGGTAGAGGCCAGGATGCTGAGAGAGAATT
TGAGAATGAGGTGGATTTGTTGACTAAAATTCAGCACCCAAATATAATTTCTCTCCTGGGTTACAG
CAGTCATGAGGAGTCAAAGTTTCTTGTCTATGAGCTGATGCAGAATGGATCTCTGGAAACTGAATT
GCACGGACCTTCTCATGGATCATCTCTAACTTGGCATATTCGAATGAAAATCGCTCTGGATGCAGC
AAGAGGATTAGAGTATCTACATGAGCACTGCAACCCACCAGTCATCCATAGAGATCTTAAATCATC
TAATATTCTTCTGGATTCAAACTTCAATGCCAAGCTTTCGGATTTTGGTCTAGCTGTAATTGATGGG
CCTCAAAACAAGAACAACTTGAAGCTTTCAGGCACCCTGGGTTATCTAGCTCCTGAGTATCTTTTAG
ATGGTAAACTGACTGATAAGAGTGATGTGTATGCATTTGGAGTGGTGCTTCTAGAGCTACTACTGG
GAAGAAAGCCTGTGGAAAAACTGGCACCAGCTCAATGCCAGTCCATTGTCACATGGGCCATGCCA
CAGCTGACTGACAGATCAAAGCTCCCAGGCATCGTTGACCCTGTGGTCAGAGACACGATGGATCTA
AAGCATTTATACCAAGTTGCTGCTGTAGCTGTGCTATGTGTGCAACCAGAACCAAGTTACCGGCCA
TTGATAACAGATGTTCTGCACTCCCTCATCCCACTCGTTCCAGTTGAGTTGGGAGGGATGCTAAAA
GTTACCCAGCAAGCGCCGCCTATCAACACCACTGCACCTTCTGCTGGAGGTTGA
>SEQ I DNO: 76
MKVIGRKGYVSFIDYKVLETATNNFQESNILGEGGFGCVYKARLDDNSHVAVKKIDGRGQDAEREFEN
EVDLLTKIQHPNIISLLGYSSHEESKFLVYELMQNGSLETELHGPSHGSSLTWHIRMKIALDAARGLEYL
HEHCNPPVIHRDLKSSNILLDSNFNAKLSDFGLAVIDGPQNKNNLKLSGTLGYLAPEYLLDGKLTDKSD
VYAFGVVLLELLLGRKPVEKLAPAQCQSIVTWAMPQLTDRSKLPGIVDPVVRDTMDLKHLYQVAAVA
VLCVQPEPSYRPLITDVLHSLIPLVPVELGGMLKVTQQAPPINTTAPSAGG
>SEQ ID NO: 77
ATGCCGCCGCCATCGCCGCTCCTCCGTTCCTCCGCCTTCGTCGTCTTGCTGCTCCTGGTGTGTCGCCC
GTTGTTGGTCGCCAATGGGAGGGCCACGCCGCCTTCTCCGGGATGGCCACCGGCGGCTCAGCCCGC
GCTGCAGCCTGCACCCACCGCCAGCGGCGGCGTGGCCTCCGTGCTTCCTTCGGCCGTGGCGCCTCC
TCCCTTAGGTGTGGTTGTGGCGGAGAGGCACCACCACCTCAGCAGGGAGCTCGTCGCTGCCATTAT
CCTCTCATCCGTCGCCAGCGTCGTGATCCCCATTGCCGCGCTGTATGCCTTCTTGCTGTGGCGACGA
TCACGGCGAGCCCTGGTGGATTCCAAGGACACCCAGAGCATAGATACCGCAAGGATTGCTTTTGCG
CCGATGTTGAACAGCTTTGGCTCGTACAAGACTACCAAGAAGAGTGCCGCGGCGATGATGGATTAC
ACATCTTTGGAGGCAGCGACAGAAAACTTCAGTGAGAGCAATGTCCTTGGATTTGGTGGGTTTGGG
TCTGTGTACAAAGCCAATTTTGATGGGAGGTTTGCTGCTGCGGTGAAGAGACTGGATGGTGGGGCA
CATGATTGCAAGAAGGAATTCGAGAATGAGCTAGACTTGCTTGGGAAGATTCGACATCCGAACATC
GTGTCCCTTGTGGGCTTCTGCATTCATGAGGAGAACCGTTTCGTTGTTTATGAGCTGATGGAGAGTG
GGTCGTTGGATTCGCAACTTCATGGGCCATCACATGGTTCAGCTCTGAGCTGGCATATTCGGATGA
AGATTGCTCTCGACACAGCAAGGGGATTAGAGTACCTGCATGAGCACTGCAACCCACCGGTTATCC
ATAGGGATCTTAAGTCATCTAACATACTTTTAGATTCAGACTTCAGCGCTAAGATTTCAGACTTTGG
CCTGGCAGTGACTAGTGGGAATCACAGCAAAGGGAATTTAAAGCTTTCTGGGACTATGGGCTATGT
GGCTCCTGAGTACTTATTAGATGGGAAGCTGACTGAGAAGAGCGATGTATACGCGTTTGGGGTAGT
ACTTCTAGAACTCCTGCTGGGAAGGAAACCTGTCGAGAAGATGGCACAATCTCAGTGCCGATCAAT
CGTTACATGGGCCATGCCTCAGCTAACTGATAGATCCAAGCTCCCGAACATAATTGATCCCATGAT
CAAGAACACAATGGATCTGAAACACTTGTACCAAGTTGCTGCAGTGGCCGTGCTCTGCGTGCAGCC
AGAGCCGAGTTACAGGCCACTGATCACCGACGTGCTTCACTCACTGGTACCTCTAGTGCCCACGGA
GCTTGGAGGAACGCTGAGGATCGGCCCGGAATCGCCCTACCTACGCTACTAA
>SEQ ID NO: 78
MPPPSPLLRSSAFVVLLLLVCRPLLVANGRATPPSPGWPPAAQPALQPAPTASGGVASVLPSAVAPPPLG
VVVAERHHHLSRELVAAIILSSVASVVIPIAALYAFLLWRRSRRALVDSKDTQSIDTARIAFAPMLNSFGS
YKTTKKSAAAMMDYTSLEAAIENFSESNVLGFGGFGSVYKANFDGRFAAAVKRLDGGAHDCKKEFE
NELDLLGKIRHPNIVSLVGFCIHEENRFVVYELMESGSLDSQLHGPSHGSALSWHIRMKIALDTARGLEY
LHEHCNPPVIHRDLKSSNILLDSDFSAKISDFGLAVTSGNHSKGNLKLSGTMGYVAPEYLLDGKLTEKSD
VYAFGVVLLELLLGRKPVEKMAQSQCRSIVTWAMPQLTDRSKLPNIIDPMIKNTMDLKHLYQVAAVAV
LCVQPEPSYRPLITDVLHSLVPLVPIELGGTLRIGPESPYLRY
>SEQ ID NO: 79
ATGTTGCTCGCGTGTCCTGCAGTGATCATCGTGGAGCGCCACCGTCATTTCCACCGTGAGCTAGTCA
TCGCCTCCATCCTCGCCTCAATCGCCATGGTCGCGATTATCCTCTCCACGCTGTACGCGTGGATCCC
GCGCAGGCGGTCCCGCCGGCTGCCCCGCGGCATGAGCGCAGACACCGCGAGGGGGATCATGCTGG
CGCCGATCCTGAGCAAGTTCAACTCGCTCAAGACGAGCAGGAAGGGGCTCGTGGCGATGATCGAG
TACCCGTCGCTGGAGGCAGCGACAGGGGGGTTCAGTGAGAGCAACGTGCTCGGCGTAGGCGGCTT
CGGTTGCGTCTACAAGGCAGTCTTCGATGGCGGCGTTACCGCGGCGGTCAAGAGGCTGGAGGGAG
GTGGCCCTGAGTGCGAGAAGGAATTCGAGAATGAGCTGGATCTGCTTGGCAGGATTCGGCACCCC
AACATCGTGTCCCTGCTGGGCTTTTGTGTTCACGAGGGGAATCACTACATTGTTTATGAGCTCATGG
AGAAGGGATCCCTGGACACACAGCTGCATGGGGCCTCACATGGATCAGCGCTGACCTGGCATATCC
GGATGAAGATCGCACTCGACATGGCCAGGGGATTAGAATACCTCCATGAGCACTGCAGTCCACCA
GTGATCCATAGGGATCTGAAGTCATCTAACATACTTTTAGATTCTGACTTCAATGCTAAGATTTCAG
ATTTTGGTCTTGCAGTGACCAGTGGGAACATTGACAAGGGAAGCATGAAGCTTTCTGGGACCTTGG
GTTATGTGGCCCCTGAGTACCTATTAGATGGGAAGCTGACTGAAAAGAGTGACGTATATGCATTTG
GAGTGGTGCTTCTTGAGCTACTAATGGGAAGGAAGCCTGTCGAGAAGATGAGTCAAACTCAGTGCC
AATCAATTGTGACGTGGGCCATGCCGCAGCTGACTGACAGAACAAAACTTCCCAACATAGTTGACC
CAGTGATCAGGGACACCATGGATCCAAAGCATTTGTACCAAGTGGCAGCAGTGGCAGTTCTATGTG
TGCAACCAGAACCAAGTTACAGACCGCTGATTACTGATGTTCTCCACTCTCTTGTCCCTCTAGTCCC
TGTGGAGCTCGGAGGGACACTGAGGGTTGTAGAGCCACCTTCCCCAAACCTAAAACATTCTCCTTGT
>SEQ ID NO: 80
MLLACPAVIIVERHRHFHRELVIASILASIAMVAIILSTLYAWIPRRRSRRLPRGMSADTARGIMLAPILSK
FNSLKTSRKGLVAMIEYPSLEAATGGFSESNVLGVGGFGCVYKAVFDGGVTAAVKRLEGGGPECEKEF
ENELDLLGRIRHPNIVSLLGFCVHEGNHYIVYELMEKGSLDTQLHGASHGSALTWHIRMKIALDMARGL
EYLHEHCSPPVIHRDLKSSNILLDSDFNAKISDFGLAVTSGNIDKGSMKLSGTLGYVAPEYLLDGKLTEK
SDVYAFGVVLLELLMGRKPVEKMSQTQCQSIVTWAMPQLTDRTKLPNIVDPVIRDTMDPKHLYQVAA
VAVLCVQPEPSYRPLITDVLHSLVPLVPVELGGTLRVVEPPSPNLKHSPC
>SEQ ID NO: 81
ATGAAGAAGAAGCTTGTGCTGCATCTGCTTCTTTTCCTTGTTTGTGCTCTTGAAAACATTGTTTTGGC
CGTACAAGGCCCTGCTTCATCACCCATTTCTACTCCCATCTCTGCTTCAATGGCTGCCTTCTCTCCAG
CTGGGATTCAACTTGGAGGTGAGGAGCACAAGAAAATGGATCCAACCAAGAAAATGTTATTAGCT
CTCATTCTTGCTTGCTCTTCATTGGGTGCAATTATCTCTTCCTTGTTCTGTTTATGGATTTATTACAG
GAAGAATTCAAGCAAATCCTCTAAAAATGGCGCTAAGAGCTCAGATGGTGAAAAAGGGAATGGTT
TGGCACCATATTTGGGTAAATTCAAGTCTATGAGGACGGTTTCCAAAGAGGGTTATGCTTCGTTTAT
GGACTATAAGATACTTGAAAAAGCTACAAACAAGTTCCATCATGGTAACATTCTGGGTGAGGGTGG
ATTTGGATGTGTTTACAAGGCTCAATTCAATGATGGTTCTTATGCTGCTGTTAAGAAGTTGGACTGT
GCAAGCCAAGATGCTGAAAAAGAATATGAGAATGAGGTGGGTTTGCTATGTAGATTTAAGCATTCC
AATATAATTTCACTGTTGGGTTATAGCAGTGATAACGATACAAGGTTTATTGTTTATGAGTTGATGG
AAAATGGTTCTTTGGAAACTCAATTACATGGACCTTCTCATGGTTCATCATTAACTTGGCATAGGAG
GATGAAAATTGCTTTGGATACAGCAAGAGGATTAGAATATCTACATGAGCATTGCAATCCACCAGT
CATCCATAGAGATCTGAAATCATCTAATATACTTTTGGATTTGGACTTCAATGCAAAGCTTTCAGAT
TTTGGTCTTGCAGTAACTGATGCGGCAACAAACAAGAATAACTTGAAGCTTTCGGGTACTTTAGGT
TATCTAGCTCCAGAATACCTTTTAGATGGTAAATTAACAGATAAGAGTGATGTTTATGCATTCGGTG
TTGTGCTGCTCGAACTTCTATTGGGACGAAAGGCTGTTGAAAAATTATCACAACTCAGTGCCAATC
TTAGGTCCATTTGGGCATAG
>SEQ ID NO: 82
MKKKLVLHLLLFLVCALENIVLAVQGPASSPISTPISASMAAFSPAGIQLGGEEHKKMDPTKKMLLALIL
ACSSLGAIISSLFCLWIYYRKNSSKSSKNGAKSSDGEKGNGLAPYLGKFKSMRTVSKEGYASFMDYKIL
EKATNKFHHGNILGEGGFGCVYKAQFNDGSYAAVKKLDCASQDAEKEYENEVGLLCRFKHSNIISLLG
YSSDNDTRFIVYELMENGSLETQLHGPSHGSSLTWHRRMKIALDTARGLEYLHEHCNPPVIHRDLKSSNI
LLDLDFNAKLSDFGLAVTDAATNKNNLKLSGTLGYLAPEYLLDGKLTDKSDVYAFGVVLLELLLGRKA
VEKLSQLSANLRSIWA
>SEQ ID NO: 83
GGAGTGGGAATTGAGAAGCAGCCACCCACCCACCCACCCTATGGATAAAAATAGAAGGCTGTTGA
TAGCACTCATTGTAGCTTCTACTGCATTAGGACTAATCTTTATCTTCATCATTTTATTCTGGATTTTT
CACAAAAGATTTCACACCTCAGATGTTGTGAAGGGAATGAGTAGGAAAACATTGGTTTCTTTAATG
GACTACAACATACTTGAATCAGCCACCAACAAATTTAAAGAAACTGAGATTTTAGGTGAGGGGGG
TTTTGGATGTGTGTACAAAGCTAAATTGGAAGACAATTTTTATGTAGCTGTCAAGAAACTAACCCA
AAATTCCATTAAAGAATTTGAGACTGAGTTAGAGTTGTTGAGTCAAATGCAACATCCCAATATTAT
TTCATTGTTGGGATATTGCATCCACAGTGAAACAAGATTGCTTGTCTATGAACTCATGCAAAATGG
ATCACTAGAAACTCAATTACATGGGCCTTCCCGTGGATCAGCATTAACTTGGCATCGCAGGATAAA
AATTGCCCTTGATGCAGCAAGAGGAATAGAATATTTACATGAGCAGCGCCATCCCCCTGTAATTCA
TAGAGATCTGAAATCATCTAATATTCTTTTAGATTCCAACTTCAATGCAAAGGTAAAACTTTTTATG
TAGAAATTATACTAGGACTAGTTTTCCCTCTATTAATCTTGTGTTGTGATTAATTTTAGCTGTCAGAT
TTTGGTCTTGCTGTGTTGAGTGGGGCTCAAAACAAAAACAATATCAAGCTTTCTGGAACTATAGGT
TATGTAGCGCCTGAATACATGTTAGATGGAAAATTAAGTGATAAAAGTGATGTTTATGGTTTTGGA
GTAGTACTTTTGGAGCTGTTATTGGGAAGGCGGCCTGTAGAAAAGGAGGCAGCCACTGAATGTCAG
TCTATAGTGACATGGGCCATGCCTCAGCTGACAGATAGATCAAAGCTTCCAAACATTGTTGATCCT
GTCATACAAAACACAATGGATTTAAAGCATNTGTATCAGGTTGCTGCAGGTGCTCTATTATGTGTTC
AGCCAGAGCCAAGCTATCGTCCCGTATAA
>SEQ ID NO: 84
GAGTATCAGTTATTGGAAGCTGCAACTGACAATTTTAGTGAGAGTAATATTTTGGGAGAAGGTGGA
TTTGGATGTGTTTACAAAGCATGTTTTGATAACAACTTTCTCGCTGCTGTCAAGAGAATGGATGTTG
GTGGGCAAGATGCAGAAAGAGAATTTGAGAAAGAAGTAGATTTGTTGAATAGAATTCAGCATCCG
GATATAATTTCCCTGTTGGGTTATTGTATTCATGATGAGACAAGGTTCATCATTTATGAACTAATGC
AGAACGGATCTTTGGAAAGACAATTACATGGACCTTCTCATGGATCGGCTTTAACTTGGCATATCC
GGATGAAAATTGCACTTGATACAGCAAGAGCATTAGAATATCTCCATGAGAATTGCAACCCTCCTG
TGATCCACAGAGATCTGAAATCATCCAATATACTTTTGGATTCTAATTTCAAGGCCAAGATTTCAGA
TTTTGGTCTTGCTGTAATTTCTGGGAGTCAAAACAAGAACAACATTAAGCTTTCAGGCACTCTTGGT
TATGTTGCTCCAGAATATCTGTTAGATGGTAAATTGACTGACAAAAGTGATGTCTATGCTTTTGGGG
TTATCCTTCTAGAACTCCTAATGGGAAGAAAACCTGTAGAGAAAATGACACGAACTCAGTGTCAAT
CTATCGTTACATGGGCCATGCCTCAACTCACTGATAGATCAAAGCTACCAAACATTGTTGATCCTGT
GATTAAAAACACAATGGATTTGAAGCATTTGTTCCAAGTTGCTGCTGTAGCTGTACTGTGTGTACA
ACCAGAACCAAGTTACCGGCCATTAATCACAGATGTCCTTCACTCCCTCGTACCCCTTGTTCCTGTC
GATCTTGGAGG
>SEQ ID NO: 85
EYQLLEAATDNFSESNILGEGGFGCVYKACFDNNFLAAVKRMDVGGQDAEREFEKEVDLLNRIQHPDII
SLLGYCIHDETRFIIYELMQNGSLERQLHGPSHGSALTWHIRMKIALDTARALEYLHENCNPPVIHRDLK
SSNILLDSNFKAKISDFGLAVISGSQNKNNIKLSGTLGYVAPEYLLDGKLTDKSDVYAFGVILLELLMGR
KPVEKMTRTQCQSIVTWAMPQLTDRSKLPNIVDPVIKNTMDLKHLFQVAAVAVLCVQPEPSYRPLITDV
LHSLVPLVPVDLGG
>SEQ ID NO: 86
TGTGCTCATGATGAGACCAAACTACTTGTTTACGAACTTATGCACAATGGTTCGTTAGAAACTCAAT
TACACGGTCCTTCTTGTGGATCCAATTTAACATGGCATTGTCGGATGAAAATTGCGCTAGATATAGC
GAGAGGATTGGAATATTTACATGAACACTGCAAACCATCTGTGATTCATAGAGATTTGAAGTCATC
TAACATCCTTTTGGATTCAAAATTCAATGCCAAGCTTTCGGATTTCGGTCTTGCTGTGATGAACGGT
GCCAATACCAAAAACATTAAGCTTTCGGGGACGTTGGGTTACGTAGCTCCCGAGTATCTTTTAAAT
GGGAAATTGACCGATAAAAGTGACGTCTACGCATTCGGAGTTGTACTTTTAGAGCTTCTACTCAAA
AGGCGGCCTGTCGAAAAACTAGCACCATCCGAGTGCCAGTCCATCGTCACTTGGGCTATGCCGCAA
CTAACAGACAGAACAAAGCTTCCGAGTGTTATAGATCCCGTGATCAGGGACACGATGGATCTTAAA
CACTTGTATCAAGTGGCGGCTGTGGCTGTGTTGTGTGTTCAACCGGAACCGGGATACCGGCCGTTG
ATAACCGACGTCTTGCATTCTCTGGTTCCTCTCGTGCCGGTTGAACTCGGAGGGACTCTACGAGTTG
CGGAAACAGGTTGCGGCACAGTTGACTTATGA
>SEQ ID NO: 87
CAHDETKLLVYELMHNGSLETQLHGPSCGSNLTWHCRMKIALDIARGLEYLHEHCKPSVIHRDLKSSNI
LLDSKFNAKLSDFGLAVMNGANTKNIKLSGTLGYVAPEYLLNGKLTDKSDVYAFGVVLLELLLKRRPV
EKLAPSECQSIVTWAMPQLTDRTKLPSVIDPVIRDTMDLKHLYQVAAVAVLCVQPEPGYRPLITDVLHS
LVPLVPVELGGTLRVAETGCGTVDL
>SEQ ID NO: 88
TGGATTTGGATGCGTTTAAAAGCTCAACTCAATGATAACTTATTAGTTGCGGTCAAACGACTAGAC
AATAAAAGTCAAAATTCCATCAAAGAATTCCAGACGGAAGTGAATATTTTGAGTAAAATTCAACAT
CCAAATATAATTAGTTTGTTGGGATATTGCGATCATGATGAAAGCAAGCTACTTGTTTACGAATTG
ATGCAAAATGGTTCTTTAGAAACTCAGTTACATGGGCCTTCTTGTGGATCCAATTTAACATGGTATT
GCCGGATGAAAATTGCCCTAGATATAGCAAGAGGATTGGAATATTTACATGAACACTCCAAACCAT
CTGTGATTCATAGAGATCTCAAATCATCTAATATACTTCTTGATTCAAATTTCAATGCAAAGCTTTC
GGATTTTGGTCTTGCGGTGATGGAAGGTGCAAATAGCAAAAACATTAAACTTTCGGGGACATTGGG
ATACGTAGCACCCGAATATCTTTTAGATGGGAAATTAACCGATAAAAGTGACGTGTATGCATTTGG
AGTCGTACTTTTTGAGCTTTTACTCAGAAGACGACACGTTGAAAAACTAGAATCATCACAATCCCG
CCAATCTATTGTCACTTGGGCGATGCCACTACTAATGGACAGATCGAAGCTTCCGAGTGTGATAGA
TCCTGTGATTAGGGATACAATGGATCTTAAACATCTTTATCAAGTGGCTGCGGTGGCGGTGTTGTGT
GTTCAATCGGAACCGAGTTACCGTCCGTTGATAACCGATGTTTTACATTCTCTTGTTCCTCTTGTCCC
GGTTGAACTTGGAGGGACACTTAGAGTTGTAGAAAAGAGTGTTGT
>SEQ ID NO: 89
WIWMRLKAQLNDNLLVAVKRLDNKSQNSIKEFQTEVNILSKIQHPNIISLLGYCDHDESKLLVYELMQN
GSLETQLHGPSCGSNLTWYCRMKIALDIARGLEYLHEHSKPSVIHRDLKSSNILLDSNFNAKLSDFGLAV
MEGANSKNIKLSGTLGYVAPEYLLDGKLTDKSDVYAFGVVLFELLLRRRHVEKLESSQSRQSIVTWAM
PLLMDRSKLPSVIDPVIRDTMDLKHLYQVAAVAVLCVQSEPSYRPLITDVLHSLVPLVPVELGGTLRVV
EKSVV
>SEQ ID NO: 90
ATTCTTTTAGATGCAAACTTCAATGCCAAGCTTTCTGATTTTGGCTTGTCTGTCATTGTTGGAGCAC
AAAACAAGAATGATATAAAGCTTTCCGGAACGATGGGTTATGTTGCTCCTGAATATCTTTTAGATG
GTAAATTGACTGATAAAAGTGATGTCTATGCTTTTGGAGTTGTGCTTTTGGAGCTTCTTTTAGGAAG
AAGGCCTGTTGAAAAACTGGCACCATCTCAATGTCAATCCATTGTCACATGGGCTATGCCTCAACT
CACTGATAGATCAAAGTTACCCGATATCGTTGATCCGGTGATCAGACACACAATGGACCCTAAACA
TTTATTTCAGGTTGCTGCTGTCGCCGTGCTGTGTGTGCAACCAGAACCGAGCTATCGTCCCCTAATA
ACAGATCTTTTGCACTCTCTTATTCCTCTTGTTCCTGTTGAGCTAGGAGGTACTCACAGATCATCAA
CATCACAAGCTCCTGTGGCTCCAGCTTAG
>SEQ ID NO: 91
ILLDANFNAKLSDFGLSVIVGAQNKNDIKLSGTMGYVAPEYLLDGKLTDKSDVYAFGVVLLELLLGRRP
VEKLAPSQCQSIVTWAMPQLTDRSKLPDIVDPVIRHTMDPKHLFQVAAVAVLCVQPEPSYRPLITDLLH
SLIPLVPVELGGTHRSSTSQAPVAPA
>SEQ ID NO: 92
GATGGGAAGCTCACCGAGAAAAGCGACGTGTACGCGTTTGGCATAGTGCTTCTTGAGCTGCTAATG
GGAAGGAAGCCTGTTGAGAAGTTGAGTCAATCTCAGTGCCAATCAATTGTGACTTGGGCCATGCCC
CAACTGACAGACAGATCAAAACTTCCCAACATAATTGACCCAGTGATCAGGGACACAATGGATCC
AAAGCACTTGTATCAGGTTGCAGCAGTGGCTGTTCTATGCGTGCAACCAGAACCGAGTTACAGACC
ACTGATAACGGATGTTCTCCACTCTTTAGTTCCTCTAGTGCCTGTGGAGCTTGGTGGGACACTAAGG
GTTGCAGAGCCACCGTCCCCAAACCAAAATCATTCTCCTCGTTGA
>SEQ ID NO: 93
DGKLTEKSDVYAFGIVLLELLMGRKPVEKLSQSQCQSIVTWAMPQLTDRSKLPNIIDPVIRDTMDPKHL
YQVAAVAVLCVQPEPSYRPLITDVLHSLVPLVPVELGGTLRVAEPPSPNQNHSPR
>SEQ ID NO: 94
GGGGTTCATGGCAAGAACAATATAAAACTTTCAGGAACTTTAGGATATGTCGCGCCGGAATACCTT
TTAGATGGTAAACTTACTGATAAAAGTGACGTTTATGCGTTTGGAGTTGTGCTTCTCGAGCTTTTGA
TAGGACGAAAACCCGTGGAGAAAATGTCACCATTTCAATGCCAATTTATCGTTACATGGGCAATGC
CTCAGCTAACGGACAGATCGAAGCTTCCTAATCTTGTGGATCCTGTGATTAGAGATACTATGGACT
TGAAGCCCTTATATCAAGTTGCGGCTGTAACTGTGTTATGTGTACAACCCGAACCAAGTTACCGCC
CATTAATAACGGATGTTTTGCATTCGTTCATCCCACTTGTACCTGCTGATCTTGGAGGGTCGTTAAA
AGTTGTCGACTTTTAA
>SEQ ID NO: 95
GVHGKNNIKLSGTLGYVAPEYLLDGKLTDKSDVYAFGVVLLELLIGRKPVEKMSPFQCQFIVTWAMPQ
LTDRSKLPNLVDPVIRDTMDLKPLYQVAAVTVLCVQPEPSYRPLITDVLHSFIPLVPADLGGSLKVVDF
>SEQ ID NO: 96
ATCGTGTTCCATTTTGGTTGTTGTCTAAAGCTTTCAGATTTTGGTCTTGCTGTAATGGATGGAGCCC
AGAACAAAAACAACATCAAGCTTTCAGGGACATTGGGTTATGTAGCTCCAGAGTATCTTTTAGATG
GAAAACTGACCGACAAAAGTGATGTATATGCATTTGGAGTTGTACTTTTAGAGCTTCTACTTGGAA
GACGGCCTGTAGAAAAACTGGCCGCATCTCAATGCCAATCTATCGTCACTTGGGCCATGCCACAGC
TAACAGACAGATCAAAGCTCCCAAATATTGTCGATCCTGTAATCAGATATACGATGGATCTCAAAC
ACTTGTACCAAGTTGCTGCCGTGGCAGTGCTGTGTGTGCAACCAGAGCCAAGTTACCGGCCATTAA
TAACCGATGTTTTGCATTCTCTTATCCCTCTTGTTCCGGTGGAGCTCGGGGGAACTCTAAAAGCTCC
ACAAACAAGGTCTTCGGTAACAAATGACCCGTGA
>SEQ ID NO: 97
IVFHFGCCLKLSDFGLAVMDGAQNKNNIKLSGTLGYVAPEYLLDGKLTDKSDVYAFGVVLLELLLGRR
PVEKLAASQCQSIVTWAMPQLTDRSKLPNIVDPVIRYTMDLKHLYQVAAVAVLCVQPEPSYRPLITDVL
HSLIPLVPVELGGTLKAPQTRSSVTNDP
>SEQ ID NO: 98
CGTGGATCAACTTTAAGTTGGCCTCTCCGAATGAAAATTGCTTTGGATATTGCAAGAGGATTAGAA
TACCTTCACGAGCGTTGCAACCCCCCTGTGATCCATAGGCATCTCAAATCGTCTAATATTCTTCTTG
ATTCCAGCTTCAACGCAAAGATTTCTGATTTTGGCCTTTCTGTAACTGGCGGAAACCTAAGCAAGA
ACATAACCAAGATTTCGGGATCACTGGGTTATCTTGCTCCAGAGTATCTCTTAGACGGTAAACTAA
CTGATAAGAGTGATGTGTATGGTTTTGGCATTATTCTTCTAGAGCTTTTGATGGGTAAAAGGCCAGT
GGAGAAAGTGGGAGAAACTAAGTGCCAATCAATAGTTACATGGGCTATGCCCCAGCTTACGGACC
GATCAAAGCTTCCGAATATTGTTGACCCTACGATCAGGAACACAATGGATGTTAAGCATTTATATC
AGGTTGCGGCTGTAGCTGTGTTATGTGTGCAACCGGAGCCAAGCTATAGGCCATTGATAACTGATG
TACTACACTCCTTCATTCCACTTGTACCAAATGAACTCGGGGGGTCGCTTAGGGTAGTGGATTCTAC
TCCCCATTGCTCATAG
>SEQ ID NO: 99
RGSTLSWPLRMKIALDIARGLEYLHERCNPPVIHRHLKSSNILLDSSFNAKISDFGLSVTGGNLSKNITKIS
GSLGYLAPEYLLDGKLTDKSDVYGFGIILLELLMGKRPVEKVGETKCQSIVTWAMPQLTDRSKLPNIVD
PTIRNTMDVKHLYQVAAVAVLCVQPEPSYRPLITDVLHSFIPLVPNELGGSLRVVDSTPHCS
>SEQ ID NO: 100
TTAGATAATGGCGGACCCGATTGTCAACGAGAATTCGAGAATGAGGTTGATTTGATGAGTAGAATT
AGGCATCCAAATGTGGTTTCTTTATTGGGTTATTGCATTCATGGAGAAACCAGGCTTCTTGTCTATG
AAATGATGCAAAACGGGACGTTGGAATCGCTATTGCATGGACCATCACATGGATCCTCACTAACTT
GGCACATTCGTATGAAGATCGCCCTCGACACAGCAAGAGGCCTCGAGTATCTGCATGAACACTGCG
ACCCCTCTGTGATCCACCGTGACCTGAAGCCTTCTAACATTCTTTTGGATTCCAACTACAATTCCAA
GCTCTCAGACTTTGGTCTTGCAGTCACTGTTGGAAGCCAGAATCAAACCAACATTAAGATTCTAGG
GACACTGGGTTACCTTGCACCAGAGTACGTTTTGAATGGCAAATTGACAGAGAAAAGTGATGTGTT
TGCTTTTGGAGTTGTCCTGTTGGAGCTTCTCATGGGCAAGAAACCAGTGGAGAAGATGGCATCCCC
TCCATGCCAATCCATTGTCACATGGGCGATGCCTCATCTTACTGACAGAATTAAGCTTCCAAATATC
ATTGATCCTGTTATTAGAAACACCATGGATCTGAAACACTTGTACCAGGTTGCAGCTGTTGCTGTTC
TCTGCGTACAACCAGAGCCCCAGTTATCGTCCTCTGATAACTGA
>SEQ ID NO: 101
LDNGGPDCQREFENEVDLMSRIRHPNVVSLLGYCIHGETRLLVYEMMQNGTLESLLHGPSHGSSLTWHI
RMKIALDTARGLEYLHEHCDPSVIHRDLKPSNILLDSNYNSKLSDFGLAVTVGSQNQTNIKILGTLGYLA
PEYVLNGKLTEKSDVFAFGVVLLELLMGKKPVEKMASPPCQSIVTWAMPHLTDRIKLPNIIDPVIRNTM
DLKHLYQVAAVAVLCVQPEPQLSSSDN
>SEQ ID NO: 102
TCGGCTCGGCCCAGAACAAGATCGCAAGAC
>SEQ ID NO: 103
CTACATTCTCTCCTCGTATTATTCCTCGTTGACT
>SEQ ID NO: 104
ACTTTCAGATGAGTGGATCATAACCCTATACA
>SEQ ID NO: 105
AGATACAATGGATCTCAAACACTTATACCAG
>SEQ ID NO: 106
AAAGGATCCATGGGAAGTGGTGAAGAAGATAGATTTGATGCT
>SEQ ID NO: 107
TTTCTGCAGTCTGTGAATCATCTTGTTAACCGGAGAGTCC
>SEQ ID NO: 108
TCTGAGTTTTAATCGAGCCAAGTCGTCTCA
>SEQ ID NO: 109
TATCCCGGGAAAATGAGAGAGCTTCTTCTTCTTCTTCTTCTTCATTTTCAGTC
>SEQ ID NO: 110
TTTGGATCCTGTGAATCATCTTGTTAACCGGAGAGTCC
>SEQ ID NO: 111
ATACCCGGGTCTGTGTCAGGAATCCAAATGGGAAGTGGTGA
>SEQ ID NO: 112
AAAGGATCCTCTGTGTCAGGAATCCAAATGGGAAGTGGTGA
>SEQ ID NO: 113
AAATCTAGACTGTGAATCATCTTGTTAACCGGAGAGTCC
>SEQ ID NO: 114
ATAGAGCTCGCAAGAACCAATCTCCAAAATCCATC
>SEQ ID NO: 115
ATAGAGCTCGAGGGTCTTGATATCGAAAAATTGCACG
>SEQ ID NO: 116
ATAGGATCCTCGCAAGAACCAATCTCCAAAATCCATC
>SEQ ID NO: 117
ATATCTAGACTCGAGGGTCTTGATATCGAAAAATTGCACG
>SEQ ID NO: 118
ATATCTAGAAAATGAGAGAGCTTCTTCTTCTTCTTCTTCTTCATTTTCAGTC
>SEQ ID NO: 119
ATAGGATCCTGTTAAAAGCGATTTATAATTTACACCGTTTTGGTGTA
>SEQ ID NO: 120
ATACCCGGGAAAAGTTTTTGATGAAATTCAATCTAAAGACT
>SEQ ID NO: 121
AAAATGAGAGAGCTTCTTCTTCTTCTTCTTCTTCATTTTCAGTCTCTAATTCTTTTGATGATCTTCATC
ACTGTCTCTGCTTCTTCTGCTTCAAATCCTTCTTTAGCTCCTGTTTACTCTTCCATGGCTACATTCTCT
CCTCGAATCCAAATGGGAAGTGGTGAAGAAGATAGATTTGATGCTCATAAGAAACTTCTGATTGGT
CTCATAATCAGTTTCTCTTCTCTTGGCCTTATAATCTTGTTCTGTTTTGGCTTTTGGGTTTATCGCAA
GAACCAATCTCCAAAATCCATCAACAACTCAGATTCTGAGAGTGGGAATTCATTTTCCTTGTTAATG
AGACGACTTGGCTCGATTAAAACTCAGAGAAGAACTTCTATCCAAAAGGGTTACGTGCAATTTTTC
GATATCAAGACCCTCGAGAAAGCGACAGGCGGTTTTAAAGAAAGTAGTGTAATCGGACAAGGCGG
TTTCGGATGCGTTTACAAGGGTTGTTTGGACAATAACGTTAAAGCAGCGGTCAAGAAGATCGAGAA
CGTTAGCCAAGAAGCAAAACGAGAATTTCAGAATGAAGTTGACTTGTTGAGCAAGATCCATCACTC
GAACGTTATATCATTGTTGGGCTCTGCAAGCGAAATCAACTCGAGTTTCATCGTTTATGAGCTTATG
GAGAAAGGATCATTAGATGAACAGTTACATGGGCCTTCTCGTGGATCAGCTCTAACATGGCACATG
CGTATGAAGATTGCTCTTGATACAGCTAGAGGACTAGAGTATCTCCATGAGCATTGTCGTCCACCA
GTTATCCACAGAGATTTGAAATCTTCGAATATTCTTCTTGATTCTTCCTTCAACGCCAAGATTTCAG
ATTTCGGTTTTGCTGTATCGCTGGATGAACATGGCAAGAACAACATTAAACTCTCTGGGACACTTG
GTTATGTTGCCCCGGAATACCTCCTTGACGGAAAACTGACGGATAAGAGTGATGTTTATGCATTTG
GGGTAGTTCTGCTTGAACTCTTGTTGGGTAGACGACCAGTTGAAAAATTAACTCCAGCTCAATGCC
AATCTCTTGTAACTTGGGCAATGCCACAACTTACCGATAGATCCAAGCTTCCAAACATTGTGGATG
CCGTTATAAAAGATACAATGGATCTCAAACACTTATACCAGGTAGCAGCCATGGCTGTGTTGTGCG
TGCAGCCAGAACCAAGTTACCGGCCGTTGATAACCGATGTTCTTCACTCACTTGTTCCACTGGTTCC
GGTAGAGCTAGGAGGGACTCTCCGGTTAACAAGATGATTCACAG
>SEQ ID NO: 122
TCGGACAAGGCGGTTTCGGATGCGT
>SEQ ID NO: 123
TAGTCCTCTAGCTGTATCAAGAGCAATCTTCA
>SEQ ID NO: 124
TATCATTGTTGGGCTCTGCAAGTGAAATCAAC
>SEQ ID NO: 125
TGGAGAAAGGATCCTTAGATGATCAGTTACAT
>SEQ ID NO: 126
TCCATGTAACTGATCATCTAAGGATCCTTTC
>SEQ ID NO: 127
ATAAACGACGAAACTCGAGTTGATTTCACTTGCAGAG
>SEQ ID NO: 128
AAAATGAAGAAACTGGTTCATCTTCAGT
>SEQ ID NO: 129
TAGACTTCTATTCTCACATTCTTACAC
>SEQ ID NO: 130
TCCAATGATCCATTATGCATCAGCTCA
>SEQ ID NO: 131
TCGTTCTCAAATTCTCTCTCAGCATGTTG
>SEQ ID NO: 132
TCCGGATATGCCAGGTCAGCGCTGATCCA
>SEQ ID NO: 133
TCCAGGGATCCCTTCTCCATGAGCTCAT
>SEQ ID NO: 134
AAAGAGCTCTCTGTGTCAGGAATCCAAATGGGAAGTGGTGA
>SEQ ID NO: 135
ATAGCTAGCTGTTAAAAGCGATTTATAATTTACACCGTTTTGGTGTA
>SEQ ID NO: 136
ATAGCTAGCAGAAAAGTTTTTGATGAAATTCAATCTAAAGACT
>SEQ ID NO: 137
TCTGGGTTTATCATCATACCAAGTATCCA
>SEQ ID NO: 138
ATTCAGTTCCATCAAGATTGTTGGCATGGAC
>SEQ ID NO: 139
TGGAGGGAGGTGGCCCTGAGTGCGAGAAGGA
>SEQ ID NO: 140
GCTGGATCTGCTTGGCAGGATTCGGCA
>SEQ ID NO: 141
ATATCTAGATGCTAGGTTATAGATCCATGCA
>SEQ ID NO: 142
ATAGGATCCACCAGAACTATATATACGAAGGCA
>SEQ ID NO: 143
AGGACGACTTGGCTCGATTAAAATCACAGGTCGTGATATG
>SEQ ID NO: 144
TAATCGAGCCAAGTCGTCCTACATATATATTCCTA
>SEQ ID NO: 145
TAATCGAGCCAAGTCGTCCTCTCTTTTGTATTCCA
>SEQ ID NO: 146
AGGACGACTTGGCTCGATTAAAATCAAAGAGAATCAATGATC
>SEQ ID NO: 147
GACGACTTGGCTCGATTAAAA
>SEQ ID NO: 148
TGCTAGGTTATAGATCCATGCAAATATGGAGTAGATGTACAAACACACGCTCGGACGCATATTACA
CATGTTCATACACTTAATACTCGCTGTTTTGAATTGATGTTTTAGGAATATATATGTAGAGAGAGCT
TCCTTGAGTCCATTCACAGGTCGTGATATGATTCAATTAGCTTCCGACTCATTCATCCAAATACCGA
GTCGCCAAAATTCAAACTAGACTCGTTAAATGAATGAATGATGCGGTAGACAAATTGGATCATTGA
TTCTCTTTGATTGGACTGAAGGGAGCTCCCTCTCTCTTTTGTATTCCAATTTTCTTGATTAATCTTTC
CTGCACAAAAACATGCTTGATCCACTAAGTGACATATATGCTGCCTTCGTATATATAGTTCTGGT
>SEQ ID NO: 149
TGCTAGGTTATAGATCCATGCAAATATGGAGTAGATGTACAAACACACGCTCGGACGCATATTACA
CATGTTCATACACTTAATACTCGCTGTTTTGAATTGATGTTTTAGGAATATATATGTAGGACGACTT
GGCTCGATTAAAATCACAGGTCGTGATATGATTCAATTAGCTTCCGACTCATTCATCCAAATACCG
AGTCGCCAAAATTCAAACTAGACTCGTTAAATGAATGAATGATGCGGTAGACAAATTGGATCATTG
ATTCTCTTTGATTTTAATCGAGCCAAGTCGTCCTCTCTTTTGTATTCCAATTTTCTTGATTAATCTTTC
CTGCACAAAAACATGCTTGATCCACTAAGTGACATATATGCTGCCTTCGTATATATAGTTCTGGT
>SEQ ID NO: 150
CTTAGCCAATGGATGAGGATGACACGATAATGATAATCAAAGATCAACATGGCACGCTCAAGACC
GCCTTTAGAAGTCCTCTCTAAATTCTTTCTTCCGATCTCCTAAATATGTTTTGTTTTGGTCAAATAAA
TTGATAGGTAATACTTAGTGATTATACTATTTGGTTTTTGTTTTATCATTGACTATTTCACTTTTATA
AATCAAATACTTATCAAAATTGTTCTTTCCGTATGTATTCATATTTTCTAATATTGTAAAGATTTGTT
TCACCTAACATCTGTACCCATCTTTGATCATTGACAAAATATATATTAGAATGGCCTTAGAACGTGT
TAGGCATCTTCCTACTATTATCATATTACCTAATCCCCAATTTTATTACATTTTTTAATTTCTAAAAG
AGCTTGAATATAATGTCATTTCGAATATCTCTGTTCATCTTTTTTTTTTTCTGTGCGACTTCTGACCC
AAAGCCTTCGACGATTTTTTCCAATCTGAAAACTTTTGAATAAGGAACTTAGTCAATGGTCAACAC
CTTGCTAATTAAACAAAGTTCCATTGATACAATAATGAGATTTTTGTACATTAACGCTTTCATATAG
TTTTTGCGATTCAACAGATAATCTTAAAATTAAGGAGTCCTATTGATAAAGTCTTGTTCAAACGTAC
AAACTCAATCCACACAAAACCTTCATAAAATACGATATAGGAAATAAAGATTGTTTTTGCGTGAGA
AAATACTATATGAACTCAAAAGATTTTAAAACAATTTGTATTAATACATAAACAATTGTTGTGATA
CACCCGTGTAAAATTTTAAGATTGTTTTTTTCTGAAATTCTTCAAGGAAACTTATAGCTTAAAATCT
ACACTTCAAATACTCTGTTTTAAAGGCATTAAAAATAACTGCGTTTCAGAAAAATATTGAAATTTTA
GCTGATCTTTTGCTACAAATTTAAGGAATCTTGGCACCTGCAGAATCTATAACATGTTCATTAAGTA
ATGCAATAGTTATACAATTATACATTATTTGCATCATACTTATATTATAGTGATATTAACAAACCCA
TGTTCTCAGCACACTTTTACGTAGAAAAACATAAAAACCCAAATAGGAAGAAGCCACTCATAAGG
ATAATGGGTTTATATAATTCACAGCAAAGAAAGCCATCGAACTATTCGATTAATTATCCATTCTTTT
TTTTTTTAGTTTGAATGTATAAGAACAAAGAGTTGTTACGCATCATGACAATGTCTTAGAAAACAA
AAGAAATGAATAAAAAAGTAAAACGAAAAATAAAAAGTGAGGATGAAGTTGTTGAATGAGTTGG
CGAGGCGGCGACTTTTTCATACATTCCATTTACTTAATTCCTAAAGTCCTTCTCACATCTCTTTGTTA
TATAATGACACCATAACCATTTCTTCTCTTCACAATCTTTACAAGAATATCTCTCTTCTACAGTAAA
CAAAAA
>SEQ ID NO: 151
ACGTAAGCTTCTTAGCCAATGGATGAGGATG
>SEQ ID NO: 152
ACGTTCTAGATTTTTGTTTACTGTAGAAGAG
>SEQ ID NO: 153
TGCTGCTTCAAATCCTTCTATAGCTCCTGTTTATACCACCATGACTACTTTCTCTCCAGGAATTCAAA
TGGGAAGTGGTGAAGAACACAGATTAGATGCACATAAGAAACTCCTGATTGGTCTTATAATCAGTT
CCTCTTCTCTTGGTATCGTAATCTTGATTTGCTTTGGCTTCTGGATGTACTGTCGCAAGAAAGCTCCC
AAACCCATCAAGATTCCGGATGCTGAGAGTGGGACTTCATCATTTTCAATGTTTGTGAGGCGGCTA
AGCTCAATCAAAACTCAGAGAACATCTAGCAATCAGGGTTATGTGCAGCGTTTCGATTCCAAGACG
CTAG
>SEQ ID NO: 154
TATGGATCCTGCTGCTTCAAATCCTTCTATAGCTCCTG
>SEQ ID NO: 155
TATTCTAGACTAGCGTCTTGGAATCGAAACGCTGCAC
>SEQ ID NO: 156
TATGAGCTCTGCTGCTTCAAATCCTTCTATAGCTCCTG
>SEQ ID NO: 157
TATGAGCTCCTAGCGTCTTGGAATCGAAACGCTGCAC
>SEQ ID NO: 158
GCAGATC GCTCCTCCCGTCGTGAT
>SEQ ID NO: 159
CGCCTAGG AGCGACGGGTACTCGATCAT
>SEQ ID NO: 160
CCTAGCTA AGCGACGGGTACTCGATCAT
>SEQ ID NO: 161
GCTCCTCCCGTCGTGATCACAGTGGTGAGGCACCACCATTACCACCGGGAGCTGGTCATCTCCGCT
GTCCTCGCCTGCGTCGCCACCGCCATGATCCTCCTCTCCACACTCTACGCCTGGACGATGTGGCGGC
GGTCTCGCCGGACCCCCCACGGCGGCAAGGGCCGCGGCCGGAGATCAGGGATCACACTGGTGCCA
ATCCTGAGCAAGTTCAATTCAGTGAAGATGAGCAGGAAGGGGGGCCTTGTGACGATGATCGAGTA
CCCGTCGCT
>SEQ ID NO: 162
CGGGATCCCGGCATAACAAACTCGTGCATCC
>SEQ ID NO: 163
CCATCGATGGCGCCAAACACAATA GCT CAA
>SEQ ID NO: 164
GTAAGTAATTTCAAGTTTAAGTTTCATAAGCATAACAAACTCGTGCATCCAATTTGAACCATTTTAC
TGTCCTGGCATCCTCTAAATATTTCCTTGATTATCAGCTTATCTTCATCCCATTGAATCAGAAAATTA
CCAACCCTTGTTTTAGCTTTAATCATTGTTATTTGTTGTCTGAGGGGCTACACTGTTTCTTTATATTG
GTGAAGGAGTTACCAGGCAAAAATTCCCACCTCCTGATATTAGCAGAGACCCCCTTTTTTGTGCCT
GTATGCATACTAACAAATAATACAGATGGAAATATGTATATTTGTTATATCATGGATTGATGCTTTA
TGTTTAGCAAGTCCATGCAATGGTAGTCAAAAGATGTAAACTTTTGAATGATATATTGGGGCTTTA
GATTAGCCATTTTTACCCTCACTTGAAAATGACAATTTTGCCCTTCCGATCTACTTTCTCTTGTCACC
TCAGGCAGGCTCTTGAAAGTTCTTATCCCTGAATTCCGTGGAAGTTTATTATTCTAATGTTATAGTT
TACTTAAAGTGTCGCATAATCTACTAGAGCCTAATGGAAGTACTGATGGACTTTGTTTTGCTACAAT
CACTGCTTGCAAGAATGACTACTTTGGGGCATTTCTAATATATTATTGATATTTCTATGATGTATTG
TTGTCCATGTACTTCAGTCCTTACAGCGACTAGTCCTATTTCTGCATTGATAAATTGTTCACTGTCAG
ACCATCTTGAGTGGCAAGAATGAGTATAACATGTCTTGTTTTTCTGTGATTTCAAGGTAAGCGCACA
TGCGCACAGTGTACACCGTCACCACATGTGAGTACACCCCCTAGTACACATGTAAAAAAAGCACAG
TCCAGTTATTAAATGGACCATTGGCATTGATTGTCGTGTTTATAGGAGTAAAGATACATGTAAACA
CTAATTCATTGGGAGATATAAATTTATACTACCATTGAATGTGACATAGGCTCTAAGGTTTTTAGTT
CAGCATTTCGAAAGAGCTTTGTTTGGTTGGCTTGGGATGGAATCAGGTGACAACATTTTTGGGTTGC
AGCAAATTTAATATTGATTGAGGAGGCATACAACGAAATCATTGAGCTATTGTGTTTGGCGTTACA
TCTATGGAATTTCTTCTAATCTGATTATTGTTTGTA
>SEQ ID NO: 165
GATCCGCTCCTCCCGTCGTGAT
>SEQ ID NO: 166
AACGCGATCGCTTGCATGCCTGCAGTAGAC
>SEQ ID NO: 167
GACTTAATTAAGAATTCGAGCTCGGGTA
>SEQ ID NO: 168
TCGTAGTGCACCACCATTTCCACCGCGAGCTGGTCATCGCCGCCGTCCTCGCCTGCATCGCCACCGT
CACGATCTTCCTTTCCACGCTCTACGCTTGGACACTATGGCGGCGATCTCGCCGGAGCACCGGCGG
CAAGGTCACCAGGAGCTCAGACGCAGCGAAGGGGATCAAGCTGGTGCCGATCTTGAGCAGGTTCA
ACTCGGTGAAGATGAGCAGGAAGAGGCTGGTTGGGATGTTCGAGTACCCGTCG
>SEQ ID NO: 169
GCAGATCTCGTAGTGCACCACCATTTC
>SEQ ID NO: 170
CGCCTAGGCGACGGGTACTCGAACATC
>SEQ ID NO: 171
CCTAGCTACGACGGGTACTCGAACATC
>SEQ ID NO: 172
GATCCTCGTAGTGCACCACCATTTC
>SEQ ID NO: 173
CTCGTAGTGCACCACCATTTC
>SEQ ID NO: 174
AATGGGACCGCCTCCGTTGCTCCGGCGGTGCCGGCGCCGCCTCCCGTCGTGATCATCGTGGAGCGG
CGCCATCATTTCCACCGCGAGCTAGTCATCGCCTCCGTTCTCGCCTCCATCGCCATCGTCGCGATTA
TCCTCTCCACGCTCTATGCGTGGATCCTGTGGCGGCGGTCTCGCCGGCTGCCCAGCGGCAAGGGCG
CCAGGAGCGCAGACACCGCGAGGGGAATCATGCTGGTGCCGATCCTGAGCAAGTTCCACTCA
>SEQ ID NO: 175
GCAGATCAATGGGACCGCCTCCGTTG
>SEQ ID NO: 176
CGCCTAGGTGAGTGGAACTTGCTCAGGA
>SEQ ID NO: 177
CCTAGCTATGAGTGGAACTTGCTCAGGA
>SEQ ID NO: 178
GTAAGTATTCTTGCAACACATTACTATTTTCAATAACCACAAGTTTAAAAGCTTGAGTCCATTTCGC
AAACCAGTTGTTCATAACCAAATTCTTAGGTAATTAGGTCCAATTGAGAAAATCTGATCATTGAAC
ACTAGCAGGAAATAACTCAGACATAGTTTCTGCATACTATAATGATGCTTAATATATTTGTTCTCTT
TTGAGATTGTATTGCATAGACATTTCTGTGTAAAATAATGTTTTACATCATGTATATATATCACTTTT
TATAG
>SEQ ID NO: 179
CGGGATCCTTCTTGCAACACATTACTATTT
>SEQ ID NO: 180
CCATCGATGAAATGTCTATGCAATACAATCTCAA
>SEQ ID NO: 181
GATCCAATGGGACCGCCTCCGTTG
>SEQ ID NO: 182
CAATGGGACCGCCTCCGTTGA
>SEQ ID NO: 183
GGCCCCGGCCGCGCGCGTCTCCGTGTCCTCCGCGACTGTGCACGTTTCGTCGGGAGCGGCGTGCCC
ACGCCCACCCCCCGTCCACCAGCCAGCAACCGACGGCACTGGTGACACGCGGCTGGTCCGCTCGGT
CCGCCCCGCGGCTCCAGATCACGGCAAGCGCGCCCGCCGCCCGCTGCTGCGCTGCGCTGCACGTCC
CGCCCTGACGCCACGCCACGCCAAGCGCGACACGACACGACACGACACGACCCGACCCCCGCCAA
CGAAACGCCGAAACGCGGCAACGCGTGACGGGCGCGCATGGTCGATGCTCTACCCGCGCGTCCGC
CCCACGCCAATCTCCCGGCGGGTCCCTCGTGGGACGGGGAACGCGATGCGGCTGCAGGCTGCGAC
CGCGACCGCGACCGCGACCGCGCCCACGTGAAGGCAGGCAGGCAGCCCCGGAGCGGGCGCGGCG
GTGGGCCAACGACGCGTTGCCGTCGCGAATCTTCTTCTGGCCACGGCCAAGGGCCAATCGCCCGCT
CCGCTCCGCTCCGCACTCCGCCTCCGCTAGGGAATATGGAACCCGATCCCACGGCCCTCTGGGTCT
GGTCGACGGGTCCTCTCGCCGTGGCAGCTGCTTCCCGGACCGGAGGATCGCTGAGCGCGGACGCCA
CTGCCATTGCCGTCCGACTATAGTTGTTAATTACCATAAAATAATTTGTTAACGATAAAACCCGTGT
CAGGCACCGTCGTCTGGACGCTGCTATGGGATAACCATTCGCGTACGTCGGTTGTATGGGTGGGAT
CCTCTGCGGCACGCCATTCTGGTGCTGCTAGTGGAATAGACAAAAAAAGGGCCGACGGTGTTTGCT
CGTGGCAGGCCACACAGAGTGACAACCAGAGTGGTTGCCGCAAAAACAACCAATCACACAAAAAG
TGTTGTACCGGTGGAGGACAGCCATTAATCAGCAGGCCGGCTTCGCGGCCAAAAGAAACGGAGAA
GAGGAAAAAGGGGGGC
>SEQ ID NO: 184
TCCCAAGCTTGCGCGTCTCCGTGTCCTC
>SEQ ID NO: 185
AGTAAAGCTTCCCCCTTTTTCCTCTTCTCC
>SEQ ID NO: 186
TAATGGTCGAGTGAGGCCCGTATAGATGTAGTTAAATAGCTAAAATTTTTGGAGAAATAAGCATTT
TTTTGGAAGAATATATTTAAACATGGGCTTGTAAAACTTGGCTGTAAAGATTTGGAATTTAGGATCT
TGGAGCCCCAAAACTGTATAAACTTGCTTAGGGACCCGTGTCTTGTGTGTTGCAGACCAAAAAATT
TAGAAAGCATCTAAACACCTATTTGAATGTAAAGTTTACAGCCAAAAGTTTTAGGATGTAAAGATT
TGGGATCTAAAAGTAGTCATTAGGAAATAACACGTTAGAGAGAGAGAGTAGATCTTCTTATTGGTT
TCTCATGCACTAATCGAACCAATCACTGGACCACTTGAACCAAACTTTATCACATTGAACTTTGTCA
GTTCAGTTCGAACGCAGGACTGGAGCTGCCCTTAAGGCCAATTGCTCAAGATTCATTCAACAATTG
AAACATCTCCCATGATTAAATCAGTATAAGGTTGCTATGGTCTTGCTTGACAAAGTTTTTTTTTTGA
GGGAATTTCAACTAAATTTTTGAGTGAAACTATCAAATACTGATTTTAAAAATTTTTTATAAAAGGA
AGCGCAGAGATAAAAGGCCATCTATGCTACAAAAGTACCCAAAAATGTAATCCTAAAGTATGAAT
TGCATTTTTTTTGTTTGGACGAAAGGAAAGGAGTATTACCACAAGAATGATATCATCTTCATATTTA
GATCTTTTTTGGGTAAAGCTTGAGATTCTCTAAATATAGAGAAATCAGAAGAAAAAAAAACCGTGT
TTTGGTGGTTTTGATTTCTAGCCTCCACAATAACTTTGACGGCGTCGACAAGTCTAACGGACACCAA
GCAGCGAACCACCAGCGCCGAGCCAAGCGAAGCAGACGGCCGAGACGTTGACACCTTCGGCGCGG
CATCTCTCGAGAGTTCCGCTCCGGCGCTCCACCTCCACCGCTGGCGGTTTCTTATTCCGTTCCGTTCC
GCCT
>SEQ ID NO: 187
AACTGCAGGGTCGAGTGAGGCCCGTA
>SEQ ID NO: 188
TTCTGCAGGGAACGGAACGGAATAAGAA
>SEQ ID NO: 189
GCCGTGGGTCGTTTAAGCTGCCGCTGTACCTGTGTCGTCTGGTGCCTTCTGGTGTACCTGGGAGGTT
GTCGTCTATCAAGTATCTGTGGTTGGTGTCATGAGTCAGTGAGTCCCAATACTGTTCGTGTCCTGTG
TGCATTATACCCAAAACTGTTATGGGCAAATCATGAATAAGCTTGATGTTCGAACTTAAAAGTCTC
TGCTCAATATGGTATTATGGTTGTTTTTGTTCGTCTCCT
>SEQ ID NO: 190
TAGGTACCGCCGTGGGTCGTTTAAGCT
>SEQ ID NO: 191
AAGGTACCAGGAGACGAACAAAAACAA
>SEQ ID NO: 192
AACGCGATCGTAATGGTCGAGTGAGGCCCGTATA
>SEQ ID NO: 193
ATGAAGAAACTGGTTCATCTTCAGTTTCTGTTTCTTGTCAAGATCTTTGCTACTCAATTC
CTCACTCCTTCTTCATCATCTTTTGCTGCTTCAAATCCTTCTATAGCTCCTGTTTATACC
ACCATGACTACTTTCTCTCCAGGAATTCAAATGGGAAGTGGTGAAGAACACAGATTAGAT
GCACATAAGAAACTCCTGATTGGTCTTATAATCAGTTCCTCTTCTCTTGGTATCGTAATC
TTGATTTGCTTTGGCTTCTGGATGTACTGTCGCAAGAAAGCTCCCAAACCCATCAAGATT
CCGGATGCTGAGAGTGGGACTTCATCATTTTCAATGTTTGTGAGGCGGCTAAGCTCAATC
AAAACTCAGAGAACATCTAGCAATCAGGGTTATGTGCAGCGTTTCGATTCCAAGACGCTA
GAGAAAGCGACAGGCGGTTTCAAAGACAGTAATGTAATCGGACAGGGCGGTTTCGGATGC
GTTTACAAGGCTTCTTTGGACAGCAACACTAAAGCAGCGGTTAAAAAGATCGAAAACGTT
AGCCAAGAAGCAAAACGAGAATTTCAGAATGAAGTTGAGCTGTTGAGCAAGATCCAGCAC
TCCAATATTATATCATTGTTGGGCTCTGCAAGTGAAATCAACTCGAGTTTCGTCGTTTAT
GAGTTGATGGAGAAAGGATCCTTAGATGATCAGTTACATGGACCTTCGTGTGGATCCGCT
CTAACATGGCATATGCGTATGAAGATTGCTCTAGATACAGCTAGAGGATTAGAGTATCTC
CATGAACATTGTCGTCCACCAGTTATCCACAGGGACCTGAAATCGTCTAATATACTTCTT
GATTCTTCCTTCAATGCCAAGATTTCAGATTTTGGTCTGGCTGTATCGGTTGGAGTGCAT
GGGAGTAACAACATTAAACTCTCTGGGACACTTGGTTATGTTGCCCCGGAATATCTCCTA
GACGGAAAGTTGACGGATAAGAGTGATGTCTATGCATTTGGGGTGGTTCTTCTTGAACTT
TTGTTGGGTAGAAGGCCGGTTGAGAAATTGAGTCCATCTCAGTGTCAATCTCTTGTGACT
TGGGCAATGCCACAACTTACCGATAGATCGAAACTCCCAAACATCGTGGATCCGGTTATA
AAAGATACAATGGATCTTAAGCACTTATACCAGGTAGCAGCCATGGCTGTGTTGTGCGTT
CAGCCAGAACCGAGTTACCGGCCGCTGATAACCGATGTTCTTCACTCACTTGTTCCATTG
GTTCCGGTCGAACTAGGAGGGACTCTCCGGTTAACCCGATGA
>SEQ ID NO: 194
MKKLVHLQFLFLVKIFATQFLTPSSSSFAASNPSIAPVYTTMTTFSPGIQMGSGEEHRLD
AHKKLLIGLIISSSSLGIVILICFGFWMYCRKKAPKPIKIPDAESGTSSFSAVVRRLSSI
KTQRTSSNQGYVQRFDSKTLEKATGGFKDSNVIGQGGFGCVYKASLDSNTKAAVKKIENV
SQEAKREFQNEVELLSKIQHSNITSLLGSASEINSSFVVYELMEKGSLDDQLHGPSCGSA
LTWHMRMKIALDTARGLEYLHEHCRPPVIHRDLKSSNILLDSSFNAKISDFGLAVSVGVH
GSNNIKLSGTLGYVAPEYLLDGKLTDKSDVYAFGVVLLELLLGRRPVEKLSPSQCQSLVT
WAMPQLTDRSKLPNIVDPVIKDTMDLKHLYQVAAMAVLCVQPEPSYRPLITDVLHSLVPL
VPVELGGTLRLTR
>SEQ ID NO: 195
AATCCAGCTCATTCTGGAATTCCTTCTCGCA
>SEQ ID NO: 196
TGAACTTGCTCAGGATTGGCACCAGTGTGATC
>SEQ ID NO: 197
MEIPAAPPPPLPVLCSYVVFLLLLSSCSLARGRIAVSSPGPSPVAAAVTANETASSSSSP
VFPAAPPVVITVVRHHHYHRELVISAVLACVATAMILLSTLYAWTMWRRSRRTPHGGKGR
GRRSGITLVPILSKFNSVKMSRKGGLVTMIEYPSLEAATGKFGESNVLGVGGFGCVYKAA
FDGGATAAVKRLEGGGPDCEKEFENELDLLGRIRHPNIVSLLGFCVHGGNHYIVYELMEK
GSLETQLHGSSHGSALSWHVRMKIALDTARGLEYLHEHCNPPVIHRDLKPSNILLDSDFN
AKIADFGLAVTGGNLNKGNLKLSGTLGYVAPEYLLDGKLTEKSDVYAFGVVLLELLMGRK
PVEKMSPSQCQSIVSWAMPQLTDRSKLPNIIDLVIKDTMDPKHLYQVAAVAVLCVQPEPS
YRPLITDVLHSLVPLVPAELGGTLRVAEPPSPSPDQRHYPC
>SEQ ID NO: 198
TATACCGGTAAAATGAGAGAGCTTCTTCTTCTTCTTCTTCTTCATTTTCAGTC
>SEQ ID NO: 199
ATATACCGGTCTTGTTAACCGGAGAGTCCCTCCTAGCTC
>SEQ ID NO: 200
CGCTCCTCCCGTCGTGAT

### LITERATURE

1. Altschul S F, Gish W, Miller W, Myers E W, Lipman D J. (1990) Basic local alignment search tool. J. Mol. Biol. 215: 403-410.
2. Altschul S F, Madden T L, Schaffer A A, Zhang J, Zhang Z, Miller W, Lipman D J. (1997) Gapped BLAST and PSI-BLAST: a new generation of protein database search programs. Nucl. Acid Res. 25: 3389-3402.
3. An G, Mitra A, Choi H K, Costa M A, An K, Thornburg R W, Ryan C A. (1989) Functional analysis of the 3′ control region of the potato wound-inducible proteinase inhibitor II gene. Plant Cell 1: 115-122.
4. Araus L J, Slafer G A, Reynolds M P, Royo C (2002) Plant Breeding and drought in C3 cereals: What should we breed for?Annals of Botany 89: 925-940.
5. Atanassvoa R, Chaubet N, Gigot C (1992) A 126 bp fragment of a plant histone gene promoter confers preferential expression in meristems of transgenic Arabidopsis. Plant Journal 2(3): 291-300.
6. Beetham P R, Kipp P B, Sawycky X L, Arntzen C J, May G D (1999) A tool for functional plant genomics: chimeric RNA/DNA oligonucleotides cause in vivo gene-specific mutations. Proceedings of the National Academy of Science USA, 96: 8774-8778.
7. Bevan M, Barnes W M, Chilton M D (1983) Structure and transcription of the nopaline synthase gene region of T-DNA. Nucl. Acids Res. 12: 369-385.
8. Bevan M, Barker R, Goldsbrough A, Jarvis M, Kavanagh T, Iturriaga G. (1986) The structure and transcription start site of a major potato tuber protein gene. Nucleic Acids Research 14: 4625-4636.
9. Christensen A H, Sharrock R A, Quail P H. (1992) Maize polyubiquitin genes: structure, thermal perturbation of expression and transcript splicing, and promoter activity following transfer to protoplasts by electroporation. Plant Mol. Biol. 18: 675-689.
10. Condon A G, Richards R A, Rebetzke G J, Farquhar G D (2002) Improving Intrinsic Water-Use Efficiency and crop yield. Crop Science 42:122-131.
11. Davies W J, Wilkinson S, Loveys B R (2002) Stomatal control by chemical signalling and the exploitation of this mechanism to increase water use efficiency in agriculture. New Phytol. 153: 449-460.
12. De Loose M, Gheysen G, Tiré C, Gielen J, Villarroel R, Genetello C, Van Montagu M, Depicker A, Inzé D (1991) The extensin signal peptide allows secretion of a heterologous protein from protoplasts. Gene 99: 95-100.
13. Dong C, Beetham P, Vincent K, Sharp P (2006) Oligonucleotide-directed gene repair in wheat using a transient plasmid gene repair assay system. Plant Cell Reports 25: 457-465.
14. Dratewka-Kos E, Rahman S, Grzelczak Z F, Kennedy T D, Murray R K, Lane B G (1989) Polypeptide structure of germin as deduced from cDNA sequencing. J. Biol.

### Chem. 264: 4896-4900.

15. Elomaa P, Mehto M, Kotilainen M, Helariutta Y, Nevalainen L, Teeri T H (1998) A bHLH transcription factor mediates organ, region and flower type specific signals on dihydroflavonol-4-reductase (dfr) gene expression in the inflorescence of Gerbera hybrida (Asteraceae). The Plant Journal 16(1): 93-99.
16. Farquhar G D and Sharky T D (1994) Photosynthesis and carbon assimilation (p187) in Physiology and Determination of Crop Yield, ASA, CSSA, SSSA, Madison Wisconsin USA.
17. Fraley R T, Rogers S G, Horsch R B, Sanders P R, Flick J S, Adams S P, Bittner M L, Brand L A, Fink C L, Fry J S, Galluppi G R, Goldberg S B, Hoffmann N L, Woo S C (1983) Expression of bacterial genes in plant cells. Proc Natl Acad Sci USA. 80(15): 4803-4807.
18. Goeddel, GENE EXPRESSION TECHNOLOGY: METHODS IN ENZYMOLOGY 185, Academic Press, San Diego, Calif (1990).
19. Goldberg R B (1986) Regulation of plant gene expression. Philos Trans R Soc London Ser B 314: 343-353.
20. Greene E A, Codomo C A, Taylor N E, Henikoff J G, Till B J, Reynolds S H, Enns L C, Burtner C, Johnson J E, Odden A R, Comai L, Henikoff S (2003) Spectrum of chemically induced mutations from a large-scale reverse-genetic screen in Arabidopsis. Genetics 164(2):731-740.
21. Gruber et al. “Vectors for Plant Transformation” in Methods in Plant Molecular Biology and Biotechnology, Glick B. R. and Thompson, J. E. Eds. (CRC Press, Inc., Boca Raton, 1993) pages 89-119.
22. Hardie D G (1999) PLANT PROTEIN SERINE/THREONINE KINASES: Classification and Functions. Annu Rev Plant Physiol Plant Mol Biol. 50:97-131.
23. Henikoff S, and Henikoff J G (1992) Amino acid substitution matrices from protein blocks. Proc. Natl. Acad. Sci. USA 89: 10915-10919.
24. Horsch R, Fry J E, Hoffmann N, Eichholtz D, Rogers S, Fraley R T (1985) A simple and general method for transferring genes into plants. Science 227:1229-1231.
25. Huang Y, Li H, Gupta R, Morris P C, Luan S, Kieber J J. (2000) ATMPK4, an Arabidopsis homolog of mitogen-activated protein kinase, is activated in vitro by AtMEK1 through threonine phosphorylation. Plant Physiol. 122(4):1301-1310.
26. Kado C I, Hooykaas P J (1991) Molecular mechanisms of crown gall tumorigenesis. Crit. Rev. Plant Sci. 10: 1-32.
27. Karaba A, Dixit S, Greco R, Aharoni A, Trijatmiko K R, Marsch-Martinez N, Krishnan A, Nataraja K N, Udayakumar M, Pereira A (2007) Improvement of water use efficiency in rice by expression of HARDY, an Arabidopsis drought and salt tolerant gene. Proc. Natl. Acad. Sci. USA 104: 15270-15272.
28. Keil M, Sanchez-Serrano J, Schell J, Willmitzer L (1986) Primary structure of a proteinase inhibitor I I gene from potato (Solanum tuberosum). Nucl. Acids Res. 14: 5641-5650.
29. Laemmli, U K (1970) Cleavage of structural proteins during the assembly of the head of bacteriophage T4. Nature. 227(259): 680-685.
30. Last D I, Brettell R I, Chamberlain D A, Chaudhury A M, Larkin P J, Marsh E L, Peacock W J, Dennis E S (1991) pEmu: an improved promoter for gene expression in cereal cells Theor. Appl. Genet. 81: 581-588.
31. Lepetit M, Ehling M, Chaubet N, Gigot C (1992) A plant histone gene promoter can direct both replication-dependent and -independent gene expression in transgenic plants. Mol. Gen. Genet., 231: 276-285.
32. Lund P, Dunsmuir P (1992) A plant signal sequence enhances the secretion of bacterial ChiA in transgenic tobacco. Plant Mol. Biol. 18: 47-53.
33. McElroy D, Zhang W, Cao J, Wu R (1990) Isolation of an efficient actin promoter for use in rice transformation. Plant Cell 2(2): 163-171.
34. Mian M A R, Bailey M A, Ashley D A, Wells R, Carter T E Jr, Parrott W A, Boerma H R (1996) Molecular markers associated with water use efficiency and leaf ash in soybean. Crop Sci. 36: 1252-1257.
35. Martin B, Nienhuis J, King G, Schaefer A (1989) Restriction Fragment Length Polymorphisms Associated with Water Use Efficiency in Tomato. Science. 243(4899):1725-1728.
36. Masle J, Gilmore S R, Farquhar G D (2005) The ERECTA gene regulates plant transpiration efficiency in Arabidopsis. Nature 436: 866-870
37. Matsuoka K, Nakamura K (1991) Propeptide of a precursor to a plant vacuolar protein required for vacuolar targeting. Proc. Nat'l Acad. Sci. USA 88: 834-838.
38. van der Meer I M, Spelt C E, Mol J N, Stuitje A R (1990) Promoter analysis of the chalcone synthase (chsA) gene of Petunia hybrida: a 67 bp promoter region directs flower-specific expression. Plant Molecular Biology 15(1): 95-109.
39. Mogen B D, MacDonald M H, Graybosch R, Hunt A G (1990) Upstream sequences other than AAUAAA are required for efficient messenger RNA 3′-end formation in plants. Plant Cell 2: 1261-1272.
40. Moloney M M, Walker J M, Sharma K K (1989) High efficiency transformation of Brassica napus using Agrobacterium vectors. Plant Cell Reports 8: 238-242.
41. Needleman S B, Wunsch C D (1970) A general method applicable to the search for similarities in the amino acid sequence of two proteins. J. Mol. Biol. 48: 443-453.
42. Odell J T, Nagy F, Chua N H (1985) Identification of DNA sequences required for activity of the cauliflower mosaic virus 35S promoter. Nature 313: 810-812.
43. Oleykowski C A, Bronson Mullins C R, Godwin A K, Yeung A T (1998) Mutation detection using a novel plant endonuclease. Nucleic Acids Res. 26(20): 4597-4602.
44. Sanford J C, Smith F D, Russell J A (1993) Optimizing the biolistic process for different biological applications. Methods Enzymol. 217: 483-509.
45. Klein T M, Arentzen R, Lewis P A, Fitzpatrick-McElligott S (1992) Transformation of microbes, plants and animals by particle bombardment. Biotechnology 10: 286-291.
46. Sambrook J, Fritsch E F, Maniatis T (1989) Molecular Cloning. A Laboratory Manual, 2^{nd} Ed., Cold Spring Harbor Laboratory Press, New York
47. Sinclair T R (1994) Limits to crop yield. in Physiology and Determination of Crop Yield, ASA, CSSA, SSSA, Madison Wisconsin USA.
48. Price A H, Cairns J E, Horton P, Jones H G, Griffiths H (2002) Linking drought-resistance mechanisms to drought avoidance in upland rice using a QTL approach: progress and new opportunities to integrate stomatal and mesophyll responses.

### Journal of Experimental Botany 53: 989-1004.

49. Schwab R, Ossowski S, Riester M, Warthmann N, Weigel D (2006) Highly specific gene silencing by artificial microRNAs in Arabidopsis. Plant Cell 18(5): 1121-1133.
50. Shiu S H, Bleecker A B (2001) Receptor-like kinases from Arabidopsis form a monophyletic gene family related to animal receptor kinases. Proc Natl Acad Sci USA. 98(19): 10763-10768.
51. Stockinger E J, Mulinix C A, Long C M, Brettin T S, Iezzoni A F (1996) A linkage map of sweet cherry based on RAPD analysis of a microspore-derived callus culture population. J. Heredity 87: 214-218.
52. Thumma B R, Naidu B P, Chandra A, Cameron D F, Bahnisch L M, Liu C (2001) Identification of causal relationship among traits related to drought resistance in Stylosanthes scabra using QTL analysis. Journal of Experimental Botany 52: 203-214.
53. Torii K U, Mitsukawa N, Oosumi T, Matsuura Y, Yokoyama R, Whittier R F, Komeda Y (1996) The Arabidopsis ERECTA gene encodes a putative receptor protein kinase with extracellular leucine-rich repeats. Plant Cell. 8(4): 735-746.
54. Velten J, Velten L, Hain R, Schell J (1984) Isolation of a dual plant promoter fragment from the Ti plasmid of Agrobacterium tumefaciens. EMBO J. 3: 2723-2730.
55. Verwoert I I, Linden K H, Nijkamp H J, Stuitje A R (1994) Developmental specific expression and organelle targeting of the Escherichia coli fabD gene, encoding malonyl coenzyme A-acyl carrier protein transacylase in transgenic rape and tobacco seeds. Plant Mol. Biol. 26: 189-202.
56. Visser R G, Stolte A, Jacobsen E (1991) Expression of a chimaeric granule-bound starch synthase-GUS gene in transgenic potato plants. Plant Molecular Biology 17: 691-699.
57. Walling L L, Chang Y C, Demmin D S, Holzer F M (1988) Isolation, characterization and evolutionary relatedness of three members from the soybean multigene family encoding chlorophyll a/b binding proteins. Nucl. Acids Res. 16: 10477-10492.
58. Weissbach A and Weissbach H Eds. (1988) Methods for plant molecular biology.

### Academic Press (San Diego).

59. Wilkins T A, Bednarek S Y, Raikhel N V (1990) Role of propeptide glycan in post-translational processing and transport of barley lectin to vacuoles in transgenic tobacco. Plant Cell 2: 301-313.
60. Yang B, Wen X, Kodali N S, Oleykowski C A, Miller C G, Kulinski J, Besack D, Yeung J A, Kowalski D, Yeung A T (2000) Purification, cloning, and characterization of the CEL I nuclease. Biochemistry. 39(13): 3533-3541.

We claim:

1. A method of increasing water use efficiency in a plant, comprising:
a) transforming a plant, a plant tissue culture or a plant cell with a vector comprising a nucleic acid construct that inhibits the expression or activity of a PK220 gene to obtain a transformed plant, a transformed plant tissue culture or a transformed plant cell;
b) growing the transformed plant or regenerating a plant from the transformed plant tissue culture or the transformed plant cell; and
c) selecting a plant having increased water use efficiency relative to a wild type plant.

2. The method of claim 1, wherein the nucleic acid construct comprises an antisense sequence, siRNA, hairpin, miRNA, artificial miRNA, each of which is complementary to a nucleic acid sequence encoding a PK220 polypeptide.

3. The method of claim 1, wherein the nucleic acid construct comprises a sequence of at least 19 nucleotides.

4. A transgenic plant produced by the method of claim 1, wherein the transgenic plant has increased water use efficiency relative to a wild type plant.

5. A transgenic seed produced by the transgenic plant of claim 4, wherein the transgenic seed produces a plant having increased water use efficiency relative to a wild type plant.

6. A method of producing a drought tolerant plant, comprising:
a) transforming a plant, a plant tissue culture or a plant cell with a vector comprising a nucleic acid construct that inhibits the expression or activity of a PK220 gene to obtain a transformed plant, a transformed plant tissue culture or a transformed plant cell;
b) growing the transformed plant or regenerating a plant from the transformed plant tissue culture or the transformed plant cell; and
c) selecting a plant having increased drought tolerance relative to a wild type plant.

7. The method of claim 6, wherein the nucleic acid construct comprises an antisense sequence, siRNA, hairpin, miRNA, artificial miRNA, each of which is complementary to a nucleic acid sequence encoding a PK220 polypeptide.

8. The method of claim 6, wherein the nucleic acid construct comprises a sequence of at least 19 nucleotides.

9. A transgenic plant produced by the method of claim 6, wherein the transgenic plant has increased drought tolerance relative to a wild type plant.

10. A transgenic seed produced by the transgenic plant of claim 9, wherein the transgenic seed produces a plant having increased drought tolerance relative to a wild type plant.

11. A method of producing a cold stress tolerant plant, comprising:
a) transforming a plant, a plant tissue culture or a plant cell with a vector comprising a nucleic acid construct that inhibits the expression or activity of a PK220 gene to obtain a transformed plant, a transformed plant tissue culture or a transformed plant cell;
b) growing the transformed plant or regenerating a plant from the transformed plant tissue culture or the transformed plant cell; and
c) selecting a plant having increased cold stress tolerance relative to a wild type plant.

12. The method of claim 11, wherein the nucleic acid construct comprises an antisense sequence, siRNA, hairpin, miRNA, artificial miRNA, each of which is complementary to a nucleic acid sequence encoding a PK220 polypeptide.

13. The method of claim 11, wherein the nucleic acid construct comprises a sequence of at least 19 nucleotides.

14. A transgenic plant produced by the method of claim 11, wherein the transgenic plant has increased cold stress tolerance relative to a wild type plant.

15. A transgenic seed produced by the transgenic plant of claim 14, wherein the transgenic seed produces a plant having increased cold stress tolerance relative to a wild type plant.

16. A method of producing a plant tolerant to low nitrogen conditions, comprising:
a) transforming a plant, a plant tissue culture or a plant cell with a vector comprising a nucleic acid construct that inhibits the expression or activity of a PK220 gene to obtain a transformed plant, a transformed plant tissue culture or a transformed plant cell;
b) growing the transformed plant or regenerating a plant from the transformed plant tissue culture or the transformed plant cell; and
c) selecting a plant having increased tolerance to low nitrogen conditions relative to a wild type plant.

17. The method of claim 16, wherein the nucleic acid construct comprises an antisense sequence, siRNA, hairpin, miRNA, artificial miRNA, each of which is complementary to a nucleic acid sequence encoding a PK220 polypeptide.

18. The method of claim 16, wherein the nucleic acid construct comprises a sequence of at least 19 nucleotides.

19. A transgenic plant produced by the method of claim 16, wherein the transgenic plant has increased tolerance to low nitrogen conditions relative to a wild type plant.

20. A transgenic seed produced by the transgenic plant of claim 19, wherein the transgenic seed produces a plant having increased tolerance to low nitrogen conditions relative to a wild type plant.


SEQUENCE LISTING (200 sequences)

SEQ ID NO: 1 - DNA, 1299 bp, Arabidopsis thaliana
  source 1..1299: mol_type = other DNA
atgagagagcttcttcttcttcttcttcttcattttcagtctctaattcttttgatgatcttcatcactgtctctgcttcttctgcttcaaatccttctttagctcctgtttactcttccatggctacattctctcctcgaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggtcttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatga

SEQ ID NO: 2 - protein, 432 aa, Arabidopsis thaliana
MRELLLLLLLHFQSLILLMIFITVSASSASNPSLAPVYSSMATFSPRIQMGSGEEDRFDAHKKLLIGLIISFSSLGLIILFCFGFWVYRKNQSPKSINNSDSESGNSFSLLMRRLGSIKTQRRTSIQKGYVQFFDIKTLEKATGGFKESSVIGQGGFGCVYKGCLDNNVKAAVKKIENVSQEAKREFQNEVDLLSKIHHSNVISLLGSASEINSSFIVYELMEKGSLDEQLHGPSRGSALTWHMRMKIALDTARGLEYLHEHCRPPVIHRDLKSSNILLDSSFNAKISDFGLAVSLDEHGKNNIKLSGTLGYVAPEYLLDGKLTDKSDVYAFGVVLLELLLGRRPVEKLTPAQCQSLVTWAMPQLTDRSKLPNIVDAVIKDTMDLKHLYQVAAMAVLCVQPEPSYRPLITDVLHSLVPLVPVELGGTLRLTR

SEQ ID NO: 3 - DNA, 1299 bp, Arabidopsis thaliana
  source 1..1299: mol_type = other DNA
atgagagagcttcttcttcttcttcttcttcattttcagtctctaattcttttgatgatcttcatcactgtctctgcttcttctgcttcaaatccttctttagctcctgtttactcttccatggctacattctctcctcgaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggttttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatga

SEQ ID NO: 4 - protein, 432 aa, Arabidopsis thaliana
MRELLLLLLLHFQSLILLMIFITVSASSASNPSLAPVYSSMATFSPRIQMGSGEEDRFDAHKKLLIGLIISFSSLGLIILFCFGFWVYRKNQSPKSINNSDSESGNSFSLLMRRLGSIKTQRRTSIQKGYVQFFDIKTLEKATGGFKESSVIGQGGFGCVYKGCLDNNVKAAVKKIENVSQEAKREFQNEVDLLSKIHHSNVISLLGSASEINSSFIVYELMEKGSLDEQLHGPSRGSALTWHMRMKIALDTARGLEYLHEHCRPPVIHRDLKSSNILLDSSFNAKISDFGFAVSLDEHGKNNIKLSGTLGYVAPEYLLDGKLTDKSDVYAFGVVLLELLLGRRPVEKLTPAQCQSLVTWAMPQLTDRSKLPNIVDAVIKDTMDLKHLYQVAAMAVLCVQPEPSYRPLITDVLHSLVPLVPVELGGTLRLTR

SEQ ID NO: 5 - DNA, 1160 bp, Arabidopsis thaliana
  source 1..1160: mol_type = other DNA
atgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggttttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacaga

SEQ ID NO: 6 - protein, 383 aa, Arabidopsis thaliana
MGSGEEDRFDAHKKLLIGLIISFSSLGLIILFCFGFWVYRKNQSPKSINNSDSESGNSFSLLMRRLGSIKTQRRTSIQKGYVQFFDIKTLEKATGGFKESSVIGQGGFGCVYKGCLDNNVKAAVKKIENVSQEAKREFQNEVDLLSKIHHSNVISLLGSASEINSSFIVYELMEKGSLDEQLHGPSRGSALTWHMRMKIALDTARGLEYLHEHCRPPVIHRDLKSSNILLDSSFNAKISDFGFAVSLDEHGKNNIKLSGTLGYVAPEYLLDGKLTDKSDVYAFGVVLLELLLGRRPVEKLTPAQCQSLVTWAMPQLTDRSKLPNIVDAVIKDTMDLKHLYQVAAMAVLCVQPEPSYRPLITDVLHSLVPLVPVELGGTLRLTR

SEQ ID NO: 7 - DNA, 1160 bp, Arabidopsis thaliana
  source 1..1160: mol_type = other DNA
atgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggtcttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacaga

SEQ ID NO: 8 - protein, 383 aa, Arabidopsis thaliana
MGSGEEDRFDAHKKLLIGLIISFSSLGLIILFCFGFWVYRKNQSPKSINNSDSESGNSFSLLMRRLGSIKTQRRTSIQKGYVQFFDIKTLEKATGGFKESSVIGQGGFGCVYKGCLDNNVKAAVKKIENVSQEAKREFQNEVDLLSKIHHSNVISLLGSASEINSSFIVYELMEKGSLDEQLHGPSRGSALTWHMRMKIALDTARGLEYLHEHCRPPVIHRDLKSSNILLDSSFNAKISDFGLAVSLDEHGKNNIKLSGTLGYVAPEYLLDGKLTDKSDVYAFGVVLLELLLGRRPVEKLTPAQCQSLVTWAMPQLTDRSKLPNIVDAVIKDTMDLKHLYQVAAMAVLCVQPEPSYRPLITDVLHSLVPLVPVELGGTLRLTR

SEQ ID NO: 9 - DNA, 1542 bp, Arabidopsis thaliana
  source 1..1542: mol_type = other DNA
atcaaaaacttttcttttcttagcaaaaaaaacaaaaaaatgagagagcttcttcttcttcttcttcttcattttcagtctctaattcttttgatgatcttcatcactgtctctgcttcttctgcttcaaatccttctttagctcctgtttactcttccatggctacattctctcctcgaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggtcttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacagaaacacgccaaaagaaatccaaagccatttagatgattttcttttatcctttgcctttatatttttttgtatagggttatgatccactcatctgaaagtttgggggtaagaatgtgagaatataagttttcagggttgttgagttctatataattatatttgtttctttttattgtcaaatataattatatttttgt

SEQ ID NO: 10 - DNA, 1309 bp, Arabidopsis thaliana
  source 1..1309: mol_type = other DNA
aaaatgagagagcttcttcttcttcttcttcttcattttcagtctctaattcttttgatgatcttcatcactgtctctgcttcttctgcttcaaatccttctttagctcctgtttactcttccatggctacattctctcctcgaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggtcttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacag

SEQ ID NO: 11 - DNA, 1177 bp, Arabidopsis thaliana
  source 1..1177: mol_type = other DNA
tctgtgtcaggaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggtcttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacag

SEQ ID NO: 12 - DNA, 154 bp, Arabidopsis thaliana
  source 1..154: mol_type = other DNA
tcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctc

SEQ ID NO: 13 - DNA, 288 bp, Arabidopsis thaliana
  source 1..288: mol_type = other DNA
tctgtgtcaggaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctc

SEQ ID NO: 14 - DNA, 1510 bp, Arabidopsis thaliana
  source 1..1510: mol_type = other DNA
tgttaaaagcgatttataatttacaccgttttggtgtatatttctatctatccttttacaagacctatatatgttatgttatggtggtgtactattttaagtgagcgacatagtattttcttcatatagctaattaatcaacaacaatttcccaacttacaactatttgcgtactttaaacttatattgaaagagaactacaaaattatttttttgtacaagagaattatggtcttcggatcaataatttctctagatataatatgtaaagccaaccctataatttgtaaaatccatgatttgatataattttcttttaaaattgtgaattggcagacaaaaacaacattacattttgatttaaattcataactttgacttgctaaggaaacaccatgattcattttttgtcatttgttacatcatcactagaaatatttgatctaactttattatgataatagactacatactacatatgcagttacgattttaaatactacatatttaagcgtgtttaaactgtaaccatatcatataaaatgacatatctaaaagtgattttcaatattttgatatgatatgtgttgtagcacggataatgatctaatttttaagtaataagcttgttcattacaaaagagaagaaagtagtattgggccatgattatgtaaggacaaaataggaagatgtggaagaagccattcgagggttttattacaaaaacagagtatataattggtcataatgttttattcacttaatttaacattattgcattatattttcatgaacacatatttctttaactaaaaatatacacatatttcttattgtagatgaagtgaaaagaacaatatttgggttcacatctatgggtgaatccttttaatcaccccctaaaataaaaaaggtgccatatttctatttttagagaaagatatagagcaccattggagtggttttgctccaaatatagagtttagagaaatatataatacaccattggagatgctctaaaatgaatttatttatttatttagatggaagattctaattggttagaaaaagaggaagtgaataataggattcacctataagagtgaacccaagtatttttaagagataatgtgtaaagtaaatagatggtcattgtgtgaattatgaatagaaccatggttttccatttttaattgcttaacatagggtaatcaacaatggggtttaatatgtcaatagacaatagtaaagaaagtatttgatctatcccaaatctttcttcgttcgttagttcatcactttctttctttttggttatattaatggtagagaactaaaaattcaactttttattcaaaagctccctttctctttccctcctttatttgccataaaagtgatttcaagaagacagcgagagagaaagtgatagttcgttcactcttcgctttctcaagaatttcaaaacaccaaaaaagtctttagattgaatttcatcaaaaacttttc

SEQ ID NO: 15 - DNA, 157 bp, Arabidopsis thaliana
  source 1..157: mol_type = other DNA
agacaagaaaaaaggaaacaaaattttatgaaagagatctccattagagaaagagagagcgagagagagattaatcttggaagagcaatctcacattctcacactgctcttagaaaatctctctttcaccattaaaaatcccaaagagtctggagaa

SEQ ID NO: 16 - DNA, 1257 bp, Arabidopsis thaliana
  source 1..1257: mol_type = other DNA
atgggaaagattcttcatcttcttcttcttcttcttaaggtctctgttcttgaattcatcattagtgtttctgcttttacttcacctgcttcacagccttctctttctcctgtttacacttccatggcttccttttctccagggatccacatgggcaaaggccaagaacacaagttagatgcacacaagaaacttctaatcgctctcataatcacctcatcttctctaggactaatacttgtatcttgtttatgcttttgggtttattggtctaagaaatctcccaaaaacaccaagaactcaggtgagagtaggatttcattatccaagaagggctttgtgcagtccttcgattacaagacactagagaaagcaacaggcggtttcaaagacggtaatcttataggacgaggcgggttcggagatgtttacaaggcctgtttaggcaacaacactctagcagcagtcaaaaagatcgaaaacgttagtcaagaagcaaaacgagaatttcagaatgaagttgatttgttgagcaagattcaccacccgaacatcatctcattgtttggatatggaaatgaactcagttcgagttttatcgtctacgagctgatggaaagcggatcattggatacacagttacacggaccttctcggggatcggctttaacatggcacatgcggatgaagattgctcttgatacagcaagagctgttgagtatctccacgagcgttgtcgtcctccggttatccacagagatcttaaatcgtcaaatattctccttgattcttccttcaacgccaagatttcggattttggtcttgcggtaatggtgggggctcacggcaaaaacaacattaaactatcaggaacacttggttatgttgctccagaatatctcctagatggaaaattgacggataagagtgatgtttatgcgtttggtgtggttttacttgaactcttgttaggaagacggccggttgagaaattgagttcggttcagtgtcaatctcttgtcacttgggcaatgccccaacttacggatagatcaaagcttccgaaaatcgtggatccggttatcaaagatacaatggatcataagcacttataccaggtggcagccgtggcagtgctttgtgtacaaccagaaccgagttatcgaccgttgataaccgatgttcttcactcactagttccattggttccggtagagctaggagggactctccggttaataccatcatcgtcttga

SEQ ID NO: 17 - protein, 418 aa, Arabidopsis thaliana
MGKILHLLLLLLKVSVLEFIISVSAFTSPASQPSLSPVYTSMASFSPGIHMGKGQEHKLDAHKKLLIALIITSSSLGLILVSCLCFWVYWSKKSPKNTKNSGESRISLSKKGFVQSFDYKTLEKATGGFKDGNLIGRGGFGDVYKACLGNNTLAAVKKIENVSQEAKREFQNEVDLLSKIHHPNIISLFGYGNELSSSFIVYELMESGSLDTQLHGPSRGSALTWHMRMKIALDTARAVEYLHERCRPPVIHRDLKSSNILLDSSFNAKISDFGLAVMVGAHGKNNIKLSGTLGYVAPEYLLDGKLTDKSDVYAFGVVLLELLLGRRPVEKLSSVQCQSLVTWAMPQLTDRSKLPKIVDPVIKDTMDHKHLYQVAAVAVLCVQPEPSYRPLITDVLHSLVPLVPVELGGTLRLIPSSS

SEQ ID NO: 18 - DNA, 1302 bp, Arabidopsis thaliana
  source 1..1302: mol_type = other DNA
atgaagcaaattgttataacagctcttgttttactacaagcttatgttcttcatcaatccacatgtgttatgtcccttactacacaagaatctccttctcctcaaccttctgctttcactcccgccttatctcctgattatcaacagagagagaaggaattgcataaacaagagagtaacaacatgagactggttatttcactagcagctacattttccttagttggtataatcttactttgctctctgctttattggttttgccataggagaagaaacctcaagagctcaggttgtgggtgtagtggaatcacattcttgaatcggtttagtcgctcaaaaacattagacaagagaactacaaagcagggaacagtgtcattgatcgattacaatatactagaagaaggaactagtggtttcaaggagagtaacattttgggtcaaggtggatttggatgtgtatattctgccacattagagaacaacatttcagctgcggttaagaagctagactgtgccaatgaagatgcagcaaaggaatttaagagtgaggttgagatattgagtaagctccagcacccgaatataatatcccttttgggttatagcacgaatgatactgcgagattcattgtctatgagctgatgccaaacgtttctctggaatctcatttacacggatcttctcagggttcggcgatcacatggcctatgaggatgaagattgctcttgatgtaacaaggggattagaatatttgcatgaacattgtcatccagcaatcattcacagggacttgaaatcatccaacatcttattagatagcaatttcaatgctaagatttcagattttggtctagctgttgttgatgggccaaagaacaagaaccataaactttccgggacagttggctacgttgcaccagagtatcttctcaacggccaattgacagaaaagagcgacgtgtatgcttttggagtagtgttattagagcttttactcgggaaaaaacctgtggagaaactagctcccggtgaatgccaatccatcatcacttgggcaatgccttatctcactgatagaaccaagttaccaagcgtcatagatcctgcgattaaagatacgatggacttgaaacacctttaccaggtagcggcagtggcgattttgtgcgtgcagccagaaccgagttatagaccgttgattacagacgtcttgcattctcttatacctttggttccaatggaacttggtggaaccttaaaaaccatcaaatgtgcttcaatggatcactgttaa

SEQ ID NO: 19 - protein, 433 aa, Arabidopsis thaliana
MKQIVITALVLLQAYVLHQSTCVMSLTTQESPSPQPSAFTPALSPDYQQREKELHKQESNNMRLVISLAATFSLVGIILLCSLLYWFCHRRRNLKSSGCGCSGITFLNRFSRSKTLDKRTTKQGTVSLIDYNILEEGTSGFKESNILGQGGFGCVYSATLENNISAAVKKLDCANEDAAKEFKSEVEILSKLQHPNIISLLGYSTNDTARFIVYELMPNVSLESHLHGSSQGSAITWPMRMKIALDVTRGLEYLHEHCHPAIIHRDLKSSNILLDSNFNAKISDFGLAVVDGPKNKNHKLSGTVGYVAPEYLLNGQLTEKSDVYAFGVVLLELLLGKKPVEKLAPGECQSIITWAMPYLTDRTKLPSVIDPAIKDTMDLKHLYQVAAVAILCVQPEPSYRPLITDVLHSLIPLVPMELGGTLKTIKCASMDHC

SEQ ID NO: 20 - DNA, 2016 bp, Arabidopsis thaliana
  source 1..2016: mol_type = other DNA
atgaagactatgtccaaatcgtctttgcgtttgcattttctctcgctactcttactttgttgtgtctccccttcaagctttgtcattataagattcattacacataatcattttgatggtctagtacgttgtcatccccacaagtttcaagcccttacgcagttcaagaacgagtttgatacccgccgttgcaaccacagtaactactttaatggaatctggtgtgataactccaaggtgcggtcacaaagctacgactacgggactgtctcagtggaactctcaaatcaaacagtagcctcttccagtttcatcatcttcgctaccttgatctctctcacaacaacttcacctcctcttccctcccttccgagtttgtttcccactttgcggaatctaaccaagctcacagttttagacctttctcataatcacttctccggaactttgaagcccaacaatagcctctttgagttacaccaccttcgttaccttaatctcgaggtcaacaacttcagttcctcactcccttccgagtttggctatctcaacaatttacagcactgtggcctcaaagagttcccaaacatattcaagacccttaaaaaaatggaggctatagacgtatccaacaatagaatcaacgggaaaatccctgagtggttatggagccttcctcttcttcatttagtgaatattttaaataattcttttgacggtttcgaaggatcaacggaagttttagtaaattcatcggttcggatattacttttggagtcaaacaactttgaaggagcacttcctagtctaccacactctatcaacgccttctccgcgggtcataacaatttcactggagagatacctctttcaatctgcaccagaacctcacttggtgtccttgatctaaactacaacaacctcattggtccggtttctcaatgtttgagtaatgtcacgtttgtaaatctccggaaaaacaatttggaaggaactattcctgagactttcattgtcggttcctcgataaggacacttgatgttggatacaatcgactaacgggaaagcttccaaggtctcttttgaactgctcatctctagagtttctaagcgttgacaacaacagaatcaaagacacatttcctttctggctcaaggctttaccaaagttacaagtccttaccctaagttcaaacaagttttatggtcctatatctcctcctcatcaaggtcctctcgggtttccagagctgagaatacttgagatatctgataataagtttactggaagcttgtcgtcaagatactttgagaattggaaagcatcgtccgccatgatgaatgaatatgtgggtttatatatggtttacgagaagaatccttatggtgtagttgtctatacctttttggatcgtatagatttgaaatacaaaggtctaaacatggagcaagcgagggttctcacttcctacagcgccattgatttttctagaaatctacttgaaggaaatattcctgaatccattggacttttaaaggcattgattgcactaaacttatcgaacaacgcttttacaggccatattcctcagtctttggcaaatcttaaggagctccagtcactagacatgtctaggaaccaactctcagggactattcctaatggactcaagcaactctcgtttttggcttacataagtgtgtctcataaccaactcaagggtgaaataccacaaggaacacaaattactgggcaattgaaatcttcctttgaagggaatgtaggactttgtggtcttcctctcgaggaaaggtgcttcgacaatagtgcatctccaacgcagcaccacaagcaagacgaagaagaagaagaagaacaagtgttacactggaaagcggtggcaatggggtatggacctggattgttggttggatttgcaattgcatatgtcattgcttcatacaagccggagtggctaaccaagataattggtccgaataagcgcagaaactag

SEQ ID NO: 21 - protein, 671 aa, Arabidopsis thaliana
MKTMSKSSLRLHFLSLLLLCCVSPSSFVIIRFITHNHFDGLVRCHPHKFQALTQFKNEFDTRRCNHSNYFNGIWCDNSKVRSQSYDYGTVSVELSNQTVASSSFIIFATLISLTTTSPPLPSLPSLFPTLRNLTKLTVLDLSHNHFSGTLKPNNSLFELHHLRYLNLEVNNFSSSLPSEFGYLNNLQHCGLKEFPNIFKTLKKMEAIDVSNNRINGKIPEWLWSLPLLHLVNILNNSFDGFEGSTEVLVNSSVRILLLESNNFEGALPSLPHSINAFSAGHNNFTGEIPLSICTRTSLGVLDLNYNNLIGPVSQCLSNVTFVNLRKNNLEGTIPETFIVGSSIRTLDVGYNRLTGKLPRSLLNCSSLEFLSVDNNRIKDTFPFWLKALPKLQVLTLSSNKFYGPISPPHQGPLGFPELRILEISDNKFTGSLSSRYFENWKASSAMMNEYVGLYMVYEKNPYGVVVYTFLDRIDLKYKGLNMEQARVLTSYSAIDFSRNLLEGNIPESIGLLKALIALNLSNNAFTGHIPQSLANLKELQSLDMSRNQLSGTIPNGLKQLSFLAYISVSHNQLKGEIPQGTQITGQLKSSFEGNVGLCGLPLEERCFDNSASPTQHHKQDEEEEEEQVLHWKAVAMGYGPGLLVGFAIAYVIASYKPEWLTKIIGPNKRRN

SEQ ID NO: 22 - DNA, 1662 bp, Arabidopsis thaliana
  source 1..1662: mol_type = other DNA
atgacttcctctcgccgtcttcttcttcctctcggagcatcgctcactagaggaagattttcttccgatcaaatccgaaatggatttctaagaaacttccgtggattcgccaccgtaacttcgtcggaaccggccttagccaatctggaagcgaaatatgccgtagcgttgccagaatgttcaacagtagaggacgagatcacgaagatccgtcatgaattcgagttagcgaaacagaggtttcttaatatccctgaagctattaatagtatgccgaagatgaatcctcaagggatatatgtgaataagaatctgagattggataatatacaagtttatggatttgattatgattacactttggcacattactcttctcacttacagagtttgatctatgatcttgccaagaaacatatggttaatgagtttagatatcctgatgtttgcactcagtttgagtatgatcctacttttccaatccgtgggttgtactatgataaactaaaaggatgcctcatgaaattggatttcttcggttcaatcgagccagatgggtgttattttggtcgtcgtaagcttagtaggaaggaaatagaaagcatgtatggaacgcggcacataggtcgtgatcaagcgagaggtttggtgggattgatggatttcttctgttttagcgaggcgtgtcttatagcagacatggtgcaatattttgttgacgccaaacttgagtttgatgcctctaacatctacaatgatgtcaatcgtgctattcaacatgtccatagaagtggattggttcatagaggaattcttgctgatcccaacagatatttgctaaaaaatggtcagcttctacgtttcctgagaatgctaaaagataaaggaaagaagctttttttgctgaccaactctccgtataattttgttgatggcggaatgcgctttctaatggaggaatcttttggcttcggagattcctggcgagaactctttgatgttgtgattgctaaagcaaataaaccagaattttacacatctgagcaccctttccgttgttatgattcggagagggataatttggcatttacaaaagtggatgcatttgacccaaagaaagtttattatcatggttgtcttaaatccttccttgaaatcacaaagtggcatggccctgaggtgatttatttcggagatcacttatttagtgatctaagagggccttcaaaagctggttggcgaactgctgccataattcatgagctcgagcgagagatacagatacaaaatgatgatagctaccggtttgagcaggccaagttccatattatccaagagttactcggtagatttcacgcgactgtatcaaacaatcagagaagtgaagcatgccaatcacttttggatgagctgaacaatgcgaggcagagagcaagagacacgatgaaacaaatgttcaacagatcgtttggagctacatttgtcacagacactggtcaagaatcagcattctcttatcacatccaccaatacgcagacgtttataccagtaaacctgagaactttctgttataccgacctgaagcctggcttcacgttccttacgatatcaagatcatgccacatcatgtcaaggttgcttcaacccttttcaaaacctga

SEQ ID NO: 23 - protein, 553 aa, Arabidopsis thaliana
MTSSRRLLLPLGASLTRGRFSSDQIRNGFLRNFRGFATVTSSEPALANLEAKYAVALPECSTVEDEITKIRHEFELAKQRFLNIPEAINSMPKMNPQGIYVNKNLRLDNIQVYGFDYDYTLAHYSSHLQSLIYDLAKKHMVNEFRYPDVCTQFEYDPTFPIRGLYYDKLKGCLMKLDFFGSIEPDGCYFGRRKLSRKEIESMYGTRHIGRDQARGLVGLMDFFCFSEACLIADMVQYFVDAKLEFDASNIYNDVNRAIQHVHRSGLVHRGILADPNRYLLKNGQLLRFLRMLKDKGKKLFLLTNSPYNFVDGGMRFLMEESFGFGDSWRELFDVVIAKANKPEFYTSEHPFRCYDSERDNLAFTKVDAFDPKKVYYHGCLKSFLEITKWHGPEVIYFGDHLFSDLRGPSKAGWRTAAIIHELEREIQIQNDDSYRFEQAKFHIIQELLGRFHATVSNNQRSEACQSLLDELNNARQRARDTMKQMFNRSFGATFVTDTGQESAFSYHIHQYADVYTSKPENFLLYRPEAWLHVPYDIKIMPHHVKVASTLFKT

SEQ ID NO: 24 - DNA, 1386 bp, Brachypodium distachyon
  source 1..1386: mol_type = other DNA
atggagattccggcggcgccgccgcctccattgccggtgctgtgctcgtacgtcgtcttcttgctgctgctgtcttcgtgctcactggccagagggaggatcgcggtttcttccccgggcccgtcgcctgtggccgccgccgttacagccaatgagaccgcttcatcctcttcttctccggtgtttccggccgctcctcccgtcgtgatcacagtggtgaggcaccaccattaccaccgggagctggtcatctccgctgtcctcgcctgcgtcgccaccgccatgatcctcctctccacactctacgcctggacgatgtggcggcggtctcgccggaccccccacggcggcaagggccgcggccggagatcagggatcacactggtgccaatcctgagcaagttcaattcagtgaagatgagcaggaaggggggccttgtgacgatgatcgagtacccgtcgctggaggcggcgacaggcaagttcggcgagagcaatgtgctcggtgtcggcggcttcggttgcgtttataaggcggcgtttgatggcggtgccaccgccgccgtgaagaggcttgaaggcggcgggccggattgcgagaaggaattcgagaatgagctggatttgcttggcaggatcaggcacccaaacatagtgtctctcctgggcttctgtgtccatggtggcaatcactacattgtttatgagctcatggagaagggatcattggagacacagctgcatgggtcttcacatggatctgctctgagctggcacgttcggatgaagatcgcgctcgatacggcgaggggattagagtatcttcatgagcactgcaatccacctgtgatccatagggatctgaaaccttctaatatacttttagattcagacttcaatgctaagattgcagattttggccttgcggtcaccggtgggaatctcaacaaagggaacctgaagctttccgggaccttgggttatgtagcccctgagtacttattagatgggaagttgactgagaagagcgatgtatacgcatttggagtagtgcttctagagctcctgatgggaaggaagcctgttgagaaaatgtcaccatctcagtgccaatcaattgtgtcatgggctatgcctcagctgaccgacagatcgaagctccccaacataattgacctggtgatcaaggacaccatggacccaaaacacttgtaccaagttgcagcagtggctgttctatgtgtgcagcccgaaccgagctacagaccactgataacagatgttctccactctcttgttcctctagtgcctgcggagctcggaggaacactcagggttgcagagccaccttcaccttctccagaccaaagacattatccttgttga

SEQ ID NO: 25 - DNA, 1302 bp, Brassica napus
  source 1..1302: mol_type = other DNA
atgaagaaactggttcatcttcagtttttgtttcttgtcaagatctttgctactcaattcctcactccttcttcatcatcttttgctgcttcaaatccttctatagctcctgtttacacctccatgactactttctctccaggaattcaaatgggaagtggtgaagaacacagattagatgcacataagaaactcctgattggtcttataatcagttcctcttctcttggtatcataatcttgatttgctttggcttctggatgtactgtcgcaagaaagctcccaaacccatcaagattccggatgccgagagtgggacttcatcattttcaatgtttgtgaggcggctaagctcaattaaaactcacagaacatctagcaatcagggttatgtgcagcgtttcgattccaagacgctagagaaagcgacaggcggtttcaaagacagtaatgtaatcggacagggcggtttcggatgcgtttacaaggcttctttggacagcaacactaaagcagcggttaaaaagatcgaaaacgttacccaagaagcaaaacgagaatttcagaatgaagttgagctgttgagcaagatccagcactccaatattatatcattgttgggctctgcaagtgaaatcaactcgagtttcgtcgtttatgagttgatggagaaaggatccttagatgatcagttacatggaccttcgtgtggatccgctctaacatggcatatgcgtatgaagattgctctagatacagctagaggactagagtatctccatgaacattgtcgtccaccagttatccacagggacctgaaatcgtctaatattcttcttgattcttccttcaatgccaagatttcagattttggtctggctgtatcggttggagtgcatgggagtaacaacattaaactctctgggacacttggttatgttgccccggaatatctcctagacggaaagttgacggataagagtgatgtctatgcatttggggtggttcttcttgaacttttgttgggtaggcggccggttgagaaattgagtccatctcagtgtcaatctcttgtgacttgggcaatgccacaacttaccgatagatcgaaactcccaaacatcgtggatccggttataaaagatacaatggatcttaagcacttataccaagtagcagccatggctgtgctgtgcgtacagccagaaccgagttaccggccgctgataaccgatgttcttcattcacttgttccattggttccggtagagctaggagggactctccggttaacccgatga

SEQ ID NO: 26 - protein, 433 aa, Brassica napus
MKKLVHLQFLFLVKIFATQFLTPSSSSFAASNPSIAPVYTSMTTFSPGIQMGSGEEHRLDAHKKLLIGLIISSSSLGIIILICFGFWMYCRKKAPKPIKIPDAESGTSSFSMFVRRLSSIKTHRTSSNQGYVQRFDSKTLEKATGGFKDSNVIGQGGFGCVYKASLDSNTKAAVKKIENVTQEAKREFQNEVELLSKIQHSNIISLLGSASEINSSFVVYELMEKGSLDDQLHGPSCGSALTWHMRMKIALDTARGLEYLHEHCRPPVIHRDLKSSNILLDSSFNAKISDFGLAVSVGVHGSNNIKLSGTLGYVAPEYLLDGKLTDKSDVYAFGVVLLELLLGRRPVEKLSPSQCQSLVTWAMPQLTDRSKLPNIVDPVIKDTMDLKHLYQVAAMAVLCVQPEPSYRPLITDVLHSLVPLVPVELGGTLRLTR

SEQ ID NO: 27 - DNA, 657 bp, Cichorium endivia
  source 1..657: mol_type = other DNA
atttttggtgttgaaatgatgcacaacggatctttggaatcccaattgcatggtccgtctcatggaactggcttaagctggcagcatcgaatgaaaattgcacttgatattgcacgaggactagagtatcttcacgagcgctgtaccccgcctgtgattcatagagatctgaaatcgtccaacattcttctaggttcgaactacaatgctaaactttctgatttcgggctcgcgattactggtgggattcagggcaagaacaacgtaaagctttcgggaacattaggttatgtagctccagaatacctcttagatggtaaacttactgataaaagtgatgtttatgcgtttggagttgtacttcttgaacttttgataggtagaaaaccagtggagaaaatgtcaccatctcaatgccaatctatcgttacatgggcaatgcctcaactaaccgaccgatcaaagcttcctaacatcgttgatcccgtgattagagatacaatggacttgaagcacttgtatcaagttgctgcggttgctgtgctatgtgtacaaccggaaccgagttacaggccattgataacagatgttttgcattcgttcatcccacttgtacctgttgagcttggagggtcgctaagagttaccgaatcttga

SEQ ID NO: 28 - protein, 218 aa, Cichorium endivia
IFGVEMMHNGSLESQLHGPSHGTGLSWQHRMKIALDIARGLEYLHERCTPPVIHRDLKSSNILLGSNYNAKLSDFGLAITGGIQGKNNVKLSGTLGYVAPEYLLDGKLTDKSDVYAFGVVLLELLIGRKPVEKMSPSQCQSIVTWAMPQLTDRSKLPNIVDPVIRDTMDLKHLYQVAAVAVLCVQPEPSYRPLITDVLHSFIPLVPVELGGSLRVTES

SEQ ID NO: 29 - DNA, 474 bp, Citrus clementina
  source 1..474: mol_type = other DNA
aattcggcacgagggctggattccagttttaatgcaaagctttcagattttggcctttctgtgactgctggaacccagagtaggaatgttaagatctctggaactctgggttatgttgccccggagtacctattagaaggaaaactaactgataaaagtgatgtatatgctttcggagttgtattgctggaacttttgatggggagaaggcctgtggaaaagatgtcaccaactcaatgtcaatcaatggtcacatgggccatgcctcagctcaccgatagatcaaagcttccaaacattgtggatccagtaattagagacacaatggatttaaagcacttataccaggtagccgctgtggcagtgctatgtatacaacctgaaccaagttataggccattgataaccgacgttctgcattccctcattcctcttgtacctaccgaccttggagggtcactccgagtgacctaa

SEQ ID NO: 30 - protein, 157 aa, Citrus clementina
NSARGLDSSFNAKLSDFGLSVTAGTQSRNVKISGTLGYVAPEYLLEGKLTDKSDVYAFGVVLLELLMGRRPVEKMSPTQCQSMVTWAMPQLTDRSKLPNIVDPVIRDTMDLKHLYQVAAVAVLCIQPEPSYRPLITDVLHSLIPLVPTDLGGSLRVT

SEQ ID NO: 31 - DNA, 770 bp, Citrus sinensis
  source 1..770: mol_type = other DNA
ggattgtgtttgtggctttatcatttgaagtactccttcaaatccagtaacaagaatgcaaagagcaaagattctgagaatggagttgtgttatcatcatttttgggcaaattcacttctgtgaggatggttagtaagaagggatctgctatttcatttattgagtataagctgttagagaaagccaccgacagttttcatgagagtaatatattgggtgagggtggatttggatgtgtttacaaggctaaattggatgataacttgcacgtcgctgtcaaaaaattagattgtgcaacacaagatgccggcagagaatttgagaatgaggtggatttgctgagtaatattcaccacccaaatgttgtttgtctgttgggttatagtgctcatgatgacacaaggtttattgtttatgaattgatggaaaatcggtcccttgatattcaattgcatggtccttctcatggatcagcattgacttggcatatgcgaatgaaaattgctcttgataccgctagaggattagaatatttacatgagcactgcaaccctgcagtcattcatagagatctgaaatcctccaatatacttctagattccaagtttaatgctaagctctcagattttggtcttgccataaccgatggatcccaaaacaagaacaatcttaagctttcgggcactttgggatatgtggctcccgagtatcttttagatggtaaattgacagacaagagtgatgtctatgcttttggagttgtgcttct

SEQ ID NO: 32 - protein, 257 aa, Citrus sinensis
GLCLWLYHLKYSFKSSNKNAKSKDSENGVVLSSFLGKFTSVRMVSKKGSAISFIEYKLLEKATDSFHESNILGEGGFGCVYKAKLDDNLHVAVKKLDCATQDAGREFENEVDLLSNIHHPNVVCLLGYSAHDDTRFIVYELMENRSLDIQLHGPSHGSALTWHMRMKIALDTARGLEYLHEHCNPAVIHRDLKSSNILLDSKFNAKLSDFGLAITDGSQNKNNLKLSGTLGYVAPEYLLDGKLTDKSDVYAFGVVLL

SEQ ID NO: 33 - DNA, 621 bp, Coffea canephora
  source 1..621: mol_type = other DNA
gcattgacatggcatcttaggatgaaaattgcccttgatgtagctagaggattagaatttttgcatgagcactgccacccagcagtgatccatagagatctgaaatcatctaatatccttctggattcaaatctcaatgctaagctatctgattttggtcttgccattcttgatggggctcaaaataagaacaacatcaagctttctggaaccttgggctatgtagctccagagtacctcttagatggtaaattgactgacaagagtgatgtttatgcttttggagtggtgcttttggagcttctcctgagaagaaagcctgtggagaagctggcaccagctcaatgccaatctatagtcacatgggctatgcctcagctgacagatagatcaaagcttccaaacatcgtggatcctgtgattagaaatgctatggatataaagcacttattccaggttgctgcagtcgctgtgctatgcgtgcagcctgaaccaagctatcgaccactgataacagatgtgttgcattcccttgttccccttgttcctatggagcttggcgggacgctcagagttgaacgacctgcttctgtgacctctctgttgattgattctacctga

SEQ ID NO: 34 - protein, 206 aa, Coffea canephora
ALTWHLRMKIALDVARGLEFLHEHCHPAVIHRDLKSSNILLDSNLNAKLSDFGLAILDGAQNKNNIKLSGTLGYVAPEYLLDGKLTDKSDVYAFGVVLLELLLRRKPVEKLAPAQCQSIVTWAMPQLTDRSKLPNIVDPVIRNAMDIKHLFQVAAVAVLCVQPEPSYRPLITDVLHSLVPLVPMELGGTLRVERPASVTSLLIDST

SEQ ID NO: 35 - DNA, 411 bp, Eucalyptus gunnii
  source 1..411: mol_type = other DNA
actgaggtgacccggaagaaaaacagggtaaagctatcgggcactttgggttatgtagccccagaatatgtcttggatggtaaattgactgataagagtgatgtctatgcctttggagttgtgcttttggagctccttttgagaagaaggcctcttgagatagtagcacccactcagtgccagtctattgttacatgggccatgcctcagctgaccgaccgaactaagcttccagatattgtggatcctgtaattagagatgcgatggatgtcaagcacttataccaggcagctgctgttgctgttttgtgtctgcaaccagaaccgatctaccggccactgataacggatgtactccactctctcattccacttgtacccgttgaacttgggggaacgctgaagacctag

SEQ ID NO: 36 - protein, 136 aa, Eucalyptus gunnii
TEVTRKKNRVKLSGTLGYVAPEYVLDGKLTDKSDVYAFGVVLLELLLRRRPLEIVAPTQCQSIVTWAMPQLTDRTKLPDIVDPVIRDAMDVKHLYQAAAVAVLCLQPEPIYRPLITDVLHSLIPLVPVELGGTLKT

SEQ ID NO: 37 - DNA, 522 bp, Festuca arundinacea
  source 1..522: mol_type = other DNA
acgaggcctcgtgccatacttttggattcagatttcaatgccaagatttcggatttcggtcttgcagtgtcaagtggaaatcgcaccaaaggtaatctgaagctttccggaactttgggctatgttgctcctgagtacttattagacgggaagttgacagagaagagtgatgtatatgcgttcggagtagtacttcttgagcttttgttaggaaggaggccaattgagaagatggccccatctcaatgccaatcaattgttacatgggccatgcctcagctaattgacagatcaaagctcccaaccataattgaccccgtgatcaggaacacgatggacctgaagcacttgtaccaagttgctgcagtggctgtgctctgtgtgcagccagaaccaagttataggccactaatcacagatgtgctccactctctgattcccctggtgcccatggagctcggagggtcactgagggctaccttggaatcgcctcgcgtatcacaacatcgttctccctgctga

SEQ ID NO: 38 - protein, 173 aa, Festuca arundinacea
TRPRAILLDSDFNAKISDFGLAVSSGNRTKGNLKLSGTLGYVAPEYLLDGKLTEKSDVYAFGVVLLELLLGRRPIEKMAPSQCQSIVTWAMPQLIDRSKLPTIIDPVIRNTMDLKHLYQVAAVAVLCVQPEPSYRPLITDVLHSLIPLVPMELGGSLRATLESPRVSQHRSPC

SEQ ID NO: 39 - DNA, 740 bp, Ginkgo biloba
  source 1..740: mol_type = other DNA
cctttattgaatagattgaactccttccgtggttctaggagaaagggatgtgcatatataattgaatattctctgctgcaagcagccacaaataattttagtacaagtgacatccttggagagggtggttttgggtgtgtatacagagctaggttagatgatgatttctttgctgctgtgaagaagttagatgagggcagcaagcaggctgagtatgaatttcagaatgaagttgaactaatgagcaaaatcagacatccaaatcttgtttctttgctggggttctgcattcatgggaagactcggttgctagtctacgagctcatgcaaaatggttctttggaagaccaattacatgggccatctcatggatccgcacttacatggtacctgcgcatgaaaatagcccttgattcagcaaggggtctagaacacttgcacgagcactgcaatcctgctgtgattcatcgtgatttcaaatcatcaaatatccttctggatgcaagcttcaatgccaagctttcagattttggtcttgcagtaacagctgcaggaggtattggtaatgctaatgtcgagctactgggcactttgggatatgtagctccagaatacctgcttgatggcaagttgacggagaaaagtgatgtctatggatttggagttgttcttttggagctaattatgggaagaaagccagttgataaatctgtggcaactgaaagtcaatcgctagtttc

SEQ ID NO: 40 - protein, 247 aa, Ginkgo biloba
PLLNRLNSFRGSRRKGCAYIIEYSLLQAATNNFSTSDILGEGGFGCVYRARLDDDFFAAVKKLDEGSKQAEYEFQNEVELMSKIRHPNLVSLLGFCIHGKTRLLVYELMQNGSLEDQLHGPSHGSALTWYLRMKIALDSARGLEHLHEHCNPAVIHRDFKSSNILLDASFNAKLSDFGLAVTAAGGIGNANVELLGTLGYVAPEYLLDGKLTEKSDVYGFGVVLLELIMGRKPVDKSVATESQSLVS

SEQ ID NO: 41 - DNA, 1254 bp, Glycine max
  source 1..1254: mol_type = other DNA
atgaaaatgaagcttctcctcatgcttcttcttcttgttcttcttcttcaccaacccatttgggctgcagaccctcctgcttcttctcctgctttatctccaggggaggagcagcatcaccggaataataaagtggtaatagctatcgtcgtagccaccactgcacttgctgcactcattttcagtttcttatgcttctgggtttatcatcataccaagtatccaacaaaatccaaattcaaatccaaaaattttcgaagtccagatgcagagaaggggatcaccttagcaccgtttgtgagtaaattcagttccatcaagattgttggcatggacgggtatgttccaataattgactataagcaaatagaaaaaacgaccaataattttcaagaaagtaacatcttgggtgagggcggttttggacgtgtttacaaggcttgtttggatcataacttggatgttgcagtcaaaaaactacattgtgagactcaacatgctgagagagaatttgagaacgaggtgaatatgttaagcaaaattcagcatccgaatataatatctttactgggttgtagcatggatggttacacgaggctcgttgtctatgagctgatgcataatggatcattggaagctcagttacatggaccttctcatggctcggcattgacttggcacatgaggatgaagattgctcttgacacagcaagaggattagaatatctgcacgagcactgtcaccctgcagtgatccatagggatatgaaatcttctaatattctcttagatgcaaacttcaatgccaagctgtctgattttggtcttgccttaactgatgggtcccaaagcaagaagaacattaaactatcgggtaccttgggatacgtagcaccggagtatcttctagatggtaaattaagtgataaaagtgatgtctatgcttttggggttgtgctattggagctcctactaggaaggaagccagtagaaaaactggtaccagctcaatgccaatctattgtcacatgggccatgccacacctcacggacagatccaagcttccaagcattgtggatccagtgattaagaatacaatggatcccaagcacttgtaccaggttgctgctgtagctgtgctgtgcgtgcaaccagaacctagttaccgtccactgatcattgatgttcttcactcactcatccctcttgttcccattgagcttggaggaacactaagagtttcacaagtaatt

SEQ ID NO: 42 - protein, 418 aa, Glycine max
MKMKLLLMLLLLVLLLHQPIWAADPPASSPALSPGEEQHHRNNKVVIAIVVATTALAALIFSFLCFWVYHHTKYPTKSKFKSKNFRSPDAEKGITLAPFVSKFSSIKIVGMDGYVPIIDYKQIEKTTNNFQESNILGEGGFGRVYKACLDHNLDVAVKKLHCETQHAEREFENEVNMLSKIQHPNIISLLGCSMDGYTRLVVYELMHNGSLEAQLHGPSHGSALTWHMRMKIALDTARGLEYLHEHCHPAVIHRDMKSSNILLDANFNAKLSDFGLALTDGSQSKKNIKLSGTLGYVAPEYLLDGKLSDKSDVYAFGVVLLELLLGRKPVEKLVPAQCQSIVTWAMPHLTDRSKLPSIVDPVIKNTMDPKHLYQVAAVAVLCVQPEPSYRPLIIDVLHSLIPLVPIELGGTLRVSQVI

SEQ ID NO: 43 - DNA, 702 bp, Helianthus argophyllus
  source 1..702: mol_type = other DNA
actcaagcatcaaaatattgtaaatcttttgggtattgtgttcatgatgacacaaggtttttggtctatgaaatgatgcatcaaggctctttggactcacaattgcatggaccaactcatggaaccgcattaacctggcatcgaagaatgaaagtcgcacttgatattgctcgaggattagagtatcttcatgaacgatgcaacccgcctgtgattcatagagatcttaagtcatcgaacattttgctagattccaatttcaatgctaaaatttcgaattttgcacttgctaccactgagctccatgcgaagaacaaagttaagctttcggctacttctggttatttggctccggaatacctatcagaaggtaaacttaccgataaaagcgacgtatatgcattcggagtagtacttcttgggcttttaatcggtagaaaaccagtggagaaaatgtcaccatctttatttcaatctattgtcacatgggcaatgcctcagttaacagaccggtcaaagcttccaaacatcgttgaccctgtgattagagatacaatggacctgaagcacttatatcaagttgctgctgtagccgtactttgcgtgcaacccgaaccaagttacagaccgttgattacagacgtactacactcattcattccactcgtacccgttgatcttggagggtcattaagagcttaa

SEQ ID NO: 44 - protein, 233 aa, Helianthus argophyllus
TQASKYCKSFGYCVHDDTRFLVYEMMHQGSLDSQLHGPTHGTALTWHRRMKVALDIARGLEYLHERCNPPVIHRDLKSSNILLDSNFNAKISNFALATTELHAKNKVKLSATSGYLAPEYLSEGKLTDKSDVYAFGVVLLGLLIGRKPVEKMSPSLFQSIVTWAMPQLTDRSKLPNIVDPVIRDTMDLKHLYQVAAVAVLCVQPEPSYRPLITDVLHSFIPLVPVDLGGSLRA

SEQ ID NO: 45 - DNA, 752 bp, Helianthus ciliaris
  source 1..752: mol_type = other DNA
cgatcatttcgttgcggctgtaaaaaactccatggtccagaaccagatgcccaaaaagggtttgagaatgaagtagattggttaggtaaactcaagcatcaaaatattgtaaattttttgggttattgtgttcatgatgacacaaggtttttggtctatgaaatgatgcatcaaggctctttggactcacaattgcatggaccaactcatggaaccgcattaacctggcatcgaagaatgaaagtcgcacttgatattgctcgaggattagagtatcttcatgaacgatgcaacccgcctgtgattcatagagatctcaagtcatcgaacattttgctagattccaatttcaatgctaaaatttcgaattttgcacttgctaccactgagctccatgcgaagaacaaagttaagctttcgggtacttctggttatttggctccggaatacctatccgaaggtaaacttaccgataaaagtgatgtatatgcattcggagtagtacttcttgagcttttaatcggtagaaaaccagtggagaaaatgtcaccatctttatttcaatctattgtcacatgggcaatgcctcagctaacagaccggtcaaagcttccaaacattgttgaccctgtgattagagatacaatggacctgaagcacttgtatcaagttgctgctgtagccgtactttgcgtgcaacccgaaccaagttacagaccgttgattacagacgtactacactcattcattcc

SEQ ID NO: 46 - protein, 251 aa, Helianthus ciliaris
RSFRCGCKKLHGPEPDAQKGFENEVDWLGKLKHQNIVNFLGYCVHDDTRFLVYEMMHQGSLDSQLHGPTHGTALTWHRRMKVALDIARGLEYLHERCNPPVIHRDLKSSNILLDSNFNAKISNFALATTELHAKNKVKLSGTSGYLAPEYLSEGKLTDKSDVYAFGVVLLELLIGRKPVEKMSPSLFQSIVTWAMPQLTDRSKLPNIVDPVIRDTMDLKHLYQVAAVAVLCVQPEPSYRPLITDVLHSFIP

SEQ ID NO: 47 - DNA, 630 bp, Helianthus exilis
  source 1..630: mol_type = other DNA
atgatgcatcaagactctttggactcacaattgcatggaccaactcatggaaccgcattaacctggcatcgaagaatgaaagtcgcacttgatattgctcgaggattagagtatcttcatgaacgatgcaacccgcctgtgattcatagagatctcaagtcatcgaacattttgctagattccaatttcaatgctaaaatttcgaattttgcacttgctaccactgagctccatgcgaagaacaaagttaagctttcgggtacttctggttatttggctccggaatacctatccgaaggtaaacttaccgataaaagtgatgtatatgcattcggagtagtacttcttgagcttttaatcggtagaaaaccagtggagaaaatgtcaccatctttatttcaatctattgtcacatgggcaatgcctcagctaacagaccggtcaaagcttccaaacattgttgaccctgtgattagagatacaatggacctgaagcacttgtatcaagttgctgctgtagccgtactttgcgtgcaacccgaaccaagttacagaccgttgattacagacgtactacactcattcattccactcgtacccgttgatcttggagggtcattaagagcttaa

SEQ ID NO: 48 - protein, 209 aa, Helianthus exilis
MMHQDSLDSQLHGPTHGTALTWHRRMKVALDIARGLEYLHERCNPPVIHRDLKSSNILLDSNFNAKISNFALATTELHAKNKVKLSGTSGYLAPEYLSEGKLTDKSDVYAFGVVLLELLIGRKPVEKMSPSLFQSIVTWAMPQLTDRSKLPNIVDPVIRDTMDLKHLYQVAAVAVLCVQPEPSYRPLITDVLHSFIPLVPVDLGGSLRA

SEQ ID NO: 49 - DNA, 780 bp, Hordeum vulgare
  source 1..780: mol_type = other DNA
aatttgagaggtgagctggatttgcttcagaggattcagcattcgaatatagtgtcccttgtgggcttctgcattcatgaggagaaccgcttcattgtttatgagctgatggtgaatggatcacttgaaacacagcttcatgggccatcacatggatcagctctgagttggcacattcggatgaagattgctcttgatacagcaaggggattggagtatcttcacgagcactgcaatccaccaatcatccatagggatctgaagtcgtctaacatacttttgaattcagactttaatgcaaagatttcagattttggccttgcagtgacaagtggaaatcgcagcaaagggaatctgaagctttccggtactttgggttatgttgcccctgagtacttactagatgggaagttgactgagaagagcgatgtatatgcatttggagtagtacttcttgagcttcttttgggaaggaggccagttgagaagatggcaccatctcagtgtcaatcaattgttacatgggccatgccccagctaattgacagatccaagctccctaccataatcgaccccgtgatcagggacacgatggatcggaagcacttgtaccaagttgctgcagtggctgtgctctgcgtgcagccagaaccaagctacaggccactgatcacagatgtcctccactctctgattcccctggtgcccatggaccttggagggacgctgaggatcaacccggaatcgccttgcacgacacgaaatcaatctccctgctga

SEQ ID NO: 50 - protein, 259 aa, Hordeum vulgare
NLRGELDLLQRIQHSNIVSLVGFCIHEENRFIVYELMVNGSLETQLHGPSHGSALSWHIRMKIALDTARGLEYLHEHCNPPIIHRDLKSSNILLNSDFNAKISDFGLAVTSGNRSKGNLKLSGTLGYVAPEYLLDGKLTEKSDVYAFGVVLLELLLGRRPVEKMAPSQCQSIVTWAMPQLIDRSKLPTIIDPVIRDTMDRKHLYQVAAVAVLCVQPEPSYRPLITDVLHSLIPLVPMDLGGTLRINPESPCTTRNQSPC

SEQ ID NO: 51 - DNA, 816 bp, Ipomoea batatas
  source 1..816: mol_type = other DNA
cgggggctcttatcactcattgctgctgctactgcactgggtacaagcttattgctcatgggttgcttctggatttatcatagaaagaaaatccacaaatctcatgacattattcatagcccagatgtagttaaaggtcttgcattatcctcatatattagcaaatacaactccttcaagtcgaattgtgtgaaacgacatgtctcgttgtgggagtacaatacactcgagtcggccacaaatagttttcaagaaagcgagatcttgggtggaggggggttcgggcttgtgtacaagggaaaactagaagacaacttgtatgtagctgtgaagaggctggaagttggaagacaaaacgcaattaaagaattcgaggctgaaatagaggtattgggcacgattcagcacccgaatataatttcgttgttgggatatagcattcatgctgacacgaggctgctagtttatgaactgatgcagaatggatctctggagtatcaactacatggaccttcccatggatcagcattagcgtggcataatagattgaaaatcgcacttgatacagcaaggggattagaatatttacatgaacattgcaaaccaccagttatccatagagatctgaaatcctccaatattcttctagatgccaacttcaatgccaagatctcagattttggtcttgctgtgcgcgatggggctcaaaacaaaaataacattaagctctcgggaaccgttggctatgtagctccagaatacctattagatggaatactaacagataaaagtgatgtttatggcttccgagttgta

SEQ ID NO: 52 - protein, 272 aa, Ipomoea batatas
RGLLSLIAAATALGTSLLLMGCFWIYHRKKIHKSHDIIHSPDVVKGLALSSYISKYNSFKSNCVKRHVSLWEYNTLESATNSFQESEILGGGGFGLVYKGKLEDNLYVAVKRLEVGRQNAIKEFEAEIEVLGTIQHPNIISLLGYSIHADTRLLVYELMQNGSLEYQLHGPSHGSALAWHNRLKIALDTARGLEYLHEHCKPPVIHRDLKSSNILLDANFNAKISDFGLAVRDGAQNKNNIKLSGTVGYVAPEYLLDGILTDKSDVYGFRVV

SEQ ID NO: 53 - DNA, 867 bp, Lactuca sativa
  source 1..867: mol_type = other DNA
ggggatatacgtgtagaatcagcaacaaataacttcggtgaaagcgagatattaggcgtaggtggatttggatgcgtgtataaagctcgactcgatgataatttgcatgtagctgttaaaagattagatggtattagtcaagacgccattaaagaattccagacggaggtggatctattgagtaaaattcatcatccgaatatcatcaccttattgggatattgtgttaatgatgaaaccaagcttcttgtttatgaactgatgcataatggatctttagaaactcaattacatgggccttccagtggatccaatttaacatggcattgcaggatgaagattgctctagatacagcaagaggattagaatatttgcatgagaactgcaaaccatcggtgattcatagagatctgaaatcatctaatatccttctggattccagcttcaatgctaagctttcagattttggtcttgctataatggatggggcccagaacaaaaacaacattaagctttcagggacattgggttatgtagctcccgagtatcttttagatggaaaattgacggataaaagtgacgtgtatgcgtttggagttgtgcttttagagcttttacttggaaggcgacctgtagaaaaattagcagagtcgcaatgccaatctattgtcacttgggctatgccacaattaacagacagatcaaagcttccgaatattgtagatcccgtgatcagatacacaatggatctcaagcacctgtaccaagttgctgcggtggctgtgttatgtgtacaacccggaccaagctaccggccatttataaaccgacgtcttgcattctctgatccctcttgttccccgtga

SEQ ID NO: 54 - protein, 288 aa, Lactuca sativa
GDIRVESATNNFGESEILGVGGFGCVYKARLDDNLHVAVKRLDGISQDAIKEFQTEVDLLSKIHHPNIITLLGYCVNDETKLLVYELMHNGSLETQLHGPSSGSNLTWHCRMKIALDTARGLEYLHENCKPSVIHRDLKSSNILLDSSFNAKLSDFGLAIMDGAQNKNNIKLSGTLGYVAPEYLLDGKLTDKSDVYAFGVVLLELLLGRRPVEKLAESQCQSIVTWAMPQLTDRSKLPNIVDPVIRYTMDLKHLYQVAAVAVLCVQPGPSYRPFINRRLAFSDPSCSP

SEQ ID NO: 55 - DNA, 804 bp, Medicago truncatula
  source 1..804: mol_type = other DNA
aagttgaactgtgaatgtcaatatgctgagagagaatttgagaatgaggtggatttgttaagtaaaattcaacatccaaatgtaatttctctactgggctgtagcagtaatgaggattcaaggtttattgtctatgagttgatgcaaaatggatcattggaaactcaattacatggaccatctcatggctcagcattgacttggcatatgaggatgaagattgctcttgacacagctagaggtttaaaatatctgcatgagcactgctaccctgcagtgatccatagagatctgaaatcttctaatattcttttagatgcaaacttcaatgccaagctttctgattttggtcttgcaataactgatgggtcccaaaacaagaataacatcaagctttcaggcacattggggtatgttgccccggagtatcttttagatggtaaattgacagataaaagtgatgtgtatgcttttggagttgtgcttcttgagcttctattaggaagaaagcctgtggaaaaacttacaccatctcaatgccagtctattgtcacatgggccatgccacagctcacagacagatccaagcttccaaacattgtggataatgtgattaagaatacaatggatcctaagcacttataccaggttgctgctgtggctgtattatgtgtgcaaccagagccgtgctaccgccctttgattgcagatgttctacactccctcatccctcttgtacctgttgagcttggaggaacactcagagttgcacaagtgacgcagcaacctaagaattctagttaa

SEQ ID NO: 56 - protein, 267 aa, Medicago truncatula
KLNCECQYAEREFENEVDLLSKIQHPNVISLLGCSSNEDSRFIVYELMQNGSLETQLHGPSHGSALTWHMRMKIALDTARGLKYLHEHCYPAVIHRDLKSSNILLDANFNAKLSDFGLAITDGSQNKNNIKLSGTLGYVAPEYLLDGKLTDKSDVYAFGVVLLELLLGRKPVEKLTPSQCQSIVTWAMPQLTDRSKLPNIVDNVIKNTMDPKHLYQVAAVAVLCVQPEPCYRPLIADVLHSLIPLVPVELGGTLRVAQVTQQPKNSS

SEQ ID NO: 57 - DNA, 636 bp, Nicotiana tabacum
  source 1..636: mol_type = other DNA
cagttgcatggacctcctcgtggatcagctttgaattggcatcttcgcatggaaattgcattggatgtggctaggggactagaatacctccatgagcgctgtaacccccctgtaatccatagagatctcaaatcgtctaatgttctattggattcctacttcaatgcaaagctttctgacttttggcctagctatagctggatggaacttaaacaagagcaccgtaaagtctttcgggaactctgggatatgtggctccagagttacctcttagatgggaaattaactgataagagtgatgtctatgctttcggcattatacttctggagcttctaatggggagaagaccattggagaaactagcaggagctcagtgccaatctatcgtcacatgggcaatgccacagcttactgacaggtcaaagctcccaaatattgttgatcctgtcatcagaaacggaatgggcctcaagcacttgtatcaagttgctgctgtagccgtgctatgtgtacaaccagaaccaagttaccgaccactgataacagatgtcctgcactccttcattccccttgtaccaattgagcttggtgggtccttgagagttgtggattctgcattatctgttaacgcataa

SEQ ID NO: 58 - protein, 211 aa, Nicotiana tabacum
QLHGPPRGSALNWHLRMEIALDVARGLEYLHERCNPPVIHRDLKSSNVLLDSYFNAKLSDFWPSYSWMELKQEHRKVFRELWDMWLQSYLLDGKLTDKSDVYAFGIILLELLMGRRPLEKLAGAQCQSIVTWAMPQLTDRSKLPNIVDPVIRNGMGLKHLYQVAAVAVLCVQPEPSYRPLITDVLHSFIPLVPIELGGSLRVVDSALSVNA

SEQ ID NO: 59 - DNA, 1437 bp, Oryza sativa
  source 1..1437: mol_type = other DNA
atggagatggcgctaactccattgccgctcctgtgttcgtccgtcttgttcttggtgctatcttcgtgctcgttggccaatgggagggatacgccttcttcttcttcttcttcttcttcttcttcttcttcttcttcttcttcttcttcttcttcttcttctccggcgacgtctactgtggccaccggcatttccgccgccgccgccgccgccgccaatgggacggccgccttgtcttcggcagttccggcgcctccgcctgttgtgatcgtagtgcaccaccatttccaccgcgagctggtcatcgccgccgtcctcgcctgcatcgccaccgtcacgatcttcctttccacgctctacgcttggacactatggcggcgatctcgccggagcaccggcggcaaggtcaccaggagctcagacgcagcgaaggggatcaagctggtgccgatcttgagcaggttcaactcggtgaagatgagcaggaagaggctggttgggatgttcgagtacccgtcgctggaggcagcgacagagaagttcagcgagagcaacatgctcggtgtcggcgggtttggccgcgtctacaaggcggcgttcgacgccggagttaccgcggcggtgaagcggctcgacggcggcgggcccgactgcgagaaggaattcgagaatgagctggatttgcttggcaggatcaggcaccccaacattgtgtccctcttgggcttctgtatccatgaggggaatcactacattgtttatgagctgatggagaagggatcactggaaacacagcttcatgggtcttcacatggatcaactctgagctggcacatccggatgaagatcgcccttgacacggccaggggattagagtaccttcatgagcactgcagtccaccagtgatccatagggatctgaaatcgtctaacatacttttggattcagacttcaatgctaagattgcagattttggtcttgctgtgtctagtgggagtgtcaacaaagggagtgtgaagctctccgggaccttgggttatgtagctcctgagtacttgttggatgggaagttgactgaaaagagcgatgtatacgcgttcggagtagtgcttctagagctccttatggggaggaagcctgttgagaagatgtcaccatctcagtgccaatcaattgtgacatgggcaatgccacagttgaccgacagatcgaagctccccagcatagttgacccagtgatcaaggacaccatggatccaaaacacctgtaccaagttgcagcagtggctgttctatgcgtgcaggctgaaccaagctacaggccactgatcacagatgtgctccactctcttgttcctctagtgccgacggagctcggaggaacactaagagctggagagccaccttccccgaacctgaggaattctccatgctga

SEQ ID NO: 60 - protein, 478 aa, Oryza sativa
MEMALTPLPLLCSSVLFLVLSSCSLANGRDTPSSSSSSSSSSSSSSSSSSSSSSPATSTVATGISAAAAAAANGTAALSSAVPAPPPVVIVVHHHFHRELVIAAVLACIATVTIFLSTLYAWTLWRRSRRSTGGKVTRSSDAAKGIKLVPILSRFNSVKMSRKRLVGMFEYPSLEAATEKFSESNMLGVGGFGRVYKAAFDAGVTAAVKRLDGGGPDCEKEFENELDLLGRIRHPNIVSLLGFCIHEGNHYIVYELMEKGSLETQLHGSSHGSTLSWHIRMKIALDTARGLEYLHEHCSPPVIHRDLKSSNILLDSDFNAKIADFGLAVSSGSVNKGSVKLSGTLGYVAPEYLLDGKLTEKSDVYAFGVVLLELLMGRKPVEKMSPSQCQSIVTWAMPQLTDRSKLPSIVDPVIKDTMDPKHLYQVAAVAVLCVQAEPSYRPLITDVLHSLVPLVPTELGGTLRAGEPPSPNLRNSPC

SEQ ID NO: 61 - DNA, 891 bp, Physcomitrella sp.
  source 1..891: mol_type = other DNA
tactctcttttacaaactgctacgaacaacttcagctcctccaatttgctgggcgagggaagtttcgggcatgtgtataaagcgagactcgattatgatgtctatgccgctgtaaagagacttaccagcgtaggaaaacagccccaaaaagaactccagggagaggtggatctgatgtgcaagataagacatcccaacttggtggctctcctgggctattcaaatgacggcccagagcccttggttgtgtacgagctcatgcagaatggttcacttcatgatcagcttcatggcccctcatgcgggagtgcactcacctggtacctacgactaaagattgctcttgaagctgccagcagaggactggagcacctgcatgaaagctgcaagcctgcaataatccacagagacttcaaggcatccaacatcctcttggacgccagcttcaatgcgaaggtgtccgactttggtatagcggtagctctggaggaaggtggcgtggtgaaagacgacgtacaagtgcaaggcaccttcgggtacattgctcctgagtacctgatggacgggacattgacagagaagagtgatgtttacggatttggagtagtattgcttgagctgctgacaggcagactgcccattgatacgtccttaccactcggatcgcaatctctagtgacatgggtaacacccatactaactaaccgagcaaagctgatggaagttatcgaccccacccttcaagatacgctgaacgtgaagcaacttcaccaggtggccgcagtggcagtcctttgcgtccaagcggaacccagctaccgccctctcatcgccgacgtggttcagtcactggctccgctggtgcctcaagagctcggcggcgcattgcga

SEQ ID NO: 62 - protein, 297 aa, Physcomitrella sp.
YSLLQTATNNFSSSNLLGEGSFGHVYKARLDYDVYAAVKRLTSVGKQPQKELQGEVDLMCKIRHPNLVALLGYSNDGPEPLVVYELMQNGSLHDQLHGPSCGSALTWYLRLKIALEAASRGLEHLHESCKPAIIHRDFKASNILLDASFNAKVSDFGIAVALEEGGVVKDDVQVQGTFGYIAPEYLMDGTLTEKSDVYGFGVVLLELLTGRLPIDTSLPLGSQSLVTWVTPILTNRAKLMEVIDPTLQDTLNVKQLHQVAAVAVLCVQAEPSYRPLIADVVQSLAPLVPQELGGALR

SEQ ID NO: 63 - DNA, 1065 bp, Picea sp.
  source 1..1065: mol_type = other DNA
acctcagatgcctataggggtattccactcatgcctctcctgaatcgtttgaactcccgtatttccaagaagaagggatgtgcaactgcaattgaatattctaagctgcaagcagctacaaataacttcagcagcaataacattcttggagagggtggatttgcgtgtgtatacaaggccatgtttgatgatgattcctttgctgctgtgaagaagctagatgagggtagcagacaggctgagcatgaatttcagaatgaagtggagctgatgagcaaaatccgacatccaaaccttgtttctttgcttgggttctgctctcatgaaaatacacggttcttagtatatgatctgatgcagaatggctctttggaagaccaattacatgggccatctcacggatctgcacttacatggtttttgcgcataaagatagcacttgattcagcaaggggtctagaacacttgcatgagcactgcaaccctgcagtgattcatcgagatttcaaatcatcaaatattcttcttgatgcaagcttcaacgccaagctttcagattttggtcttgcagtaacaagtgcaggatgtgctggcaatacaaatattgatctagtagggacattgggatatgtagctccagaatacctacttgatggtaaattgacagagaaaagtgatgtctatgcatatggagttgttttgttggagctactttttggaagaaagccaattgataaatctctaccaagtgaatgccaatctctcatttcttgggcaatgccacagctaacagatagagaaaagctcccaactatagtagaccccatgatcaaaggcacaatgaacttgaaacacctatatcaagtagcagctgttgcaatgctatgtgtgcagccagaacccagttacaggccattaatagctgacgttgtgcactctctcattcctctcgtaccaatagaactcgggggaactttaaagctctctaatgcacgacccactgagatgaagttatttacttcttcccaatgcagtgttgagattgcttccaacccaaaattgtga

SEQ ID NO: 64 - protein, 354 aa, Picea sp.
TSDAYRGIPLMPLLNRLNSRISKKKGCATAIEYSKLQAATNNFSSNNILGEGGFACVYKAMFDDDSFAAVKKLDEGSRQAEHEFQNEVELMSKIRHPNLVSLLGFCSHENTRFLVYDLMQNGSLEDQLHGPSHGSALTWFLRIKIALDSARGLEHLHEHCNPAVIHRDFKSSNILLDASFNAKLSDFGLAVTSAGCAGNTNIDLVGTLGYVAPEYLLDGKLTEKSDVYAYGVVLLELLFGRKPIDKSLPSECQSLISWAMPQLTDREKLPTIVDPMIKGTMNLKHLYQVAAVAMLCVQPEPSYRPLIADVVHSLIPLVPIELGGTLKLSNARPTEMKLFTSSQCSVEIASNPKL

SEQ ID NO: 65 - DNA, 596 bp, Pinus sp.
  source 1..596: mol_type = other DNA
aattcggcacgaggagaacacttgcacgagcactgcaaccctgcagtgattcaccgagatttcaaatcatcaaatattcttcttgatgcaagcttcaacgccaagctttcagattttggtcttgcagtaaaaagtgcaggatgtgctggtaacacaaatattgatctagtagggacattgggatatgtagctccagaatacatgcttgatggtaaattgacagagaaaagtgatgtctatgcatatggagttgttttgttagagctactttttggaagaaagccaattgataaatctctaccaagtgaatgccaatctctcatttcttgggcaatgccacagctaacagatagagaaaagctcccgactataatagatcccatgatcaaaggcgcaatgaacttgaaacacctatatcaagtggcagctgttgcagtgctatgtgtgcagccagaacccagttacaggccattaatagctgacgttgtgcactctctcattcctctcgtaccagtagaacttgggggaacattaaagtcatcacccactgagatgaagtcatttgcttcttcccaatgcagtgcccacgttgcttc

SEQ ID NO: 66 - protein, 199 aa, Pinus sp.
NSARGEHLHEHCNPAVIHRDFKSSNILLDASFNAKLSDFGLAVKSAGCAGNTNIDLVGTLGYVAPEYMLDGKLTEKSDVYAYGVVLLELLFGRKPIDKSLPSECQSLISWAMPQLTDREKLPTIIDPMIKGAMNLKHLYQVAAVAVLCVQPEPSYRPLIADVVHSLIPLVPVELGGTLKSSPTEMKSFASSQCSAHVAS

SEQ ID NO: 67 - DNA, 1377 bp, Populus sp.
  source 1..1377: mol_type = other DNA
atgttcttgtttcctaaaacagttcctatttggttttttcatctgtgtctagtagcagttcatgccatacaagaagacccacctgtcccttcaccatctccctctctcatttctcctatttcaacttcaatggctgccttctctccaggggttgaatcggaaatgggaatcaaagaccacccccagcatgatgacctccacaggaaaataatcttgttgctcactgttgcttgttgcatacttgttatcatccttctttctttgtgttcttgtttcatttactataagaagtcctcacaaaagaaaaaagctactcggtgttcagatgtggagaaagggctttcattggcaccatttttgggcaaattcagttccttgaaaatggttagtaataggggatctgtttcattaattgagtataagatactagagaaaggaacaaacaattttggcgatgataaattgttgggaaagggaggatttggacgtgtatataaggctgtaatggaagatgactcaagtgctgcagtcaagaaactagactgcgcaactgatgatgcgcagagagaatttgagaatgaggtggatttgttaagcaaatttcaccatccaaatataatttctattgtgggttttagtgttcatgaggagatggggttcattatttatgagttaatgccaaatgggtgccttgaagatctactgcatggaccttctcgtggatcttcactaaattggcatttaaggttgaaaattgctcttgatacagcaagaggattagaatatctgcatgaattctgcaagccagcagtgatccatagagatctgaaatcatcgaatattcttttggacgccaacttcaatgccaagctgtcagattttggtcttgctgtagctgatagctctcataacaagaaaaagctcaagctttcaggcactgtgggttatgtagccccagagtatatgttagatggtgaattgacggataagagtgatgtctatgcttttggagttgtgcttctagagcttctattaggaagaaggcctgtagaaaaactgacaccagctcattgccaatctatagtaacatgggccatgcctcagctcactaacagagctgtgcttccaacccttgtggatcctgtgatcagagattcagtagatgagaagtacttgttccaggttgcagcagtagccgtgttgtgtattcaaccagagccaagttaccgccctctcataacagatgttgtgcactctctcgtcccattagttcctcttgagcttggagggacactaagagttccacagcctacaactcccagaggtcaacgacaaggcccatcaaagaaactgtttttggatggtgctgcctctgct

SEQ ID NO: 68 - protein, 459 aa, Populus sp.
MFLFPKTVPIWFFHLCLVAVHAIQEDPPVPSPSPSLISPISTSMAAFSPGVESEMGIKDHPQHDDLHRKIILLLTVACCILVIILLSLCSCFIYYKKSSQKKKATRCSDVEKGLSLAPFLGKFSSLKMVSNRGSVSLIEYKILEKGTNNFGDDKLLGKGGFGRVYKAVMEDDSSAAVKKLDCATDDAQREFENEVDLLSKFHHPNIISIVGFSVHEEMGFIIYELMPNGCLEDLLHGPSRGSSLNWHLRLKIALDTARGLEYLHEFCKPAVIHRDLKSSNILLDANFNAKLSDFGLAVADSSHNKKKLKLSGTVGYVAPEYMLDGELTDKSDVYAFGVVLLELLLGRRPVEKLTPAHCQSIVTWAMPQLTNRAVLPTLVDPVIRDSVDEKYLFQVAAVAVLCIQPEPSYRPLITDVVHSLVPLVPLELGGTLRVPQPTTPRGQRQGPSKKLFLDGAASA

SEQ ID NO: 69 - DNA, 693 bp, Saccharum officinarum
  source 1..693: mol_type = other DNA
gctgctgcggtgaagagattggatggtggggctggggcacatgattgcgagaaggaattcgagaatgagttagatttgcttggaaagattcggcatccgaacattgtgtcccttgtgggcttctgtattcatgaggagaaccgtttcattgtttatgagctgatagagaatgggtcgttggattcacaacttcatgggccatcacatggttcagctctgagctggcatattcggatgaagattgctcttgacacggcaaggggattagagtacctgcatgagcactgcaacccaccagttatccatagggatctgaagtcatctaacatacttttagattcagacttcagtgctaagatttcagattttggccttgcggtgattagtgggaatcacagcaaagggaatttaaagctttctgggactatgggctatgtggcccctgagtacttattggatgggaagttgactgagaagagcgatgtatatgcgtttggggtggtacttctagaacttctactgggaaggaaacctgttgagaagatggcacaatctcaatgccaatcaattgttacatgggccatgcctcagctaactgatagatccaaactccctaacataattgatcccatgatcaagaacacaatggatctgaaacacttgtaccaagttgctgcaatggctgtgctctga

SEQ ID NO: 70 - protein, 230 aa, Saccharum officinarum
AAAVKRLDGGAGAHDCEKEFENELDLLGKIRHPNIVSLVGFCIHEENRFIVYELIENGSLDSQLHGPSHGSALSWHIRMKIALDTARGLEYLHEHCNPPVIHRDLKSSNILLDSDFSAKISDFGLAVISGNHSKGNLKLSGTMGYVAPEYLLDGKLTEKSDVYAFGVVLLELLLGRKPVEKMAQSQCQSIVTWAMPQLTDRSKLPNIIDPMIKNTMDLKHLYQVAAMAVL

SEQ ID NO: 71 - DNA, 414 bp, Triphysaria versicolor
  source 1..414: mol_type = other DNA
accctcggttatgtagctcctgagtatctgttagatggtaagttaacagagaaaagcgatgtgtatgggtttggagtagtgttactcgagcttctgcttgggaagaagcctatggagaaagtggcaacaacagcaactcagtgccagatgatagtcacatggaccatgcctcagctcactgacagaacgaaacttccgaatatcgtggatccggtgatcagaaactccatggatttaaagcacttgtaccaggttgctgctgtggcagtattgtgtgtgcagccagaaccgagttatcggccattgataactgatattttgcattctcttgtgccccttgtccctgttgagcttggtgggacgctcaggaactcgataacaatggctacaacaacaatatctcctgaaagctaa

SEQ ID NO: 72 - protein, 137 aa, Triphysaria versicolor
TLGYVAPEYLLDGKLTEKSDVYGFGVVLLELLLGKKPMEKVATTATQCQMIVTWTMPQLTDRTKLPNIVDPVIRNSMDLKHLYQVAAVAVLCVQPEPSYRPLITDILHSLVPLVPVELGGTLRNSITMATTTISPES

SEQ ID NO: 73 - DNA, 1140 bp, Triticum aestivum
  variation 1000: note = n is a, c, g, or t
  variation 1080: note = n is a, c, g, or t
  variation 1088: note = n is a, c, g, or t
  source 1..1140: mol_type = other DNA
cggcacgaggggctggtggccatgatcgagtacccgtcgctggaggcggcgacgggcaagttcagcgagagcaacgtgctcggcgtcggcgggttcggctgcgtctacaaggcggcgttcgacggcggcgccaccgccgccgtgaagaggctcgaaggcggcgagccggactgcgagaaggagttcgagaatgagctggacttgcttggcaggatcaggcacccaaacatagtgtccctcctgggcttctgcgtccatggtggcaatcactacattgtttatgagctcatggagaagggatcattggagacacaactgcatgggccttcacatggatcggctatgagctggcacgtccggatgaagatcgcgctcgacacggcgaggggattagagtatcttcatgagcactgcaatccaccagtcatccatagggatctgaaatcgtctaatatactcttggattcagacttcaatgctaagattgcagattttggccttgcagtgacaagtgggaatcttgacaaagggaacctgaagatctctgggaccttgggatatgtagctcccgagtacttattagatgggaagttgaccgagaagagcgacgtctacgcgtttggagtagtgcttctagagctcctgatggggaggaagcctgttgagaagatgtcaccatctcagtgccaatcaattgtgtcatgggccatgcctcagctaaccgacagatcgaagctacccaacatcatcgacccggtgatcaaggacacaatggacccaaagcatttataccaagttgcggcggtggccgttctatgcgtgcagcccgaaccgagttacagaccgctgataacagacgttctccactcccttgttcctctggtacccgcggatctcggggggaacgctcagagttacagagccgcattctccacaccaaatgtaccatccctcttgagaagtgatcctacaagtttcgtcgaagcggggaaagcgaatntatacggtccagcggtagatggctgttattttggtacttatatctcaccctgtcctgctgcttatcttaggatgagtgangagctccnacctgctgcttttgctggttgggcagagagaatacagttctggttaggattg

SEQ ID NO: 74 - protein, 380 aa, Triticum aestivum
  VAR_SEQ 334: note = X can be any naturally occurring amino acid
  VAR_SEQ 360: note = X can be any naturally occurring amino acid
  VAR_SEQ 363: note = X can be any naturally occurring amino acid
RHEGLVAMIEYPSLEAATGKFSESNVLGVGGFGCVYKAAFDGGATAAVKRLEGGEPDCEKEFENELDLLGRIRHPNIVSLLGFCVHGGNHYIVYELMEKGSLETQLHGPSHGSAMSWHVRMKIALDTARGLEYLHEHCNPPVIHRDLKSSNILLDSDFNAKIADFGLAVTSGNLDKGNLKISGTLGYVAPEYLLDGKLTEKSDVYAFGVVLLELLMGRKPVEKMSPSQCQSIVSWAMPQLTDRSKLPNIIDPVIKDTMDPKHLYQVAAVAVLCVQPEPSYRPLITDVLHSLVPLVPADLGGNAQSYRAAFSTPNVPSLLRSDPTSFVEAGKANXYGPAVDGCYFGTYISPCPAAYLRMSXELXPAAFAGWAERIQFWLGL

SEQ ID NO: 75 - DNA, 978 bp, Vitis vinifera
  source 1..978: mol_type = other DNA
atgaaagtgattgggagaaagggttatgtctcttttattgattataaggtactagaaactgcaacaaacaattttcaggaaagtaatatcctgggtgagggcgggtttggttgcgtctacaaggcgcggttggatgataactcccatgtggctgtgaagaagatagatggtagaggccaggatgctgagagagaatttgagaatgaggtggatttgttgactaaaattcagcacccaaatataatttctctcctgggttacagcagtcatgaggagtcaaagtttcttgtctatgagctgatgcagaatggatctctggaaactgaattgcacggaccttctcatggatcatctctaacttggcatattcgaatgaaaatcgctctggatgcagcaagaggattagagtatctacatgagcactgcaacccaccagtcatccatagagatcttaaatcatctaatattcttctggattcaaacttcaatgccaagctttcggattttggtctagctgtaattgatgggcctcaaaacaagaacaacttgaagctttcaggcaccctgggttatctagctcctgagtatcttttagatggtaaactgactgataagagtgatgtgtatgcatttggagtggtgcttctagagctactactgggaagaaagcctgtggaaaaactggcaccagctcaatgccagtccattgtcacatgggccatgccacagctgactgacagatcaaagctcccaggcatcgttgaccctgtggtcagagacacgatggatctaaagcatttataccaagttgctgctgtagctgtgctatgtgtgcaaccagaaccaagttaccggccattgataacagatgttctgcactccctcatcccactcgttccagttgagttgggagggatgctaaaagttacccagcaagcgccgcctatcaacaccactgcaccttctgctggaggttga

SEQ ID NO: 76 - protein, 325 aa, Vitis vinifera
MKVIGRKGYVSFIDYKVLETATNNFQESNILGEGGFGCVYKARLDDNSHVAVKKIDGRGQDAEREFENEVDLLTKIQHPNIISLLGYSSHEESKFLVYELMQNGSLETELHGPSHGSSLTWHIRMKIALDAARGLEYLHEHCNPPVIHRDLKSSNILLDSNFNAKLSDFGLAVIDGPQNKNNLKLSGTLGYLAPEYLLDGKLTDKSDVYAFGVVLLELLLGRKPVEKLAPAQCQSIVTWAMPQLTDRSKLPGIVDPVVRDTMDLKHLYQVAAVAVLCVQPEPSYRPLITDVLHSLIPLVPVELGGMLKVTQQAPPINTTAPSAGG

SEQ ID NO: 77 - DNA, 1377 bp, Zea mays
  source 1..1377: mol_type = other DNA
atgccgccgccatcgccgctcctccgttcctccgccttcgtcgtcttgctgctcctggtgtgtcgcccgttgttggtcgccaatgggagggccacgccgccttctccgggatggccaccggcggctcagcccgcgctgcagcctgcacccaccgccagcggcggcgtggcctccgtgcttccttcggccgtggcgcctcctcccttaggtgtggttgtggcggagaggcaccaccacctcagcagggagctcgtcgctgccattatcctctcatccgtcgccagcgtcgtgatccccattgccgcgctgtatgccttcttgctgtggcgacgatcacggcgagccctggtggattccaaggacacccagagcatagataccgcaaggattgcttttgcgccgatgttgaacagctttggctcgtacaagactaccaagaagagtgccgcggcgatgatggattacacatctttggaggcagcgacagaaaacttcagtgagagcaatgtccttggatttggtgggtttgggtctgtgtacaaagccaattttgatgggaggtttgctgctgcggtgaagagactggatggtggggcacatgattgcaagaaggaattcgagaatgagctagacttgcttgggaagattcgacatccgaacatcgtgtcccttgtgggcttctgcattcatgaggagaaccgtttcgttgtttatgagctgatggagagtgggtcgttggattcgcaacttcatgggccatcacatggttcagctctgagctggcatattcggatgaagattgctctcgacacagcaaggggattagagtacctgcatgagcactgcaacccaccggttatccatagggatcttaagtcatctaacatacttttagattcagacttcagcgctaagatttcagactttggcctggcagtgactagtgggaatcacagcaaagggaatttaaagctttctgggactatgggctatgtggctcctgagtacttattagatgggaagctgactgagaagagcgatgtatacgcgtttggggtagtacttctagaactcctgctgggaaggaaacctgtcgagaagatggcacaatctcagtgccgatcaatcgttacatgggccatgcctcagctaactgatagatccaagctcccgaacataattgatcccatgatcaagaacacaatggatctgaaacacttgtaccaagttgctgcagtggccgtgctctgcgtgcagccagagccgagttacaggccactgatcaccgacgtgcttcactcactggtacctctagtgcccacggagcttggaggaacgctgaggatcggcccggaatcgccctacctacgctactaa

SEQ ID NO: 78 - protein, 458 aa, Zea mays
MPPPSPLLRSSAFVVLLLLVCRPLLVANGRATPPSPGWPPAAQPALQPAPTASGGVASVLPSAVAPPPLGVVVAERHHHLSRELVAAIILSSVASVVIPIAALYAFLLWRRSRRALVDSKDTQSIDTARIAFAPMLNSFGSYKTTKKSAAAMMDYTSLEAATENFSESNVLGFGGFGSVYKANFDGRFAAAVKRLDGGAHDCKKEFENELDLLGKIRHPNIVSLVGFCIHEENRFVVYELMESGSLDSQLHGPSHGSALSWHIRMKIALDTARGLEYLHEHCNPPVIHRDLKSSNILLDSDFSAKISDFGLAVTSGNHSKGNLKLSGTMGYVAPEYLLDGKLTEKSDVYAFGVVLLELLLGRKPVEKMAQSQCRSIVTWAMPQLTDRSKLPNIIDPMIKNTMDLKHLYQVAAVAVLCVQPEPSYRPLITDVLHSLVPLVPTELGGTLRIGPESPYLRY

SEQ ID NO: 79 - DNA, 1188 bp, Zea mays
  source 1..1188: mol_type = other DNA
atgttgctcgcgtgtcctgcagtgatcatcgtggagcgccaccgtcatttccaccgtgagctagtcatcgcctccatcctcgcctcaatcgccatggtcgcgattatcctctccacgctgtacgcgtggatcccgcgcaggcggtcccgccggctgccccgcggcatgagcgcagacaccgcgagggggatcatgctggcgccgatcctgagcaagttcaactcgctcaagacgagcaggaaggggctcgtggcgatgatcgagtacccgtcgctggaggcagcgacaggggggttcagtgagagcaacgtgctcggcgtaggcggcttcggttgcgtctacaaggcagtcttcgatggcggcgttaccgcggcggtcaagaggctggagggaggtggccctgagtgcgagaaggaattcgagaatgagctggatctgcttggcaggattcggcaccccaacatcgtgtccctgctgggcttttgtgttcacgaggggaatcactacattgtttatgagctcatggagaagggatccctggacacacagctgcatggggcctcacatggatcagcgctgacctggcatatccggatgaagatcgcactcgacatggccaggggattagaatacctccatgagcactgcagtccaccagtgatccatagggatctgaagtcatctaacatacttttagattctgacttcaatgctaagatttcagattttggtcttgcagtgaccagtgggaacattgacaagggaagcatgaagctttctgggaccttgggttatgtggcccctgagtacctattagatgggaagctgactgaaaagagtgacgtatatgcatttggagtggtgcttcttgagctactaatgggaaggaagcctgtcgagaagatgagtcaaactcagtgccaatcaattgtgacgtgggccatgccgcagctgactgacagaacaaaacttcccaacatagttgacccagtgatcagggacaccatggatccaaagcatttgtaccaagtggcagcagtggcagttctatgtgtgcaaccagaaccaagttacagaccgctgattactgatgttctccactctcttgtccctctagtccctgtggagctcggagggacactgagggttgtagagccaccttccccaaacctaaaacattctccttgt

SEQ ID NO: 80 - protein, 396 aa, Zea mays
MLLACPAVIIVERHRHFHRELVIASILASIAMVAIILSTLYAWIPRRRSRRLPRGMSADTARGIMLAPILSKFNSLKTSRKGLVAMIEYPSLEAATGGFSESNVLGVGGFGCVYKAVFDGGVTAAVKRLEGGGPECEKEFENELDLLGRIRHPNIVSLLGFCVHEGNHYIVYELMEKGSLDTQLHGASHGSALTWHIRMKIALDMARGLEYLHEHCSPPVIHRDLKSSNILLDSDFNAKISDFGLAVTSGNIDKGSMKLSGTLGYVAPEYLLDGKLTEKSDVYAFGVVLLELLMGRKPVEKMSQTQCQSIVTWAMPQLTDRTKLPNIVDPVIRDTMDPKHLYQVAAVAVLCVQPEPSYRPLITDVLHSLVPLVPVELGGTLRVVEPPSPNLKHSPC

SEQ ID NO: 81 - DNA, 1086 bp, Gossypium sp.
  source 1..1086: mol_type = other DNA
atgaagaagaagcttgtgctgcatctgcttcttttccttgtttgtgctcttgaaaacattgttttggccgtacaaggccctgcttcatcacccatttctactcccatctctgcttcaatggctgccttctctccagctgggattcaacttggaggtgaggagcacaagaaaatggatccaaccaagaaaatgttattagctctcattcttgcttgctcttcattgggtgcaattatctcttccttgttctgtttatggatttattacaggaagaattcaagcaaatcctctaaaaatggcgctaagagctcagatggtgaaaaagggaatggtttggcaccatatttgggtaaattcaagtctatgaggacggtttccaaagagggttatgcttcgtttatggactataagatacttgaaaaagctacaaacaagttccatcatggtaacattctgggtgagggtggatttggatgtgtttacaaggctcaattcaatgatggttcttatgctgctgttaagaagttggactgtgcaagccaagatgctgaaaaagaatatgagaatgaggtgggtttgctatgtagatttaagcattccaatataatttcactgttgggttatagcagtgataacgatacaaggtttattgtttatgagttgatggaaaatggttctttggaaactcaattacatggaccttctcatggttcatcattaacttggcataggaggatgaaaattgctttggatacagcaagaggattagaatatctacatgagcattgcaatccaccagtcatccatagagatctgaaatcatctaatatacttttggatttggacttcaatgcaaagctttcagattttggtcttgcagtaactgatgcggcaacaaacaagaataacttgaagctttcgggtactttaggttatctagctccagaataccttttagatggtaaattaacagataagagtgatgtttatgcattcggtgttgtgctgctcgaacttctattgggacgaaaggctgttgaaaaattatcacaactcagtgccaatcttaggtccatttgggcatag

SEQ ID NO: 82 - protein, 361 aa, Gossypium sp.
MKKKLVLHLLLFLVCALENIVLAVQGPASSPISTPISASMAAFSPAGIQLGGEEHKKMDPTKKMLLALILACSSLGAIISSLFCLWIYYRKNSSKSSKNGAKSSDGEKGNGLAPYLGKFKSMRTVSKEGYASFMDYKILEKATNKFHHGNILGEGGFGCVYKAQFNDGSYAAVKKLDCASQDAEKEYENEVGLLCRFKHSNIISLLGYSSDNDTRFIVYELMENGSLETQLHGPSHGSSLTWHRRMKIALDTARGLEYLHEHCNPPVIHRDLKSSNILLDLDFNAKLSDFGLAVTDAATNKNNLKLSGTLGYLAPEYLLDGKLTDKSDVYAFGVVLLELLLGRKAVEKLSQLSANLRSIWA

SEQ ID NO: 83 - DNA, 1089 bp, Solanum lycopersicum
  variation 1024: note = n is a, c, g, or t
  source 1..1089: mol_type = other DNA
ggagtgggaattgagaagcagccacccacccacccaccctatggataaaaatagaaggctgttgatagcactcattgtagcttctactgcattaggactaatctttatcttcatcattttattctggatttttcacaaaagatttcacacctcagatgttgtgaagggaatgagtaggaaaacattggtttctttaatggactacaacatacttgaatcagccaccaacaaatttaaagaaactgagattttaggtgaggggggttttggatgtgtgtacaaagctaaattggaagacaatttttatgtagctgtcaagaaactaacccaaaattccattaaagaatttgagactgagttagagttgttgagtcaaatgcaacatcccaatattatttcattgttgggatattgcatccacagtgaaacaagattgcttgtctatgaactcatgcaaaatggatcactagaaactcaattacatgggccttcccgtggatcagcattaacttggcatcgcaggataaaaattgcccttgatgcagcaagaggaatagaatatttacatgagcagcgccatccccctgtaattcatagagatctgaaatcatctaatattcttttagattccaacttcaatgcaaaggtaaaactttttatgtagaaattatactaggactagttttccctctattaatcttgtgttgtgattaattttagctgtcagattttggtcttgctgtgttgagtggggctcaaaacaaaaacaatatcaagctttctggaactataggttatgtagcgcctgaatacatgttagatggaaaattaagtgataaaagtgatgtttatggttttggagtagtacttttggagctgttattgggaaggcggcctgtagaaaaggaggcagccactgaatgtcagtctatagtgacatgggccatgcctcagctgacagatagatcaaagcttccaaacattgttgatcctgtcatacaaaacacaatggatttaaagcatntgtatcaggttgctgcaggtgctctattatgtgttcagccagagccaagctatcgtcccgtataa

SEQ ID NO: 84 - DNA, 875 bp, Aquilegia sp.
  source 1..875: mol_type = other DNA
gagtatcagttattggaagctgcaactgacaattttagtgagagtaatattttgggagaaggtggatttggatgtgtttacaaagcatgttttgataacaactttctcgctgctgtcaagagaatggatgttggtgggcaagatgcagaaagagaatttgagaaagaagtagatttgttgaatagaattcagcatccggatataatttccctgttgggttattgtattcatgatgagacaaggttcatcatttatgaactaatgcagaacggatctttggaaagacaattacatggaccttctcatggatcggctttaacttggcatatccggatgaaaattgcacttgatacagcaagagcattagaatatctccatgagaattgcaaccctcctgtgatccacagagatctgaaatcatccaatatacttttggattctaatttcaaggccaagatttcagattttggtcttgctgtaatttctgggagtcaaaacaagaacaacattaagctttcaggcactcttggttatgttgctccagaatatctgttagatggtaaattgactgacaaaagtgatgtctatgcttttggggttatccttctagaactcctaatgggaagaaaacctgtagagaaaatgacacgaactcagtgtcaatctatcgttacatgggccatgcctcaactcactgatagatcaaagctaccaaacattgttgatcctgtgattaaaaacacaatggatttgaagcatttgttccaagttgctgctgtagctgtactgtgtgtacaaccagaaccaagttaccggccattaatcacagatgtccttcactccctcgtaccccttgttcctgtcgatcttggagg

SEQ ID NO: 85 - protein, 292 aa, Aquilegia sp.
EYQLLEAATDNFSESNILGEGGFGCVYKACFDNNFLAAVKRMDVGGQDAEREFEKEVDLLNRIQHPDIISLLGYCIHDETRFIIYELMQNGSLERQLHGPSHGSALTWHIRMKIALDTARALEYLHENCNPPVIHRDLKSSNILLDSNFKAKISDFGLAVISGSQNKNNIKLSGTLGYVAPEYLLDGKLTDKSDVYAFGVILLELLMGRKPVEKMTRTQCQSIVTWAMPQLTDRSKLPNIVDPVIKNTMDLKHLFQVAAVAVLCVQPEPSYRPLITDVLHSLVPLVPVDLGG

SEQ ID NO: 86 - DNA, 696 bp, Centaurea maculosa
  source 1..696: mol_type = other DNA
tgtgctcatgatgagaccaaactacttgtttacgaacttatgcacaatggttcgttagaaactcaattacacggtccttcttgtggatccaatttaacatggcattgtcggatgaaaattgcgctagatatagcgagaggattggaatatttacatgaacactgcaaaccatctgtgattcatagagatttgaagtcatctaacatccttttggattcaaaattcaatgccaagctttcggatttcggtcttgctgtgatgaacggtgccaataccaaaaacattaagctttcggggacgttgggttacgtagctcccgagtatcttttaaatgggaaattgaccgataaaagtgacgtctacgcattcggagttgtacttttagagcttctactcaaaaggcggcctgtcgaaaaactagcaccatccgagtgccagtccatcgtcacttgggctatgccgcaactaacagacagaacaaagcttccgagtgttatagatcccgtgatcagggacacgatggatcttaaacacttgtatcaagtggcggctgtggctgtgttgtgtgttcaaccggaaccgggataccggccgttgataaccgacgtcttgcattctctggttcctctcgtgccggttgaactcggagggactctacgagttgcggaaacaggttgcggcacagttgacttatga

SEQ ID NO: 87 - protein, 231 aa, Centaurea maculosa
CAHDETKLLVYELMHNGSLETQLHGPSCGSNLTWHCRMKIALDIARGLEYLHEHCKPSVIHRDLKSSNILLDSKFNAKLSDFGLAVMNGANTKNIKLSGTLGYVAPEYLLNGKLTDKSDVYAFGVVLLELLLKRRPVEKLAPSECQSIVTWAMPQLTDRTKLPSVIDPVIRDTMDLKHLYQVAAVAVLCVQPEPGYRPLITDVLHSLVPLVPVELGGTLRVAETGCGTVDL

SEQ ID NO: 88 - DNA, 842 bp, Cichorium intybus
  source 1..842: mol_type = other DNA
tggatttggatgcgtttaaaagctcaactcaatgataacttattagttgcggtcaaacgactagacaataaaagtcaaaattccatcaaagaattccagacggaagtgaatattttgagtaaaattcaacatccaaatataattagtttgttgggatattgcgatcatgatgaaagcaagctacttgtttacgaattgatgcaaaatggttctttagaaactcagttacatgggccttcttgtggatccaatttaacatggtattgccggatgaaaattgccctagatatagcaagaggattggaatatttacatgaacactccaaaccatctgtgattcatagagatctcaaatcatctaatatacttcttgattcaaatttcaatgcaaagctttcggattttggtcttgcggtgatggaaggtgcaaatagcaaaaacattaaactttcggggacattgggatacgtagcacccgaatatcttttagatgggaaattaaccgataaaagtgacgtgtatgcatttggagtcgtactttttgagcttttactcagaagacgacacgttgaaaaactagaatcatcacaatcccgccaatctattgtcacttgggcgatgccactactaatggacagatcgaagcttccgagtgtgatagatcctgtgattagggatacaatggatcttaaacatctttatcaagtggctgcggtggcggtgttgtgtgttcaatcggaaccgagttaccgtccgttgataaccgatgttttacattctcttgttcctcttgtcccggttgaacttggagggacacttagagttgtagaaaagagtgttgt

SEQ ID NO: 89 - protein, 281 aa, Cichorium intybus
WIWMRLKAQLNDNLLVAVKRLDNKSQNSIKEFQTEVNILSKIQHPNIISLLGYCDHDESKLLVYELMQNGSLETQLHGPSCGSNLTWYCRMKIALDIARGLEYLHEHSKPSVIHRDLKSSNILLDSNFNAKLSDFGLAVMEGANSKNIKLSGTLGYVAPEYLLDGKLTDKSDVYAFGVVLFELLLRRRHVEKLESSQSRQSIVTWAMPLLMDRSKLPSVIDPVIRDTMDLKHLYQVAAVAVLCVQSEPSYRPLITDVLHSLVPLVPVELGGTLRVVEKSVV

SEQ ID NO: 90 - DNA, 495 bp, Cucumis melo
  source 1..495: mol_type = other DNA
attcttttagatgcaaacttcaatgccaagctttctgattttggcttgtctgtcattgttggagcacaaaacaagaatgatataaagctttccggaacgatgggttatgttgctcctgaatatcttttagatggtaaattgactgataaaagtgatgtctatgcttttggagttgtgcttttggagcttcttttaggaagaaggcctgttgaaaaactggcaccatctcaatgtcaatccattgtcacatgggctatgcctcaactcactgatagatcaaagttacccgatatcgttgatccggtgatcagacacacaatggaccctaaacatttatttcaggttgctgctgtcgccgtgctgtgtgtgcaaccagaaccgagctatcgtcccctaataacagatcttttgcactctcttattcctcttgttcctgttgagctaggaggtactcacagatcatcaacatcacaagctcctgtggctccagcttag

SEQ ID NO: 91 - protein, 164 aa, Cucumis melo
ILLDANFNAKLSDFGLSVIVGAQNKNDIKLSGTMGYVAPEYLLDGKLTDKSDVYAFGVVLLELLLGRRPVEKLAPSQCQSIVTWAMPQLTDRSKLPDIVDPVIRHTMDPKHLFQVAAVAVLCVQPEPSYRPLITDLLHSLIPLVPVELGGTHRSSTSQAPVAPA

SEQ ID NO: 92 - DNA, 375 bp, Eragrostis curvula
  source 1..375: mol_type = other DNA
gatgggaagctcaccgagaaaagcgacgtgtacgcgtttggcatagtgcttcttgagctgctaatgggaaggaagcctgttgagaagttgagtcaatctcagtgccaatcaattgtgacttgggccatgccccaactgacagacagatcaaaacttcccaacataattgacccagtgatcagggacacaatggatccaaagcacttgtatcaggttgcagcagtggctgttctatgcgtgcaaccagaaccgagttacagaccactgataacggatgttctccactctttagttcctctagtgcctgtggagcttggtgggacactaagggttgcagagccaccgtccccaaaccaaaatcattctcctcgttga

SEQ ID NO: 93 - protein, 124 aa, Eragrostis curvula
DGKLTEKSDVYAFGIVLLELLMGRKPVEKLSQSQCQSIVTWAMPQLTDRSKLPNIIDPVIRDTMDPKHLYQVAAVAVLCVQPEPSYRPLITDVLHSLVPLVPVELGGTLRVAEPPSPNQNHSPR

SEQ ID NO: 94 - DNA, 414 bp, Gerbera sp.
  misc_feature 1..414: note = Gerbera hybrid
  source 1..414: mol_type = other DNA
ggggttcatggcaagaacaatataaaactttcaggaactttaggatatgtcgcgccggaataccttttagatggtaaacttactgataaaagtgacgtttatgcgtttggagttgtgcttctcgagcttttgataggacgaaaacccgtggagaaaatgtcaccatttcaatgccaatttatcgttacatgggcaatgcctcagctaacggacagatcgaagcttcctaatcttgtggatcctgtgattagagatactatggacttgaagcccttatatcaagttgcggctgtaactgtgttatgtgtacaacccgaaccaagttaccgcccattaataacggatgttttgcattcgttcatcccacttgtacctgctgatcttggagggtcgttaaaagttgtcgacttttaa

SEQ ID NO: 95 - protein, 137 aa, Gerbera sp.
  REGION 1..137: note = Gerbera hybrid
GVHGKNNIKLSGTLGYVAPEYLLDGKLTDKSDVYAFGVVLLELLIGRKPVEKMSPFQCQFIVTWAMPQLTDRSKLPNLVDPVIRDTMDLKPLYQVAAVTVLCVQPEPSYRPLITDVLHSFIPLVPADLGGSLKVVDF

SEQ ID NO: 96 - DNA, 498 bp, Helianthus paradoxus
  source 1..498: mol_type = other DNA
atcgtgttccattttggttgttgtctaaagctttcagattttggtcttgctgtaatggatggagcccagaacaaaaacaacatcaagctttcagggacattgggttatgtagctccagagtatcttttagatggaaaactgaccgacaaaagtgatgtatatgcatttggagttgtacttttagagcttctacttggaagacggcctgtagaaaaactggccgcatctcaatgccaatctatcgtcacttgggccatgccacagctaacagacagatcaaagctcccaaatattgtcgatcctgtaatcagatatacgatggatctcaaacacttgtaccaagttgctgccgtggcagtgctgtgtgtgcaaccagagccaagttaccggccattaataaccgatgttttgcattctcttatccctcttgttccggtggagctcgggggaactctaaaagctccacaaacaaggtcttcggtaacaaatgacccgtga

SEQ ID NO: 97 - protein, 165 aa, Helianthus paradoxus
IVFHFGCCLKLSDFGLAVMDGAQNKNNIKLSGTLGYVAPEYLLDGKLTDKSDVYAFGVVLLELLLGRRPVEKLAASQCQSIVTWAMPQLTDRSKLPNIVDPVIRYTMDLKHLYQVAAVAVLCVQPEPSYRPLITDVLHSLIPLVPVELGGTLKAPQTRSSVTNDP

SEQ ID NO: 98 - DNA, 612 bp, Ipomoea nil
  source 1..612: mol_type = other DNA
cgtggatcaactttaagttggcctctccgaatgaaaattgctttggatattgcaagaggattagaataccttcacgagcgttgcaacccccctgtgatccataggcatctcaaatcgtctaatattcttcttgattccagcttcaacgcaaagatttctgattttggcctttctgtaactggcggaaacctaagcaagaacataaccaagatttcgggatcactgggttatcttgctccagagtatctcttagacggtaaactaactgataagagtgatgtgtatggttttggcattattcttctagagcttttgatgggtaaaaggccagtggagaaagtgggagaaactaagtgccaatcaatagttacatgggctatgccccagcttacggaccgatcaaagcttccgaatattgttgaccctacgatcaggaacacaatggatgttaagcatttatatcaggttgcggctgtagctgtgttatgtgtgcaaccggagccaagctataggccattgataactgatgtactacactccttcattccacttgtaccaaatgaactcggggggtcgcttagggtagtggattctactccccattgctcatag

SEQ ID NO: 99 - protein, 203 aa, Ipomoea nil
RGSTLSWPLRMKIALDIARGLEYLHERCNPPVIHRHLKSSNILLDSSFNAKISDFGLSVTGGNLSKNITKISGSLGYLAPEYLLDGKLTDKSDVYGFGIILLELLMGKRPVEKVGETKCQSIVTWAMPQLTDRSKLPNIVDPTIRNTMDVKHLYQVAAVAVLCVQPEPSYRPLITDVLHSFIPLVPNELGGSLRVVDSTPHCS

SEQ ID NO: 100 - DNA, 708 bp, Nuphar advena
  source 1..708: mol_type = other DNA
ttagataatggcggacccgattgtcaacgagaattcgagaatgaggttgatttgatgagtagaattaggcatccaaatgtggtttctttattgggttattgcattcatggagaaaccaggcttcttgtctatgaaatgatgcaaaacgggacgttggaatcgctattgcatggaccatcacatggatcctcactaacttggcacattcgtatgaagatcgccctcgacacagcaagaggcctcgagtatctgcatgaacactgcgacccctctgtgatccaccgtgacctgaagccttctaacattcttttggattccaactacaattccaagctctcagactttggtcttgcagtcactgttggaagccagaatcaaaccaacattaagattctagggacactgggttaccttgcaccagagtacgttttgaatggcaaattgacagagaaaagtgatgtgtttgcttttggagttgtcctgttggagcttctcatgggcaagaaaccagtggagaagatggcatcccctccatgccaatccattgtcacatgggcgatgcctcatcttactgacagaattaagcttccaaatatcattgatcctgttattagaaacaccatggatctgaaacacttgtaccaggttgcagctgttgctgttctctgcgtacaaccagagccccagttatcgtcctctgataactga

SEQ ID NO: 101 - protein, 235 aa, Nuphar advena
LDNGGPDCQREFENEVDLMSRIRHPNVVSLLGYCIHGETRLLVYEMMQNGTLESLLHGPSHGSSLTWHIRMKIALDTARGLEYLHEHCDPSVIHRDLKPSNILLDSNYNSKLSDFGLAVTVGSQNQTNIKILGTLGYLAPEYVLNGKLTEKSDVFAFGVVLLELLMGKKPVEKMASPPCQSIVTWAMPHLTDRIKLPNIIDPVIRNTMDLKHLYQVAAVAVLCVQPEPQLSSSDN

SEQ ID NO: 102 - DNA, 30 bp, synthetic construct
  misc_feature 1..30: note = Synthetic Primer
  source 1..30: mol_type = other DNA
tcggctcggcccagaacaagatcgcaagac

SEQ ID NO: 103 - DNA, 34 bp, synthetic construct
  misc_feature 1..34: note = Synthetic Primer
  source 1..34: mol_type = other DNA
ctacattctctcctcgtattattcctcgttgact

SEQ ID NO: 104 - DNA, 32 bp, synthetic construct
  misc_feature 1..32: note = Synthetic Primer
  source 1..32: mol_type = other DNA
actttcagatgagtggatcataaccctataca

SEQ ID NO: 105 - DNA, 31 bp, synthetic construct
  misc_feature 1..31: note = Synthetic Primer
  source 1..31: mol_type = other DNA
agatacaatggatctcaaacacttataccag

SEQ ID NO: 106 - DNA, 42 bp, synthetic construct
  misc_feature 1..42: note = Synthetic Primer
  source 1..42: mol_type = other DNA
aaaggatccatgggaagtggtgaagaagatagatttgatgct

SEQ ID NO: 107 - DNA, 40 bp, synthetic construct
  misc_feature 1..40: note = Synthetic Primer
  source 1..40: mol_type = other DNA
tttctgcagtctgtgaatcatcttgttaaccggagagtcc

SEQ ID NO: 108 - DNA, 30 bp, synthetic construct
  misc_feature 1..30: note = Synthetic Primer
  source 1..30: mol_type = other DNA
tctgagttttaatcgagccaagtcgtctca

SEQ ID NO: 109 - DNA, 53 bp, synthetic construct
  misc_feature 1..53: note = Synthetic Primer
  source 1..53: mol_type = other DNA
tatcccgggaaaatgagagagcttcttcttcttcttcttcttcattttcagtc

SEQ ID NO: 110 - DNA, 38 bp, synthetic construct
  misc_feature 1..38: note = Synthetic Primer
  source 1..38: mol_type = other DNA
tttggatcctgtgaatcatcttgttaaccggagagtcc

SEQ ID NO: 111 - DNA, 41 bp, synthetic construct
  misc_feature 1..41: note = Synthetic Primer
  source 1..41: mol_type = other DNA
atacccgggtctgtgtcaggaatccaaatgggaagtggtga

SEQ ID NO: 112 - DNA, 41 bp, synthetic construct
  misc_feature 1..41: note = Synthetic Primer
  source 1..41: mol_type = other DNA
aaaggatcctctgtgtcaggaatccaaatgggaagtggtga

SEQ ID NO: 113 - DNA, 39 bp, synthetic construct
  misc_feature 1..39: note = Synthetic Primer
  source 1..39: mol_type = other DNA
aaatctagactgtgaatcatcttgttaaccggagagtcc

SEQ ID NO: 114 - DNA, 35 bp, synthetic construct
  misc_feature 1..35: note = Synthetic Primer
  source 1..35: mol_type = other DNA
atagagctcgcaagaaccaatctccaaaatccatc

SEQ ID NO: 115 - DNA, 37 bp, synthetic construct
  misc_feature 1..37: note = Synthetic Primer
  source 1..37: mol_type = other DNA
atagagctcgagggtcttgatatcgaaaaattgcacg

SEQ ID NO: 116 - DNA, 37 bp, synthetic construct
  misc_feature 1..37: note = Synthetic Primer
  source 1..37: mol_type = other DNA
ataggatcctcgcaagaaccaatctccaaaatccatc

SEQ ID NO: 117 - DNA, 40 bp, synthetic construct
  misc_feature 1..40: note = Synthetic Primer
  source 1..40: mol_type = other DNA
atatctagactcgagggtcttgatatcgaaaaattgcacg

SEQ ID NO: 118 - DNA, 52 bp, synthetic construct
  misc_feature 1..52: note = Synthetic Primer
  source 1..52: mol_type = other DNA
atatctagaaaatgagagagcttcttcttcttcttcttcttcattttcagtc

SEQ ID NO: 119 - DNA, 47 bp, synthetic construct
  misc_feature 1..47: note = Synthetic Primer
  source 1..47: mol_type = other DNA
ataggatcctgttaaaagcgatttataatttacaccgttttggtgta

SEQ ID NO: 120 - DNA, 41 bp, synthetic construct
  misc_feature 1..41: note = Synthetic Primer
  source 1..41: mol_type = other DNA
atacccgggaaaagtttttgatgaaattcaatctaaagact

SEQ ID NO: 121 - DNA, 1309 bp, Arabidopsis thaliana
  source 1..1309: mol_type = other DNA
aaaatgagagagcttcttcttcttcttcttcttcattttcagtctctaattcttttgatgatcttcatcactgtctctgcttcttctgcttcaaatccttctttagctcctgtttactcttccatggctacattctctcctcgaatccaaatgggaagtggtgaagaagatagatttgatgctcataagaaacttctgattggtctcataatcagtttctcttctcttggccttataatcttgttctgttttggcttttgggtttatcgcaagaaccaatctccaaaatccatcaacaactcagattctgagagtgggaattcattttccttgttaatgagacgacttggctcgattaaaactcagagaagaacttctatccaaaagggttacgtgcaatttttcgatatcaagaccctcgagaaagcgacaggcggttttaaagaaagtagtgtaatcggacaaggcggtttcggatgcgtttacaagggttgtttggacaataacgttaaagcagcggtcaagaagatcgagaacgttagccaagaagcaaaacgagaatttcagaatgaagttgacttgttgagcaagatccatcactcgaacgttatatcattgttgggctctgcaagcgaaatcaactcgagtttcatcgtttatgagcttatggagaaaggatcattagatgaacagttacatgggccttctcgtggatcagctctaacatggcacatgcgtatgaagattgctcttgatacagctagaggactagagtatctccatgagcattgtcgtccaccagttatccacagagatttgaaatcttcgaatattcttcttgattcttccttcaacgccaagatttcagatttcggttttgctgtatcgctggatgaacatggcaagaacaacattaaactctctgggacacttggttatgttgccccggaatacctccttgacggaaaactgacggataagagtgatgtttatgcatttggggtagttctgcttgaactcttgttgggtagacgaccagttgaaaaattaactccagctcaatgccaatctcttgtaacttgggcaatgccacaacttaccgatagatccaagcttccaaacattgtggatgccgttataaaagatacaatggatctcaaacacttataccaggtagcagccatggctgtgttgtgcgtgcagccagaaccaagttaccggccgttgataaccgatgttcttcactcacttgttccactggttccggtagagctaggagggactctccggttaacaagatgattcacag

SEQ ID NO: 122 - DNA, 25 bp, synthetic construct
  misc_feature 1..25: note = Synthetic Primer
  source 1..25: mol_type = other DNA
tcggacaaggcggtttcggatgcgt

SEQ ID NO: 123 - DNA, 32 bp, synthetic construct
  misc_feature 1..32: note = Synthetic Primer
  source 1..32: mol_type = other DNA
tagtcctctagctgtatcaagagcaatcttca

SEQ ID NO: 124 - DNA, 32 bp, synthetic construct
  misc_feature 1..32: note = Synthetic Primer
  source 1..32: mol_type = other DNA
tatcattgttgggctctgcaagtgaaatcaac

SEQ ID NO: 125 - DNA, 32 bp, synthetic construct
  misc_feature 1..32: note = Synthetic Primer
  source 1..32: mol_type = other DNA
tggagaaaggatccttagatgatcagttacat

SEQ ID NO: 126 - DNA, 31 bp, synthetic construct
  misc_feature 1..31: note = Synthetic Primer
  source 1..31: mol_type = other DNA
tccatgtaactgatcatctaaggatcctttc

SEQ ID NO: 127 - DNA, 37 bp, synthetic construct
  misc_feature 1..37: note = Synthetic Primer
  source 1..37: mol_type = other DNA
ataaacgacgaaactcgagttgatttcacttgcagag

SEQ ID NO: 128 - DNA, 28 bp, synthetic construct
  misc_feature 1..28: note = Synthetic Primer
  source 1..28: mol_type = other DNA
aaaatgaagaaactggttcatcttcagt

SEQ ID NO: 129 - DNA, 27 bp, synthetic construct
  misc_feature 1..27: note = Synthetic Primer
  source 1..27: mol_type = other DNA
tagacttctattctcacattcttacac

SEQ ID NO: 130 - DNA, 27 bp, synthetic construct
  misc_feature 1..27: note = Synthetic Primer
  source 1..27: mol_type = other DNA
tccaatgatccattatgcatcagctca

SEQ ID NO: 131 - DNA, 29 bp, synthetic construct
  misc_feature 1..29: note = Synthetic Primer
  source 1..29: mol_type = other DNA
tcgttctcaaattctctctcagcatgttg

SEQ ID NO: 132 - DNA, 29 bp, synthetic construct
  misc_feature 1..29: note = Synthetic Primer
  source 1..29: mol_type = other DNA
tccggatatgccaggtcagcgctgatcca

SEQ ID NO: 133 - DNA, 28 bp, synthetic construct
  misc_feature 1..28: note = Synthetic Primer
  source 1..28: mol_type = other DNA
tccagggatcccttctccatgagctcat

SEQ ID NO: 134 - DNA, 41 bp, synthetic construct
  misc_feature 1..41: note = Synthetic Primer
  source 1..41: mol_type = other DNA
aaagagctctctgtgtcaggaatccaaatgggaagtggtga

SEQ ID NO: 135 - DNA, 47 bp, synthetic construct
  misc_feature 1..47: note = Synthetic Primer
  source 1..47: mol_type = other DNA
atagctagctgttaaaagcgatttataatttacaccgttttggtgta

SEQ ID NO: 136 - DNA, 43 bp, synthetic construct
  misc_feature 1..43: note = Synthetic Primer
  source 1..43: mol_type = other DNA
atagctagcagaaaagtttttgatgaaattcaatctaaagact

SEQ ID NO: 137 - DNA, 29 bp, synthetic construct
  misc_feature 1..29: note = Synthetic Primer
  source 1..29: mol_type = other DNA
tctgggtttatcatcataccaagtatcca

SEQ ID NO: 138 - DNA, 31 bp, synthetic construct
  misc_feature 1..31: note = Synthetic Primer
  source 1..31: mol_type = other DNA
attcagttccatcaagattgttggcatggac

SEQ ID NO: 139 - DNA, 31 bp, synthetic construct
  misc_feature 1..31: note = Synthetic Primer
  source 1..31: mol_type = other DNA
tggagggaggtggccctgagtgcgagaagga

SEQ ID NO: 140 - DNA, 27 bp, synthetic construct
  misc_feature 1..27: note = Synthetic Primer
  source 1..27: mol_type = other DNA
gctggatctgcttggcaggattcggca

SEQ ID NO: 141 - DNA, 31 bp, synthetic construct
  misc_feature 1..31: note = Synthetic Primer
  source 1..31: mol_type = other DNA
atatctagatgctaggttatagatccatgca

SEQ ID NO: 142 - DNA, 33 bp, synthetic construct
  misc_feature 1..33: note = Synthetic Primer
  source 1..33: mol_type = other DNA
ataggatccaccagaactatatatacgaaggca

SEQ ID NO: 143 - DNA, 40 bp, synthetic construct
  misc_feature 1..40: note = Synthetic Primer
  source 1..40: mol_type = other DNA
aggacgacttggctcgattaaaatcacaggtcgtgatatg

SEQ ID NO: 144 - DNA, 35 bp, synthetic construct
  misc_feature 1..35: note = Synthetic Primer
  source 1..35: mol_type = other DNA
taatcgagccaagtcgtcctacatatatattccta

SEQ ID NO: 145 - DNA, 35 bp, synthetic construct
  misc_feature 1..35: note = Synthetic Primer
  source 1..35: mol_type = other DNA
taatcgagccaagtcgtcctctcttttgtattcca

SEQ ID NO: 146 - DNA, 42 bp, synthetic construct
  misc_feature 1..42: note = Synthetic Primer
  source 1..42: mol_type = other DNA
aggacgacttggctcgattaaaatcaaagagaatcaatgatc

SEQ ID NO: 147 - DNA, 21 bp, synthetic construct
  misc_feature 1..21: note = Synthesized gene fragment
  source 1..21: mol_type = other DNA
gacgacttggctcgattaaaa

SEQ ID NO: 148 - DNA, 399 bp, Arabidopsis thaliana
  source 1..399: mol_type = other DNA
tgctaggttatagatccatgcaaatatggagtagatgtacaaacacacgctcggacgcatattacacatgttcatacacttaatactcgctgttttgaattgatgttttaggaatatatatgtagagagagcttccttgagtccattcacaggtcgtgatatgattcaattagcttccgactcattcatccaaataccgagtcgccaaaattcaaactagactcgttaaatgaatgaatgatgcggtagacaaattggatcattgattctctttgattggactgaagggagctccctctctcttttgtattccaattttcttgattaatctttcctgcacaaaaacatgcttgatccactaagtgacatatatgctgccttcgtatatatagttctggt

SEQ ID NO: 149 - DNA, 399 bp, synthetic construct
  misc_feature 1..399: note = Artificial microRNA construct
  source 1..399: mol_type = other DNA
tgctaggttatagatccatgcaaatatggagtagatgtacaaacacacgctcggacgcatattacacatgttcatacacttaatactcgctgttttgaattgatgttttaggaatatatatgtaggacgacttggctcgattaaaatcacaggtcgtgatatgattcaattagcttccgactcattcatccaaataccgagtcgccaaaattcaaactagactcgttaaatgaatgaatgatgcggtagacaaattggatcattgattctctttgattttaatcgagccaagtcgtcctctcttttgtattccaattttcttgattaatctttcctgcacaaaaacatgcttgatccactaagtgacatatatgctgccttcgtatatatagttctggt

SEQ ID NO: 150 - DNA, 1475 bp, Arabidopsis thaliana
  source 1..1475: mol_type = other DNA
cttagccaatggatgaggatgacacgataatgataatcaaagatcaacatggcacgctcaagaccgcctttagaagtcctctctaaattctttcttccgatctcctaaatatgttttgttttggtcaaataaattgataggtaatacttagtgattatactatttggtttttgttttatcattgactatttcacttttataaatcaaatacttatcaaaattgttctttccgtatgtattcatattttctaatattgtaaagatttgtttcacctaacatctgtacccatctttgatcattgacaaaatatatattagaatggccttagaacgtgttaggcatcttcctactattatcatattacctaatccccaattttattacattttttaatttctaaaagagcttgaatataatgtcatttcgaatatctctgttcatctttttttttttctgtgcgacttctgacccaaagccttcgacgattttttccaatctgaaaacttttgaataaggaacttagtcaatggtcaacaccttgctaattaaacaaagttccattgatacaataatgagatttttgtacattaacgctttcatatagtttttgcgattcaacagataatcttaaaattaaggagtcctattgataaagtcttgttcaaacgtacaaactcaatccacacaaaaccttcataaaatacgatataggaaataaagattgtttttgcgtgagaaaatactatatgaactcaaaagattttaaaacaatttgtattaatacataaacaattgttgtgatacacccgtgtaaaattttaagattgtttttttctgaaattcttcaaggaaacttatagcttaaaatctacacttcaaatactctgttttaaaggcattaaaaataactgcgtttcagaaaaatattgaaattttagctgatcttttgctacaaatttaaggaatcttggcacctgcagaatctataacatgttcattaagtaatgcaatagttatacaattatacattatttgcatcatacttatattatagtgatattaacaaacccatgttctcagcacacttttacgtagaaaaacataaaaacccaaataggaagaagccactcataaggataatgggtttatataattcacagcaaagaaagccatcgaactattcgattaattatccattctttttttttttagtttgaatgtataagaacaaagagttgttacgcatcatgacaatgtcttagaaaacaaaagaaatgaataaaaaagtaaaacgaaaaataaaaagtgaggatgaagttgttgaatgagttggcgaggcggcgactttttcatacattccatttacttaattcctaaagtccttctcacatctctttgttatataatgacaccataaccatttcttctcttcacaatctttacaagaatatctctcttctacagtaaacaaaaa

SEQ ID NO: 151 - DNA, 31 bp, synthetic construct
  misc_feature 1..31: note = Synthetic Primer
  source 1..31: mol_type = other DNA
acgtaagcttcttagccaatggatgaggatg

SEQ ID NO: 152 - DNA, 31 bp, synthetic construct
  misc_feature 1..31: note = Synthetic Primer
  source 1..31: mol_type = other DNA
acgttctagatttttgtttactgtagaagag

SEQ ID NO: 153 - DNA, 338 bp, Brassica napus
  source 1..338: mol_type = other DNA
tgctgcttcaaatccttctatagctcctgtttataccaccatgactactttctctccaggaattcaaatgggaagtggtgaagaacacagattagatgcacataagaaactcctgattggtcttataatcagttcctcttctcttggtatcgtaatcttgatttgctttggcttctggatgtactgtcgcaagaaagctcccaaacccatcaagattccggatgctgagagtgggacttcatcattttcaatgtttgtgaggcggctaagctcaatcaaaactcagagaacatctagcaatcagggttatgtgcagcgtttcgattccaagacgctag

SEQ ID NO: 154 - DNA, 38 bp, synthetic construct
  misc_feature 1..38: note = Synthetic Primer
  source 1..38: mol_type = other DNA
tatggatcctgctgcttcaaatccttctatagctcctg

SEQ ID NO: 155 - DNA, 37 bp, synthetic construct
  misc_feature 1..37: note = Synthetic Primer
  source 1..37: mol_type = other DNA
tattctagactagcgtcttggaatcgaaacgctgcac

SEQ ID NO: 156 - DNA, 38 bp, synthetic construct
  misc_feature 1..38: note = Synthetic Primer
  source 1..38: mol_type = other DNA
tatgagctctgctgcttcaaatccttctatagctcctg

SEQ ID NO: 157 - DNA, 37 bp, synthetic construct
  misc_feature 1..37: note = Synthetic Primer
  source 1..37: mol_type = other DNA
tatgagctcctagcgtcttggaatcgaaacgctgcac

SEQ ID NO: 158 - DNA, 24 bp, synthetic construct
  misc_feature 1..24: note = Synthetic Primer
  source 1..24: mol_type = other DNA
gcagatcgctcctcccgtcgtgat

SEQ ID NO: 159 - DNA, 28 bp, synthetic construct
  misc_feature 1..28: note = Synthetic Primer
  source 1..28: mol_type = other DNA
cgcctaggagcgacgggtactcgatcat

SEQ ID NO: 160 - DNA, 28 bp, synthetic construct
  misc_feature 1..28: note = Synthetic Primer
  source 1..28: mol_type = other DNA
cctagctaagcgacgggtactcgatcat

SEQ ID NO: 161 - DNA, 272 bp, Brachypodium distachyon
  source 1..272: mol_type = other DNA
gctcctcccgtcgtgatcacagtggtgaggcaccaccattaccaccgggagctggtcatctccgctgtcctcgcctgcgtcgccaccgccatgatcctcctctccacactctacgcctggacgatgtggcggcggtctcgccggaccccccacggcggcaagggccgcggccggagatcagggatcacactggtgccaatcctgagcaagttcaattcagtgaagatgagcaggaaggggggccttgtgacgatgatcgagtacccgtcgct

SEQ ID NO: 162 - DNA, 31 bp, synthetic construct
  misc_feature 1..31: note = Synthetic Primer
  source 1..31: mol_type = other DNA
cgggatcccggcataacaaactcgtgcatcc

SEQ ID NO: 163 - DNA, 30 bp, synthetic construct
  misc_feature 1..30: note = Synthetic Primer
  source 1..30: mol_type = other DNA
ccatcgatggcgccaaacacaatagctcaa

SEQ ID NO: 164 - DNA, 1174 bp, Brachypodium distachyon
  source 1..1174: mol_type = other DNA
gtaagtaatttcaagtttaagtttcataagcataacaaactcgtgcatccaatttgaaccattttactgtcctggcatcctctaaatatttccttgattatcagcttatcttcatcccattgaatcagaaaattaccaacccttgttttagctttaatcattgttatttgttgtctgaggggctacactgtttctttatattggtgaaggagttaccaggcaaaaattcccacctcctgatattagcagagacccccttttttgtgcctgtatgcatactaacaaataatacagatggaaatatgtatatttgttatatcatggattgatgctttatgtttagcaagtccatgcaatggtagtcaaaagatgtaaacttttgaatgatatattggggctttagattagccatttttaccctcacttgaaaatgacaattttgcccttccgatctactttctcttgtcacctcaggcaggctcttgaaagttcttatccctgaattccgtggaagtttattattctaatgttatagtttacttaaagtgtcgcataatctactagagcctaatggaagtactgatggactttgttttgctacaatcactgcttgcaagaatgactactttggggcatttctaatatattattgatatttctatgatgtattgttgtccatgtacttcagtccttacagcgactagtcctatttctgcattgataaattgttcactgtcagaccatcttgagtggcaagaatgagtataacatgtcttgtttttctgtgatttcaaggtaagcgcacatgcgcacagtgtacaccgtcaccacatgtgagtacaccccctagtacacatgtaaaaaaagcacagtccagttattaaatggaccattggcattgattgtcgtgtttataggagtaaagatacatgtaaacactaattcattgggagatataaatttatactaccattgaatgtgacataggctctaaggtttttagttcagcatttcgaaagagctttgtttggttggcttgggatggaatcaggtgacaacatttttgggttgcagcaaatttaatattgattgaggaggcatacaacgaaatcattgagctattgtgtttggcgttacatctatggaatttcttctaatctgattattgtttgta

SEQ ID NO: 165 - DNA, 22 bp, synthetic construct
  misc_feature 1..22: note = Synthetic Primer
  source 1..22: mol_type = other DNA
gatccgctcctcccgtcgtgat

SEQ ID NO: 166 - DNA, 30 bp, synthetic construct
  misc_feature 1..30: note = Synthetic Primer
  source 1..30: mol_type = other DNA
aacgcgatcgcttgcatgcctgcagtagac

SEQ ID NO: 167 - DNA, 28 bp, synthetic construct
  misc_feature 1..28: note = Synthetic Primer
  source 1..28: mol_type = other DNA
gacttaattaagaattcgagctcgggta

SEQ ID NO: 168 - DNA, 251 bp, Panicum virgatum
  source 1..251: mol_type = other DNA
tcgtagtgcaccaccatttccaccgcgagctggtcatcgccgccgtcctcgcctgcatcgccaccgtcacgatcttcctttccacgctctacgcttggacactatggcggcgatctcgccggagcaccggcggcaaggtcaccaggagctcagacgcagcgaaggggatcaagctggtgccgatcttgagcaggttcaactcggtgaagatgagcaggaagaggctggttgggatgttcgagtacccgtcg

SEQ ID NO: 169 - DNA, 27 bp, synthetic construct
  misc_feature 1..27: note = Synthetic Primer
  source 1..27: mol_type = other DNA
gcagatctcgtagtgcaccaccatttc

SEQ ID NO: 170 - DNA, 27 bp, synthetic construct
  misc_feature 1..27: note = Synthetic Primer
  source 1..27: mol_type = other DNA
cgcctaggcgacgggtactcgaacatc

SEQ ID NO: 171 - DNA, 27 bp, synthetic construct
  misc_feature 1..27: note = Synthetic Primer
  source 1..27: mol_type = other DNA
cctagctacgacgggtactcgaacatc

SEQ ID NO: 172 - DNA, 25 bp, synthetic construct
  misc_feature 1..25: note = Synthetic Primer
  source 1..25: mol_type = other DNA
gatcctcgtagtgcaccaccatttc

SEQ ID NO: 173 - DNA, 21 bp, synthetic construct
  misc_feature 1..21: note = Synthetic Primer
  source 1..21: mol_type = other DNA
ctcgtagtgcaccaccatttc

SEQ ID NO: 174 - DNA, 261 bp, Sorghum bicolor
  source 1..261: mol_type = other DNA
aatgggaccgcctccgttgctccggcggtgccggcgccgcctcccgtcgtgatcatcgtggagcggcgccatcatttccaccgcgagctagtcatcgcctccgttctcgcctccatcgccatcgtcgcgattatcctctccacgctctatgcgtggatcctgtggcggcggtctcgccggctgcccagcggcaagggcgccaggagcgcagacaccgcgaggggaatcatgctggtgccgatcctgagcaagttccactca

SEQ ID NO: 175 - DNA, 26 bp, synthetic construct
  misc_feature 1..26: note = Synthetic Primer
  source 1..26: mol_type = other DNA
gcagatcaatgggaccgcctccgttg

SEQ ID NO: 176 - DNA, 28 bp, synthetic construct
  misc_feature 1..28: note = Synthetic Primer
  source 1..28: mol_type = other DNA
cgcctaggtgagtggaacttgctcagga

SEQ ID NO: 177 - DNA, 28 bp, synthetic construct
  misc_feature 1..28: note = Synthetic Primer
  source 1..28: mol_type = other DNA
cctagctatgagtggaacttgctcagga

SEQ ID NO: 178 - DNA, 273 bp, Sorghum bicolor
  source 1..273: mol_type = other DNA
gtaagtattcttgcaacacattactattttcaataaccacaagtttaaaagcttgagtccatttcgcaaaccagttgttcataaccaaattcttaggtaattaggtccaattgagaaaatctgatcattgaacactagcaggaaataactcagacatagtttctgcatactataatgatgcttaatatatttgttctcttttgagattgtattgcatagacatttctgtgtaaaataatgttttacatcatgtatatatatcactttttatag

SEQ ID NO: 179 - DNA, 30 bp, synthetic construct
  misc_feature 1..30: note = Synthetic Primer
  source 1..30: mol_type = other DNA
cgggatccttcttgcaacacattactattt

SEQ ID NO: 180 - DNA, 34 bp, synthetic construct
  misc_feature 1..34: note = Synthetic Primer
  source 1..34: mol_type = other DNA
ccatcgatgaaatgtctatgcaatacaatctcaa

SEQ ID NO: 181 - DNA, 24 bp, synthetic construct
  misc_feature 1..24: note = Synthetic Primer
  source 1..24: mol_type = other DNA
gatccaatgggaccgcctccgttg

SEQ ID NO: 182 - DNA, 21 bp, synthetic construct
  misc_feature 1..21: note = Synthetic Primer
  source 1..21: mol_type = other DNA
caatgggaccgcctccgttga

SEQ ID NO: 183 - DNA, 1000 bp, Sorghum bicolor
  source 1..1000: mol_type = other DNA
ggccccggccgcgcgcgtctccgtgtcctccgcgactgtgcacgtttcgtcgggagcggcgtgcccacgcccaccccccgtccaccagccagcaaccgacggcactggtgacacgcggctggtccgctcggtccgccccgcggctccagatcacggcaagcgcgcccgccgcccgctgctgcgctgcgctgcacgtcccgccctgacgccacgccacgccaagcgcgacacgacacgacacgacacgacccgacccccgccaacgaaacgccgaaacgcggcaacgcgtgacgggcgcgcatggtcgatgctctacccgcgcgtccgccccacgccaatctcccggcgggtccctcgtgggacggggaacgcgatgcggctgcaggctgcgaccgcgaccgcgaccgcgaccgcgcccacgtgaaggcaggcaggcagccccggagcgggcgcggcggtgggccaacgacgcgttgccgtcgcgaatcttcttctggccacggccaagggccaatcgcccgctccgctccgctccgcactccgcctccgctagggaatatggaacccgatcccacggccctctgggtctggtcgacgggtcctctcgccgtggcagctgcttcccggaccggaggatcgctgagcgcggacgccactgccattgccgtccgactatagttgttaattaccataaaataatttgttaacgataaaacccgtgtcaggcaccgtcgtctggacgctgctatgggataaccattcgcgtacgtcggttgtatgggtgggatcctctgcggcacgccattctggtgctgctagtggaatagacaaaaaaagggccgacggtgtttgctcgtggcaggccacacagagtgacaaccagagtggttgccgcaaaaacaaccaatcacacaaaaagtgttgtaccggtggaggacagccattaatcagcaggccggcttcgcggccaaaagaaacggagaagaggaaaaaggggggc

SEQ ID NO: 184 - DNA, 28 bp, synthetic construct
  misc_feature 1..28: note = Synthetic Primer
  source 1..28: mol_type = other DNA
tcccaagcttgcgcgtctccgtgtcctc

SEQ ID NO: 185 - DNA, 30 bp, synthetic construct
  misc_feature 1..30: note = Synthetic Primer
  source 1..30: mol_type = other DNA
agtaaagcttccccctttttcctcttctcc

SEQ ID NO: 186 - DNA, 1000 bp, Sorghum bicolor
  source 1..1000: mol_type = other DNA
taatggtcgagtgaggcccgtatagatgtagttaaatagctaaaatttttggagaaataagcatttttttggaagaatatatttaaacatgggcttgtaaaacttggctgtaaagatttggaatttaggatcttggagccccaaaactgtataaacttgcttagggacccgtgtcttgtgtgttgcagaccaaaaaatttagaaagcatctaaacacctatttgaatgtaaagtttacagccaaaagttttaggatgtaaagatttgggatctaaaagtagtcattaggaaataacacgttagagagagagagtagatcttcttattggtttctcatgcactaatcgaaccaatcactggaccacttgaaccaaactttatcacattgaactttgtcagttcagttcgaacgcaggactggagctgcccttaaggccaattgctcaagattcattcaacaattgaaacatctcccatgattaaatcagtataaggttgctatggtcttgcttgacaaagttttttttttgagggaatttcaactaaatttttgagtgaaactatcaaatactgattttaaaaattttttataaaaggaagcgcagagataaaaggccatctatgctacaaaagtacccaaaaatgtaatcctaaagtatgaattgcattttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcct

SEQ ID NO: 187 - DNA, 26 bp, synthetic construct
  misc_feature 1..26: note = Synthetic Primer
  source 1..26: mol_type = other DNA
aactgcagggtcgagtgaggcccgta

SEQ ID NO: 188 - DNA, 28 bp, synthetic construct
  misc_feature 1..28: note = Synthetic Primer
  source 1..28: mol_type = other DNA
ttctgcagggaacggaacggaataagaa

SEQ ID NO: 189 - DNA, 239 bp, Sorghum bicolor
  source 1..239: mol_type = other DNA
gccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcct

SEQ ID NO: 190 - DNA, 27 bp, synthetic construct
  misc_feature 1..27: note = Synthetic Primer
  source 1..27: mol_type = other DNA
taggtaccgccgtgggtcgtttaagct

SEQ ID NO: 191 - DNA, 27 bp, synthetic construct
  misc_feature 1..27: note = Synthetic Primer
  source 1..27: mol_type = other DNA
aaggtaccaggagacgaacaaaaacaa

SEQ ID NO: 192 - DNA, 34 bp, synthetic construct
  misc_feature 1..34: note = Synthetic Primer
  source 1..34: mol_type = other DNA
aacgcgatcgtaatggtcgagtgaggcccgtata

SEQ ID NO: 193 - DNA, 1302 bp, Brassica napus
  source 1..1302: mol_type = other DNA
atgaagaaactggttcatcttcagtttctgtttcttgtcaagatctttgctactcaattcctcactccttcttcatcatcttttgctgcttcaaatccttctatagctcctgtttataccaccatgactactttctctccaggaattcaaatgggaagtggtgaagaacacagattagatgcacataagaaactcctgattggtcttataatcagttcctcttctcttggtatcgtaatcttgatttgctttggcttctggatgtactgtcgcaagaaagctcccaaacccatcaagattccggatgctgagagtgggacttcatcattttcaatgtttgtgaggcggctaagctcaatcaaaactcagagaacatctagcaatcagggttatgtgcagcgtttcgattccaagacgctagagaaagcgacaggcggtttcaaagacagtaatgtaatcggacagggcggtttcggatgcgtttacaaggcttctttggacagcaacactaaagcagcggttaaaaagatcgaaaacgttagccaagaagcaaaacgagaatttcagaatgaagttgagctgttgagcaagatccagcactccaatattatatcattgttgggctctgcaagtgaaatcaactcgagtttcgtcgtttatgagttgatggagaaaggatccttagatgatcagttacatggaccttcgtgtggatccgctctaacatggcatatgcgtatgaagattgctctagatacagctagaggattagagtatctccatgaacattgtcgtccaccagttatccacagggacctgaaatcgtctaatatacttcttgattcttccttcaatgccaagatttcagattttggtctggctgtatcggttggagtgcatgggagtaacaacattaaactctctgggacacttggttatgttgccccggaatatctcctagacggaaagttgacggataagagtgatgtctatgcatttggggtggttcttcttgaacttttgttgggtagaaggccggttgagaaattgagtccatctcagtgtcaatctcttgtgacttgggcaatgccacaacttaccgatagatcgaaactcccaaacatcgtggatccggttataaaagatacaatggatcttaagcacttataccaggtagcagccatggctgtgttgtgcgttcagccagaaccgagttaccggccgctgataaccgatgttcttcactcacttgttccattggttccggtcgaactaggagggactctccggttaacccgatga

SEQ ID NO: 194 - protein, 433 aa, Brassica napus
MKKLVHLQFLFLVKIFATQFLTPSSSSFAASNPSIAPVYTTMTTFSPGIQMGSGEEHRLDAHKKLLIGLIISSSSLGIVILICFGFWMYCRKKAPKPIKIPDAESGTSSFSMFVRRLSSIKTQRTSSNQGYVQRFDSKTLEKATGGFKDSNVIGQGGFGCVYKASLDSNTKAAVKKIENVSQEAKREFQNEVELLSKIQHSNIISLLGSASEINSSFVVYELMEKGSLDDQLHGPSCGSALTWHMRMKIALDTARGLEYLHEHCRPPVIHRDLKSSNILLDSSFNAKISDFGLAVSVGVHGSNNIKLSGTLGYVAPEYLLDGKLTDKSDVYAFGVVLLELLLGRRPVEKLSPSQCQSLVTWAMPQLTDRSKLPNIVDPVIKDTMDLKHLYQVAAMAVLCVQPEPSYRPLITDVLHSLVPLVPVELGGTLRLTR

SEQ ID NO: 195 - DNA, 31 bp, synthetic construct
  misc_feature 1..31: note = Synthetic Primer
  source 1..31: mol_type = other DNA
aatccagctcattctggaattccttctcgca

SEQ ID NO: 196 - DNA, 32 bp, synthetic construct
  misc_feature 1..32: note = Synthetic Primer
  source 1..32: mol_type = other DNA
tgaacttgctcaggattggcaccagtgtgatc

SEQ ID NO: 197 - protein, 461 aa, Brachypodium distachyon
MEIPAAPPPPLPVLCSYVVFLLLLSSCSLARGRIAVSSPGPSPVAAAVTANETASSSSSPVFPAAPPVVITVVRHHHYHRELVISAVLACVATAMILLSTLYAWTMWRRSRRTPHGGKGRGRRSGITLVPILSKFNSVKMSRKGGLVTMIEYPSLEAATGKFGESNVLGVGGFGCVYKAAFDGGATAAVKRLEGGGPDCEKEFENELDLLGRIRHPNIVSLLGFCVHGGNHYIVYELMEKGSLETQLHGSSHGSALSWHVRMKIALDTARGLEYLHEHCNPPVIHRDLKPSNILLDSDFNAKIADFGLAVTGGNLNKGNLKLSGTLGYVAPEYLLDGKLTEKSDVYAFGVVLLELLMGRKPVEKMSPSQCQSIVSWAMPQLTDRSKLPNIIDLVIKDTMDPKHLYQVAAVAVLCVQPEPSYRPLITDVLHSLVPLVPAELGGTLRVAEPPSPSPDQRHYPC

SEQ ID NO: 198 - DNA, 53 bp, synthetic construct
  misc_feature 1..53: note = Synthetic Primer
  source 1..53: mol_type = other DNA
tataccggtaaaatgagagagcttcttcttcttcttcttcttcattttcagtc

SEQ ID NO: 199 - DNA, 39 bp, synthetic construct
  misc_feature 1..39: note = Synthetic Primer
  source 1..39: mol_type = other DNA
atataccggtcttgttaaccggagagtccctcctagctc

SEQ ID NO: 200 - DNA, 18 bp, synthetic construct
  misc_feature 1..18: note = Synthetic Primer
  source 1..18: mol_type = other DNA
cgctcctcccgtcgtgat